(19) United States
(12) Patent Application Publication
(10) Pub. No.: US 2025/0152574 A1
(43) Pub. Date: May 15, 2025

(54) EMOPAMIL-BINDING PROTEIN INHIBITORS AND USES THEREOF

(71) Applicant: BIOGEN MA INC., Cambridge, MA (US)

(72) Inventors: Martin Himmelbauer, Cambridge, MA (US); Felix Gonzalez Lopez de Turiso, Cambridge, MA (US); John H. Jones, Framingham, MA (US); Edward Yin Shiang Lin, Ashland, MA (US); Robin Prince, Sharon, MA (US); Vatee Pattaropong, Bedford, MA (US); Zhili Xin, Lexington, MA (US); TeYu Chen, Charlestown, MA (US); Nupur Bansal, Cambridge, MA (US)

(21) Appl. No.: 18/837,245

(22) PCT Filed: Feb. 13, 2023
(86) PCT No.: PCT/US2023/012879

§ 371 Date: Aug. 9, 2024

Related U.S. Application Data
(60) Provisional application No. 63/309,923, filed on Feb. 14, 2022.

(51) Int. Cl. A61K 31/4709; A61K 31/438; A61P 25/28; C07D 221/20; C07D 401/12; C07D 491/107

(52) U.S. Cl. CPC A61K 31/4709; A61K 31/438; A61P 25/28; C07D 221/20; C07D 401/12; C07D 491/107

(57) ABSTRACT

Provided are compounds of the Formula (I) or pharmaceutically acceptable salts thereof, which are useful for the inhibition of EBP and in the treatment of a variety of EBP mediated conditions or diseases, such as multiple sclerosis.

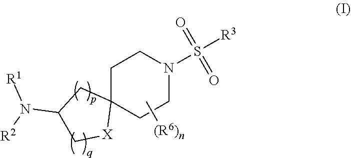

### RELATED APPLICATIONS

This application claims the benefit of the filing date, under 35 U.S.C. § 119(e), to U.S. Provisional Application No. 63/309,923, filed on Feb. 14, 2022. The entire contents of the foregoing application are expressly incorporated herein by reference.

### FIELD OF THE INVENTION

The present disclosure relates to inhibitors of Emopamil-Binding Protein (EBP), and pharmaceutically acceptable salts thereof, compositions of these compounds, processes for their preparation, their use in the treatment of diseases, their use in optional combination with a pharmaceutically acceptable carrier for the manufacture of pharmaceutical preparations, the use of the pharmaceutical preparations in the treatment of diseases, and methods of treating diseases comprising administering the EBP inhibitor to a warm-blooded animal, especially a human.

### BACKGROUND OF THE INVENTION

Emopamil-Binding Protein (EBP) is a Δ8-Δ7 sterol isomerase enzyme which isomerizes the double bond in sterol molecules, moving the double bond from the 8-9 position to the 7-8 position. Specifically, EBP converts either zymostenol to lathosterol, or zymosterol to dehydrolathosterol, during the biosynthesis of cholesterol (Silve et al., 1996, J Biol Chem. 271 (37), 22434-22440). It has been shown that an accumulation of 8-9 unsaturated sterols activates oligodendrocyte formation and remyelination (Hubler et al., 2019, Nature 560 (7718), 372-376).

Myelin is lipid-based molecule which forms protective layers (myelin sheathes) around nerve cell axons and insulates the axons. Demyelinating diseases, or myelin-related diseases, are a result of these myelin sheathes being damaged, degraded, or reduced in thickness. The loss of the myelin sheathes disrupts the electronic signals from the brain and can lead to nerve damage, vision loss, numbness, muscle weakness, cognitive decline, loss of motor functions, and other similar symptoms. In some myelin-related diseases, such as multiple sclerosis, a subject's immune system targets and breaks down their own myelin sheathes. The ability to repair and regenerate the myelin sheathes is key to treating these myelin-related diseases. Due to its function converting 8-9 sterols, inhibition of EBP is a potential target for activating remyelination, as its inhibition leads to an increase of these 8-9 sterol starting materials (Theodoropoulous et al, 2020, J Am. Chem. Soc., 142, (13), 6128-6138).

In addition to its role in remyeliniation, EBP has also been shown to be a key enzyme in certain colorectal cancers due to the reduction in essential lipids such as cholesterol (Theodoropoulous et al, 2020, J Am. Chem. Soc., 142, (13), 6128-6138).

Thus, there is a need for EBP inhibitors as potential therapeutic agents for treating diseases or disorders that are responsive to EBP inhibition.

### SUMMARY OF THE INVENTION

The present disclosure provides compounds that are EBP inhibitors. In a first aspect, the present disclosure relates to compounds having the Formula I:

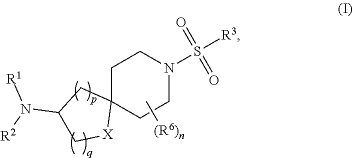

or a pharmaceutically acceptable salt thereof, wherein:
X is O, CH₂, or a bond, provided when X is a bond, p and q are 1;
p is 0 or 1, provided that when p is 0, q is 1;
q is 0 or 1, provided that when q is 0, p is 1;
R¹ and R² are each independently selected from H, C₁₋₆alkyl, C₃₋₈cycloalkyl, C₃₋₈cycloalkenyl, 6 to 10-membered bicyclic heterocycyl, and 4 to 6-membered monocyclic heterocyclyl, wherein the C₁₋₆alkyl, C₃₋₈cycloalkyl, C₃₋₈cycloalkenyl, 6 to 10-membered bicyclic heterocycyl, and 4 to 6-membered monocyclic heterocyclyl are each optionally substituted with one or more R⁴, provided at least one of R¹ and R² is not H;
or R¹ and R² together with the N atom from which they are attached form a 4 to 7-membered monocyclic heterocycle or 6 to 10-membered bicyclic heterocycle, each of which is optionally substituted with one or more R⁴;
each R⁴ is independently selected from OR^{4a}, halo, C₁₋₃alkyl, C₃₋₈cycloalkyl, and 4 to 6-membered monocyclic heterocyclyl;
R^{4a} is selected from H and C₁₋₃alkyl, wherein the C₁₋₃alkyl is optionally substituted with one or more halo;
R³ is phenyl, 5 or 6-membered monocyclic heteroaryl, 9 or 10-membered bicyclic heteroaryl, or 6 to 10 membered bicyclic heterocycle, wherein the phenyl, 5 or 6-membered monocyclic heteroaryl, 9 or 10-membered bicyclic heteroaryl, and 6 to 10 membered bicyclic heterocycle are each optionally substituted with one or more R⁵;
each R⁵ is independently selected from C₁₋₃alkyl, C₃₋₆cycloalkyl, OR^{5a}, cyano, halo, and 5 or 6-membered monocyclic heteroaryl, wherein the C₁₋₃alkyl, C₃₋₆cycloalkyl, and heteroaryl are optionally substituted with one or more R^{5b};
R^{5a} is selected from H, C₁₋₃alkyl, and C₃₋₆cycloalkyl, wherein the C₁₋₃alkyl is optionally substituted with one or more halo;
R^{5b} is selected from OR^{5a}, C₁₋₃alkyl, cyano, and halo;
R⁶, for each occurrence, is independently halo or C₁₋₃alkyl, or two R⁶ together to form a —C₁₋₃alkylene
n is 0, 1, or 2;
m is 0, 1, or 2.

In some embodiments, the compound of Formula (I) is not any one of the compounds listed in Table I below.

Another aspect of the disclosure relates to pharmaceutical compositions comprising compounds of Formula (I) or pharmaceutically acceptable salts thereof, and a pharmaceutical carrier.

In yet another aspect, the present disclosure provides a method of treating a disease or disorder that is responsive to inhibition of EBP in a subject comprising administering to said subject an effective amount of at least one compound described herein or a pharmaceutically acceptable salt thereof. In some embodiments, the present disclosure provides a method for treating multiple sclerosis. In some embodiments, the present disclosure provides a method for promoting myelination in a subject with a myelin-related disorder.

Another aspect of the present disclosure relates to the use of at least one compound described herein or a pharmaceutically acceptable salt thereof, for the manufacture of a medicament for the treatment of a disease or disorder responsive to inhibition of EBP. Also provided is a compound described herein or a pharmaceutically acceptable salt thereof for use in treating a disease or disorder responsive to inhibition of EBP.

### DETAILED DESCRIPTION OF THE INVENTION

The present disclosure provides compounds and pharmaceutical compositions thereof that may be useful in the treatment of diseases or disorders through mediation of EBP function/activity, such as multiple sclerosis or other myelin-related disorders. In some embodiments, the compounds of present disclosure are EBP inhibitors.

### Compounds and Compositions

In a first aspect, the present disclosure provides a compound of Formula (I):

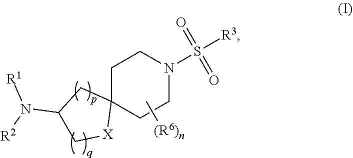

or a pharmaceutically acceptable salt thereof, wherein the variables in Formula (I) are as defined in the first embodiment above. In some embodiments, the compound of Formula (I) is not any one of the compounds listed in Table I below.

In a first embodiment, the present disclosure relates to compounds having the Formula

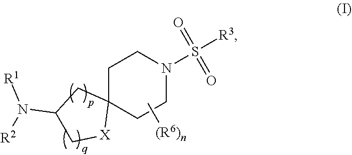

or a pharmaceutically acceptable salt thereof, wherein:
X is O, CH₂, or a bond, provided when X is a bond, p and q are 1;
p is 0 or 1, provided that when p is 0, q is 1;
q is 0 or 1, provided that when q is 0, p is 1;
R¹ and R² are each independently selected from H, C₁₋₆alkyl, C₃₋₈cycloalkyl, C₃₋₈cycloalkenyl, and 4 to 6-membered monocyclic heterocyclyl, wherein the C₁₋₆alkyl, C₃₋₈cycloalkyl, C₃₋₈cycloalkenyl, and 4 to 6-membered monocyclic heterocyclyl are each optionally substituted with one or more R⁴, provided at least one of R¹ and R² is not H; or R¹ and R² together with the N atom from which they are attached form a 4 to 7-membered monocyclic heterocycle or 6 to 10-membered bicyclic heterocycle, each of which is optionally substituted with one or more R⁴;
R⁴ is selected from OR^{4a}, halo, C₁₋₃alkyl, or C₃₋₈cycloalkyl;
R^{4a} is selected from H and C₁₋₃alkyl, wherein the C₁₋₃alkyl is optionally substituted with one or more halo;
R³ is phenyl, 5 or 6-membered monocyclic heteroaryl, 9 or 10-membered bicyclic heteroaryl, or 6 to 10 membered bicyclic heterocycle, wherein the phenyl, 5 or 6-membered monocyclic heteroaryl, 9 or 10-membered bicyclic heteroaryl, and 6 to 10 membered bicyclic heterocycle are each optionally substituted with one or more substituent R⁵;
R⁵ is selected from C₁₋₃alkyl, C₃₋₆cycloalkyl, OR^{5a}, cyano, halo, and 5 or 6-membered monocyclic heteroaryl, wherein the C₁₋₃alkyl, C₃₋₆cycloalkyl, and heteroaryl are optionally substituted with one or more R^{5b};
R^{5a} is selected from H, C₁₋₃alkyl, and C₃₋₆cycloalkyl, wherein the C₁₋₃alkyl is optionally substituted with one or more halo;
R^{5b} is selected from OR^{5a}, C₁₋₃alkyl, cyano, and halo;
R⁶, for each occurrence, is independently halo or C₁₋₃alkyl, or two R⁶ together to form a —C₁₋₃alkylene;
n is 0, 1, or 2;
m is 0, 1, or 2.

In some embodiments, the compound of Formula (I) described in the first aspect or the first embodiments is not any one of the compounds listed in Table I below.

In a second embodiment, for the compounds of Formula (I), or a pharmaceutically acceptable salt thereof, (i) when X is O, and R¹ and R² together with the N atom from which they are attached form unsubstituted morpholine, unsubstituted pyrrolidine, or unsubstituted N-methylpiperazine, then R³ is selected from a 9 or 10-membered bicyclic heteroaryl or a 6 to 10 membered bicyclic heterocycle each optionally substituted with one or more substituent R⁵ or a phenyl or a 5 or 6-membered monocyclic heteroaryl each substituted with at least two R⁵ groups, or one R⁵ group that is OR^{5a}; (ii) when X is O, and R¹ and R² are both C₁₋₆alkyl, then at least one of the C₁₋₆alkyl represented by R¹ and R² is substituted by one or more R⁴ selected from OR^{4a}, halo, and C₃₋₈cycloalkyl; (iii) when X is a bond, and R¹ and R² are both C₁₋₆alkyl, then at least one of the C₁₋₆alkyl represented by R¹ and R² is substituted by one or more R⁴; or (iv) when p is 0 and q is 1, then X is O. In an alternative second embodiment, for the compounds of Formula (I), or a pharmaceutically acceptable salt thereof, wherein (i) when X is O, and R¹ and R² together with the N atom from which they are attached form unsubstituted morpholine, unsubstituted pyrrolidine, or unsubstituted N-methylpiperazine, then R³ is selected from a 9 or 10-membered bicyclic heteroaryl or a 6 to 10 membered bicyclic heterocycle each optionally substituted with one or more substituent R⁵ or a phenyl or a 5 or 6-membered monocyclic heteroaryl each substituted with at least two R⁵ groups, or one R⁵ group that is OR^{5a}; (ii) when X is O, and R¹ and R² are both C₁₋₆alkyl, then at least one of the C₁₋₆alkyl represented by R¹ and R² is substituted by one or more R⁴ selected from OR^{4a}, halo, C₃₋₈cycloalkyl, and 4 to 6-membered monocyclic heterocyclyl; (iii) when X is a bond, and R¹ and R² are both C₁₋₆alkyl, then at least one of the C₁₋₆alkyl represented by R¹ and R² is substituted by one or more R⁴; or (iv) when p is 0 and q is 1, then X is O.

In a third embodiment, the compound of the present disclosure is represented by Formula (II):

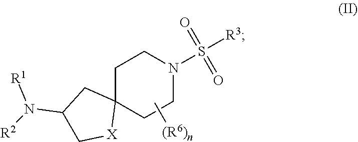

or a pharmaceutically acceptable salt thereof, wherein the variables in Formula (II) are as defined in the first aspect or the first or second embodiment above.

In a fourth embodiment, the compound of the present disclosure is represented by Formula (IIA) or (IIB):

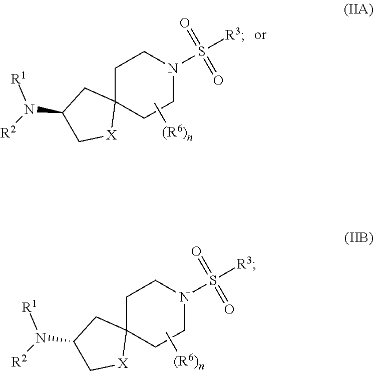

or a pharmaceutically acceptable salt thereof, wherein the variables in Formula (IIA) and (IIB) are as defined in the first aspect or the first or second embodiment above.

In a fifth embodiment, the compound of the present disclosure is represented by Formula (III) or (IV):

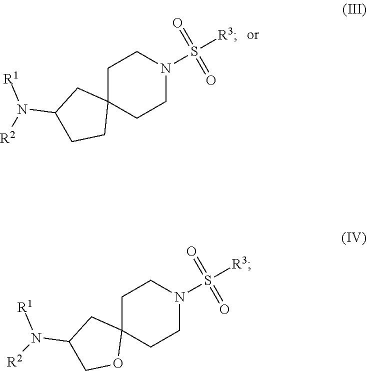

or a pharmaceutically acceptable salt thereof, wherein the variables in Formula (III) or (IV) are as defined in the first aspect or the first or second embodiment above.

In a sixth embodiment, the compound of the present disclosure is represented by Formula (IIIA), (IIIB), (IVA), or (IVB):

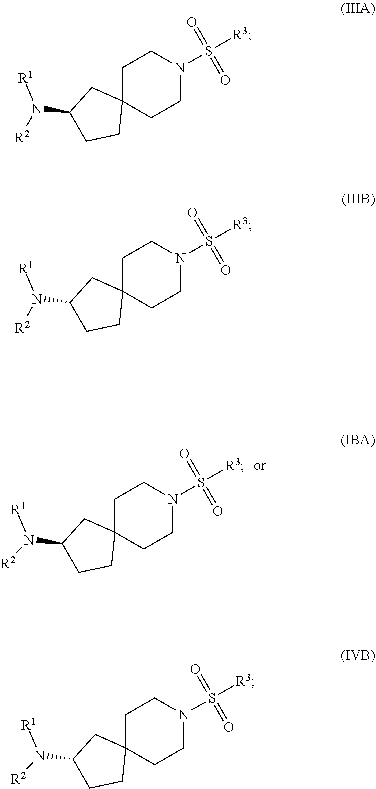

or a pharmaceutically acceptable salt thereof, wherein the variables in Formula (IIIA), (IIIB), (IVA), or (IVB) are as defined in the first aspect or the first or second embodiment above.

In a seventh embodiment, for the compounds of Formula (I), (II), (IIA), (IIB), (III), (IV), (IIIA), (IIIB), (IVA), or (IVB), or a pharmaceutically acceptable salt thereof, R³ is phenyl, 5 or 6-membered monocyclic heteroaryl, 9 to 10 membered bicyclic heteroaryl or 8 to 10 membered bicyclic heterocycle, wherein the phenyl, 5 or 6-membered monocyclic heteroaryl, 9 to 10 membered bicyclic heteroaryl and 8 to 10 membered bicyclic heterocycle are each optionally substituted with one to three R⁵; and the remaining variables are as described in the first aspect or the first or second embodiment.

In an eighth embodiment, for the compounds of Formula (I), (II), (IIA), (IIB), (III), (IV), (IIIA), (IIIB), (IVA), or (IVB), or a pharmaceutically acceptable salt thereof, R³ is pyridyl, phenyl, thiazolyl, pyrazolyl, pyrazinyl, imidazopyridinyl, quinolinyl, tetrahydropyranopyrazolyl, thiophenyl, benzothiophenyl, furanyl, indazolyl, indolizinyl, or benzofuranyl, each of which are each optionally substituted with one to three R⁵; and the remaining variables are as described in the first aspect or the first or second embodiment. In an alternative eighth embodiment, for the compounds of Formula (I), (II), (IIA), (IIB), (III), (IV), (IIIA), (IIIB), (IVA), or (IVB), or a pharmaceutically acceptable salt thereof, R³ is selected from the group consisting of pyridyl, pyrimidinyl, phenyl, thiazolyl, pyrazolyl, pyrazinyl, triazoyl, imidazopyridinyl, quinolinyl, tetrahydropyranopyrazolyl, thiophenyl, benzothiophenyl, furanyl, indazolyl, indolizinyl, pyrazolo[1,5-a]pyridinyl, 2,3-dihydrobenzo[b][1,4]dioxinyl, and benzofuranyl, each of which are each optionally substituted with one to three R⁵; and the remaining variables are as described in the first aspect or the first or second embodiment.

In a ninth embodiment, for the compounds of Formula (I), (II), (IIA), (IIB), (III), (IV), (IIIA), (IIIB), (IVA), or (IVB), or a pharmaceutically acceptable salt thereof, R³ is pyridyl, phenyl, pyrazoyl, thiophenyl, thiazolyl, quinolinyl, tetrahydropyranopyrazolyl, or benzofuranyl; each of which are each optionally substituted with one to three R⁵, and the remaining variables are as described in the first aspect or the first or second embodiment.

In a tenth embodiment, for the compounds of Formula (I), (II), (IIA), (IIB), (III), (IV), (IIIA), (IIIB), (IVA), or (IVB), or a pharmaceutically acceptable salt thereof, R³ is

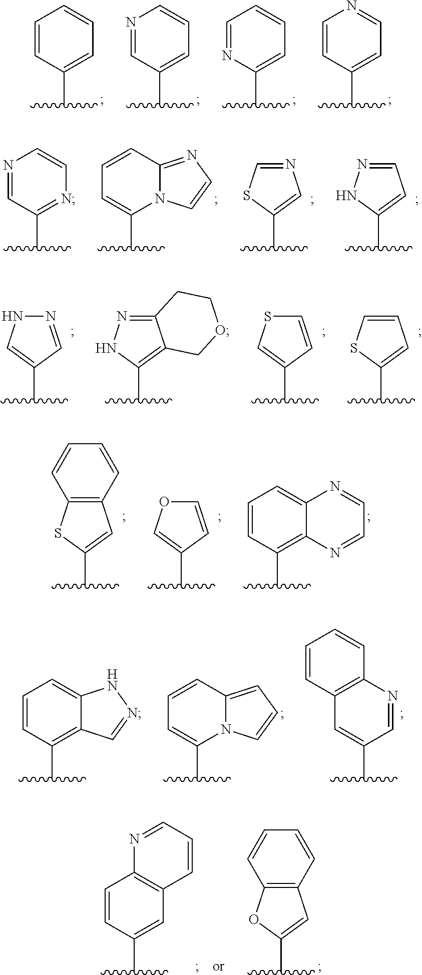

each of which is optionally substituted with one to three R⁵; and the remaining variables are as described in the first aspect or the first or second embodiment. In an alternative tenth embodiment, for the compounds of Formula (I), (II), (IIA), (IIB), (III), (IV), (IIIA), (IIIB), (IVA), or (IVB), or a pharmaceutically acceptable salt thereof, R³ is represented by the following formula:

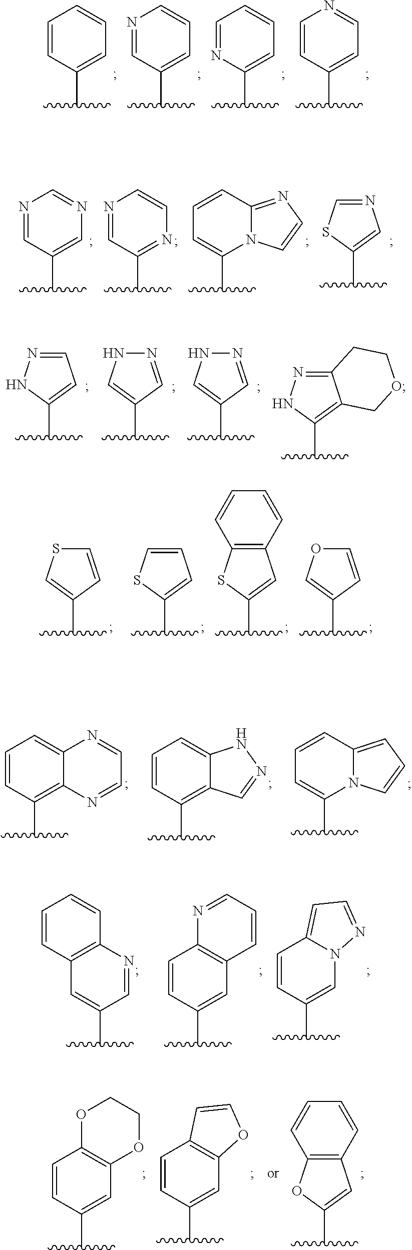

wherein each of the formula depicted above is optionally substituted with one to three R⁵; and the remaining variables are as described in the first aspect or the first or second embodiment.

In an eleventh embodiment, for the compounds of Formula (I), (II), (IIA), (IIB), (III), (IV), (IIIA), (IIIB), (IVA), or (IVB), or a pharmaceutically acceptable salt thereof, R³ is

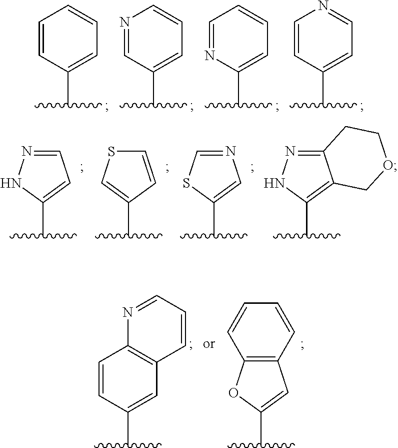

each of which is optionally substituted with one to three R⁵; and the remaining variables are as described in the first aspect or the first or second embodiment.

In a twelfth embodiment, for the compounds of Formula (I), (II), (IIA), (IIB), (III), (IV), (IIIA), (IIIB), (IVA), or (IVB), or a pharmaceutically acceptable salt thereof, R³ is

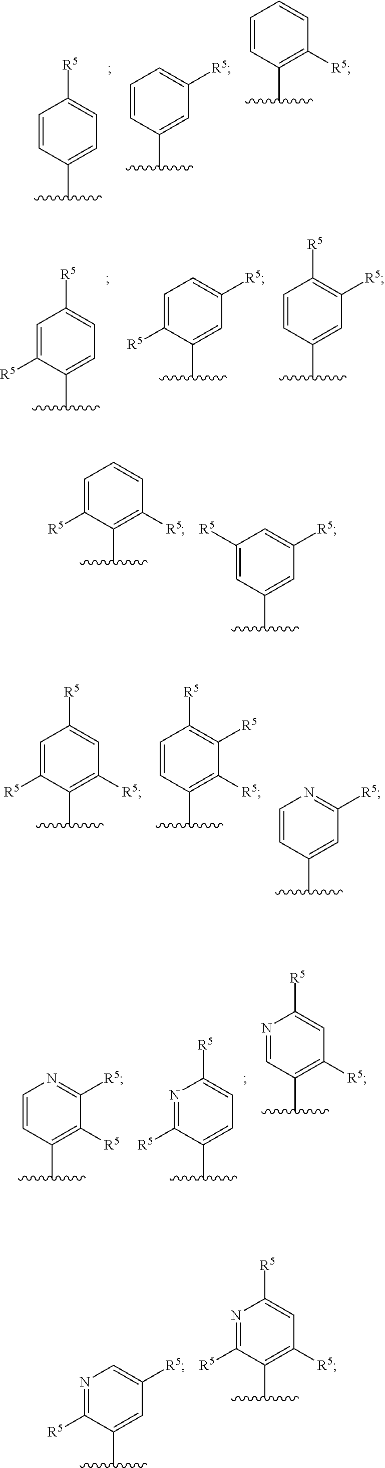

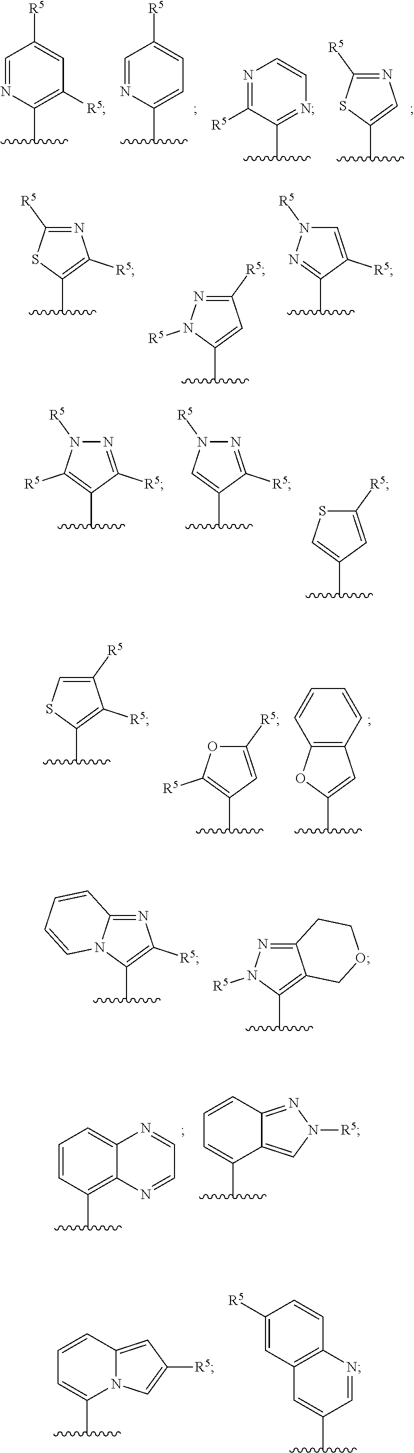

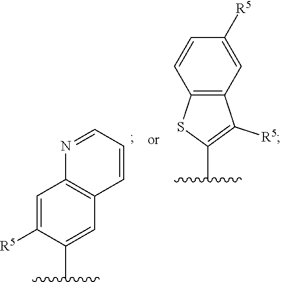

and the remaining variables are as described in the first aspect or the first or second embodiment. In an alternative twelfth embodiment, for the compounds of Formula (I), (II), (IIA), (IIB), (III), (IV), (IIIA), (IIIB), (IVA), or (IVB), or a pharmaceutically acceptable salt thereof, R³ is represented by the following formula:

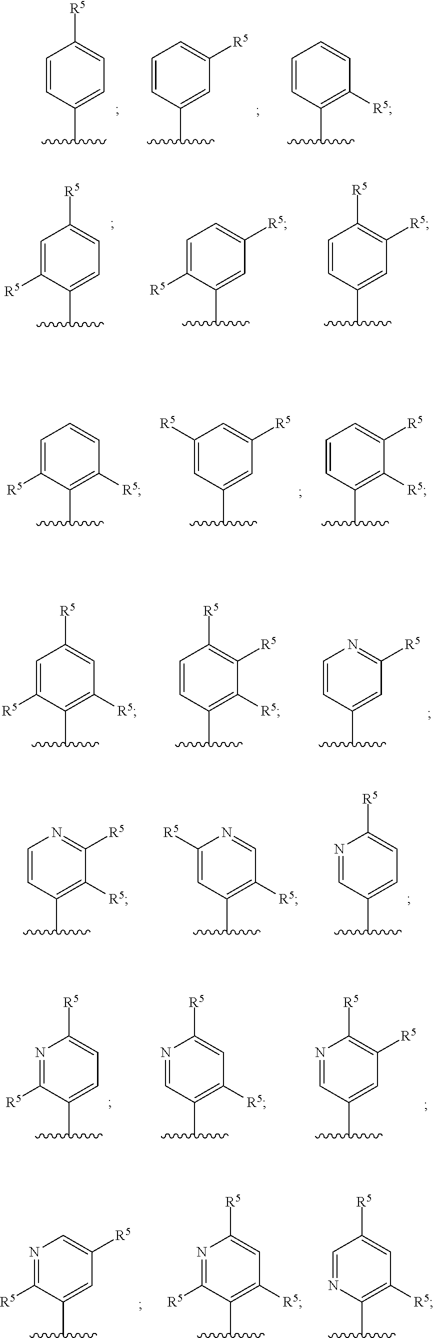

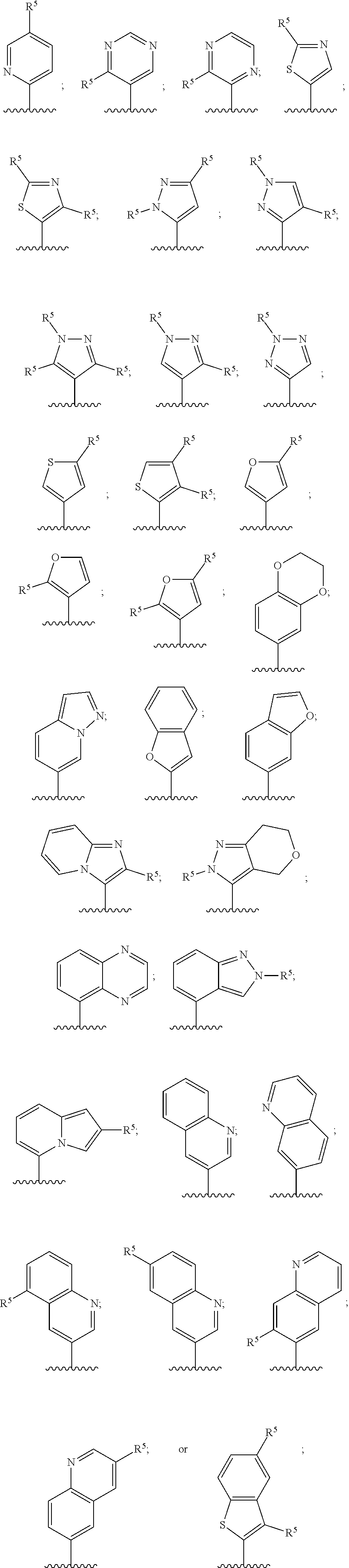

and the remaining variables are as described in the first aspect or the first or second embodiment

In a thirteenth embodiment, for the compounds of Formula (I), (II), (IIA), (IIB), (III), (IV), (IIIA), (IIIB), (IVA), or (IVB), or a pharmaceutically acceptable salt thereof, R³ is

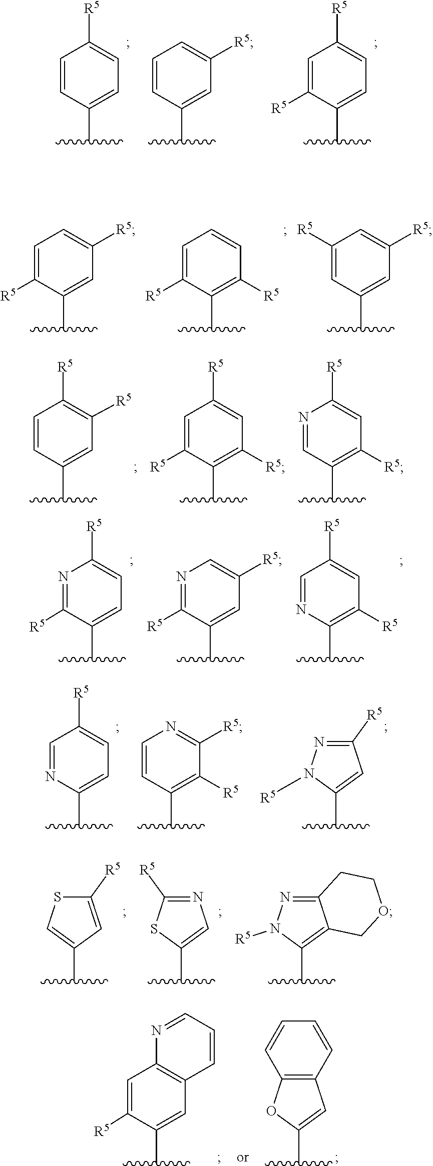

and the remaining variables are as described in the first aspect or the first or second embodiment.

In a fourteenth embodiment, for the compounds of Formula (I), (II), (IIA), (IIB), (III), (IV), (IIIA), (IIIB), (IVA), or (IVB), or a pharmaceutically acceptable salt thereof, R⁵, for each occurrence, is independently selected from cyano, C₁₋₄alkyl, C₃₋₆cycloalkyl, 5 or 6-membered monocyclic heteroaryl, OR^{5a}, and halo, wherein the C₁₋₄alkyl is optionally substituted with one to three R^{5b}, and the 5 or 6-membered monocyclic heteroaryl is optionally substituted with C₁₋₃ alkyl; R^{5a} is H, C₁₋₃alkyl or C₃₋₆cycloalkyl, wherein C₁₋₃alkyl is optionally substituted with one to three halo; R^{5b}, for each occurrence, is independently selected from halo and —OR^{5a}; and the remaining variables are as described in the first aspect or the first, second, seventh, eighth, ninth, tenth, eleventh, twelfth, or thirteenth embodiment or any alternative embodiments described therein.

In a fifteenth embodiment, for the compounds of Formula (I), (II), (IIA), (IIB), (III), (IV), (IIIA), (IIIB), (IVA), or (IVB), or a pharmaceutically acceptable salt thereof, R⁵, for each occurrence, is independently selected from —CH₃, —C(CH₃)₃, —CH₂CH₃, —CH₂CN, —CF₃, —CH₂OCH₃, —OCH₃, —OCHF₂, —OCF₃, —OCH₂CH₃, —OH, —F, —Cl, cyclopropyl, cyclopropyloxy, 4-methyloxazol-2-yl, and —CN; and the remaining variables are as described in the first aspect or the first, second, seventh, eighth, ninth, tenth, eleventh, twelfth, or thirteenth embodiment or any alternative embodiments described therein. In an alternative fifteenth embodiment, for the compounds of Formula (I), (II), (IIA), (IIB), (III), (IV), (IIIA), (IIIB), (IVA), or (IVB), or a pharmaceutically acceptable salt thereof, R⁵, for each occurrence, is independently selected from —CH₃, —CHF₂, —C(CH₃)₃, —CH₂CH₃, —CH₂CN, —CF₃, —CH₂OCH₃, —OCH₃, —OCHF₂, —OCF₃, —OCH₂CH₃, —OCH(CH₃)₂, —OH, —F, —Cl, cyclopropyl, cyclopropyloxy, 4-methyloxazol-2-yl, and —CN; and the remaining variables are as described in the first aspect or the first, second, seventh, eighth, ninth, tenth, eleventh, twelfth, or thirteenth embodiment or any alternative embodiments described therein.

In a sixteenth embodiment, for the compounds of Formula (I), (II), (IIA), (IIB), (III), (IV), (IIIA), (IIIB), (IVA), or (IVB), or a pharmaceutically acceptable salt thereof, R¹ and R² together with the N atom from which they are attached form a 4 to 6-membered monocyclic heterocycle or 6 to 10-membered bicyclic heterocycle, each of which is optionally substituted with one or two R⁴; and the remaining variables are as described in the first aspect or the first, second, seventh, eighth, ninth, tenth, eleventh, twelfth, thirteenth, fourteenth, or fifteenth embodiment or any alternative embodiments described therein.

In a seventeenth embodiment, for the compounds of Formula (I), (II), (IIA), (IIB), (III), (IV), (IIIA), (IIIB), (IVA), or (IVB), or a pharmaceutically acceptable salt thereof, R¹ and R² together with the N atom from which they are attached are

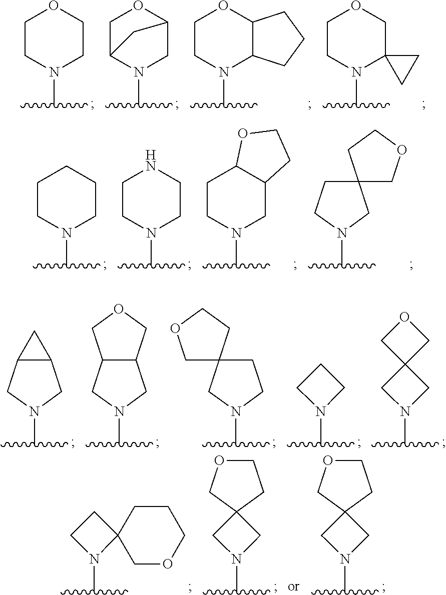

each of which is optionally substituted with one or two R; and the remaining variables are as described in the first aspect or the first, second, seventh, eighth, ninth, tenth, eleventh, twelfth, thirteenth, fourteenth, or fifteenth embodiment or any alternative embodiments described therein. In an alternative seventeenth embodiment, for the compounds of Formula (I), (II), (IIA), (IIB), (III), (IV), (IIIA), (IIIB), (IVA), or (IVB), or a pharmaceutically acceptable salt thereof, R¹ and R² together with the nitrogen atom from which they are attached form groups represented by the following formula:

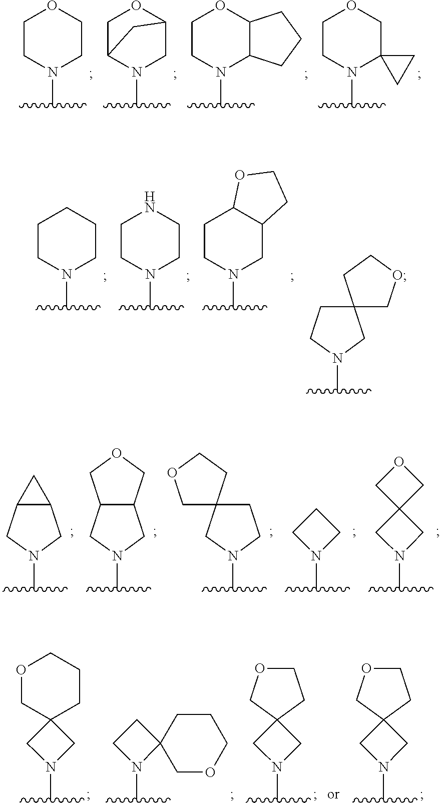

wherein each of the formula depicted above is optionally substituted with one or two R⁴; and the remaining variables are as described in the first aspect or the first, second, seventh, eighth, ninth, tenth, eleventh, twelfth, thirteenth, fourteenth, or fifteenth embodiment or any alternative embodiments described therein.

In an eighteenth embodiment, for the compounds of Formula (I), (II), (IIA), (IIB), (III), (IV), (IIIA), (IIIB), (IVA), or (IVB), or a pharmaceutically acceptable salt thereof, R¹ and R² together with the N atom from which they are attached are

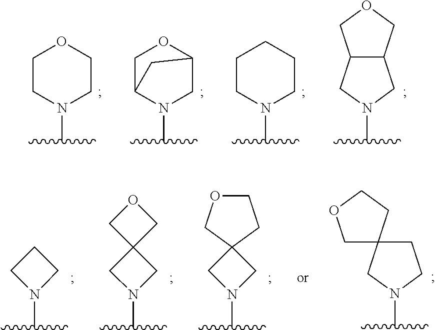

each of which is optionally substituted with one or two R⁴; and the remaining variables are as described in the first aspect or the first, second, seventh, eighth, ninth, tenth, eleventh, twelfth, thirteenth, fourteenth, or fifteenth embodiment or any alternative embodiments described therein.

In a nineteenth embodiment, for the compounds of Formula (I), (II), (IIA), (IIB), (III), (IV), (IIIA), (IIIB), (IVA), or (IVB), or a pharmaceutically acceptable salt thereof, R¹ and R² together with the N atom from which they are attached are

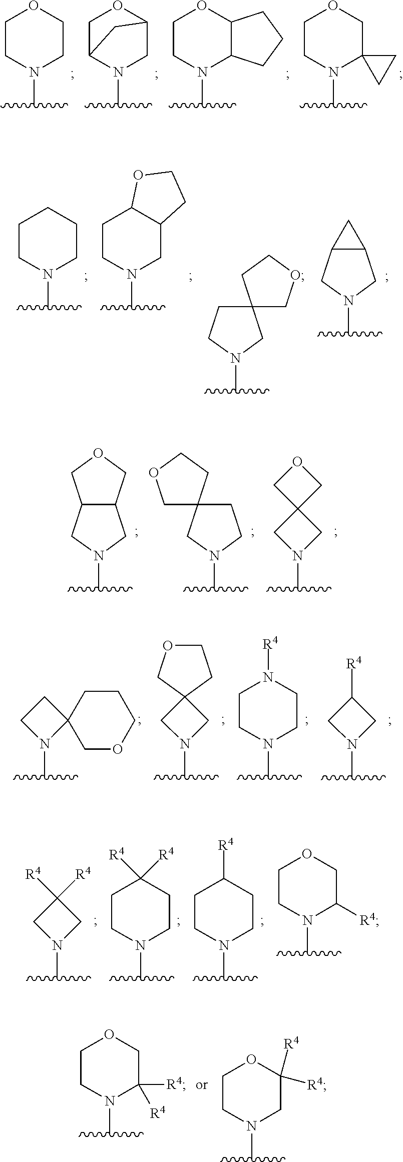

and the remaining variables are as described in the first aspect or the first, second, seventh, eighth, ninth, tenth, eleventh, twelfth, thirteenth, fourteenth, or fifteenth embodiment or any alternative embodiments described therein. In an alternative nineteenth embodiment, for the compounds of Formula (I), (II), (IIA), (IIB), (III), (IV), (IIIA), (IIIB), (IVA), or (IVB), or a pharmaceutically acceptable salt thereof, R¹ and R² together with the nitrogen atom from which they are attached form groups represented by the following formula:

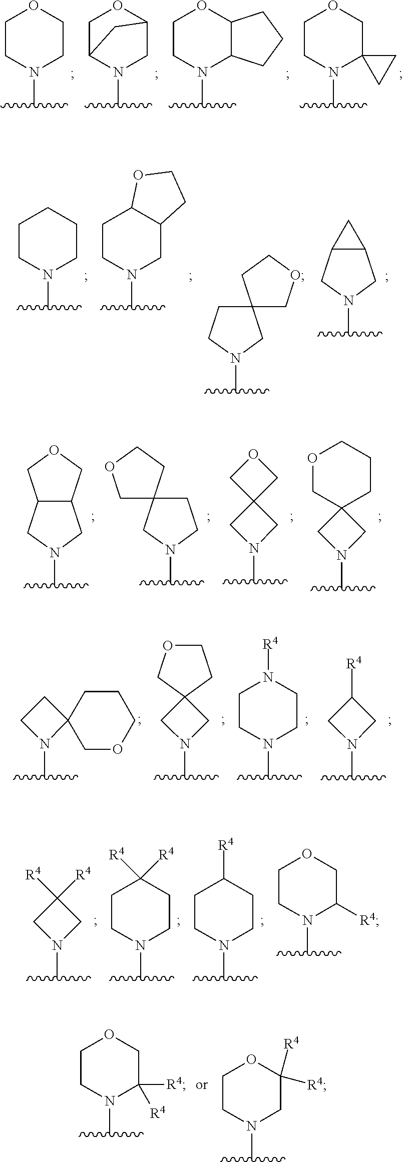

and the remaining variables are as described in the first aspect or the first, second, seventh, eighth, ninth, tenth, eleventh, twelfth, thirteenth, fourteenth, or fifteenth embodiment or any alternative embodiments described therein.

In a twentieth embodiment, for the compounds of Formula (I), (II), (IIA), (IIB), (III), (IV), (IIIA), (IIIB), (IVA), or (IVB), or a pharmaceutically acceptable salt thereof, R¹ and R² together with the N atom from which they are attached are

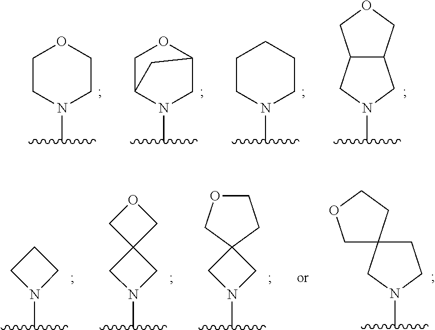

and the remaining variables are as described in the first aspect or the first, second, seventh, eighth, ninth, tenth, eleventh, twelfth, thirteenth, fourteenth, or fifteenth embodiment or any alternative embodiments described therein.

In a twenty-first embodiment, for the compounds of Formula (I), (II), (IIA), (IIB), (III), (IV), (IIIA), (IIIB), (IVA), or (IVB), or a pharmaceutically acceptable salt thereof, R⁴, for each occurrence, is independently selected from halo, C₁₋₃alkyl and —OR^{4a}; and R^{4a} is H or C₁₋₃alkyl; and the remaining variables are as described in the first aspect or the first, second, seventh, eighth, ninth, tenth, eleventh, twelfth, thirteenth, fourteenth, fifteenth, sixteenth, seventeenth, eighteenth, nineteenth, or twentieth embodiment or any alternative embodiments described therein.

In a twenty-second embodiment, for the compounds of Formula (I), (II), (IIA), (IIB), (III), (IV), (IIIA), (IIIB), (IVA), or (IVB), or a pharmaceutically acceptable salt thereof, R⁴, for each occurrence, is independently selected from —OCH₃, F, —OH, and —CH₃; and the remaining variables are as described in the first aspect or the first, second, seventh, eighth, ninth, tenth, eleventh, twelfth, thirteenth, fourteenth, fifteenth, sixteenth, seventeenth, eighteenth, nineteenth, or twentieth embodiment or any alternative embodiments described therein.

In a twenty-third embodiment, for the compounds of Formula (I), (II), (IIA), (IIB), (III), (IV), (IIIA), (IIIB), (IVA), or (IVB), or a pharmaceutically acceptable salt thereof, R¹ is H or C₁₋₃alkyl optionally substituted with one to three R⁴; R² is C₁₋₃alkyl, C₃₋₆cycloalkyl, C₃₋₆cycloalkenyl or 4 to 6-membered monocyclic heterocyclyl, wherein the C₁₋₃alkyl, C₃₋₆cycloalkyl, C₃₋₆cycloalkenyl and 4 to 6-membered monocyclic heterocyclyl are each optionally substituted with one or two substituents independently selected from C₁₋₃alkyl, C₁₋₃ alkoxy, and halo; and the remaining variables are as described in the first aspect or the first, second, seventh, eighth, ninth, tenth, eleventh, twelfth, thirteenth, fourteenth, or fifteenth embodiment or any alternative embodiments describe therein. In an alternative twenty-third embodiment, for the compounds of Formula (I), (II), (IIA), (IIB), (III), (IV), (IIIA), (IIIB), (IVA), or (IVB), or a pharmaceutically acceptable salt thereof, R¹ is H or C₁₋₃alkyl optionally substituted with one to three R⁴; and R² is C₁₋₃alkyl, C₃₋₆cycloalkyl, C₃₋₆cycloalkenyl 6 to 10-membered bicyclic heterocyclyl, or 4 to 6-membered monocyclic heterocyclyl, wherein the C₁₋₃alkyl, C₃₋₆cycloalkyl, C₃₋₆cycloalkenyl, 6 to 10-membered bicyclic heterocyclyl, and 4 to 6-membered monocyclic heterocyclyl are each optionally substituted with one or two substituents independently selected from C₁₋₃alkyl, C₁₋₃alkoxy, halo, and 4 to 6-membered monocyclic heterocyclyl; and the remaining variables are as described in the first aspect or the first, second, seventh, eighth, ninth, tenth, eleventh, twelfth, thirteenth, fourteenth, or fifteenth embodiment or any alternative embodiments described therein.

In a twenty-fourth embodiment, for the compounds of Formula (I), (II), (IIA), (IIB), (III), (IV), (IIIA), (IIIB), (IVA), or (IVB), or a pharmaceutically acceptable salt thereof, R¹ is H or C₁₋₃alkyl optionally substituted with one to three R⁴; R² is C₁₋₃alkyl substituted with C₁₋₃alkoxy, C₃₋₆cycloalkyl optionally substituted with one to two halo, or a 4 to 6-membered monocyclic heterocyclyl optionally substituted with one or two C₁₋₃alkyl; and the remaining variables are as described in the first aspect or the first, second, seventh, eighth, ninth, tenth, eleventh, twelfth, thirteenth, fourteenth, or fifteenth embodiment or any alternative embodiments described therein. In an alternative twenty-fourth embodiment, for the compounds of Formula (I), (II), (IIA), (IIB), (III), (IV), (IIIA), (IIIB), (IVA), or (IVB), or a pharmaceutically acceptable salt thereof, R¹ is H or C₁₋₃alkyl optionally substituted one to three R⁴; and R² is C₁₋₃alkyl substituted with C₁₋₃alkoxy or a 4 to 6-membered monocyclic heterocyclyl, C₃₋₆cycloalkyl optionally substituted with one to two halo, 6 to 10-membered bicyclic heterocyclyl, or a 4 to 6-membered monocyclic heterocyclyl optionally substituted with one or two C₁₋₃alkyl; and the remaining variables are as described in the first aspect or the first, second, seventh, eighth, ninth, tenth, eleventh, twelfth, thirteenth, fourteenth, or fifteenth embodiment or any alternative embodiments described therein.

In a twenty-fifth embodiment, for the compounds of Formula (I), (II), (IIA), (IIB), (III), (IV), (IIIA), (IIIB), (IVA), or (IVB), or a pharmaceutically acceptable salt thereof, R¹ is H or methyl, R² is 2-methoxyethyl, 4,4-difluorocyclohexyl, 4-fluorocyclohex-3-en-1-yl, 2,2-difluoroethyl, 4-methylpiperidinyl, tetrahydro-2H-pyran-4-yl, 3-methyloxetan-3-yl, oxatan-3-ylmethyl, tetrahydrofuran-3-yl and 2-oxaspiro[3.3]heptan-6-yl; and the remaining variables are as described in the first aspect or the first, second, seventh, eighth, ninth, tenth, eleventh, twelfth, thirteenth, fourteenth, or fifteenth embodiment or any alternative embodiments described therein. In an alternative twenty-fifth embodiment, for the compounds of Formula (I), (II), (IIA), (IIB), (III), (IV), (IIIA), (III), (IVA), or (IVB), or a pharmaceutically acceptable salt thereof, R¹ is H or methyl; and R² is 2-methoxyethyl, —CH₂-tetrahydropyranyl, 4,4-difluorocyclohexyl, 4-fluorocyclohex-3-en-1-yl, 2,2-difluoroethyl, 4-methylpiperidinyl, tetrahydro-2H-pyran-4-yl, oxetan-3-yl, 3-methyloxetan-3-yl, tetrahydrofuran-3-yl, 3-methyltetrahydrofuran-3-yl, and 2-oxaspiro[3.3]heptan-6-yl; and the remaining variables are as described in the first aspect or the first, second, seventh, eighth, ninth, tenth, eleventh, twelfth, thirteenth, fourteenth, or fifteenth embodiment or any alterative embodiments described therein.

In a twenty-sixth embodiment, the compound of the present disclosure is represented by the following Formula:

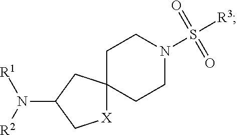

or a pharmaceutically acceptable salt thereof, wherein:
X is O or CH₂;
R¹ is H or C₁₋₃alkyl optionally substituted with one to three R⁴;
R² is C₁₋₃alkyl substituted with C₁₋₃alkoxy, C₃₋₆cycloalkyl optionally substituted with one to two halo, or a 4 to 6-membered monocyclic heterocyclyl optionally substituted with one or two C₁₋₃alkyl;
or R¹ and R² together with the N atom from which they are attached form a 4 to 6-membered monocyclic heterocycle, 7 to 9-membered bicyclic heterocycle, each of which is optionally substituted with one or two R⁴;
R⁴, for each occurrence, is independently selected from C₁₋₃alkoxy, OH, halo, and C₁₋₃alkyl;
R³ is phenyl, 5 or 6-membered monocyclic heteroaryl, 9 or 10-membered bicyclic heteroaryl, or 9 or 10-membered bicyclic heterocycle, wherein the phenyl, 5 or 6-membered monocyclic heteroaryl, 9 or 10-membered bicyclic heteroaryl and 9 or 10-membered bicyclic heterocycle are each optionally substituted with one to three R⁵;
R⁵ is cyano, C₁₋₄alkyl, C₃₋₆cycloalkyl, OR^{5a}, and halo, wherein the C₁₋₄alkyl is optionally substituted with one to three R^{5b};
R^{5a} is C₁₋₃alkyl is optionally substituted with one to three halo.

In some embodiments, for the compounds of the twenty-sixth embodiment, or a pharmaceutically acceptable salt thereof, (i) when X is O, and R¹ and R² together with the N atom from which they are attached form unsubstituted morpholine, unsubstituted pyrrolidine, or unsubstituted N-methylpiperazine, then R³ is selected from a 9 or 10-membered bicyclic heteroaryl or a 6 to 10 membered bicyclic heterocycle each optionally substituted with one or more substituent R⁵ or a phenyl or a 5 or 6-membered monocyclic heteroaryl each substituted with at least two R⁵ groups, or one R⁵ group that is OR^{5a}; (ii) when X is O, and R¹ and R² are both C₁₋₆alkyl, then at least one of the C₁₋₆alkyl represented by R¹ and R² is substituted by one or more R⁴ selected from OR^{4a}, halo, and C₃₋₈cycloalkyl; (iii) when X is a bond, and R¹ and R² are both C₁₋₆alkyl, then at least one of the C₁₋₆alkyl represented by R¹ and R² is substituted by one or more R⁴; or (iv) when p is 0 and q is 1, then X is O.

In an alternative twenty-sixth embodiment, the compound of the present disclosure is represented by the following Formula:

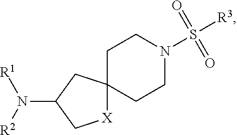

or a pharmaceutically acceptable salt thereof, wherein:
X is O or CH₂;
R¹ is H or C₁₋₃alkyl optionally substituted with one to three R⁴; R² is C₁₋₃alkyl substituted with C₁₋₃alkoxy or a 4 to 6-membered monocyclic heterocyclyl, C₃₋₆ cycloalkyl optionally substituted with one to two halo, 6 to 10-membered bicyclic heterocyclyl, or a 4 to 6-membered monocyclic heterocyclyl optionally substituted with one or two C₁₋₃alkyl;
or R¹ and R² together with the N atom from which they are attached form a 4 to 6-membered monocyclic heterocycle, 7 to 9-membered bicyclic heterocycle, each of which is optionally substituted with one or two R⁴;
R⁴, for each occurrence, is independently selected from C₁₋₃alkoxy, OH, halo, and C₁₋₃alkyl;
R³ is phenyl, 5 or 6-membered monocyclic heteroaryl, 9 or 10-membered bicyclic heteroaryl, or 9 or 10-membered bicyclic heterocycle, wherein the phenyl, 5 or 6-membered monocyclic heteroaryl, 9 or 10-membered bicyclic heteroaryl and 9 or 10-membered bicyclic heterocycle are each optionally substituted with one to three R⁵;
R⁵ is cyano, C₁₋₄alkyl, C₃₋₆cycloalkyl, OR^{5a}, and halo, wherein the C₁₋₄alkyl is optionally substituted with one to three R^{5b};
R^{5a} is C₁₋₃alkyl is optionally substituted with one to three halo.

In a twenty-seventh embodiment, for the compounds of twenty-sixth embodiment, or a pharmaceutically acceptable salt thereof, R¹ is H or C₁₋₃alkyl optionally substituted one to three R⁴ and R² is C₁₋₃alkyl substituted with C₁₋₃alkoxy, or a 4 to 6-membered monocyclic heterocyclyl optionally substituted with one or two substituents independently selected from halo and C₁₋₃ alkyl;
or R¹ and R² together with the N atom from which they are attached form the groups represented by the following formula:

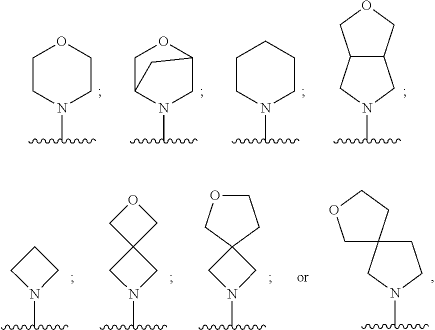

each of which is optionally substituted with one or two R⁴;
R³ is

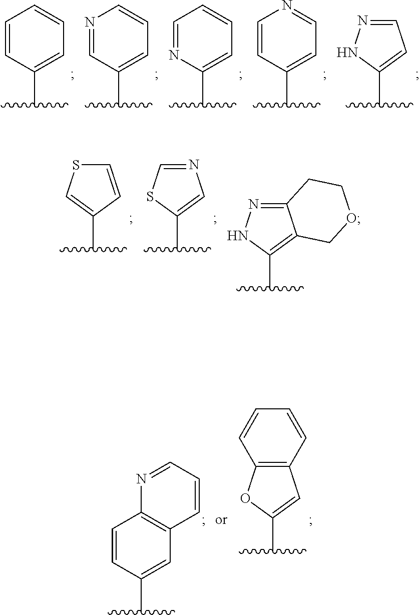

each of which is optionally substituted with one to three R⁵; and the remaining variables are as described in the twenty-sixth embodiment.

In an alternative twenty-seventh embodiment, for the compounds of twenty-sixth embodiment, or a pharmaceutically acceptable salt thereof, R¹ is H or C₁₋₃alkyl optionally substituted one to three R⁴ and R² is C₁₋₃alkyl substituted with C₁₋₃alkoxy or a 4 to 6-membered monocyclic heterocyclyl, 6 to 10-membered bicyclic heterocyclyl, or a 4 to 6-membered monocyclic heterocyclyl optionally substituted with one or two substituents independently selected from halo and C₁₋₃ alkyl;
or R¹ and R² together with the N atom from which they are attached form the groups represented by the following formula:

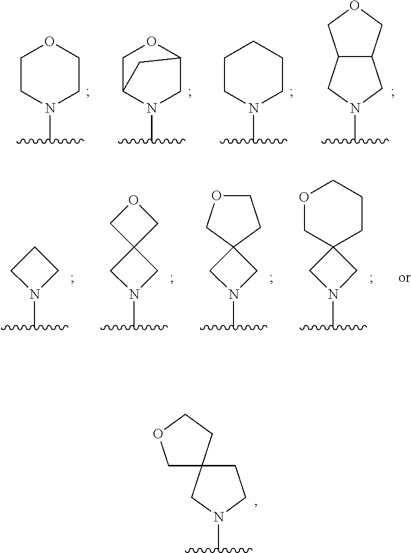

each of which is optionally substituted with one or two R⁴;
R³ is

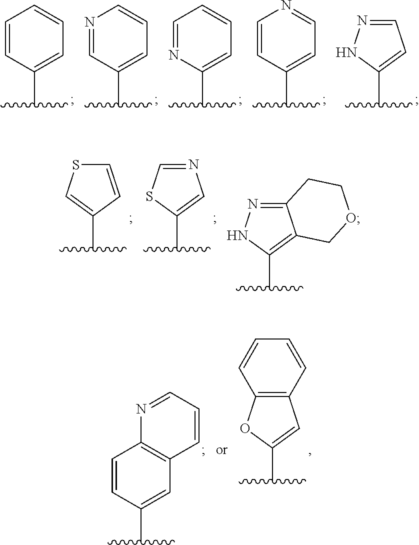

each of which is optionally substituted with one to three R⁵; and the remaining variables are as described in the twenty-sixth embodiment.

In a twenty-eighth embodiment, for the compounds of twenty-sixth embodiment, or a pharmaceutically acceptable salt thereof, R³ is

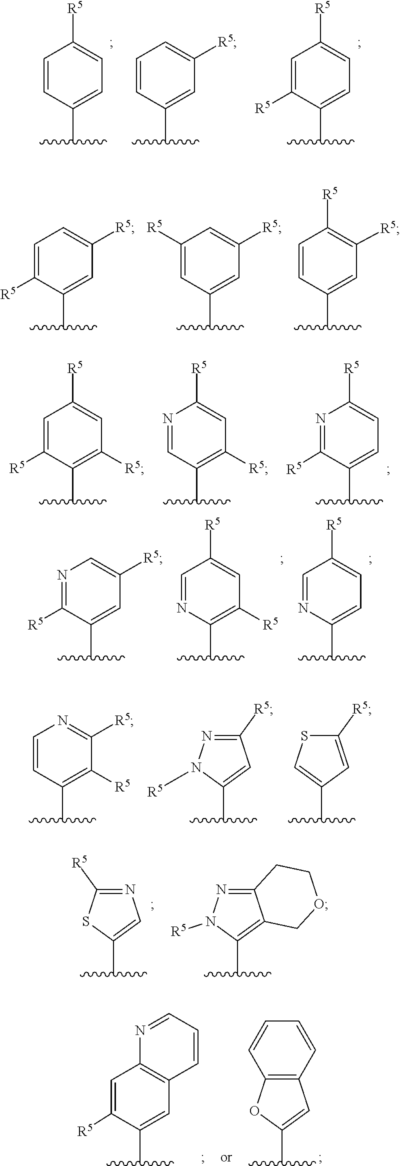

R¹ and R² together with the nitrogen atom from which they are

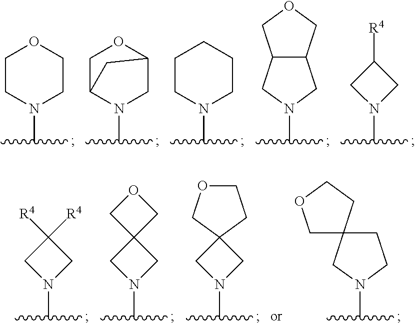

and the remaining variables are as described in the twenty-sixth or twenty-seventh embodiment or any alternative embodiments described therein.

In an alternative twenty-eighth embodiment, for the compounds of twenty-sixth embodiment, or a pharmaceutically acceptable salt thereof, R³ is

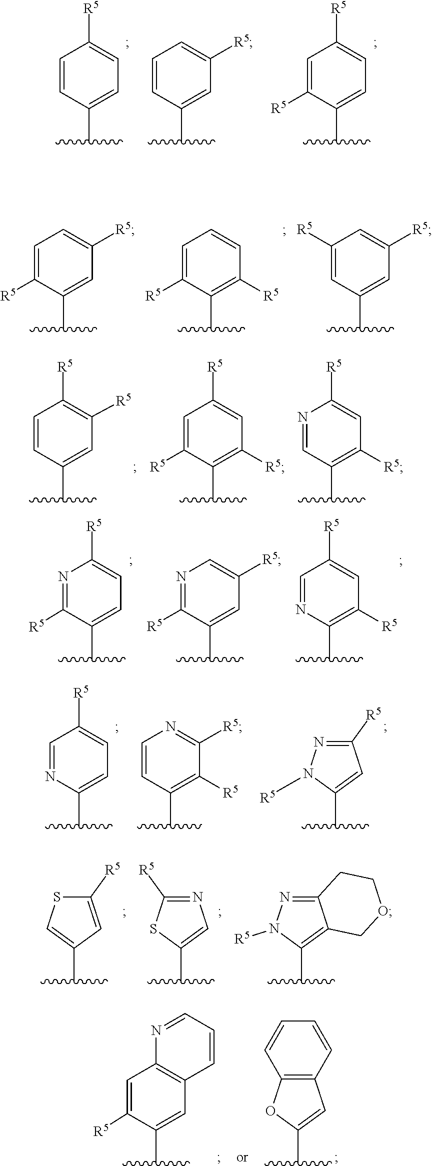

and
(1) R¹ is H or CH₃ and R² is 2-methoxyethyl, oxatan-3-ylmethyl, 3-methyltetrahydrofuran-3-yl, tetrahydro-2H-pyran-4-yl, —CH₂-tetrahydropyranyl, or 2-oxaspiro[3.3]heptan-6-yl; or
(2) R¹ and R² together with the nitrogen atom from which they are

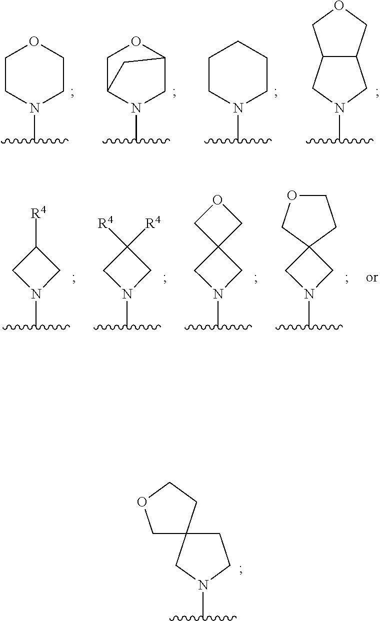

and the remaining variables are as described in the twenty-sixth or twenty-seventh embodiment or any alternative embodiments described therein.

In a twenty-ninth embodiment, for the compounds of twenty-sixth embodiment, or a pharmaceutically acceptable salt thereof, R⁴, for each occurrence, is independently selected from halo, C₁₋₃alkyl and —OR^{4a}; and R^{4a} is H or C₁₋₃alkyl; and the remaining variables are as described in the twenty-sixth, twenty-seventh, or twenty-eighth embodiment or any alternative embodiments described therein.

In a thirtieth embodiment, for the compounds of twenty-sixth embodiment, or a pharmaceutically acceptable salt thereof, R⁴, for each occurrence, is independently selected from —OCH₃, F, —OH, or —CH₃; and the remaining variables are as described in the twenty-sixth, twenty-seventh, or twenty-eighth embodiment or any alternative embodiments described therein.

In a thirty-first embodiment, for the compounds of twenty-sixth embodiment, or a pharmaceutically acceptable salt thereof, R⁵, for each occurrence, is independently selected from cyano, C₁₋₄alkyl, C₃₋₆cycloalkyl, OR^{5a}, and halo, wherein the C₁₋₄alkyl is optionally substituted with one to three R^{5b}; R^{5a} is H, C₃₋₆cycloalkyl or C₁₋₃alkyl optionally substituted with one to three halo; R^{5b}, for each occurrence, is independently selected from halo and C₁₋₃ alkoxy; and the remaining variables are as described in the twenty-sixth, twenty-seventh, twenty-eighth, twenty-ninth, or thirtieth embodiment or any alternative embodiments described therein. In some embodiments, R⁵ is C₁₋₃alkyl substituted with one to three halo.

In a thirty-second embodiment, for the compounds of twenty-sixth embodiment, or a pharmaceutically acceptable salt thereof, R⁵, for each occurrence, is independently selected from —CH₃, —CF₃, —OCH₃, —OCHF₂, —OCF₃, —OH, —CN, F, Cl, —CH₂OCH₃, cyclopropyl, and cyclopropyloxy; and the remaining variables are as described in the twenty-sixth, twenty-seventh, twenty-eighth, twenty-ninth, or thirtieth embodiment or any alternative embodiments described therein.

In a thirty-third embodiment, the compound of the present disclosure is represented by Formula (V),

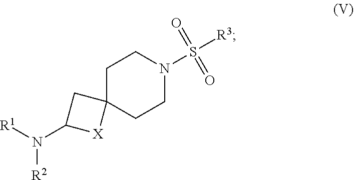

or a pharmaceutically acceptable salt thereof, wherein the variables in Formula (V) are as defined in the first aspect or the first or second embodiment above.

In a thirty-fourth embodiment, the compound of the present disclosure is represented by Formula (VA) or (VB),

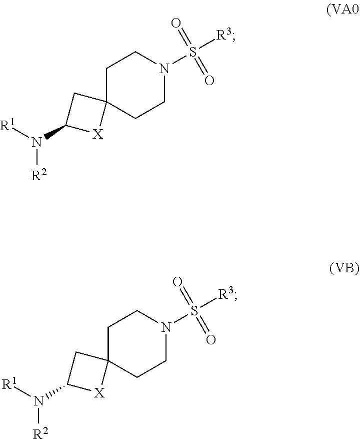

or a pharmaceutically acceptable salt thereof, wherein the variables in Formula (VA) or (VB) are as defined in in the first aspect or the first or second embodiment above.

In a thirty-fifth embodiment, for the compounds of Formula (V), (VA), or (VB), or a pharmaceutically acceptable salt thereof, X is CH₂; and the remaining variables are as described in the first aspect or the first or second embodiment.

In a thirty-sixth embodiment, for the compounds of Formula (V), (VA), or (VB), or a pharmaceutically acceptable salt thereof, R³ is phenyl or 5 or 6-membered monocyclic heteroaryl, wherein the phenyl or 5 or 6-membered monocyclic heteroaryl are each optionally substituted with one to two substituent R⁵; and the remaining variables are as described in the first aspect or the first, second, or thirty-fifth embodiment.

In a thirty-seventh embodiment, for the compounds of Formula (V), (VA), or (VB), or a pharmaceutically acceptable salt thereof, R³ is pyridyl, phenyl, or pyrazolyl; and the remaining variables are as described in the first aspect or the first, second, or thirty-fifth embodiment.

In a thirty-eighth embodiment, for the compounds of Formula (V), (VA), or (VB), or a pharmaceutically acceptable salt thereof, R³ is

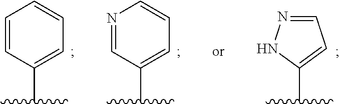 [SMILES: *c1ccccc1 ; *c1cccnc1 ; *c1ccn[nH]1]

each of which is optionally substituted with one to two R⁵; and the remaining variables are as described in the first aspect or the first, second, or thirty-fifth embodiment.

In a thirty-ninth embodiment, for the compounds of Formula (V), (VA), or (VB), or a pharmaceutically acceptable salt thereof, R³ is

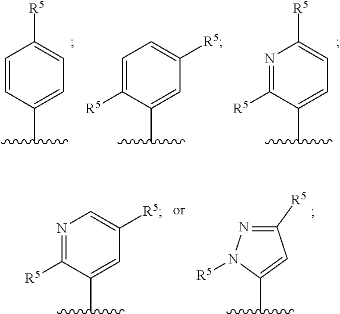

and the remaining variables are as described in the first aspect or the first, second, or thirty-fifth embodiment.

In a fortieth embodiment, for the compounds of Formula (V), (VA), or (VB), or a pharmaceutically acceptable salt thereof, R⁵, for each occurrence, is independently selected from cyano, C₁₋₄alkyl, OR^{5a}, and halo, wherein the C₁₋₄alkyl is optionally substituted with one to three R^{5b}, R^{5a} is C₁₋₃alkyl optionally substituted with one to three halo, R^{5b}, for each occurrence, is halo; and the remaining variables are as described in the first aspect or the first, second, thirty-fifth, thirty-sixth, thirty-seventh, thirty-eighth, or thirty-ninth embodiment. In an alternative fortieth embodiment, for the compounds of Formula (V), (VA), or (VB), or a pharmaceutically acceptable salt thereof, R⁵, for each occurrence, is independently selected from cyano, C₁₋₄alkyl, OR^{5a}, C₃₋₄cycloalkyl, and halo, wherein the C₁₋₄alkyl is optionally substituted with one to three R^{5b}, R^{5a} is C₁₋₃alkyl optionally substituted with one to three halo, R^{5b}, for each occurrence, is halo; and the remaining variables are as described in first aspect or the first, second, thirty-fifth, thirty-sixth, thirty-seventh, thirty-eighth, or thirty-ninth embodiment.

In a forty-first embodiment, for the compounds of Formula (V), (VA), or (VB), or a pharmaceutically acceptable salt thereof, R⁵, for each occurrence, is independently selected from —CH₃, —CH₂CH₃, —CF₃, —OCH₃, —OCHF₂, —OCF₃, —F, and —CN; and the remaining variables are as described in the first aspect or the first, second, thirty-fifth, thirty-sixth, thirty-seventh, thirty-eighth, or thirty-ninth embodiment. In an alternative forty-first embodiment, for the compounds of Formula (V), (VA), or (VB), or a pharmaceutically acceptable salt thereof, R⁵, for each occurrence, is independently selected from —CH₃, —CHF₂, —CH₂CH₃, —CF₃, —OCH₃, —OCHF₂, —OCF₃, —F, —Cl, cyclopropyl, and —CN; and the remaining variables are as described in the first aspect or the first, second, thirty-fifth, thirty-sixth, thirty-seventh, thirty-eighth, or thirty-ninth embodiment.

In a forty-second embodiment, for the compounds of Formula (V), (VA), or (VB), or a pharmaceutically acceptable salt thereof, if R¹ and R² are both C₁₋₆alkyl, at least one of said C₁₋₆alkyl is further substituted by at least one R⁴; and the remaining variables are as described in the first aspect or the first, second, thirty-fifth, thirty-sixth, thirty-seventh, thirty-eighth, thirty-ninth, fortieth, or forty-first embodiment or any alternative embodiments described therein.

In a forty-third embodiment, for the compounds of Formula (V), (VA), or (VB), or a pharmaceutically acceptable salt thereof, R¹ is H or C₁₋₃alkyl, and R² is C₁₋₃alkyl optionally substituted with C₁₋₃alkoxy; and the remaining variables are as described in the first aspect or the first, second, thirty-fifth, thirty-sixth, thirty-seventh, thirty-eighth, thirty-ninth, fortieth, or forty-first embodiment or any alternative embodiments described therein. In an alternative forty-third embodiment, for the compounds of Formula (V), (VA), or (VB), or a pharmaceutically acceptable salt thereof, R¹ is H or C₁₋₃alkyl, and R² is C₁₋₃alkyl optionally substituted with C₁₋₃alkoxy or 4- to 6-membered heterocyclyl; and the remaining variables are as described in the first aspect or the first, second, thirty-fifth, thirty-sixth, thirty-seventh, thirty-eighth, thirty-ninth, fortieth, or forty-first embodiment or any alternative embodiments described therein.

In a forty-fourth embodiment, for the compounds of Formula (V), (VA), or (VB), or a pharmaceutically acceptable salt thereof, R¹ is —CH₃ and R² is —CH₂OCH₃; and the remaining variables are as described in the first aspect or the first, second, thirty-fifth, thirty-sixth, thirty-seventh, thirty-eighth, thirty-ninth, fortieth, or forty-first embodiment or any alternative embodiments described therein. In an alternative forty-fourth embodiment, for the compounds of Formula (V), (VA), or (VB), or a pharmaceutically acceptable salt thereof, R¹ is —CH₃ and R² is —CH₂CH₂OCH₃ or tetrahydropyranyl; and the remaining variables are as described in the first aspect or the first, second, thirty-fifth, thirty-sixth, thirty-seventh, thirty-eighth, thirty-ninth, fortieth, or forty-first embodiment or any alternative embodiments described therein.

In a forty-fifth embodiment, for the compounds of Formula (V), (VA), or (VB), or a pharmaceutically acceptable salt thereof, R¹ and R² together with the N atom from which they are attached form a 4 to 6-membered monocyclic heterocycle or a 6 or 7-membered bicyclic heterocycle, each of which is optionally substituted with one or two R⁴; and the remaining variables are as described in the first aspect or the first, second, thirty-fifth, thirty-sixth, thirty-seventh, thirty-eighth, thirty-ninth, fortieth, or forty-first embodiment or any alternative embodiments described therein.

In a forty-sixth embodiment, for the compounds of Formula (V), (VA), or (VB), or a pharmaceutically acceptable salt thereof, R¹ and R² together with the N atom from which they are attached are

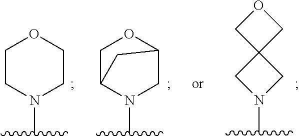

and the remaining variables are as described in the first aspect or the first, second, thirty-fifth, thirty-sixth, thirty-seventh, thirty-eighth, thirty-ninth, fortieth, or forty-first embodiment or any alternative embodiments described therein. In an alternative forty-sixth embodiment, for the compounds of Formula (V), (VA), or (VB), or a pharmaceutically acceptable salt thereof, R¹ and R² together with the N atom from which they are attached are

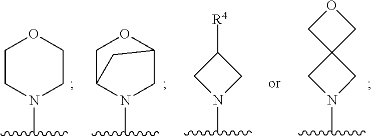

and the remaining variables are as described in the first aspect or the first, second, thirty-fifth, thirty-sixth, thirty-seventh, thirty-eighth, thirty-ninth, fortieth, or forty-first embodiment or any alternative embodiments described therein.

In a forty-seventh embodiment, the compound of the present disclosure is represented by Formula (VI),

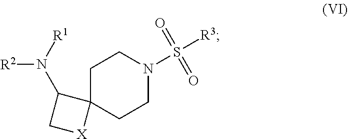

or a pharmaceutically acceptable salt thereof, wherein the variables in Formula (VI) are as defined in the first aspect or the first or second embodiment above.

In a forty-eighth embodiment, the compound of the present disclosure is represented by Formula (VIA) or (VIB),

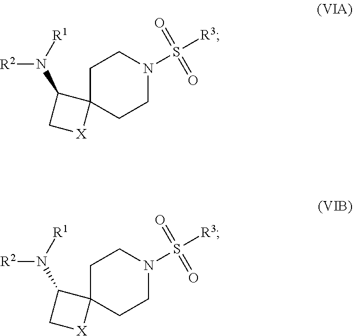

or a pharmaceutically acceptable salt thereof, wherein the variables in Formula (VIA) or (VIB) are as defined in the first aspect or the first or second embodiment above.

In a forty-ninth embodiment, for the compounds of Formula (VIA) or (VIB), or a pharmaceutically acceptable salt thereof, X is O; and the remaining variables are as described in the first aspect or the first or second embodiment.

In a fiftieth embodiment, for the compounds of Formula (VIA) or (VIB), or a pharmaceutically acceptable salt thereof, R³ is phenyl optionally substituted with one to two R⁴; and the remaining variables are as described in the first aspect or the first, second, or forty-ninth embodiment.

In a fifty-first embodiment, for the compounds of Formula (VIA) or (VIB), or a pharmaceutically acceptable salt thereof, R³ is

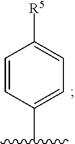

and the remaining variables are as described in the first aspect or the first, second, or forty-ninth embodiment.

In a fifty-second embodiment, for the compounds of Formula (VIA) or (VIB), or a pharmaceutically acceptable salt thereof, R¹ and R², together with the nitrogen atom from which they are attached are

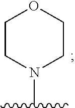 [SMILES: *N1CCOCC1]

and the remaining variables are as described in the first aspect or the first, second, forty-ninth, fiftieth, or fifty-first embodiment.

In a fifty-third embodiment, the compound of the present disclosure is represented by Formula (VII):

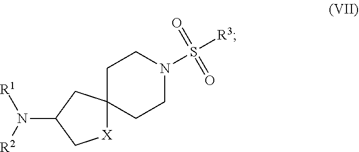

or a pharmaceutically acceptable salt thereof, wherein:
X is O, CH₂, or a bond;
R¹ is H and R² is 4 to 6-membered monocyclic heterocyclyl or 6 to 10-membered bicyclic heterocyclyl;
or R¹ and R² together with the N atom from which they are attached form a 4 to 7-membered monocyclic heterocycle or 6 to 10-membered bicyclic heterocycle;
R³ is phenyl or 5 or 6-membered monocyclic heteroaryl, each of which is optionally substituted with one or two R⁵;
each R⁵ is independently selected from C₁₋₃alkyl, C₁₋₃haloalkyl, C₃₋₄cycloalkyl, OR^{5a}, cyano, and halo; and
R^{5a} is C₁₋₃haloalkyl.

In a fifty-fourth embodiment, the compound of the present disclosure is represented by Formula (III) or (IV):

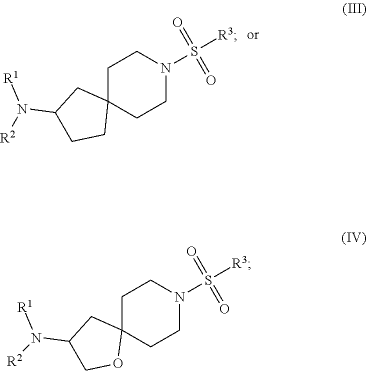

or a pharmaceutically acceptable salt thereof, and the remaining variables are as described in the fifty-third embodiment.

In a fifty-fifth embodiment, the compound of the present disclosure is represented by Formula (IIIA), (IIIB), (IVA), or (IVB):

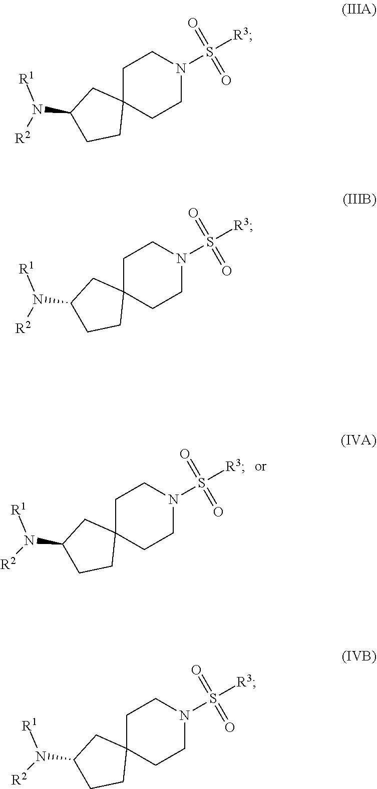

or a pharmaceutically acceptable salt thereof, and the remaining variables are as described in the fifty-third embodiment.

In a fifty-sixth embodiment, for the compound of Formula (VII), (III), (IV), (IIIA), (IIIB), (IVA), or (IVB), or a pharmaceutically acceptable salt thereof, R³ is phenyl, pyrazoyl, or pyridinyl, each of which is optionally substituted by one or two R⁵ and the remaining variables are as described in the fifty-third, fifty-fourth, or fifty-fifth embodiment.

In a fifty-seventh embodiment, for the compound of Formula (VII), (III), (IV), (IIIA), (IIIB), (IVA), or (IVB), or a pharmaceutically acceptable salt thereof, R³ represented by the following formula:

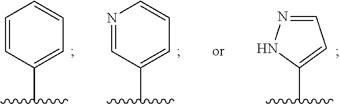 [SMILES: *c1ccccc1 ; *c1cccnc1 ; *c1ccn[nH]1]

wherein each of the formula depicted above is optionally substituted with one to two R⁵; and the remaining variables are as described in the fifty-sixth embodiment.

In a fifty-eighth embodiment, for the compound of Formula (VII), (III), (IV), (IIIA), (IIIB), (IVA), or (IVB), or a pharmaceutically acceptable salt thereof, R³ represented by the following formula:

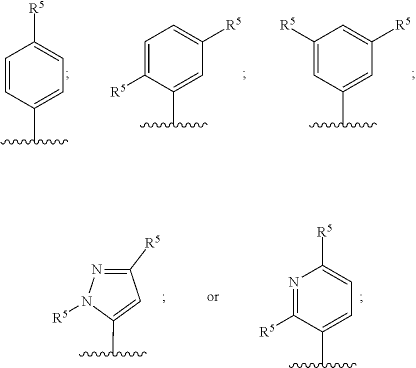

and the remaining variables are as described in the fifty-sixth embodiment.

In a fifty-ninth embodiment, for the compound of Formula (VII), (III), (IV), (IIIA), (IIIB), (IVA), or (IVB), or a pharmaceutically acceptable salt thereof:
(1) R² is represented by the following formula:

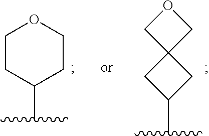 [SMILES: *C1CCOCC1 ; *C1CC2(COC2)C1]

or
(2) R¹ and R² together with the N atom from which they are attached form a group represented by the following formula:

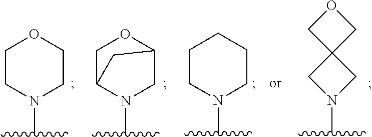

and the remaining variables are as described in the fifty-third, fifty-fourth, fifty-fifth, fifty-sixth, fifty-seventh, or fifty-eighth embodiment.

In a sixtieth embodiment, for the compound of Formula (VII), (III), (IV), (IIIA), (IIIB), (IVA), or (IVB), or a pharmaceutically acceptable salt thereof, each R⁵ is independently selected from —CH₃, —CF₃, —F, —CN, and —OCHF₂; and the remaining variables are as described in the fifty-third, fifty-fourth, fifty-fifth, fifty-sixth, fifty-seventh, fifty-eighth, or fifty-ninth embodiment.

In a sixty-first embodiment, the compound of the present disclosure is represented by Formula (VIII):

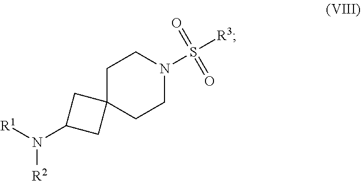

and the remaining variables are as described in the fifty-third embodiment.

In a sixty-second embodiment, the compound of the present disclosure is represented by Formula (VIIIA) or (VIIIB):

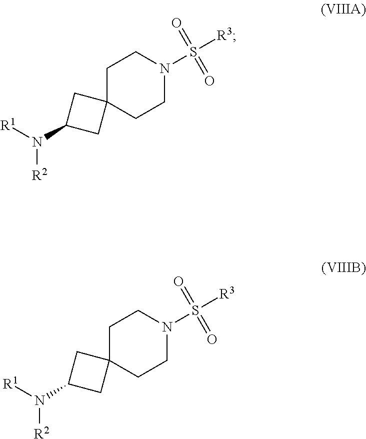

and the remaining variables are as described in the fifty-third or sixty-first embodiment.

In a sixty-third embodiment, for the compound of Formula (VII), (VIII), (VIIIA), (VIIIB), or a pharmaceutically acceptable salt thereof, R³ is pyrazoyl or pyridinyl, each of which is optionally substituted by one or two R⁵; and the remaining variables are as described in the fifty-third, sixty-first, or sixty-second embodiment.

In a sixty-fourth embodiment, for the compound of Formula (VII), (VIII), (VIIIA), (VIIIB), or a pharmaceutically acceptable salt thereof, R³ is represented by the following formula:

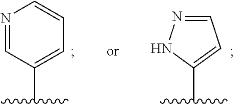 [SMILES: *c1cccnc1 ; *c1ccn[nH]1]

wherein each of the formula depicted above is optionally substituted with one to two R⁵; and the remaining variables are as described in the fifty-third, sixty-first, sixty-second, or sixty-third embodiment.

In a sixty-fifth embodiment, for the compound of Formula (VII), (VIII), (VIIIA), (VIIIB), or a pharmaceutically acceptable salt thereof, R³ is represented by the following formula:

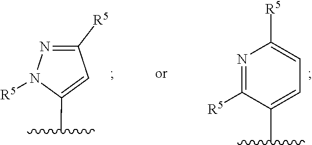

and the remaining variables are as described in the fifty-third, sixty-first, sixty-second, sixty-third, or sixty-fourth embodiment.

In a sixty-sixth embodiment, for the compound of Formula (VII), (VIII), (VIIIA), (VIIIB), or a pharmaceutically acceptable salt thereof:
(1) R² is represented by the following formula:

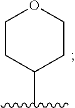 [SMILES: *C1CCOCC1]

or
(2) R¹ and R² together with the N atom from which they are attached form a group represented by the following formula:

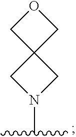 [SMILES: *N1CC2(COC2)C1]

and the remaining variables are as described in the fifty-third, sixty-first, sixty-second, sixty-third, sixty-fourth, or sixty-fifth embodiment.

In a sixty-seventh embodiment, for the compound of Formula (VII), (VIII), (VIIIA), (VIIIB), or a pharmaceutically acceptable salt thereof, each R⁵ is independently selected from —CH₃, —CF₃, and cyclopropyl; and the remaining variables are as described in the fifty-third, sixty-first, sixty-second, sixty-third, sixty-fourth, sixty-fifth, or sixty-sixth embodiment.

In a sixty-eighth embodiment, the present disclosure provides a compound described herein (e.g., a compound of any one of Examples 1 to 144), or a pharmaceutically acceptable salt thereof.

In a sixty-ninth embodiment, the present disclosure provides a compound selected from the group consisting of:
8-(Benzofuran-2-ylsulfonyl)-3-morpholino-1-oxa-8-azaspiro[4.5]decane;
8-((3-Chloro-5-(methoxymethyl)phenyl)sulfonyl)-3-morpholino-1-oxa-8-azaspiro[4.5]decane;
2-((3-Morpholino-1-oxa-8-azaspiro[4.5]decan-8-yl)sulfonyl)-3-(trifluoromethoxy)phenol;
8-((4-Methyl-6-(trifluoromethyl)pyridin-3-yl)sulfonyl)-3-morpholino-1-oxa-8-azaspiro[4.5]decane;
8-((5-methylthiophen-3-yl)sulfonyl)-3-morpholino-1-oxa-8-azaspiro[4.5]decane;
(R)- or (S)-8-((1-methyl-3-(trifluoromethyl)-1H-pyrazol-5-yl)sulfonyl)-3-morpholino-1-oxa-8-azaspiro[4.5]decane;
8-((4-Fluorophenyl)sulfonyl)-3-(2-oxa-6-azaspiro[3.3]heptan-6-yl)-1-oxa-8-azaspiro[4.5]decane;
4-((3-(2-Oxa-6-azaspiro[3.3]heptan-6-yl)-1-oxa-8-azaspiro[4.5]decan-8-yl)sulfonyl)-3-chlorobenzonitrile;
6-(8-((3-Fluoro-4-methoxyphenyl)sulfonyl)-8-azaspiro[4.5]decan-2-yl)-2-oxa-6-azaspiro[3.3]heptane;
3-((2-(2-Oxa-6-azaspiro[3.3]heptan-6-yl)-8-azaspiro[4.5]decan-8-yl)sulfonyl)benzonitrile;
6-(8-((4-Fluorophenyl)sulfonyl)-8-azaspiro[4.5]decan-2-yl)-2-oxa-6-azaspiro[3.3]heptane;
3-((2-(2-Oxa-6-azaspiro[3.3]heptan-6-yl)-8-azaspiro[4.5]decan-8-yl)sulfonyl)-5-fluorobenzonitrile;
8-((4-(Difluoromethoxy)phenyl)sulfonyl)-3-(piperidin-1-yl)-1-oxa-8-azaspiro[4.5]decane;
4-Methyl-3-((3-morpholino-1-oxa-8-azaspiro[4.5]decan-8-yl)sulfonyl)benzonitrile;
(R)-4-Methyl-3-((3-morpholino-1-oxa-8-azaspiro[4.5]decan-8-yl)sulfonyl)benzonitrile;
(S)-4-methyl-3-((3-morpholino-1-oxa-8-azaspiro[4.5]decan-8-yl)sulfonyl)benzonitrile;
4-Fluoro-3-((3-(piperidin-1-yl)-1-oxa-8-azaspiro[4.5]decan-8-yl)sulfonyl)benzonitrile;
8-((2-Methyl-4-(trifluoromethyl)phenyl)sulfonyl)-3-morpholino-1-oxa-8-azaspiro[4.5]decane;
8-((2-Fluoro-5-methylphenyl)sulfonyl)-3-morpholino-1-oxa-8-azaspiro[4.5]decane;
8-((2,5-Difluorophenyl)sulfonyl)-3-morpholino-1-oxa-8-azaspiro[4.5]decane;
8-((6-Methoxy-2-methylpyridin-3-yl)sulfonyl)-3-morpholino-1-oxa-8-azaspiro[4.5]decane;
4-(8-((1,3-Dimethyl-1H-pyrazol-5-yl)sulfonyl)-8-azaspiro[4.5]decan-2-yl)morpholine;
(R)-4-(8-((1,3-Dimethyl-1H-pyrazol-5-yl)sulfonyl)-8-azaspiro[4.5]decan-2-yl)morpholine;
(S)-4-(8-((1,3-dimethyl-1H-pyrazol-5-yl)sulfonyl)-8-azaspiro[4.5]decan-2-yl)morpholine;
4-(8-((4-(Difluoromethoxy)phenyl)sulfonyl)-8-azaspiro[4.5]decan-2-yl)morpholine;
2-Methyl-3-((2-morpholino-8-azaspiro[4.5]decan-8-yl)sulfonyl)-2,4,6,7-tetrahydropyrano[4,3-c]pyrazole;
4-(8-((3,5-Dimethylpyridin-2-yl)sulfonyl)-8-azaspiro[4.5]decan-2-yl)morpholine;
4-(8-((3-Methoxy-1-methyl-1H-pyrazol-5-yl)sulfonyl)-8-azaspiro[4.5]decan-2-yl)morpholine;
5-(8-((4-(Difluoromethoxy)phenyl)sulfonyl)-8-azaspiro[4.5]decan-2-yl)-2-oxa-5-azabicyclo[2.2.1]heptane;
(1R,4R)-5-(8-((4-(Difluoromethoxy)phenyl)sulfonyl)-8-azaspiro[4.5]decan-2-yl)-2-oxa-5-azabicyclo[2.2.1]heptane;
8-((4,6-Dimethylpyridin-3-yl)sulfonyl)-3-(2-oxa-7-azaspiro[4.4]nonan-7-yl)-1-oxa-8-azaspiro[4.5]decane;
8-((1,3-Dimethyl-1H-pyrazol-5-yl)sulfonyl)-3-(2-oxa-7-azaspiro[4.4]nonan-7-yl)-1-oxa-8-azaspiro[4.5]decane;
8-((4-(Difluoromethoxy)phenyl)sulfonyl)-3-(2-oxa-7-azaspiro[4.4]nonan-7-yl)-1-oxa-8-azaspiro[4.5]decane;
(S)-8-((4-(Difluoromethoxy)phenyl)sulfonyl)-3-((R)-2-oxa-7-azaspiro[4.4]nonan-7-yl)-1-oxa-8-azaspiro[4.5]decane;
(S)-8-((4-(difluoromethoxy)phenyl)sulfonyl)-3-((S)-2-oxa-7-azaspiro[4.4]nonan-7-yl)-1-oxa-8-azaspiro[4.5]decane;
(R)-8-((4-(difluoromethoxy)phenyl)sulfonyl)-3-((R)-2-oxa-7-azaspiro[4.4]nonan-7-yl)-1-oxa-8-azaspiro[4.5]decane;
(R)-8-((4-(difluoromethoxy)phenyl)sulfonyl)-3-((S)-2-oxa-7-azaspiro[4.4]nonan-7-yl)-1-oxa-8-azaspiro[4.5]decane;
4-(8-((7-fluoroquinolin-6-yl)sulfonyl)-8-azaspiro[4.5]decan-2-yl)morpholine;
4-(8-((5-Cyclopropoxypyridin-2-yl)sulfonyl)-8-azaspiro[4.5]decan-2-yl)morpholine;
4-(8-((3-Fluoro-2-methoxypyridin-4-yl)sulfonyl)-8-azaspiro[4.5]decan-2-yl)morpholine;
4-(7-((1-Ethyl-3-methyl-1H-pyrazol-5-yl)sulfonyl)-7-azaspiro[3.5]nonan-2-yl)morpholine;
4-(7-((2-Methyl-6-(trifluoromethyl)pyridin-3-yl)sulfonyl)-7-azaspiro[3.5]nonan-2-yl)morpholine;
4-(7-((2-Methoxy-5-methylpyridin-3-yl)sulfonyl)-7-azaspiro[3.5]nonan-2-yl)morpholine;
4-(7-((3-Cyclopropyl-1-methyl-1H-pyrazol-5-yl)sulfonyl)-7-azaspiro[3.5]nonan-2-yl)morpholine;
4-(8-((2-Methyl-6-(trifluoromethyl)pyridin-3-yl)sulfonyl)-8-azaspiro[bicyclo[3.2.1]octane-3,1′-cyclobutan]-3′-yl)morpholine;
7-((4-(Difluoromethoxy)phenyl)sulfonyl)-3-morpholino-1-oxa-7-azaspiro[3.5]nonane;
8-((2-Methoxy-5-methylpyridin-3-yl)sulfonyl)-N-(2-methoxyethyl)-N-methyl-8-azaspiro[4.5]decan-2-amine;
8-((2-Methoxy-5-methylpyridin-3-yl)sulfonyl)-N-(3-methyloxetan-3-yl)-8-azaspiro[4.5]decan-2-amine;
8-((2-Methoxy-5-methylpyridin-3-yl)sulfonyl)-N-(3-methyltetrahydrofuran-3-yl)-8-azaspiro[4.5]decan-2-amine;
2-(8-((2-Methoxy-5-methylpyridin-3-yl)sulfonyl)-8-azaspiro[4.5]decan-2-yl)-6-oxa-2-azaspiro[3.4]octane;
4-Fluoro-3-((3-((2-methoxyethyl)amino)-1-oxa-8-azaspiro[4.5]decan-8-yl)sulfonyl)benzonitrile;
3-((3-((1R,4R)-2-oxa-5-azabicyclo[2.2.1]heptan-5-yl)-1-oxa-8-azaspiro[4.5]decan-8-yl)sulfonyl)-4-fluorobenzonitrile;
3-((3-(2-Oxa-6-azaspiro[3.3]heptan-6-yl)-1-oxa-8-azaspiro[4.5]decan-8-yl)sulfonyl)-4-fluorobenzonitrile;
4-Fluoro-3-((3-(piperidin-1-yl)-1-oxa-8-azaspiro[4.5]decan-8-yl)sulfonyl)benzonitrile;
3-((3-((4,4-Difluorocyclohexyl)amino)-1-oxa-8-azaspiro[4.5]decan-8-yl)sulfonyl)-4-fluorobenzonitrile;
8-((2-Chloro-4-methylphenyl)sulfonyl)-3-(2-oxa-6-azaspiro[3.3]heptan-6-yl)-1-oxa-8-azaspiro[4.5]decane;
1-(8-((2-Chloro-4-methylphenyl)sulfonyl)-1-oxa-8-azaspiro[4.5]decan-3-yl)-3-methylazetidin-3-ol;
8-((2-Chloro-4-methylphenyl)sulfonyl)-N-(tetrahydro-2H-pyran-4-yl)-1-oxa-8-azaspiro[4.5]decan-3-amine;
1-(8-((2-Chloro-4-methylphenyl)sulfonyl)-1-oxa-8-azaspiro[4.5]decan-3-yl)azetidin-3-ol;
(R)-1-(8-((2-Chloro-4-methylphenyl)sulfonyl)-1-oxa-8-azaspiro[4.5]decan-3-yl)azetidin-3-ol;
(S)-1-(8-((2-chloro-4-methylphenyl)sulfonyl)-1-oxa-8-azaspiro[4.5]decan-3-yl)azetidin-3-ol;
8-((2-Methyl-4-(trifluoromethoxy)phenyl)sulfonyl)-3-morpholino-1-oxa-8-azaspiro[4.5]decane;
8-((2-Cyclopropylthiazol-5-yl)sulfonyl)-3-(3-methoxyazetidin-1-yl)-1-oxa-8-azaspiro[4.5]decane;
8-((3-Cyclopropyl-1-methyl-1H-pyrazol-5-yl)sulfonyl)-3-(2-oxa-6-azaspiro[3.3]heptan-6-yl)-1-oxa-8-azaspiro[4.5]decane;
(R)-6-(8-((1,3-dimethyl-1H-pyrazol-5-yl)sulfonyl)-8-azaspiro[4.5]decan-2-yl)-2-oxa-6-azaspiro[3.3]heptane;
(S)-6-(8-((1,3-dimethyl-1H-pyrazol-5-yl)sulfonyl)-8-azaspiro[4.5]decan-2-yl)-2-oxa-6-azaspiro[3.3]heptane;
8-((3-Cyclopropyl-1-methyl-1H-pyrazol-5-yl)sulfonyl)-3-(3-methoxyazetidin-1-yl)-1-oxa-8-azaspiro[4.5]decane;
8-((3-Cyclopropyl-1-methyl-1H-pyrazol-5-yl)sulfonyl)-N-(2-methoxyethyl)-1-oxa-8-azaspiro[4.5]decan-3-amine;
8-((3-Cyclopropyl-1-methyl-1H-pyrazol-5-yl)sulfonyl)-N-(2-methoxyethyl)-N-methyl-1-oxa-8-azaspiro[4.5]decan-3-amine;
8-((5-Chloro-2-methoxypyridin-3-yl)sulfonyl)-3-(3-methoxyazetidin-1-yl)-1-oxa-8-azaspiro[4.5]decane;
8-((5-Chloro-2-methoxypyridin-3-yl)sulfonyl)-3-(2-oxa-6-azaspiro[3.3]heptan-6-yl)-1-oxa-8-azaspiro[4.5]decane;
(R)-8-((5-Chloro-2-methoxypyridin-3-yl)sulfonyl)-3-(2-oxa-6-azaspiro[3.3]heptan-6-yl)-1-oxa-8-azaspiro[4.5]decane;
(S)-8-((5-chloro-2-methoxypyridin-3-yl)sulfonyl)-3-(2-oxa-6-azaspiro[3.3]heptan-6-yl)-1-oxa-8-azaspiro[4.5]decane;
3-((3-(2-Oxa-6-azaspiro[3.3]heptan-6-yl)-1-oxa-8-azaspiro[4.5]decan-8-yl)sulfonyl)-4-chlorobenzonitrile;
8-((2-Methoxy-5-methylpyridin-3-yl)sulfonyl)-N-(2-methoxyethyl)-1-oxa-8-azaspiro[4.5]decan-3-amine;
8-((2-Methoxy-5-methylpyridin-3-yl)sulfonyl)-N-(2-methoxyethyl)-N-(methyl-d3)-1-oxa-8-azaspiro[4.5]decan-3-amine;
8-((2-Methoxy-5-methylpyridin-3-yl)sulfonyl)-N-(2-methoxyethyl)-N-methyl-1-oxa-8-azaspiro[4.5]decan-3-amine;
6-(8-((1,3-dimethyl-1H-pyrazol-5-yl)sulfonyl)-8-azaspiro[4.5]decan-2-yl)-2-oxa-6-azaspiro[3.3]heptane;
(R)-6-(8-((1,3-dimethyl-1H-pyrazol-5-yl)sulfonyl)-8-azaspiro[4.5]decan-2-yl)-2-oxa-6-azaspiro[3.3]heptane;
(S)-6-(8-((1,3-dimethyl-1H-pyrazol-5-yl)sulfonyl)-8-azaspiro[4.5]decan-2-yl)-2-oxa-6-azaspiro[3.3]heptane;
8-((4-(Difluoromethoxy)phenyl)sulfonyl)-3-(6-oxa-2-azaspiro[3.4]octan-2-yl)-1-oxa-8-azaspiro[4.5]decane;
8-((4-(Difluoromethoxy)phenyl)sulfonyl)-3-(2-oxa-7-azaspiro[4.4]nonan-7-yl)-1-oxa-8-azaspiro[4.5]decane;
8-((4-(Difluoromethoxy)phenyl)sulfonyl)-3-((3aR,6aS)-tetrahydro-1H-furo[3,4-c]pyrrol-5(3H)-yl)-1-oxa-8-azaspiro[4.5]decane;
8-((1,3-Dimethyl-1H-pyrazol-5-yl)sulfonyl)-3-(2-oxa-7-azaspiro[4.4]nonan-7-yl)-1-oxa-8-azaspiro[4.5]decane;
(S)-8-((1,3-Dimethyl-1H-pyrazol-5-yl)sulfonyl)-3-((R)-2-oxa-7-azaspiro[4.4]nonan-7-yl)-1-oxa-8-azaspiro[4.5]decane;
(S)-8-((1,3-dimethyl-1H-pyrazol-5-yl)sulfonyl)-3-((S)-2-oxa-7-azaspiro[4.4]nonan-7-yl)-1-oxa-8-azaspiro[4.5]decane;
(R)-8-((1,3-dimethyl-1H-pyrazol-5-yl)sulfonyl)-3-((R)-2-oxa-7-azaspiro[4.4]nonan-7-yl)-1-oxa-8-azaspiro[4.5]decane;
(S)-8-((1,3-dimethyl-1H-pyrazol-5-yl)sulfonyl)-3-((S)-2-oxa-7-azaspiro[4.4]nonan-7-yl)-1-oxa-8-azaspiro[4.5]decane;
4-(7-((4-(Difluoromethoxy)phenyl)sulfonyl)-7-azaspiro[3.5]nonan-2-yl)morpholine;
4-(7-((1,3-Dimethyl-1H-pyrazol-5-yl)sulfonyl)-7-azaspiro[3.5]nonan-2-yl)morpholine;
6-(7-((6-Methoxy-2-methylpyridin-3-yl)sulfonyl)-7-azaspiro[3.5]nonan-2-yl)-2-oxa-6-azaspiro[3.3]heptane;
6-(7-((2-Methyl-6-(trifluoromethyl)pyridin-3-yl)sulfonyl)-7-azaspiro[3.5]nonan-2-yl)-2-oxa-6-azaspiro[3.3]heptane;
6-(7-((6-Chloro-2-methylpyridin-3-yl)sulfonyl)-7-azaspiro[3.5]nonan-2-yl)-2-oxa-6-azaspiro[3.3]heptane;
6-(7-((4-Methyl-6-(trifluoromethyl)pyridin-3-yl)sulfonyl)-7-azaspiro[3.5]nonan-2-yl)-2-oxa-6-azaspiro[3.3]heptane;
6-(7-((1-Methyl-3-(trifluoromethyl)-1H-pyrazol-5-yl)sulfonyl)-7-azaspiro[3.5]nonan-2-yl)-2-oxa-6-azaspiro[3.3]heptane;
6-(7-((3-Cyclopropyl-1-methyl-1H-pyrazol-5-yl)sulfonyl)-7-azaspiro[3.5]nonan-2-yl)-2-oxa-6-azaspiro[3.3]heptane;
5-(7-((6-Methoxy-2-methylpyridin-3-yl)sulfonyl)-7-azaspiro[3.5]nonan-2-yl)-2-oxa-5-azabicyclo[2.2.1]heptane;
(1R,4R)-5-(7-((6-Methoxy-2-methylpyridin-3-yl)sulfonyl)-7-azaspiro[3.5]nonan-2-yl)-2-oxa-5-azabicyclo[2.2.1]heptane;
5-(7-((1-methyl-3-(trifluoromethyl)-1H-pyrazol-5-yl)sulfonyl)-7-azaspiro[3.5]nonan-2-yl)-2-oxa-5-azabicyclo[2.2.1]heptane;
(1R,4R)-5-(7-((1-methyl-3-(trifluoromethyl)-1H-pyrazol-5-yl)sulfonyl)-7-azaspiro[3.5]nonan-2-yl)-2-oxa-5-azabicyclo[2.2.1]heptane;
7-((1-Methyl-3-(trifluoromethyl)-1H-pyrazol-5-yl)sulfonyl)-N-(tetrahydro-2H-pyran-4-yl)-7-azaspiro[3.5]nonan-2-amine;
7-((4-Methyl-6-(trifluoromethyl)pyridin-3-yl)sulfonyl)-N-(tetrahydro-2H-pyran-4-yl)-7-azaspiro[3.5]nonan-2-amine;
1-(8-((1,3-dimethyl-1H-pyrazol-5-yl)sulfonyl)-8-azaspiro[4.5]decan-2-yl)-4-methylpiperidin-4-ol;
(R)-3-((2-(2-oxa-6-azaspiro[3.3]heptan-6-yl)-8-azaspiro[4.5]decan-8-yl)sulfonyl)-5-fluorobenzonitrile;
(S)-3-((2-(2-oxa-6-azaspiro[3.3]heptan-6-yl)-8-azaspiro[4.5]decan-8-yl)sulfonyl)-5-fluorobenzonitrile;
(1R,4R)-5-((R)-8-((4-(difluoromethoxy)phenyl)sulfonyl)-8-azaspiro[4.5]decan-2-yl)-2-oxa-5-azabicyclo[2.2.1]heptane;
(1S,4S)-5-((R)-8-((1,3-dimethyl-1H-pyrazol-5-yl)sulfonyl)-8-azaspiro[4.5]decan-2-yl)-2-oxa-5-azabicyclo[2.2.1]heptane;
(1R,4R)-5-((R)-8-((1,3-dimethyl-1H-pyrazol-5-yl)sulfonyl)-8-azaspiro[4.5]decan-2-yl)-2-oxa-5-azabicyclo[2.2.1]heptane;
(1S,4S)-5-((R)-8-((4-(difluoromethoxy)phenyl)sulfonyl)-8-azaspiro[4.5]decan-2-yl)-2-oxa-5-azabicyclo[2.2.1]heptane;
(1S,4S)-5-((S)-8-((4-(difluoromethoxy)phenyl)sulfonyl)-8-azaspiro[4.5]decan-2-yl)-2-oxa-5-azabicyclo[2.2.1]heptane;
(1S,4S)-5-((R)-8-((4-(difluoromethoxy)phenyl)sulfonyl)-8-azaspiro[4.5]decan-2-yl)-2-oxa-5-azabicyclo[2.2.1]heptane;
7-((1,3-dimethyl-1H-pyrazol-5-yl)sulfonyl)-N-(2-methoxyethyl)-N-methyl-7-azaspiro[3.5]nonan-2-amine;
6-(7-((1,3-dimethyl-1H-pyrazol-5-yl)sulfonyl)-7-azaspiro[3.5]nonan-2-yl)-2-oxa-6-azaspiro[3.3]heptane;
6-(7-((2-methoxy-5-methylpyridin-3-yl)sulfonyl)-7-azaspiro[3.5]nonan-2-yl)-2-oxa-6-azaspiro[3.3]heptane;
(1R,4R)-5-(7-((6-chloro-2-methylpyridin-3-yl)sulfonyl)-7-azaspiro[3.5]nonan-2-yl)-2-oxa-5-azabicyclo[2.2.1]heptane;
1-(7-((2-methyl-6-(trifluoromethyl)pyridin-3-yl)sulfonyl)-7-azaspiro[3.5]nonan-2-yl)azetidin-3-ol;
3-((2-(2-oxa-6-azaspiro[3.3]heptan-6-yl)-7-azaspiro[3.5]nonan-7-yl)sulfonyl)-5-fluorobenzonitrile;
3-(((S)-2-((1R,4R)-2-oxa-5-azabicyclo[2.2.1]heptan-5-yl)-8-azaspiro[4.5]decan-8-yl)sulfonyl)-5-fluorobenzonitrile;
3-fluoro-5-((3-(((tetrahydro-2H-pyran-4-yl)methyl)amino)-1-oxa-8-azaspiro[4.5]decan-8-yl)sulfonyl)benzonitrile;
1-(8-((2-methoxy-6-methyl-2H-112-pyridin-5-yl)sulfonyl)-8-azaspiro[4.5]decan-2-yl)azetidin-3-ol;
8-((2-methoxy-6-methyl-2H-112-pyridin-5-yl)sulfonyl)-N-(2-methoxyethyl)-N-methyl-8-azaspiro[4.5]decan-2-amine;
6-(8-((5-(trifluoromethyl)furan-3-yl)sulfonyl)-8-azaspiro[4.5]decan-2-yl)-2-oxa-6-azaspiro[3.3]heptane;
6-(8-((7-fluoroquinolin-6-yl)sulfonyl)-8-azaspiro[4.5]decan-2-yl)-2-oxa-6-azaspiro[3.3]heptane;
6-(8-(quinolin-3-ylsulfonyl)-8-azaspiro[4.5]decan-2-yl)-2-oxa-6-azaspiro[3.3]heptane;
3-((2-(2-oxa-6-azaspiro[3.3]heptan-6-yl)-8-azaspiro[4.5]decan-8-yl)sulfonyl)-2-fluorobenzonitrile;
6-(8-((2-methylfuran-3-yl)sulfonyl)-8-azaspiro[4.5]decan-2-yl)-2-oxa-6-azaspiro[3.3]heptane;
6-(8-((5-chloro-6-methoxypyridin-3-yl)sulfonyl)-8-azaspiro[4.5]decan-2-yl)-2-oxa-6-azaspiro[3.3]heptane;
6-(8-((2,3-dihydrobenzo[b][1,4]dioxin-6-yl)sulfonyl)-8-azaspiro[4.5]decan-2-yl)-2-oxa-6-azaspiro[3.3]heptane;
6-(8-((4-ethoxyphenyl)sulfonyl)-8-azaspiro[4.5]decan-2-yl)-2-oxa-6-azaspiro[3.3]heptane;
6-(8-((5,6-dimethylpyridin-3-yl)sulfonyl)-8-azaspiro[4.5]decan-2-yl)-2-oxa-6-azaspiro[3.3]heptane;
6-(8-((6-methoxypyridin-3-yl)sulfonyl)-8-azaspiro[4.5]decan-2-yl)-2-oxa-6-azaspiro[3.3]heptane;
6-(8-((2-ethyl-2H-1,2,3-triazol-4-yl)sulfonyl)-8-azaspiro[4.5]decan-2-yl)-2-oxa-6-azaspiro[3.3]heptane;
6-(8-((2-methoxypyridin-4-yl)sulfonyl)-8-azaspiro[4.5]decan-2-yl)-2-oxa-6-azaspiro[3.3]heptane;
6-(8-((5-fluoro-2-methoxypyridin-4-yl)sulfonyl)-8-azaspiro[4.5]decan-2-yl)-2-oxa-6-azaspiro[3.3]heptane;
8-((5-chloro-6-methoxypyridin-3-yl)sulfonyl)-3-(2-oxa-6-azaspiro[3.3]heptan-6-yl)-1-oxa-8-azaspiro[4.5]decane;
6-(8-((5-(trifluoromethoxy)pyridin-2-yl)sulfonyl)-8-azaspiro[4.5]decan-2-yl)-2-oxa-6-azaspiro[3.3]heptane;
8-((5-fluoro-2-methoxypyridin-4-yl)sulfonyl)-3-(2-oxa-6-azaspiro[3.3]heptan-6-yl)-1-oxa-8-azaspiro[4.5]decane;
8-((2,4-dimethylphenyl)sulfonyl)-N-(2-methoxyethyl)-1-oxa-8-azaspiro[4.5]decan-3-amine;
8-((2-methoxy-5-methylpyridin-3-yl)sulfonyl)-3-(2-oxa-6-azaspiro[3.3]heptan-6-yl)-1-oxa-8-azaspiro[4.5]decane;
Rac-8-((3,5-difluorophenyl)sulfonyl)-3-(2-oxa-6-azaspiro[3.3]heptan-6-yl)-1-oxa-8-azaspiro[4.5]decane;
(R)-8-((3,5-difluorophenyl)sulfonyl)-3-(2-oxa-6-azaspiro[3.3]heptan-6-yl)-1-oxa-8-azaspiro[4.5]decane;
(S)-8-((3,5-difluorophenyl)sulfonyl)-3-(2-oxa-6-azaspiro[3.3]heptan-6-yl)-1-oxa-8-azaspiro[4.5]decane;
7-((3-cyclopropyl-1-methyl-1H-pyrazol-5-yl)sulfonyl)-N-(tetrahydro-2H-pyran-4-yl)-7-azaspiro[3.5]nonan-2-amine;
6-(7-((6-(difluoromethoxy)-2-methyl-2,3,4,5-tetrahydropyridin-3-yl)sulfonyl)-7-azaspiro[3.5]nonan-2-yl)-2-oxa-6-azaspiro[3.3]heptane;
6-(7-((3-(difluoromethyl)-1-methyl-1H-pyrazol-5-yl)sulfonyl)-7-azaspiro[3.5]nonan-2-yl)-2-oxa-6-azaspiro[3.3]heptane;
6-(7-((1-cyclopropyl-3-(difluoromethyl)-1H-pyrazol-5-yl)sulfonyl)-7-azaspiro[3.5]nonan-2-yl)-2-oxa-6-azaspiro[3.3]heptane;
6-(8-((3-methoxy-1-methyl-1H-pyrazol-5-yl)sulfonyl)-8-azaspiro[4.5]decan-2-yl)-2-oxa-6-azaspiro[3.3]heptane;
6-(8-((3-(difluoromethyl)-1-methyl-1H-pyrazol-5-yl)sulfonyl)-8-azaspiro[4.5]decan-2-yl)-2-oxa-6-azaspiro[3.3]heptane;
4-((2-(2-oxa-6-azaspiro[3.3]heptan-6-yl)-8-azaspiro[4.5]decan-8-yl)sulfonyl)-3-chlorobenzonitrile;
5-((2-(2-oxa-6-azaspiro[3.3]heptan-6-yl)-8-azaspiro[4.5]decan-8-yl)sulfonyl)-2-fluorobenzonitrile;
5-((2-(2-oxa-6-azaspiro[3.3]heptan-6-yl)-8-azaspiro[4.5]decan-8-yl)sulfonyl)-2-methylbenzonitrile;
6-(8-((2-fluoro-4-methoxyphenyl)sulfonyl)-8-azaspiro[4.5]decan-2-yl)-2-oxa-6-azaspiro[3.3]heptane;
6-(8-((6-isopropoxypyridin-3-yl)sulfonyl)-8-azaspiro[4.5]decan-2-yl)-2-oxa-6-azaspiro[3.3]heptane;
6-(8-(pyrazolo[1,5-a]pyridin-6-ylsulfonyl)-8-azaspiro[4.5]decan-2-yl)-2-oxa-6-azaspiro[3.3]heptane;
6-(8-((5-fluoroquinolin-3-yl)sulfonyl)-8-azaspiro[4.5]decan-2-yl)-2-oxa-6-azaspiro[3.3]heptane;
6-(8-((3-fluoroquinolin-6-yl)sulfonyl)-8-azaspiro[4.5]decan-2-yl)-2-oxa-6-azaspiro[3.3]heptane;
6-(8-((4-cyclopropoxyphenyl)sulfonyl)-8-azaspiro[4.5]decan-2-yl)-2-oxa-6-azaspiro[3.3]heptane;
6-(8-(benzofuran-6-ylsulfonyl)-8-azaspiro[4.5]decan-2-yl)-2-oxa-6-azaspiro[3.3]heptane;
8-((2-fluoro-4-methoxyphenyl)sulfonyl)-N-methyl-N-(2-oxaspiro[3.3]heptan-6-yl)-1-oxa-8-azaspiro[4.5]decan-3-amine;
8-((2-methyl-6-(trifluoromethyl)pyridin-3-yl)sulfonyl)-N-(2-oxaspiro[3.3]heptan-6-yl)-1-oxa-8-azaspiro[4.5]decan-3-amine;
8-((2-methyl-6-(trifluoromethyl)pyridin-3-yl)sulfonyl)-N-(tetrahydro-2H-pyran-4-yl)-1-oxa-8-azaspiro[4.5]decan-3-amine;
8-((1-methyl-3-(trifluoromethyl)-1H-pyrazol-5-yl)sulfonyl)-N-(2-oxaspiro[3.3]heptan-6-yl)-1-oxa-8-azaspiro[4.5]decan-3-amine;
N-methyl-8-((4-methyl-2-(trifluoromethyl)pyrimidin-5-yl)sulfonyl)-N-(2-oxaspiro[3.3]heptan-6-yl)-1-oxa-8-azaspiro[4.5]decan-3-amine;
(S)-4-fluoro-3-((3-(4-hydroxy-4-methylpiperidin-1-yl)-1-oxa-8-azaspiro[4.5]decan-8-yl)sulfonyl)benzonitrile;
6-(8-((1,3-dimethyl-H-pyrazol-5-yl)sulfonyl)-8-azaspiro[4.5]decan-2-yl)-2-oxa-6-azaspiro[3.3]heptane;
8-((4-(difluoromethoxy)phenyl)sulfonyl)-3-(6-oxa-2-azaspiro[3.5]nonan-2-yl)-1-oxa-8-azaspiro[4.5]decane; and
6-(8-(quinolin-7-ylsulfonyl)-8-azaspiro[4.5]decan-2-yl)-2-oxa-6-azaspiro[3.3]heptane;
   or a or a pharmaceutically acceptable salt thereof.

In some embodiments, the compounds of Table I or pharmaceutically acceptable salts thereof are excluded from the compounds of the present disclosure (e.g., compounds of Formula (I)).

TABLE I
N,N-dimethyl-7-(o-tolylsulfonyl)-7-azaspiro[3.5]nonan-2-amine
N,N-dimethyl-7-((3-methylthiophen-2-yl)sulfonyl)-7-azaspiro[3.5]nonan-2-amine
N,N-dimethyl-7-((1-methyl-1H-imidazol-4-yl)sulfonyl)-7-azaspiro[3.5]nonan-2-amine
4-(7-(o-tolylsulfonyl)-7-azaspiro[3.5]nonan-1-yl)morpholine
4-(7-((1,3,5-trimethyl-1H-pyrazol-4-yl)sulfonyl)-7-azaspiro[3.5]nonan-1-yl)morpholine
4-(7-((3,5-dimethylisoxazol-4-yl)sulfonyl)-7-azaspiro[3.5]nonan-1-yl)morpholine
4-(7-((2,5-dimethoxyphenyl)sulfonyl)-7-azaspiro[3.5]nonan-1-yl)morpholine
4-(7-((1H-3l2-benzo[c][1,2,5]thiadiazol-4-yl)sulfonyl)-7-azaspiro[3.5]nonan-1-
yl)morpholine
4-(7-((3-methoxyphenyl)sulfonyl)-7-azaspiro[3.5]nonan-1-yl)morpholine
4-(7-((2,3-dihydro-1H-inden-5-yl)sulfonyl)-7-azaspiro[3.5]nonan-1-yl)morpholine
4-(7-(thiophen-2-ylsulfonyl)-7-azaspiro[3.5]nonan-1-yl)morpholine
4-(7-((1-ethyl-1H-pyrazol-4-yl)sulfonyl)-7-azaspiro[3.5]nonan-1-yl)morpholine
N,N-dimethyl-8-(m-tolylsulfonyl)-1-oxa-8-azaspiro[4.5]decan-3-amine
8-((4-fluorophenyl)sulfonyl)-N,N-dimethyl-1-oxa-8-azaspiro[4.5]decan-3-amine
N,N-dimethyl-8-((1-methyl-1H-imidazol-4-yl)sulfonyl)-1-oxa-8-azaspiro[4.5]decan-3-
amine
4-((3-(ethyl(methyl)amino)-1-oxa-8-azaspiro[4.5]decan-8-yl)sulfonyl)benzonitrile
N-ethyl-8-((4-isopropylphenyl)sulfonyl)-N-methyl-1-oxa-8-azaspiro[4.5]decan-3-amine
N-ethyl-8-((4-ethylphenyl)sulfonyl)-N-methyl-1-oxa-8-azaspiro[4.5]decan-3-amine
8-((3-methylthiophen-2-yl)sulfonyl)-3-(pyrrolidin-1-yl)-1-oxa-8-azaspiro[4.5]decane
8-((1-methyl-1H-imidazol-4-yl)sulfonyl)-3-(pyrrolidin-1-yl)-1-oxa-8-azaspiro[4.5]decane
3-(4-methylpiperazin-1-yl)-8-(m-tolylsulfonyl)-1-oxa-8-azaspiro[4.5]decane
8-((4-isopropylphenyl)sulfonyl)-3-(4-methylpiperazin-1-yl)-1-oxa-8-azaspiro[4.5]decane
8-((3-methylthiophen-2-yl)sulfonyl)-3-morpholino-1-oxa-8-azaspiro[4.5]decane
3-morpholino-8-tosyl-1-oxa-8-azaspiro[4.5]decane

In a seventieth embodiment, the present disclosure provides a pharmaceutical composition comprising a compound of the present disclosure (e.g., according to any one of the preceding embodiments), or a pharmaceutically acceptable salt thereof.

In a seventy-first embodiment, the present disclosure provides a method of treating a disease or disorder mediated by EBP comprising administering to a subject an effective amount of a compound of the present disclosure (e.g., a compound of Formula (I) according to any one of embodiments one to sixty-nine), or a pharmaceutically acceptable salt thereof, or the pharmaceutical composition of the fifty-fifth embodiment.

In a seventy-second embodiment, the present disclosure provides a compound of the present disclosure (e.g., a compound of Formula (I) according to any one of embodiments one to sixty-nine), or a pharmaceutically acceptable salt thereof, for use in the treatment of a disease or disorder mediated by EBP.

In a seventy-third embodiment, the present disclosure provides the use of a compound of the present disclosure (e.g., a compound of Formula (I) according to any one of embodiments one to sixty-nine), or a pharmaceutically acceptable salt thereof, in the manufacture of a medicament for the treatment of a disease or disorder mediated by EBP.

In some embodiments, the compound can be used in the methods and uses disclosed herein is any one of the compounds in Table I or a pharmaceutically acceptable salt thereof.

The compounds and intermediates described herein may be isolated and used as the compound per se. Alternatively, when a moiety is present that is capable of forming a salt, the compound or intermediate may be isolated and used as its corresponding salt. As used herein, the terms “salt” or “salts” refers to an acid addition or base addition salt of a compound described herein. “Salts” include in particular “pharmaceutical acceptable salts”. The term “pharmaceutically acceptable salts” refers to salts that retain the biological effectiveness and properties of the compounds described herein and, which typically are not biologically or otherwise undesirable. In many cases, the compounds of the present disclosure are capable of forming acid and/or base salts by virtue of the presence of amino and/or carboxyl groups or groups similar thereto.

Pharmaceutically acceptable acid addition salts can be formed with inorganic acids or organic acids, e.g., acetate, aspartate, benzoate, besylate, bromide/hydrobromide, bicarbonate/carbonate, bisulfate/sulfate, camphorsulfornate, chloride/hydrochloride, chlortheophyllonate, citrate, ethandisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, sulfate, sulfosalicylate, tartrate, tosylate and trifluoroacetate salts.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

The salts can be synthesized by conventional chemical methods from a compound containing a basic or acidic moiety. Generally, such salts can be prepared by reacting free acid forms of these compounds with a stoichiometric amount of the appropriate base (such as Na, Ca, Mg, or K hydroxide, carbonate, bicarbonate or the like), or by reacting free base forms of these compounds with a stoichiometric amount of the appropriate acid. Such reactions are typically carried out in water or in an organic solvent, or in a mixture of the two. Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

Isotopically-labeled compounds of Formula (I) can generally be prepared by conventional techniques known to those skilled in the art or by processes analogous to those described in the accompanying Examples and Preparations using an appropriate isotopically-labeled reagents in place of the non-labeled reagent previously employed. In one embodiment, the present disclosure provides deuterated compounds described herein or a pharmaceutically acceptable salt thereof.

Pharmaceutically acceptable solvates in accordance with the invention include those wherein the solvent of crystallization may be isotopically substituted, e.g. D₂O, d₆-acetone, d₆-DMSO.

It will be recognized by those skilled in the art that the compounds of the present invention may contain chiral centers and as such may exist in different stereoisomeric forms. As used herein, the term “an optical isomer” or “a stereoisomer” refers to any of the various stereo isomeric configurations which may exist for a given compound of the present disclosure.

It is understood that a substituent may be attached at a chiral center of a carbon atom. Therefore, the disclosure includes enantiomers, diastereomers or racemates of the compound.

“Enantiomers” are a pair of stereoisomers that are non-superimposable mirror images of each other. A 1:1 mixture of a pair of enantiomers is a “racemic” mixture. The term “racemic” or “rac” is used to designate a racemic mixture where appropriate. When designating the stereochemistry for the compounds of the present invention, a single stereoisomer with known relative and absolute configuration of the two chiral centers is designated using the conventional RS system (e.g., (1S,2S)). “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Alternatively, the resolved compounds can be defined by the respective retention times for the corresponding enantiomers/diastereomers via chiral HPLC.

Certain of the compounds described herein contain one or more asymmetric centers or axes and may thus give rise to enantiomers, diastereomers, and other stereoisomeric forms that may be defined, in terms of absolute stereochemistry, as (R)- or (S)-.

Unless specified otherwise, the compounds of the present disclosure are meant to include all such possible stereoisomers, including racemic mixtures, optically pure forms and intermediate mixtures. Optically active (R)- and (S)-stereoisomers may be prepared using chiral synthons or chiral reagents, or resolved using conventional techniques (e.g., separated on chiral SFC or HPLC chromatography columns, such as CHIRALPAK™ and CHIRALCEL™ available from DAICEL Corp. using the appropriate solvent or mixture of solvents to achieve good separation). If the compound contains a double bond, the substituent may be E or Z configuration. If the compound contains a disubstituted cycloalkyl, the cycloalkyl substituent may have a cis- or trans-configuration. All tautomeric forms are also intended to be included.

### Methods of Use

The compounds disclosed herein have EBP inhibitory activity. As used herein, “EBP inhibitory activity” refers to the ability of a compound or composition to induce a detectable decrease in EBP activity in vivo or in vitro (e.g., at least 10% decrease in EBP activity as measured by a given assay such as the bioassay described in the examples and known in the art).

In certain embodiments, the present disclosure provides a method of treating a disease or disorder responsive to inhibition of EBP activity (referred herein as “EBP mediated disease or disorder” or “disease or disorder mediated by EBP”) in a subject in need of the treatment.

The method comprises administering to the subject a compound described herein (e.g., a compound described in any one of the first to sixty-ninth embodiments) or a pharmaceutically acceptable salt thereof or a pharmaceutical composition thereof.

In certain embodiments, the present disclosure provides the use of a compound described herein (e.g., a compound described in any one of the first to sixty-ninth embodiments) or a pharmaceutically acceptable salt thereof or a pharmaceutical composition comprising a compound described herein or a pharmaceutically acceptable salt thereof for the manufacture of a medicament for the treatment of a EBP mediated disorder or disease in a subject in need of the treatment.

In certain embodiments, the present disclosure provides a compound described herein (e.g., a compound described in any one of the first to sixty-ninth embodiments) or a pharmaceutically acceptable salt thereof or a pharmaceutical composition comprising a compound described herein or a pharmaceutically acceptable salt thereof for use in the treatment of a EBP mediated disorder or disease in a subject in need of the treatment.

In certain embodiments, the EBP mediated disorder is colorectal cancer.

In certain embodiments, the present disclosure provides a method of treating an autoimmune disease in a subject in need of the treatment. The method comprises administering to the subject a compound described herein (e.g., a compound described in any one of the first to sixty-ninth embodiments) or a pharmaceutically acceptable salt thereof or a pharmaceutical composition thereof.

In certain embodiments, the present disclosure provides the use of a compound described herein (e.g., a compound described in any one of the first to sixty-ninth embodiments) or a pharmaceutically acceptable salt thereof or a pharmaceutical composition comprising a compound described herein or a pharmaceutically acceptable salt thereof for the manufacture of a medicament for the treatment of an autoimmune disease in a subject in need of the treatment.

In certain embodiments, the present disclosure provides a compound described herein (e.g., a compound described in any one of the first to sixty-ninth embodiments) or a pharmaceutically acceptable salt thereof or a pharmaceutical composition comprising a compound described herein or a pharmaceutically acceptable salt thereof for use in the treatment of an autoimmune disease in a subject in need of the treatment.

In certain embodiments, the autoimmune disease is multiple sclerosis (MS). The compounds of the present disclosure can be used for treating all stages of MS, including relapsing multiple sclerosis (or relapsing form(s) of multiple sclerosis), relapsing-remitting multiple sclerosis, primary progress multiple sclerosis, secondary progressive multiple sclerosis and clinically isolated syndrome (hereinafter “CIS”).

Relapsing multiple sclerosis (or relapsing form(s) of multiple sclerosis) includes clinically isolated syndrome, relapsing-remitting multiple sclerosis and active secondary progressive multiple sclerosis.

Relapsing-remitting multiple sclerosis is a stage of MS characterized by unpredictable relapses followed by periods of months to years of relative quiet (remission) with no new signs of disease activity. Deficits that occur during attacks may either resolve or leave problems, the latter in about 40% of attacks and being more common the longer a person has had the disease. This describes the initial course of 80% of individuals with multiple sclerosis.

Secondary progressive multiple sclerosis occurs in around 65% of those with initial relapsing-remitting multiple sclerosis, who eventually have progressive neurologic decline between acute attacks without any definite periods of remission. Occasional relapses and minor remissions may appear. The most common length of time between disease onset and conversion from relapsing-remitting to secondary progressive multiple sclerosis is 19 years.

Primary progressive multiple sclerosis is characterized by the same symptoms of secondary progressive multiple sclerosis, i.e., progressive neurologic decline between acute attacks without any definite periods of remission, without the prior relapsing-remitting stage.

CIS is a first episode of neurologic symptoms caused by inflammation and demyelination in the central nervous system. The episode, which by definition must last for at least 24 hours, is characteristic of multiple sclerosis but does not yet meet the criteria for a diagnosis of MS because people who experience a CIS may or may not go on to develop MS. When CIS is accompanied by lesions on a brain MRI (magnetic resonance imaging) that are similar to those seen in MS, the person has a high likelihood of a second episode of neurologic symptoms and diagnosis of relapsing-remitting MS. When CIS is not accompanied by MS-like lesions on a brain MRI, the person has a much lower likelihood of developing MS.

In certain embodiments, the present disclosure provides a method of promoting myelination in a subject with a myelin-related disease or disorder in a subject in need of the treatment. The method comprises administering to the subject a compound described herein (e.g., a compound described in any one of the first to sixty-ninth embodiments) or a pharmaceutically acceptable salt thereof or a pharmaceutical composition thereof.

In certain embodiments, the present disclosure provides the use of a compound described herein (e.g., a compound described in any one of the first to sixty-ninth embodiments) or a pharmaceutically acceptable salt thereof or a pharmaceutical composition comprising a compound described herein or a pharmaceutically acceptable salt thereof for the manufacture of a medicament for promoting myelination in a subject with a myelin-related disease or disorder in a subject in need of the treatment.

In certain embodiments, the present disclosure provides a compound described herein (e.g., a compound described in any one of the first to sixty-ninth embodiments) or a pharmaceutically acceptable salt thereof or a pharmaceutical composition comprising a compound described herein or a pharmaceutically acceptable salt thereof for use in promoting myelination in a subject with a myelin-related disease or disorder in a subject in need of the treatment.

In certain embodiments, the myelin-related disease or disorder is selected from multiple sclerosis (MS), neuromyelitis optica (NMO), optic neuritis, pediatric leukodystrophies, neonatal white matter injury, age-related dementia, schizophrenia, progressive multifocal leukoencephalopathy (PML), encephalomyelitis (EPL), acute disseminated encephalomyelitis (ADEM), central pontine myelolysis (CPM), adrenoleukodystrophy, Alexander's disease, Pelizaeus Merzbacher disease (PMD), Vanishing White Matter Disease, Wallerian Degeneration, transverse myelitis, amylotrophic lateral sclerosis (ALS), Huntington's disease, Alzheimer's disease, Parkinson's disease, spinal cord injury, traumatic brain injury, post radiation injury, neurologic complications of chemotherapy, stroke, acute ischemic optic neuropathy, vitamin E deficiency, isolated vitamin E deficiency syndrome, Bassen-Kornzweig syndrome, Marchiafava-Bignami syndrome, autism, metachromatic leukodystrophy, trigeminal neuralgia, acute disseminated encephalitis, chronic inflammatory demyelinating polyneuropathy, Guillian-Barre syndrome, Charcot-Marie-Tooth disease, Bell's palsy and radiation-induced demyelination, for example, neuromyelitis optica (NMO), optic neuritis, pediatric leukodystrophies, neonatal white matter injury, age-related dementia, and schizophrenia.

In certain embodiments, the present disclosure provides a method of treating cancer in a subject in need of the treatment. The method comprises administering to the subject a compound described herein (e.g., a compound described in any one of the first to sixty-ninth embodiments) or a pharmaceutically acceptable salt thereof or a pharmaceutical composition thereof.

In certain embodiments, the present disclosure provides the use of a compound described herein (e.g., a compound described in any one of the first to sixty-ninth embodiments) or a pharmaceutically acceptable salt thereof or a pharmaceutical composition comprising a compound described herein or a pharmaceutically acceptable salt thereof for the manufacture of a medicament for the treatment of cancer in a subject in need of the treatment.

In certain embodiments, the present disclosure provides a compound described herein (e.g., a compound described in any one of the first to sixty-ninth embodiments) or a pharmaceutically acceptable salt thereof or a pharmaceutical composition comprising a compound described herein or a pharmaceutically acceptable salt thereof for use in treating cancer in a subject in need of the treatment.

In certain embodiments, the cancer is colorectal cancer.

In certain embodiments, the present disclosure relates to the aforementioned methods, wherein said subject is a mammal. In certain embodiments, the subject is a primate. In certain embodiments, the subject is a human.

As used herein, an “effective amount” and a “therapeutically effective amount” can used interchangeably. It means an amount effective for treating or lessening the severity of one or more of the diseases, disorders or conditions as recited herein. In some embodiments, the effective dose can be between 10 g and 500 mg.

The compounds and compositions, according to the methods of the present disclosure, may be administered using any amount and any route of administration effective for treating or lessening the severity of one or more of the diseases, disorders or conditions recited above.

In certain embodiments, the present disclosure relates to the aforementioned methods, wherein said compound is administered parenterally. In certain embodiments, the present disclosure relates to the aforementioned methods, wherein said compound is administered intramuscularly, intravenously, subcutaneously, orally, pulmonary, rectally, intrathecally, topically or intranasally. In certain embodiments, the present disclosure relates to the aforementioned methods, wherein said compound is administered systemically.

The compounds of the present invention can be used as a pharmaceutical composition (e.g., a compound of the present invention and at least one pharmaceutically acceptable carrier). As used herein, the term “pharmaceutically acceptable carrier” includes generally recognized as safe (GRAS) solvents, dispersion media, surfactants, antioxidants, preservatives (e.g., antibacterial agents, antifungal agents), isotonic agents, salts, preservatives, drug stabilizers, buffering agents (e.g., maleic acid, tartaric acid, lactic acid, citric acid, acetic acid, sodium bicarbonate, sodium phosphate, and the like), and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated. For purposes of this disclosure, solvates and hydrates are considered pharmaceutical compositions comprising a compound of the present invention and a solvent (i.e., solvate) or water (i.e., hydrate).

The formulations may be prepared using conventional dissolution and mixing procedures. For example, the bulk drug substance (i.e., compound of the present invention or stabilized form of the compound (e.g., complex with a cyclodextrin derivative or other known complexation agent)) is dissolved in a suitable solvent in the presence of one or more of the excipients described above. The compound of the present invention is typically formulated into pharmaceutical dosage forms to provide an easily controllable dosage of the drug and to give the patient an elegant and easily handleable product.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

The pharmaceutical composition comprising a compound of the present disclosure is generally formulated for use as a parenteral or oral administration or alternatively suppositories.

For example, the pharmaceutical oral compositions of the present disclosure can be made up in a solid form (including without limitation capsules, tablets, pills, granules, powders or suppositories), or in a liquid form (including without limitation solutions, suspensions or emulsions). The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifiers and buffers, etc.

Typically, the pharmaceutical compositions are tablets or gelatin capsules comprising the active ingredient together with
a) diluents, e.g., lactose, dextrose, sucrose, mannitol, sorbitol, cellulose and/or glycine;
b) lubricants, e.g., silica, talcum, stearic acid, its magnesium or calcium salt and/or polyethylene glycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and/or
e) absorbents, colorants, flavors and sweeteners.

Tablets may be either film coated or enteric coated according to methods known in the art.

Suitable compositions for oral administration include a compound of the disclosure in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

The parenteral compositions (e.g, intravenous (IV) formulation) are aqueous isotonic solutions or suspensions. The parenteral compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. The compositions are generally prepared according to conventional mixing, granulating or coating methods, respectively, and contain about 0.1-75%, or contain about 1-50%, of the active ingredient.

The compound of the present disclosure or pharmaceutical composition thereof for use in a subject (e.g., human) is typically administered orally or parenterally at a therapeutic dose. When administered intravenously via infusion, the dosage may depend upon the infusion rate at which an IV formulation is administered. In general, the therapeutically effective dosage of a compound, the pharmaceutical composition, or the combinations thereof, is dependent on the species of the subject, the body weight, age and individual condition, the disorder or disease or the severity thereof being treated. A physician, pharmacist, clinician or veterinarian of ordinary skill can readily determine the effective amount of each of the active ingredients necessary to prevent, treat or inhibit the progress of the disorder or disease.

The above-cited dosage properties are demonstrable in vitro and in vivo tests using advantageously mammals, e.g., mice, rats, dogs, monkeys or isolated organs, tissues and preparations thereof. The compounds of the present invention can be applied in vitro in the form of solutions, e.g., aqueous solutions, and in vivo either enterally, parenterally, advantageously intravenously, e.g., as a suspension or in aqueous solution. The dosage in vitro may range between about 10-3 molar and 10-9 molar concentrations.

### Definitions

As used herein, a “patient,” “subject” or “individual” are used interchangeably and refer to either a human or non-human animal. The term includes mammals such as humans. Typically, the animal is a mammal. A subject also refers to for example, primates (e.g., humans, male or female), cows, sheep, goats, horses, dogs, cats, rabbits, rats, mice, fish, birds and the like. In certain embodiments, the subject is a primate. Preferably, the subject is a human.

As used herein, the term “inhibit”, “inhibition” or “inhibiting” refers to the reduction or suppression of a given condition, symptom, or disorder, or disease, or a significant decrease in the baseline activity of a biological activity or process.

As used herein, the term “treat”, “treating” or “treatment” of any disease, condition or disorder, refers to the management and care of a patient for the purpose of combating the disease, condition, or disorder and includes the administration of a compound of the present invention to obtaining desired pharmacological and/or physiological effect. The effect can be therapeutic, which includes achieving, partially or substantially, one or more of the following results: partially or totally reducing the extent of the disease, condition or disorder; ameliorating or improving a clinical symptom, complications or indicator associated with the disease, condition or disorder; or delaying, inhibiting or decreasing the likelihood of the progression of the disease, condition or disorder; or eliminating the disease, condition or disorder. In certain embodiments, the effect can be to prevent the onset of the symptoms or complications of the disease, condition or disorder.

As used herein, the term “cancer” has the meaning normally accepted in the art. The term can broadly refer to abnormal cell growth.

As used herein, the term “autoimmune disease” has the meaning normally accepted the art. The term can broadly refer to a disease where the host's immune system targets or attacks normal or healthy tissue of the host.

As used herein, the term “myelination” has the meaning normally accepted in the art. The term can broadly mean the process by which myelin is produced.

As used herein, the term “myelin-related disease or disorder”, “demyelinating disorder”, or “demyelation disorder” has the meaning normally accepted in the art. These terms can broadly refer to diseases or disorders which involve damage to myelin.

As used herein, a subject is “in need of” a treatment if such subject would benefit biologically, medically or in quality of life from such treatment (preferably, a human).

As used herein, the phrase “optionally substituted” is used interchangeably with the phrase “substituted or unsubstituted.” In general the term “optionally substituted” refers to the replacement of hydrogen radicals in a given structure with the radical of a specified substituent.

Specific substituents are described in the definitions and in the description of compounds and examples thereof. Unless otherwise indicated, an optionally substituted group can have a substituent at each substitutable position of the group, and when more than one position in any given structure can be substituted with more than one substituent selected from a specified group, the substituent can be either the same or different at every position.

As used herein, the term “alkyl” refers to a fully saturated branched or unbranched hydrocarbon moiety. The term “C₁₋₄alkyl” refers to an alkyl having 1 to 4 carbon atoms. The terms “C₁₋₃alkyl” and “C₁₋₂alkyl” are to be construed accordingly. Representative examples of “C₁₋₄alkyl” include, but are not limited to, methyl, ethyl, n-propyl, iso-propyl, n-butyl, sec-butyl, iso-butyl, and tert-butyl. Similarly, the alkyl portion (i.e., alkyl moiety) of an alkoxy have the same definition as above. When indicated as being “optionally substituted”, the alkane radical or alkyl moiety may be unsubstituted or substituted with one or more substituents (generally, one to three substituents except in the case of halogen substituents such as perchloro or perfluoroalkyls).

As used herein, the term “alkoxy” refers to a fully saturated branched or unbranched alkyl moiety attached through an oxygen bridge (i.e. a —O—C₁₋₄ alkyl group wherein C₁₋₄ alkyl is as defined herein). Representative examples of alkoxy include, but are not limited to, methoxy, ethoxy, propoxy, 2-propoxy, butoxy, tert-butoxy and the like. Preferably, alkoxy groups have about 1-4 carbons, more preferably about 1-2 carbons. The term “C₁₋₂ alkoxy” is to be construed accordingly.

As used herein, the term “C₁₋₄ alkoxyC₁₋₄ alkyl” refers to a C₁₋₄ alkyl group as defined herein, wherein at least of the hydrogen atoms is replaced by an C₁₋₄ alkoxy. The C₁₋₄alkoxyC₁₋₄ alkyl group is connected through the rest of the molecule described herein through the alkyl group.

The number of carbon atoms in a group is specified herein by the prefix “Cx-xx”, wherein x and xx are integers. For example, “C₁₋₃ alkyl” is an alkyl group which has from 1 to 3 carbon atoms.

“Halogen” or “halo” may be fluorine, chlorine, bromine or iodine.

As used herein, the term “halo-substituted-C₁₋₄alkyl” or “C₁₋₄haloalkyl” refers to a C₁₋₄ alkyl group as defined herein, wherein at least one of the hydrogen atoms is replaced by a halo atom. The C₁₋₄haloalkyl group can be monohalo-C₁₋₄alkyl, dihalo-C₁₋₄alkyl or polyhalo-C₁₋₄ alkyl including perhalo-C₁₋₄alkyl. A monohalo-C₁₋₄alkyl can have one iodo, bromo, chloro or fluoro within the alkyl group. Dihalo-C₁₋₄alkyl and polyhalo-C₁₋₄alkyl groups can have two or more of the same halo atoms or a combination of different halo groups within the alkyl. Typically the polyhalo-C₁₋₄alkyl group contains up to 9, or 8, or 7, or 6, or 5, or 4, or 3, or 2 halo groups. Non-limiting examples of C₁₋₄haloalkyl include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. A perhalo-C₁₋₄alkyl group refers to a C₁₋₄alkyl group having all hydrogen atoms replaced with halo atoms.

The term “aryl” refers to an aromatic carbocyclic single ring or two fused ring system containing 6 to 10 carbon atoms. Examples include phenyl and naphthyl.

The term “heteroaryl” refers to a 5- to 12-membered aromatic radical containing 1-4 heteroatoms selected from N, O, and S. In some instances, nitrogen atoms in a heteroaryl may be quaternized. The term “heteroaryl” may be used interchangeably with the terms “heteroaryl ring”, “heteroaryl group”, or “heteroaromatic”. A heteroaryl group may be mono- or bi-cyclic. Monocyclic heteroaryl includes, for example, pyrazolyl, imidazolyl, oxazolyl, pyridinyl, furanyl, oxadiazolyl, thiophenyl, and the like. Bi-cyclic heteroaryls include groups in which a monocyclic heteroaryl ring is fused to one or more aryl or heteroaryl rings. Non-limiting examples include pyrazolopyridinyl, pyrazolopyridinyl, benzotriazolyl, imidazopyridinyl, and indoyl.

The term “carbocyclic ring” or “carbocyclyl” refers to a 4- to 12-membered saturated or partially unsaturated hydrocarbon ring and may exist as a single ring, bicyclic ring (including fused, spiral or bridged carbocyclic rings) or a spiral ring. Bi-cyclic carbocyclyl groups include, e.g., unsaturated carbocyclic radicals fused to another unsaturated carbocyclic radical, cycloalkyl, or aryl, such as, for example, 2,3-dihydroindenyl, decahydronaphthalenyl, and 1,2,3,4-tetrahydronaphthalenyl. Unless specified otherwise, the carbocyclic ring generally contains 4- to 10-ring members.

The term “C₃₋₆ cycloalkyl” refers to a carbocyclic ring which is fully saturated (e.g., cyclopropyl, cyclobutyl, cyclopentyl, and cyclohexyl).

The term “heterocycle” or “heterocyclyl” refers to a 4- to 12-membered saturated or partially unsaturated heterocyclic ring containing 1 to 4 heteroatoms independently selected from N, O, and S. A heterocyclyl group may be mono- or bicyclic (e.g., a bridged, fused, or spiro bicyclic ring). Examples of monocyclic saturated or partially unsaturated heterocyclic radicals include, without limitation, oxetanyl, tetrahydrofuranyl, tetrahydropyranyl, and piperdinyl. Bi-cyclic heterocyclyl groups include, e.g., unsaturated heterocyclic radicals fused to another unsaturated heterocyclic radical, cycloalkyl, aryl, or heteroaryl ring, such as, for example, tetrahydro-3H-[1,2,3]triazolo[4,5-c]pyridinyl, 2-oxa-6-azaspiro[3.3]heptanyl, 5-oxabicyclo[2.1.1]hexanyl and 9-azabicyclo[3.3.1]nonanyl. In some embodiments, the heterocyclyl group is a 4 to 6 membered monocyclic heterocyclyl group. In some embodiments, the heterocyclyl group is a 4 to 6 membered monocyclic saturated heterocyclyl group. In some embodiments, the heterocyclyl group is a 8 to 10 membered bicyclic heterocyclyl group. In some embodiments, the heterocyclyl group is a 8 to 10 membered bicyclic saturated heterocyclyl group.

As used herein the term “spiral” ring means a two-ring system wherein both rings share one common atom. Examples of spiral rings include, 2-oxa-6-azaspiro[3.3]heptanyl and the like.

The term “fused” ring refers to two ring systems share two adjacent ring atoms. Fused heterocycles have at least one the ring systems contain a ring atom that is a heteroatom selected from O, N and S (e.g., 3-oxabicyclo[3.1.0]hexane).

As used herein the term “bridged” refers to a 5 to 10 membered cyclic moiety connected at two non-adjacent ring atoms (e.g. 5-oxabicyclo[2.1.1]hexane).

The phrase “pharmaceutically acceptable” indicates that the substance, composition or dosage form must be compatible chemically and/or toxicologically, with the other ingredients comprising a formulation, and/or the mammal being treated therewith.

Unless specified otherwise, the term “compounds of the present disclosure” refers to compounds of Formula (I), as well as all stereoisomers (including diastereoisomers and enantiomers), rotamers, tautomers, isotopically labeled compounds (including deuterium substitutions). When a moiety is present that is capable of forming a salt, then salts are included as well, in particular pharmaceutically acceptable salts.

As used herein, the term “a,” “an,” “the” and similar terms used in the context of the present invention (especially in the context of the claims) are to be construed to cover both the singular and plural unless otherwise indicated herein or clearly contradicted by the context. The use of any and all examples, or exemplary language (e.g., “such as”) provided herein is intended merely to better illuminate the invention and does not pose a limitation on the scope of the invention otherwise claimed.

It is also possible that the intermediates and compounds of the present invention may exist in different tautomeric forms, and all such forms are embraced within the scope of the invention. The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. A specific example of a proton tautomer is the imidazole moiety where the proton may migrate between the two ring nitrogens. Valence tautomers include interconversions by reorganization of some of the bonding electrons.

In one embodiment, the present disclosure relates to a compound of the Formula (I) as defined herein, in free form. In another embodiment, the present disclosure relates to a compound of the Formula (I) as defined herein, in salt form. In another embodiment, the present disclosure relates to a compound of the Formula (I) as defined herein, in acid addition salt form. In a further embodiment, the present disclosure relates to a compound of the Formula (I) as defined herein, in pharmaceutically acceptable salt form. In yet a further embodiment, the present disclosure relates to a compound of the Formula (I) as defined herein, in pharmaceutically acceptable acid addition salt form. In yet a further embodiment, the present disclosure relates to any one of the compounds of the Examples in free form. In yet a further embodiment, the present disclosure relates to any one of the compounds of the Examples in salt form. In yet a further embodiment, the present disclosure relates to any one of the compounds of the Examples in acid addition salt form. In yet a further embodiment, the present disclosure relates to any one of the compounds of the Examples in pharmaceutically acceptable salt form. In still another embodiment, the present disclosure relates to any one of the compounds of the Examples in pharmaceutically acceptable acid addition salt form.

Compounds of the present disclosure may be synthesized by synthetic routes that include processes analogous to those well-known in the chemical arts, particularly in light of the description contained herein. The starting materials are generally available from commercial sources such as Sigma-Aldrich or are readily prepared using methods well known to those skilled in the art (e.g., prepared by methods generally described in Louis F. Fieser and Mary Fieser, Reagents for Organic Synthesis, v. 1-19, Wiley, New York (1967-1999 ed.), or Beilsteins Handbuch der organischen Chemie, 4, Aufl. ed. Springer-Verlag, Berlin, including supplements (also available via the Beilstein online database)).

For illustrative purposes, the reaction schemes depicted below provide potential routes for synthesizing the compounds of the present disclosure as well as key intermediates. For a more detailed description of the individual reaction steps, see the Examples section below. Although specific starting materials and reagents are depicted in the schemes and discussed below, other starting materials and reagents can be easily substituted to provide a variety of derivatives and/or reaction conditions.

### Exemplification

### Abbreviations:

PE=petroleum ether
EtOAc=EA=ethyl acetate
ESI=electrospray ionisation
MeOH=methanol
EtOH=ethanol
DCE=1,2-dichloroethane
DCM=dichloromethane
CHCl₃=chloroform
HCl=hydrochloric acid
H₂O=water
IPA=isopropyl alcohol
LCMS=liquid chromatography mass spectrometry
HFLP=hexafluoro-2-propanol
HPLC=high pressure liquid chromatography
THF=tetrahydrofuran
MeCN=ACN=acetonitrile
MgSO₄=magnesium sulfate
DMSO=dimethylsulfoxide
AcOH=acetic acid
TFA=trifluoroacetic acid
DIPEA=diisopropylethyl amine
N₂=Nitrogen
NH₄HCO₃=Ammonium Bicarbonate
t-BuOH=tert-butanol
NH₄C₁=ammonium chloride
NaH=sodium hydride
Na₂SO₄=sodium sulfate
K₂CO₃=potassium carbonate
NaHCO₃=sodium bicarbonate
NaBH(OAc)₃=STAB=sodium triacetoxyborohydride
SiO₂=silicon dioxide or silica
PDA=Photo Diode Array Detection

### General Methods

### LCMS Instrumentation Specifications:

Agilent Technologies 1200 Series LC/MSD system: DAD/ELSD Alltech 3300 and Agilent LC/MSD G6130A, G6120B mass-spectrometer.
Agilent Technologies 1260 Infinity LC/MSD system: DAD/ELSD Alltech 3300 and Agilent LC/MSD G6120B mass-spectrometer.
Agilent Technologies 1260 Infinity II LC/MSD system: DAD/ELSD G7102A 1290 Infinity II and Agilent LC/MSD G6120B mass-spectrometer.
Agilent 1260 Series LC/MSD system: DAD/ELSD and Agilent LC/MSD (G6120B) mass-spectrometer.
UHPLC Agilent 1290 Series LC/MSD system: DAD/ELSD and Agilent LC/MSD (G6125B) mass-spectrometer.

### HPLC Analytical Method Specifications:

Column: Agilent Poroshell 120 SB-C18 4.6×30 mm 2.7 μm, with UHPLC Guard Infinity Lab Poroshell 120 SB-C18 4.6×5 mm 2.7 μm
Column Temperature, 60° C.
Injection volume 0.5 μL
Modifier: Formic acid 0.1% (v/v) conc.
Method: 99% water/1% MeCN (initial conditions), HOLD initial conditions for 0.1 min, linear gradient to 0% water/100% MeCN at 1.5 min, HOLD 0% water/100% MeCN to 1.73 min, linear gradient to 99% water/1% MeCN at 1.74 min. Flow rate, 3.0 mL/min.
UV scan: 207-223 nm, 246-262 nm, 272-288 nm

### QC Analysis LC/MS Method Conditions:

### Ammonium Hydroxide (Basic pH) Conditions

MS mode: MS:ESI+ scan range 165-650 daltons
PDA: 200-400 nm scan range
Column: Waters ACQUITY UPLC BEH C18 2.1×50 mm, 1.7 μm; Part No. 186002350
Modifier: Ammonium hydroxide 0.2% (v/v) conc.
Method: 95% water/5% MeCN (initial conditions) linear gradient to 5% water/95% MeCN at 3.75 min, HOLD 5% water/95% MeCN to 4 min. Flow rate, 0.8 mL/min.

### Trifluoroacetic Acid (Acidic pH) Conditions

MS mode: MS:ESI+ scan range 165-650 daltons
PDA: 200-400 nm scan range
Column: Waters ACQUITY UPLC BEH C18 2.1×50 mm, 1.7 μm; Part No. 186002350
Modifier: Trifluoroacetic acid 0.1% (v/v) conc.
Method: 95% water/5% MeCN (initial conditions) linear gradient to 5% water/95% MeCN at 3.75 min, HOLD 5% water/95% MeCN to 4 min. Flow rate, 0.8 mL/min.

### General Prep HPLC Conditions:

### Ammonium Hydroxide (Basic pH) Conditions

Flow rate: 30 mL/min
MS mode: MS:ESI+ scan range 165-650 daltons
PDA: 200-400 nm scan range
Column: Waters XSELECT CSH C18 PREP 19×100 mm, 5 μm; Part No. 186005421
Modifier: 0.2% Ammonium hydroxide (v/v) conc.
Method: A % water/B % MeCN (initial conditions) linear gradient to A % water/B % MeCN at 8 min, ramp to 5% water/95% MeCN at 8.5 min, HOLD 5% water/95% MeCN to 10 min.
Flow rate: 50 mL/min
MS mode: MS:ESI+ scan range 165-650 daltons
PDA: 200-400 nm scan range
Column: Waters XSELECT CSH C18 PREP 30×100 mm, 5 μm; Part No. 186005425
Modifier: 0.2% NH₄OH (v/v) conc.
Method: A % water/B % MeCN (initial conditions) linear gradient to A % water/B % MeCN at 8 min, ramp to 5% water/95% MeCN at 8.5 min, HOLD 5% water/95% MeCN to 10 min.
Flow rate, 60 mL/min
MS mode: MS:ESI+ scan range 165-650 daltons
PDA: 200-400 nm scan range
Column: Waters XSELECT CSH C18 PREP 30×50 mm, 5 μm; Part No. 186005423
Modifier: 0.2% NH₄OH (v/v) conc.
Method: A % water/B % MeCN (initial conditions) linear gradient to A % water/B % MeCN at 8 min, ramp to 5% water/95% MeCN at 8.5 min, HOLD 5% water/95% MeCN to 10 min.

### Trifluoroacetic Acid (Acidic pH) Conditions

Flow rate, 30 mL/min
MS mode: MS:ESI+ scan range 165-650 daltons
PDA: 200-400 nm scan range
Column: Waters Sunfire OBD C18 PREP 19×100 mm, 5 μm; Part No. 186002567
Modifier: 0.1% Trifluoroacetic acid (v/v) conc.
Method: A % water/B % MeCN (initial conditions) linear gradient to A % water/B % MeCN at 8 min, ramp to 5% water/95% MeCN at 8.5 min, HOLD 5% water/95% MeCN to 10 min.
Flow rate, 50 mL/min
MS mode: MS:ESI+ scan range 165-650 daltons
PDA: 200-400 nm scan range
Column: Waters Sunfire OBD C18 PREP 30×100 mm, 5 μm; Part No. 186002572
Modifier: 0.1% Trifluoroacetic acid (v/v) conc.
Method: A % water/B % MeCN (initial conditions) linear gradient to A % water/B % MeCN at 8 min, ramp to 5% water/95% MeCN at 8.5 min, HOLD 5% water/95% MeCN to 10 min.
Flow rate, 60 mL/min
MS mode: MS:ESI+ scan range 165-650 daltons
PDA: 200-400 nm scan range
Column: Waters Sunfire OBD C18 PREP 30×50 mm, 5 μm; Part No. 186002570
Modifier: 0.1% Trifluoroacetic acid (v/v) conc.
Method: A % water/B % MeCN (initial conditions) linear gradient to A % water/B % MeCN at 8 min, ramp to 5% water/95% MeCN at 8.5 min, HOLD 5% water/95% MeCN to 10 min.

### Preparation of Intermediates

### Intermediate A: 3-Morpholino-1-oxa-8-azaspiro[4.5]decane hydrochloride

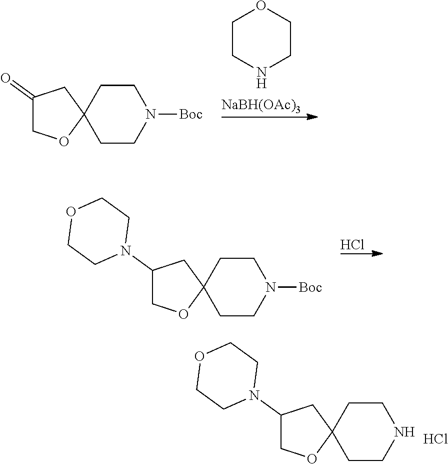

### 1. Synthesis of tert-butyl 3-morpholino-1-oxa-8-azaspiro[4.5]decane-8-carboxylate

A reaction vessel was charged with tert-butyl 3-oxo-1-oxa-8-azaspiro[4.5]decane-8-carboxylate (2 g, 7.8 mmol), morpholine (1.0 mL, 11.8 mmol) and NaBH(OAc)₃ (3.3 g, 15.7 mmol) in DCE (20 mL) and the resulting solution was stirred at room temperature for 24 h. The reaction mixture was evaporated and diluted with 2M aqueous K₂CO₃ (50 mL) solution and DCM (50 mL) and the layers separated. The organic layer was evaporated under reduced pressure and the crude tert-butyl 3-morpholino-1-oxa-8-azaspiro[4.5]decane-8-carboxylate was used in the next step without further purification. LCMS m/z=327.1 [M+H]⁺.

### 2. Synthesis of 3-morpholino-1-oxa-8-azaspiro[4.5]decane

A reaction vessel was charged with tert-butyl 3-morpholino-1-oxa-8-azaspiro[4.5]decane-8-carboxylate (2.4 g, 7.4 mmol) and 10% HCl in dioxane (10.4 mL, 29.4 mmol) in dioxane (10 mL) and the resulting solution was stirred at 50° C. for 24 h. The reaction mixture was filtered, the precipitate was washed with EtOAc and dried at 70° C. to give 3-morpholino-1-oxa-8-azaspiro[4.5]decane hydrochloride (2 g) which was used without further purification. LCMS m/z=227.2 [M+H]⁺.

### Examples 1 to 5

The title compounds were prepared in a single step library on an approximately 50 mg target product scale using the following protocol.

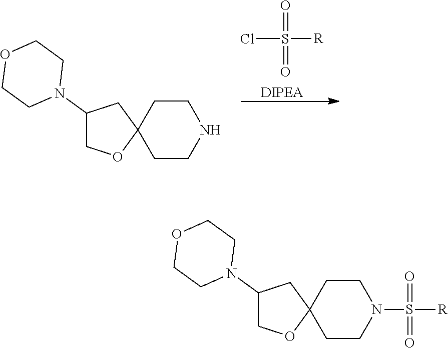

The appropriate sulfonyl chloride (1.1 equiv.) was added to a solution of 3-(morpholin-4-yl)-1-oxa-8-azaspiro[4.5]decane dihydrochloride (1.0 equiv.) and DIPEA (4.5 equiv.+1.1 equiv. per each acid equiv. for sulfonyl chloride building block salts) in dry MeCN (1.2 mL) and the reaction mixture was stirred at room temperature for 16 h. The solvent was evaporated in vacuo and the residue was dissolved in DMSO (0.8 mL) and purified by prep. PLC (Column: YMC Actus Trial C18 20×100 5 mm; Method water—MeOH—NH₃ 0.1% as a mobile phase) to afford pure product.

| Example | | |
|---|---|---|
| No. | Name/Structure/Sulfonyl chloride | HPLC gradient/Yield/Data |
| 1 | 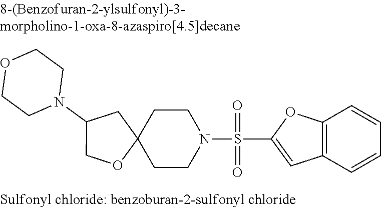 [SMILES: O=S(=O)(c1cc2ccccc2o1)N1CCC2(CC1)CC(N1CCOCC1)CO2] | HPLC gradient: 30-80% 16.1 mg (32%) LCMS m/z = 407.2 [M + H]⁺. ¹H-NMR (500 MHz, DMSO-d₆) δ (ppm): 7.81 (br d, J = 7.8 Hz, 1H), 7.75 (br d, J = 8.5 Hz, 1H), 7.65 (s, 1H), 7.54 (br t, J = 7.8 Hz, 1H), 7.40 (t, J = 7.5 Hz, 1H), 3.78 (br t, J = 7.5 Hz, 1H), 3.50 (br s, 4H), 3.45 (br t, J = 8.2 Hz, 1H), 3.40 (br d, J = 11.8 Hz, 2H), 3.03-2.91 (m, 2H), 2.82 (quin, J = 7.3 Hz, 1H), 2.37-2.16 (m, 4H), 1.88 (br dd, J = 7.6, 12.3 Hz, 1H), 1.72-1.43 (m, 5H). |
| 2 | 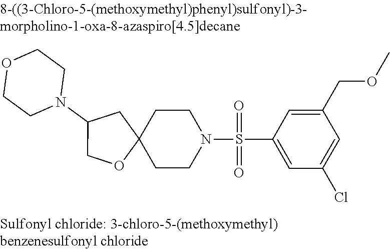 [SMILES: COCc1cc(Cl)cc(S(=O)(=O)N2CCC3(CC2)CC(N2CCOCC2)CO3)c1] | HPLC gradient: 45-75% 8.4 mg (17%) LCMS m/z = 445.2 [M + H]⁺. ¹H-NMR (600 MHZ, DMSO-d₆) δ (ppm): 7.71 (s, 1H), 7.64 (s, 1H), 7.60 (s, 1H), 4.52 (s, 2H), 3.77 (t, J = 7.59 Hz, 1H), 3.50 (br s, 4H), 3.44 (br t, J = 8.0 Hz, 1H), 3.32 (s, 3H), 3.27 (br d, J = 7.9 Hz, 2H), 2.82 (br s, 1H), 2.64-2.54 (m, 2H), 2.35-2.17 (m, 4H), 1.86 (dd, J = 7.8, 12.4 Hz, 1H), 1.71-1.64 (m, 2H), 1.64-1.52 (m, 2H), 1.48 (br dd, J = 8.4, 12.3 Hz, 1H) |
| 3 | 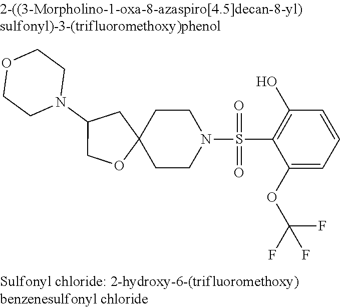 [SMILES: O=S(=O)(c1c(O)cccc1OC(F)(F)F)N1CCC2(CC1)CC(N1CCOCC1)CO2] | HPLC gradient: 30-55% 1.3 mg (3%) LCMS m/z = 467.0 [M + H]⁺. R_{f} = 0.856 min |
| 4 | 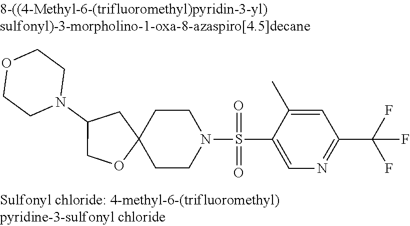 [SMILES: Cc1cc(C(F)(F)F)ncc1S(=O)(=O)N1CCC2(CC1)CC(N1CCOCC1)CO2] | HPLC gradient: 45-75% 8.3 mg (17%) LCMS m/z = 450.2 [M + H]⁺. R_{f} = 1.058 min |
| 5 | 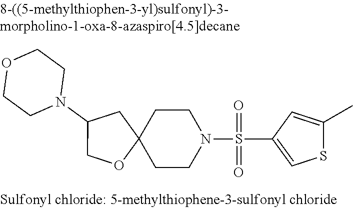 [SMILES: Cc1cc(S(=O)(=O)N2CCC3(CC2)CC(N2CCOCC2)CO3)cs1] | HPLC gradient: 40-70% 8.7 mg (17%) LCMS m/z = 387.2 [M + H]⁺. ¹H-NMR (600 MHZ, DMSO-d₆) δ (ppm): 7.40 (d, J = 3.6 Hz, 1H), 6.98 (d, J = 3.6 Hz, 1H), 3.79 (dd, J = 6.8, 8. 5 Hz, 1H), 3.51 (br t, J = 4.4 Hz, 4H), 3.49-3.43 (m, 1H), 3.26-3.19 (m, 2H), 2.83 (br t, J = 7.4 Hz, 1H), 2.67-2.56 (m, 2H), 2.51 (s, 3H), 2.35-2.19 (m, 4H), 1.87 (dd, J = 7.8, 12.4 Hz, 1H), 1.74-1.56 (m, 4H), 1.49 (dd, J = 8.4, 12.4 Hz, 1H) |

### Example 6: 7-((4-Methyl-6-(trifluoromethyl)pyridin-3-yl)sulfonyl)-N-(tetrahydro-2H-pyran-4-yl)-7-azaspiro[3.5]nonan-2-amine

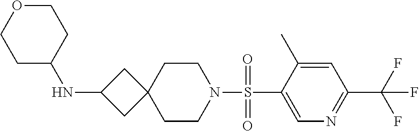 [SMILES: Cc1cc(C(F)(F)F)ncc1S(=O)(=O)N1CCC2(CC1)CC(NC1CCOCC1)C2]

### 1. Synthesis of 7-((4-methyl-6-(trifluoromethyl)pyridin-3-yl)sulfonyl)-7-azaspiro[3.5]nonan-2-one

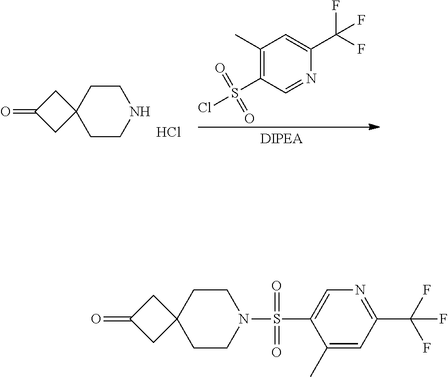 [SMILES: O=C1CC2(CCNCC2)C1 ; Cl ; Cc1cc(C(F)(F)F)ncc1S(=O)(=O)Cl ; Cc1cc(C(F)(F)F)ncc1S(=O)(=O)N1CCC2(CC1)CC(=O)C2]

To a vial containing 7-azaspiro[3.5]nonan-2-one hydrochloride (184 mg, 1.1 mmol) in anhydrous DCM (4 mL) was added DIPEA (0.6 mL, 3.4 mmol) dropwise at <5° C. After 5 min, 4-methyl-6-(trifluoromethyl)pyridine-3-sulfonyl chloride (351 mg, 1.4 mmol) was added. The reaction was brought to room temperature stirred for 30 min and quenched by slow addition of aqueous 1 M NaOH solution and stirred for another 10 min. The biphasic mixture was directly loaded onto silica gel and purified by column chromatography (20-70% EtOAc in heptane) to afford 7-((4-methyl-6-(trifluoromethyl)pyridin-3-yl)sulfonyl)-7-azaspiro[3.5]nonan-2-one as a white solid (285 mg, 75%) that was used without further purification in the next step. LCMS m/z=363.2 (M+H)⁺. ¹H-NMR (500 MHz, DCM-d₂) δ (ppm): 9.06 (s, 1H), 7.67 (s, 1H), 3.28-3.25 (m, 4H), 2.80 (s, 4H), 2.72 (s, 3H), 1.86-1.83 (m, 4H).

### 2. Synthesis of 7-((4-methyl-6-(trifluoromethyl)pyridin-3-yl)sulfonyl)-N-(tetrahydro-2H-pyran-4-yl)-7-azaspiro[3.5]nonan-2-amine

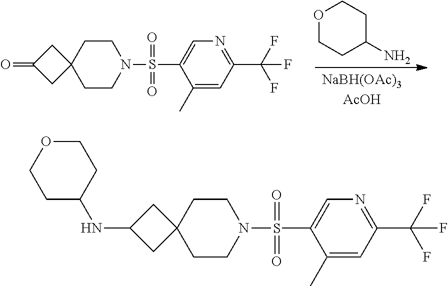

To a vial containing 7-((4-methyl-6-(trifluoromethyl)pyridin-3-yl)sulfonyl)-7-azaspiro[3.5]nonan-2-one (85 mg, 0.2 mmol) in anhydrous DCM (6 mL) was added tetrahydropyran-4-amine (24 mg, 0.2 mmol) followed by AcOH (30 μL, 0.5 mmol) dropwise at room temperature. After 15 min, NaBH(OAc)₃ (199 mg, 0.94 mmol) was added. After 6 h, the reaction mixture was quenched with saturated aqueous NH₄Cl, stirred for 10 min, and extracted with DCM (3×). The organic layer was dried over anhydrous MgSO₄. After filtration and concentration under reduced pressure, the residue was loaded onto a silica gel column and purified (0-100% 3:1 EtOAc:EtOH in heptane with 2% aqueous ammonia) to afford 7-((4-methyl-6-(trifluoromethyl)pyridin-3-yl)sulfonyl)-N-(tetrahydro-2H-pyran-4-yl)-7-azaspiro[3.5]nonan-2-amine as a colorless film (30 mg, 28%). LCMS m/z=448.2 (M+H)⁺. ¹H-NMR (500 MHz, CDCl₃) δ (ppm): 9.05 (s, 1H), 7.65 (s, 1H), 3.98-3.90 (m, 2H), 3.45-3.38 (m, 3H), 3.25-3.20 (m, 2H), 3.17-3.13 (m, 2H), 2.67 (s, 3H), 2.65-2.62 (m, 1H), 2.20-2.15 (m, 2H), 1.9-1.8 (broad m, 1H), 1.75-1.70 (m, 4H), 1.74-1.60 (m, 2H), 1.52-1.48 (m, 2H), 1.42-1.35 (m, 2H).

### Example 7: (R)- or (S)-8-((1-methyl-3-(trifluoromethyl)-1H-pyrazol-5-yl)sulfonyl)-3-morpholino-1-oxa-8-azaspiro[4.5]decane

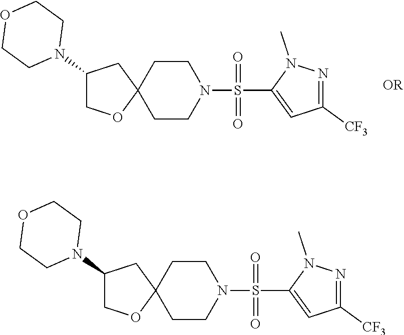 [SMILES: Cn1nc(C(F)(F)F)cc1S(=O)(=O)N1CCC2(CC1)C[C@@H](N1CCOCC1)CO2 ; Cn1nc(C(F)(F)F)cc1S(=O)(=O)N1CCC2(CC1)C[C@H](N1CCOCC1)CO2]

### 1. Synthesis of tert-butyl(R)-3-morpholino-1-oxa-8-azaspiro[4.5]decane-8-carboxylate and tert-butyl(S)-3-morpholino-1-oxa-8-azaspiro[4.5]decane-8-carboxylate

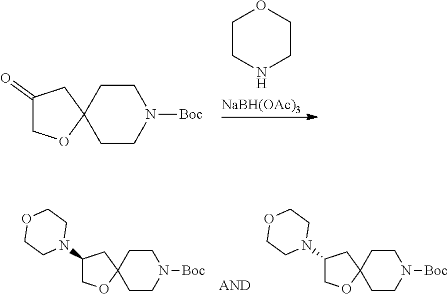 [SMILES: C1COCCN1 ; CC(C)(C)OC(=O)N1CCC2(CC1)CC(=O)CO2 ; CC(C)(C)OC(=O)N1CCC2(CC1)C[C@H](N1CCOCC1)CO2 ; CC(C)(C)OC(=O)N1CCC2(CC1)C[C@@H](N1CCOCC1)CO2 ; "NaBH(OAc)3"]

NaBH(OAc)₃ (13.3 g, 211.5 mmol) was added in portions to a solution of tert-butyl 3-oxo-1-oxa-8-azaspiro[4.5]decane-8-carboxylate (18 g, 70.5 mmol) and morpholine (18.2 mL, 211.5 mmol) in MeOH (450 mL) and acetic acid (1.21 mL, 21.2 mmol) and the reaction mixture was stirred at 20° C. for 12 h under N₂. The reaction mixture was concentrated in vacuo and the residue was purified by silica gel column chromatography (from 0% to 60%, EtOAc in PE) to give tert-butyl 3-morpholino-1-oxa-8-azaspiro[4.5]decane-8-carboxylate (20 g, 87%). This was further purified by prep-SFC (Column: ChiralPak AD-3 150×4.6 mm I.D., 3 μm; Mobile phase: A: CO₂ B: Ethanol (0.05% DEA); Gradient: from 5% to 40% of B in 4.5 min, then 5% of B for 1.5 min; Flow rate: 2.5 mL/min; Column temp.: 40° C.; pressure 100 bar) to give: The first eluting peak, E1 (5.1 g, 22%) as a white solid. LCMS m/z=327.2 [M+H]⁺. ^{v}(400 MHz, CDCl₃) δ (ppm): 4.00 (dd, J=6.8, 8.4 Hz, 1H), 3.74-3.66 (m, 5H), 3.56 (s, 2H), 3.35-3.24 (m, 2H), 2.96 (d, J=7.6 Hz, 1H), 2.54-2.43 (m, 2H), 2.42-2.32 (m, 2H), 1.96 (dd, J=7.6, 12.0 Hz, 1H), 1.66-1.58 (m, 4H), 1.53-1.46 (m, 1H), 1.44 (s, 9H). and the second eluting peak, E2, (6.2 g, 27%) as a white solid. LCMS m/z=327.2 [M+H]⁺. ¹H-NMR: (400 MHz, CDCl₃) δ (ppm): 4.00 (dd, J=6.8, 8.4 Hz, 1H), 3.75-3.67 (m, 5H), 3.63-3.51 (m, 2H), 3.36-3.25 (m, 2H), 2.96 (d, J=7.6 Hz, 1H), 2.54-2.44 (m, 2H), 2.43-2.34 (m, 2H), 1.96 (dd, J=7.6, 12.0 Hz, 1H), 1.65-1.59 (m, 4H), 1.53-1.47 (m, 1H), 1.44 (s, 9H).

### 2. Synthesis of (R) or (S)-3-morpholino-1-oxa-8-azaspiro[4.5]decane hydrochloride

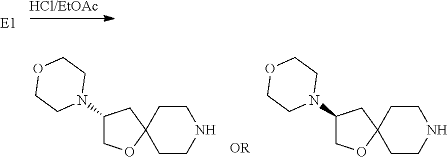 [SMILES: "HCl/EtOAc" ; C1CC2(CCN1)C[C@@H](N1CCOCC1)CO2 ; C1CC2(CCN1)C[C@H](N1CCOCC1)CO2]

To a solution of tert-butyl(R)- or (S)-3-morpholino-1-oxa-8-azaspiro[4.5]decane-8-carboxylate (326 mg, 1.0 mmol) in EtOAc (2 mL) was added HCl (1 M, 3.0 mL) in EtOAc and the reaction stirred at room temperature overnight. The resulting white suspension was filtered and washed with Et₂O to give (R)- or (S)-3-morpholino-1-oxa-8-azaspiro[4.5]decane hydrochloride as a white solid. LCMS m/z=227.1 [M+H]⁺.

### 3. Synthesis of (R) or (S)-8-((1-methyl-3-(trifluoromethyl)-1H-pyrazol-5-yl)sulfonyl)-3-morpholino-1-oxa-8-azaspiro[4.5]decane

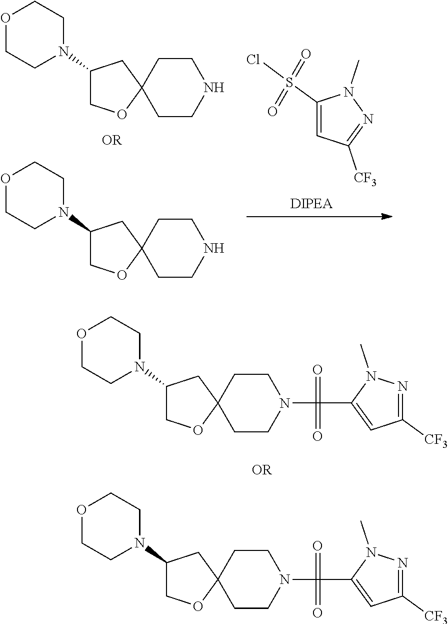

To a mixture of (R)- or (S)-3-morpholino-1-oxa-8-azaspiro[4.5]decane hydrochloride (41 mg, 0.1 mmol) and 2-methyl-5-(trifluoromethyl)pyrazole-3-sulfonyl chloride (31 mg, 0.1 mmol) in DCM (2 mL) was added DIPEA (85 mL, 0.5 mmol). The reaction mixture was stirred at room temperature for 2 h. The organic phase was washed with satd. NaHCO₃ and water, dried over MgSO₄, filtered and concentrated. The crude residue was purified by silica gel column chromatography (50-100% EtOAc in heptane) to give (R)- or (S)-8-((1-methyl-3-(trifluoromethyl)-1H-pyrazol-5-yl)sulfonyl)-3-morpholino-1-oxa-8-azaspiro[4.5]decane (28 mg, 52%) as a colorless oil. LCMS m/z=439.2 (M+H)⁺. ¹H-NMR (400 MHz, CD₃OD): δ (ppm): 7.11 (s, 1H), 4.14 (s, 3H), 3.98 (dd, J=8.7, 6.9 Hz, 1H), 3.67 (t, J=4.8 Hz, 5H), 3.58-3.46 (m, 2H), 2.99 (s, 3H), 2.57-2.32 (m, 4H), 2.09-2.01 (m, 1H), 1.86-1.75 (m, 3H), 1.73-1.60 (m, 2H).

### Intermediate B: Synthesis of 3-(2-oxa-6-azaspiro[3.3]heptan-6-yl)-1-oxa-8-azaspiro[4.5]decane

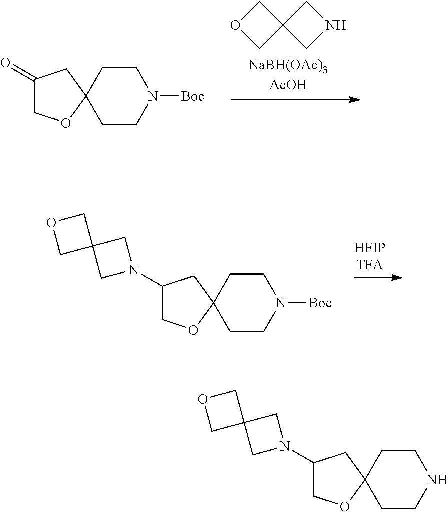 [SMILES: C1NCC12COC2 ; CC(C)(C)OC(=O)N1CCC2(CC1)CC(=O)CO2 ; CC(C)(C)OC(=O)N1CCC2(CC1)CC(N1CC3(COC3)C1)CO2 ; C1CC2(CCN1)CC(N1CC3(COC3)C1)CO2]

### 1. Synthesis of tert-butyl 3-(2-oxa-6-azaspiro[3.3]heptan-6-yl)-1-oxa-8-azaspiro[4.5]decane-8-carboxylate

2-Oxa-6-azaspiro[3.3]heptane hydrochloride (186 mg, 1.9 mmol) was added to a solution of tert-butyl 3-oxo-1-oxa-8-azaspiro[4.5]decane-8-carboxylate (400 mg, 1.6 mmol) in DCM (40 mL) and the reaction was stirred at room temperature for 15 min. Acetic acid (180 μL, 3.1 mmol) was added dropwise followed by NaBH(OAc)₃ (1.3 g, 6.3 mmol) after 30 min. The reaction mixture was stirred at room temperature until the starting material had been consumed.

Brine (20 mL) and DCM (40 mL) were added, the layers separated, the organic layer was washed with water (20 mL) and brine (20 mL), dried over Na₂SO₄, filtered and evaporated under reduced pressure to give tert-butyl 3-(2-oxa-6-azaspiro[3.3]heptan-6-yl)-1-oxa-8-azaspiro[4.5]decane-8-carboxylate. LCMS m/z=339.1 [M+H]⁺.

### 2. Synthesis of 3-(2-oxa-6-azaspiro[3.3]heptan-6-yl)-1-oxa-8-azaspiro[4.5]decane

To a solution of tert-butyl 3-(2-oxa-6-azaspiro[3.3]heptan-6-yl)-1-oxa-8-azaspiro[4.5]decane-8-carboxylate (400 mg, 1.2 mmol) in DCM (5 mL) was added TFA (270 μL, 3.6 mmol) and 1,1,1,3,3,3-hexafluoropropan-2-ol (10 mL, 95.0 mmol) and the mixture was stirred at 25° C. for 1 h. DIPEA (0.5 mL, 2.9 mmol) was added and the resulting mixture was concentrated under reduced pressure to give 3-(2-oxa-6-azaspiro[3.3]heptan-6-yl)-1-oxa-8-azaspiro[4.5]decane (450 mg) as a colorless oil which was used without further purification in the next step. LCMS m/z=239.1 [M+H]⁺.

### Intermediate C: Synthesis of 6-(8-azaspiro[4.5]decan-2-yl)-2-oxa-6-azaspiro[3.3]heptane

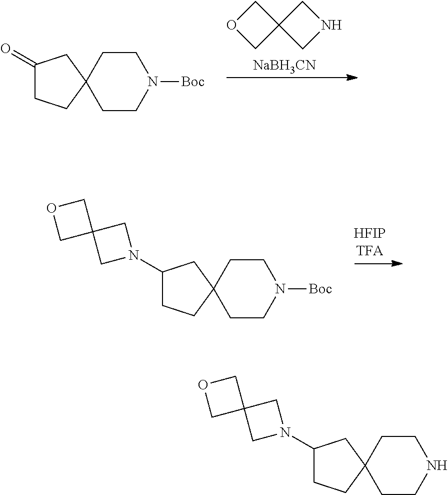

### 1. Synthesis of tert-butyl 3-(2-oxa-6-azaspiro[3.3]heptan-6-yl)-1-oxa-8-azaspiro[4.5]decane-8-carboxylate

To a solution of tert-butyl 3-oxo-1-oxa-8-azaspiro[4.5]decane-8-carboxylate (950 mg, 3.8 mmol) and 2-oxa-6-azaspiro[3.3]heptane (1.1 g, 3.8 mmol) in MeOH (20 mL) was added sodium cyanoborohydride (707 mg, 11.3 mmol) and the mixture was stirred at 25° C. for 12 h. The reaction mixture was concentrated under reduced pressure to give a residue which was purified by prep-HPLC (Column: Welch Xtimate C18 150×25 mm×5 μm; Condition: water (NH₄HCO₃)−MeCN; Begin B: 36; End B: 66; Gradient Time (min): 10; Flow Rate (mL/min): 25, to give tert-butyl 3-(2-oxa-6-azaspiro[3.3]heptan-6-yl)-1-oxa-8-azaspiro[4.5]decane-8-carboxylate (1.0 g, 81%) as a light yellow oil. LCMS m/z=337.2 [M+H]⁺.

### 2. Synthesis of 6-(8-azaspiro[4.5]decan-3-yl)-2-oxa-6-azaspiro[3.3]heptane

To a solution of tert-butyl 3-(2-oxa-6-azaspiro[3.3]heptan-6-yl)-8-azaspiro[4.5]decane-8-carboxylate (600 mg, 1.8 mmol) in 1,1,1,3,3,3-hexafluoropropan-2-ol (12 mL, 114.0 mmol) was added TFA (410 μL, 5.4 mmol) and the mixture was stirred at 25° C. for 1 h. DIPEA (3 mL, 17.3 mmol) was added and the resulting mixture was concentrated under reduced pressure to give 6-(8-azaspiro[4.5]decan-3-yl)-2-oxa-6-azaspiro[3.3]heptane (650 mg) as a colorless oil which was used without further purification in the next step. LCMS m/z=237.2 [M+H]⁺.

### Examples 8 to 13

The title compounds were prepared in a single step library on an approximately 60 mg target product scale using the following protocol.

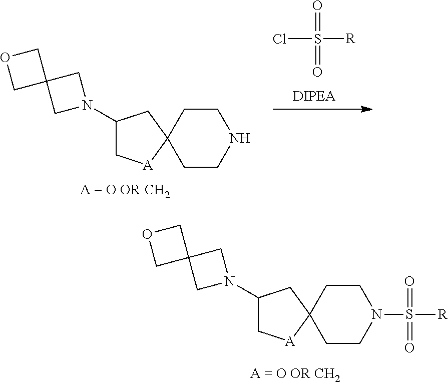

The appropriate sulfonyl chloride (1.0 equiv.) was added to a solution of 3-(2-oxa-6-azaspiro[3.3]heptan-6-yl)-1-oxa-8-azaspiro[4.5]decane (1.0 equiv.) or azaspiro[4.5]decan-2-yl)-2-oxa-6-azaspiro[3.3]heptane (1.0 equiv.) and DIPEA (2.0 equiv.) in dry DCM (10 mL) and the reaction mixture was stirred at room temperature for 30 min. The solids were filtered off and the filtrate was concentrated under reduced pressure. The resulting residue was purified by prep HPLC (Column: Welch Xtimate C18 150×25 mm×5 μm; Condition: water (10 mM NH₄HCO₃)−MeCN) at an appropriate gradient to afford the desired product.

| Example | | General procedure/HPLC |
|---|---|---|
| No. | Name/Structure/Sulfonyl chloride | gradient/Yield/Data |
| 8 | 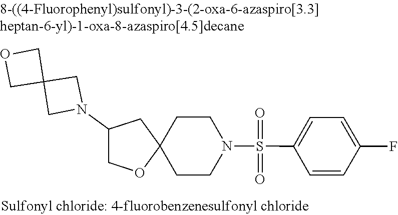 [SMILES: O=S(=O)(c1ccc(F)cc1)N1CCC2(CC1)CC(N1CC3(COC3)C1)CO2] | HPLC gradient: 25-55% B 27.6 mg (32%) LCMS m/z = 397.1 [M + H]⁺. R_{f} = 1.510 min |
| 9 | 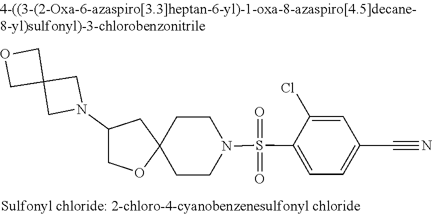 [SMILES: N#Cc1ccc(S(=O)(=O)N2CCC3(CC2)CC(N2CC4(COC4)C2)CO3)c(Cl)c1] | HPLC gradient: 25-55% B 18.6 mg (30%) LCMS m/z = 438.0 [M + H]⁺. R_{f} = 1.574 min |
| 10 | 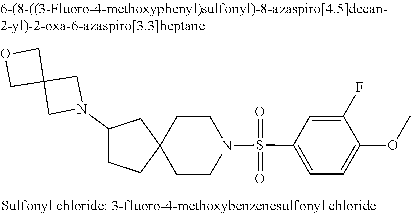 [SMILES: COc1ccc(S(=O)(=O)N2CCC3(CCC(N4CC5(COC5)C4)C3)CC2)cc1F] | HPLC gradient: 34-64% B 29.6 mg (33%) LCMS m/z = 425.1 [M + H]⁺. R_{f} = 1.757 min |
| 11 | 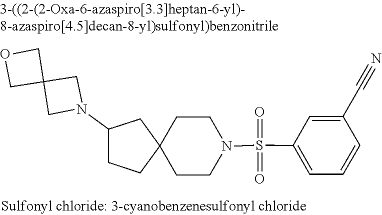 [SMILES: N#Cc1cccc(S(=O)(=O)N2CCC3(CCC(N4CC5(COC5)C4)C3)CC2)c1] | HPLC gradient: 20-50% B 22.5 mg (27%) LCMS m/z = 402.1 [M + H]⁺. R_{f} = 1.670 min |
| 12 | 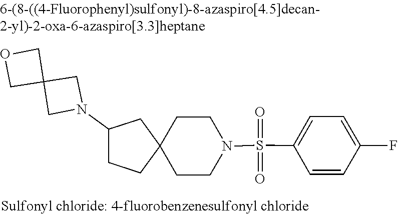 [SMILES: O=S(=O)(c1ccc(F)cc1)N1CCC2(CCC(N3CC4(COC4)C3)C2)CC1] | HPLC gradient: 34-64% B 10.2 mg (10%) LCMS m/z = 395.1 [M + H]⁺. R_{f} = 1.713 min |
| 13 | 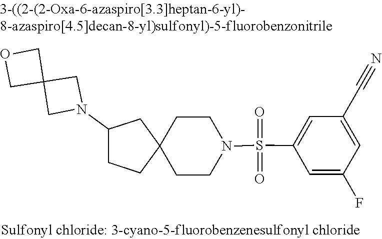 [SMILES: N#Cc1cc(F)cc(S(=O)(=O)N2CCC3(CCC(N4CC5(COC5)C4)C3)CC2)c1] | HPLC gradient: 31-61% B 26.3 mg (30%) LCMS m/z = 420.1 [M + H]⁺. ¹H-NMR: (400 MHZ, DMSO- d₆) δ (ppm): 8.28-8.24 (m, 1H), 8.06 (s, 1H), 7.97-7.94 (m, 1H), 4.53 (s, 4H), 3.11 (s, 4H), 2.96-2.94 (m, 4H), 1.52- 1.00 (m, 11H) |

### Example 14: 8-((4-(Difluoromethoxy)phenyl)sulfonyl)-3-(piperidin-1-yl)-1-oxa-8-azaspiro[4.5]decane

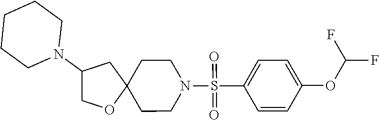 [SMILES: O=S(=O)(c1ccc(OC(F)F)cc1)N1CCC2(CC1)CC(N1CCCCC1)CO2]

### 1. Synthesis of tert-butyl 3-(piperidin-1-yl)-1-oxa-8-azaspiro[4.5]decane-8-carboxylate

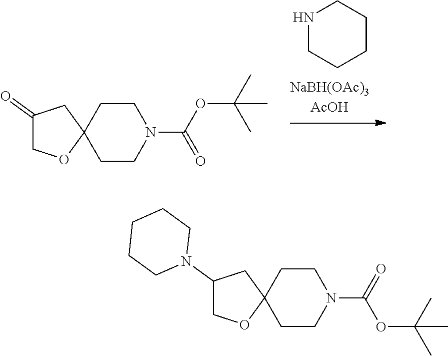 [SMILES: C1CCNCC1 ; CC(C)(C)OC(=O)N1CCC2(CC1)CC(=O)CO2 ; CC(C)(C)OC(=O)N1CCC2(CC1)CC(N1CCCCC1)CO2]

Piperidine hydrochloride (160 mg, 1.9 mmol) was added to a solution of tert-butyl 3-oxo-1-oxa-8-azaspiro[4.5]decane-8-carboxylate (400 mg, 1.6 mmol) in DCM (40 mL) and the reaction was stirred at room temperature for 15 min. Acetic acid (180 μL, 3.1 mmol) was added dropwise, the solution was stirred for an additional 30 min, NaBH(OAc)₃ (1.3 g, 6.3 mmol) was added and the reaction stirred at room temperature for 6 h. The reaction was quenched with saturated aqueous NH₄Cl and diluted with DCM. The organics were washed with water and brine then concentrated in vacuo to afford the desired product (450 mg, 70%) that was not purified further. LCMS m/z=325.2 [M+H]⁺.

### 2. Synthesis of 8-((4-(difluoromethoxy)phenyl)sulfonyl)-3-(piperidin-1-yl)-1-oxa-8-azaspiro[4.5]decane

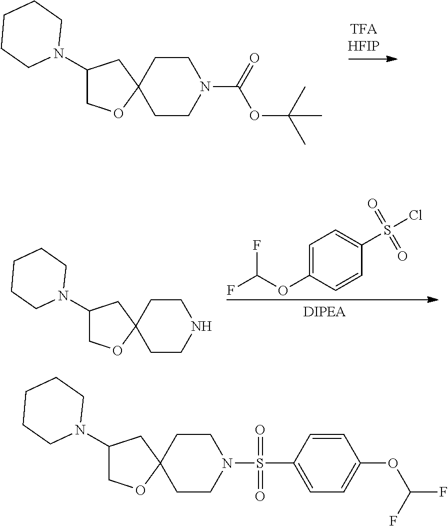 [SMILES: CC(C)(C)OC(=O)N1CCC2(CC1)CC(N1CCCCC1)CO2 ; C1CCN(C2COC3(CCNCC3)C2)CC1 ; O=S(=O)(Cl)c1ccc(OC(F)F)cc1 ; O=S(=O)(c1ccc(OC(F)F)cc1)N1CCC2(CC1)CC(N1CCCCC1)CO2]

Step A: TFA (180 μL, 2.3 mmol) was added to a solution of tert-butyl 3-(1-piperidyl)-1-oxa-8-azaspiro[4.5]decane-8-carboxylate (340 mg, 1.1 mmol) in 1,1,1,3,3,3-hexafluoropropan-2-ol (10 mL, 95 mmol) at 0° C. and the solution allowed to warm to room temperature over 90 min. The reaction mixture was concentrated in vacuo then diluted with DMF (8 mL). The material was filtered through a 10 g hyperSep SCX column (eluting with 2N NH₃ in MeOH) and the solvent was removed in vacuo to give 3-(piperidin-1-yl)-1-oxa-8-azaspiro[4.5]decane, that was used without further purification.

Step B: 3-(Piperidin-1-yl)-1-oxa-8-azaspiro[4.5]decane (85 mg, 0.4 mmol) was dissolved in DMF (2 mL), DIPEA (660 μL, 3.8 mmol) and 4-(difluoromethoxy)benzenesulfonyl chloride (92 mg, 0.4 mmol) were added and the reaction stirred for 30 min. The reaction was quenched with water and the mixture was extracted with EtOAc (3×). The combined organics were washed with saturated aqueous NaHCO₃ solution (2×), then dried over anhydrous Na₂SO₄. The crude material was purified by silica gel chromatography (0-65% EtOAc to 3:1 EtOAc:EtOH) to afford 8-((4-(difluoromethoxy)phenyl)sulfonyl)-3-(piperidin-1-yl)-1-oxa-8-azaspiro[4.5]decane (27 mg, 16%). LCMS m/z=431.2 [M+H]⁺. ¹H-NMR (400 MHz, CDCl₃) δ (ppm): 7.80-7.70 (m, 2H), 7.30-7.20 (m, 2H), 6.62 (t, J=7.3 Hz, 1H), 3.92 (dd, J=6.8, 8.5 Hz, 1H), 3.62 (t, J=8.4 Hz, 1H), 3.47 (tdd, J=2.0, 4.1, 11.5 Hz, 2H), 2.88 (quin, J=8.0 Hz, 1H), 2.72 (dq, J=4.0, 11.1 Hz, 2H), 2.50-2.20 (m, 4H), 1.94 (dd, J=7.8, 12.3 Hz, 1H), 1.80-1.50 (m, 9H), 1.50-1.40 (m, 2H).

### Examples 15a and 15b: (R)-4-Methyl-3-((3-morpholino-1-oxa-8-azaspiro[4.5]decan-8-yl)sulfonyl)benzonitrile and (S)-4-methyl-3-((3-morpholino-1-oxa-8-azaspiro[4.5]decan-8-yl)sulfonyl)benzonitrile

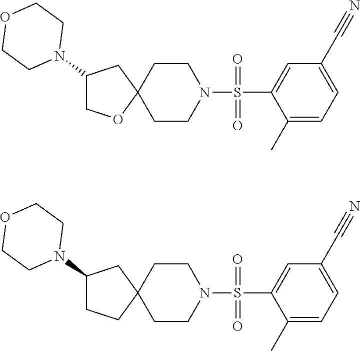

### 1. Synthesis of 4-methyl-3-((3-morpholino-1-oxa-8-azaspiro[4.5]decan-8-yl)sulfonyl)benzonitrile

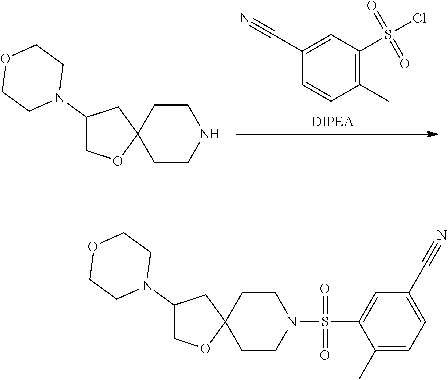 [SMILES: C1CC2(CCN1)CC(N1CCOCC1)CO2 ; Cc1ccc(C#N)cc1S(=O)(=O)Cl ; Cc1ccc(C#N)cc1S(=O)(=O)N1CCC2(CC1)CC(N1CCOCC1)CO2]

DIPEA (335 μL, 1.9 mmol) was added to 3-morpholino-1-oxa-8-azaspiro[4.5]decane hydrochloride (Intermediate A, 75 mg, 0.3 mmol) in anhydrous DMF (1.5 mL). After 5 min, 5-cyano-2-methyl-benzenesulfonyl chloride (71.5 mg, 0.3 mmol) was added and the resulting reaction mixture was stirred at room temperature for 1 h. The reaction was quenched with water, the aqueous phase was separated and extracted with EtOAc (3×). The combined organics were washed with saturated aqueous NaHCO₃ solution (2×), dried over Na₂SO₄ and concentrated in vacuo. The crude material was purified by silica gel chromatography (0-65% EtOAc to 3:1 EtOAc:EtOH) to afford 4-methyl-3-((3-morpholino-1-oxa-8-azaspiro[4.5]decan-8-yl)sulfonyl)benzonitrile (27 mg, 16%).

### 2. Separation of 4-methyl-3-((3-morpholino-1-oxa-8-azaspiro[4.5]decan-8-yl)sulfonyl)benzonitrile

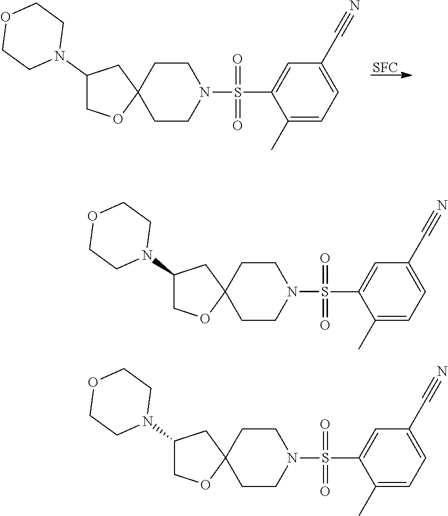

4-Methyl-3-((3-morpholino-1-oxa-8-azaspiro[4.5]decan-8-yl)sulfonyl)benzonitrile was further purified by SFC (LUX Cellulose-2 LC 30×250 mm, 5 μm column. Method: 30% MeOH in CO₂ (flow rate: 100 mL/min, ABPR 120 bar, MBPR 40 psi, column temp 40° C.) to provide two enantiomers of arbitrarily assigned stereochemistry:
Peak 1, (R)-4-methyl-3-((3-morpholino-1-oxa-8-azaspiro[4.5]decan-8-yl)sulfonyl)benzonitrile: LCMS m/z=406.2 [M+H]⁺. R_{f}=4.01 min., ee=98.86%
Peak 2, (S)-4-methyl-3-((3-morpholino-1-oxa-8-azaspiro[4.5]decan-8-yl)sulfonyl)benzonitrile the stereochemistry of which was arbitrarily assigned: LCMS m/z=406.2 [M+H]⁺. R_{f}=4.31 min., ee=90.62%

### Example 16: 4-Fluoro-3-((3-(piperidin-1-yl)-1-oxa-8-azaspiro[4.5]decan-8-yl)sulfonyl)benzonitrile

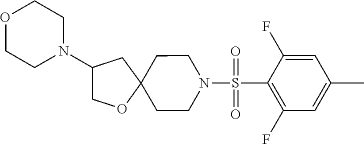 [SMILES: Cc1cc(F)c(S(=O)(=O)N2CCC3(CC2)CC(N2CCOCC2)CO3)c(F)c1]

4-Fluoro-3-((3-(piperidin-1-yl)-1-oxa-8-azaspiro[4.5]decan-8-yl)sulfonyl)benzonitrile was obtained (15 mg, 35%) from 2,6-difluoro-4-methyl-benzenesulfonyl chloride and 3-morpholino-1-oxa-8-azaspiro[4.5]decane hydrochloride (Intermediate A), following the procedure described in Example 16, step 1. LCMS m/z=417.2 [M+H]⁺. 1H-NMR (400 MHz, CDCl₃) δ (ppm): 6.82 (d, J=9.8 Hz, 2H), 4.13 (q, J=7.0 Hz, 1H), 3.95 (dd, J=6.9, 8.4 Hz, 1H), 3.70 (t, J=4.6 Hz, 4H), 3.68-3.58 (m, 2H), 3.02-2.88 (m, 2H), 2.54-2.29 (m, 7H), 1.95 (dd, J=7.7, 12.4 Hz, 1H), 1.85-1.58 (m, 6H).

### Example 17: 8-((2-Methyl-4-(trifluoromethyl)phenyl)sulfonyl)-3-morpholino-1-oxa-8-azaspiro[4.5]decane

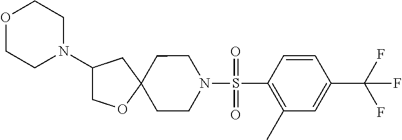 [SMILES: Cc1cc(C(F)(F)F)ccc1S(=O)(=O)N1CCC2(CC1)CC(N1CCOCC1)CO2]

8-((2-Methyl-4-(trifluoromethyl)phenyl)sulfonyl)-3-morpholino-1-oxa-8-azaspiro[4.5]decane was obtained (26 mg, 46%) from 2-methyl-4-(trifluoromethyl)benzenesulfonyl chloride and 3-morpholino-1-oxa-8-azaspiro[4.5]decane hydrochloride (Intermediate A), following the procedure described in Example 16, step 1. LCMS m/z=449.1 [M+H]⁺. ¹H-NMR (400 MHz, CDCl₃) δ (ppm): 8.10-7.90 (m, 1H), 7.58 (br s, 2H), 3.98 (dd, J=6.8, 8.3 Hz, 1H), 3.80-3.60 (m, 5H), 3.60-3.50 (m, 2H), 3.20-2.90 (m, 3H), 2.70 (s, 3H), 2.60-2.30 (m, 4H), 1.90-1.60 (m, 6H).

### Example 18: 8-((2-Fluoro-5-methylphenyl)sulfonyl)-3-morpholino-1-oxa-8-azaspiro[4.5]decane

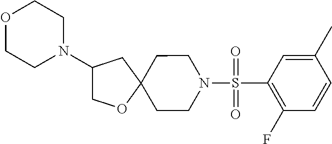 [SMILES: Cc1ccc(F)c(S(=O)(=O)N2CCC3(CC2)CC(N2CCOCC2)CO3)c1]

8-((2-Fluoro-5-methylphenyl)sulfonyl)-3-morpholino-1-oxa-8-azaspiro[4.5]decane was obtained (41 mg, 61%) from 3-morpholino-1-oxa-8-azaspiro[4.5]decane hydrochloride (Intermediate A) and 2-fluoro-5-methyl-benzenesulfonyl chloride following the procedure described in Example 16, step 1. LCMS m/z=399.2 [M+H]⁺. ¹H-NMR (400 MHz, CDCl₃) δ (ppm): 7.62 (dd, J=1.6, 6.7 Hz, 1H), 7.50-7.30 (m, 1H), 7.09 (dd, J=8.5, 9.8 Hz, 1H), 3.95 (dd, J=6.8, 8.5 Hz, 1H), 3.80-3.40 (m, 6H), 3.20-2.80 (m, 4H), 2.60-2.30 (m, 7H), 1.90-1.60 (m, 6H).

### Example 19: 8-((2,5-Difluorophenyl)sulfonyl)-3-morpholino-1-oxa-8-azaspiro[4.5]decane

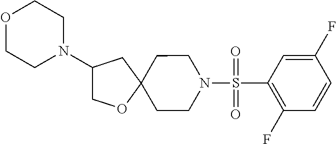 [SMILES: O=S(=O)(c1cc(F)ccc1F)N1CCC2(CC1)CC(N1CCOCC1)CO2]

8-((2,5-Difluorophenyl)sulfonyl)-3-morpholino-1-oxa-8-azaspiro[4.5]decane was obtained (41 mg, 61%) from 2,5-difluorobenzenesulfonyl chloride and 3-morpholino-1-oxa-8-azaspiro[4.5]decane hydrochloride (Intermediate A), following the procedure described in Example 16, step 1. LCMS m/z=403.1[M+H]⁺. ¹H-NMR (400 MHz, CDCl₃) δ (ppm): 7.56 (ddd, J=3.1, 5.0, 7.7 Hz, 1H), 7.30-7.10 (m, 2H), 3.96 (dd, J=6.8, 8.5 Hz, 1H), 3.80-3.50 (m, 6H), 3.10-2.90 (m, 4H), 2.60-2.30 (m, 4H), 2.00-1.60 (m, 6H).

### Example 20: 8-((6-Methoxy-2-methylpyridin-3-yl)sulfonyl)-3-morpholino-1-oxa-8-azaspiro[4.5]decane

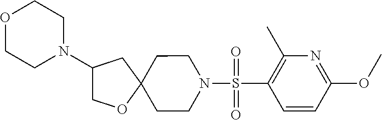 [SMILES: COc1ccc(S(=O)(=O)N2CCC3(CC2)CC(N2CCOCC2)CO3)c(C)n1]

8-((6-Methoxy-2-methylpyridin-3-yl)sulfonyl)-3-morpholino-1-oxa-8-azaspiro[4.5]decane was obtained (42 mg, 61%) from 6-methoxy-2-methyl-pyridine-3-sulfonyl chloride and 3-morpholino-1-oxa-8-azaspiro[4.5]decane hydrochloride (Intermediate A), following the procedure described in Example 16, step 1. LCMS m/z=412.2 [M+H]⁺. ¹H-NMR (400 MHz, CDCl₃) δ (ppm): 8.00 (d, J=8.8 Hz, 1H), 6.64 (d, J=8.5 Hz, 1H), 4.00-3.90 (m, 3H), 3.80-3.60 (m, 6H), 3.50-3.40 (m, 2H), 3.10-2.90 (m, 3H), 2.75 (s, 3H), 2.60-2.30 (m, 4H), 2.00-1.60 (m, 6H).

### Example 21: 4-(8-((1,3-Dimethyl-1H-pyrazol-5-yl)sulfonyl)-8-azaspiro[4.5]decan-2-yl)morpholine

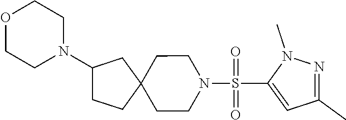 [SMILES: Cc1cc(S(=O)(=O)N2CCC3(CCC(N4CCOCC4)C3)CC2)n(C)n1]

### 1. Synthesis of 2-morpholino-8-azaspiro[4.5]decane-8-carboxylate

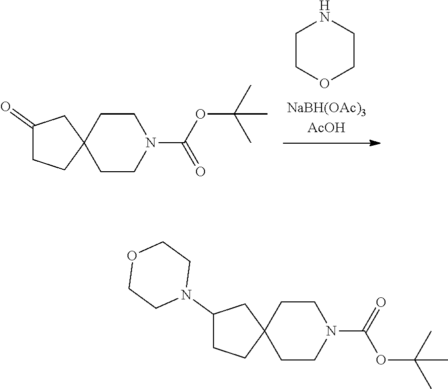 [SMILES: C1COCCN1 ; CC(C)(C)OC(=O)N1CCC2(CCC(=O)C2)CC1 ; CC(C)(C)OC(=O)N1CCC2(CCC(N3CCOCC3)C2)CC1]

Morpholine (2 mL, 22.9 mmol) was added dropwise to a solution of tert-butyl 3-oxo-8-azaspiro[4.5]decane-8-carboxylate (2.75 g, 10.9 mmol) in anhydrous DCM (40 mL) and acetic acid (0.67 mL, 11.7 mmol) at 0° C. After 15 min, NaBH(OAc)₃ (7.0 g, 33 mmol) was added in portions. Upon complete addition the reaction was warmed to room temperature and stirred for 5 h. The reaction was quenched with aqueous 2M NaOH, the mixture was stirred at room temperature for 20 min. The phases were separated and the aqueous phase was extracted with DCM (3×). The combined organic extracts were dried over MgSO₄, filtered and concentrated under reduced pressure to give 2-morpholino-8-azaspiro[4.5]decane-8-carboxylate. LCMS m/z=325.2 (M+H)⁺.

### 2. Synthesis of 4-(8-azaspiro[4.5]decan-2-yl)morpholine hydrochloride

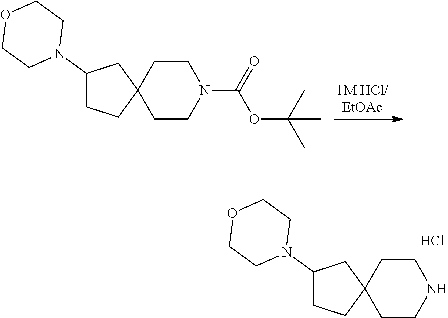 [SMILES: CC(C)(C)OC(=O)N1CCC2(CCC(N3CCOCC3)C2)CC1 ; C1CC2(CCN1)CCC(N1CCOCC1)C2 ; Cl]

1M HCl in EtOAc (35 mL) was added dropwise to solution of tert-butyl 2-morpholino-8-azaspiro[4.5]decane-8-carboxylate (3.5 g, 10.8 mmol) in MeOH (25 mL) at 0° C. The reaction mixture was warmed to room temperature and stirred for 7 days. The resulting mixture was concentrated under reduced pressure. The residue was diluted with EtOAc and filtered to afford 4-(8-azaspiro[4.5]decan-2-yl)morpholine hydrochloride as a white solid. (2.79 g, 99%). LCMS m/z=225.1 (M+H)⁺. ¹H-NMR (500 MHz, CD₃OD) δ (ppm): 4.06 (br d, J=12.8 Hz, 2H), 3.83 (br t, J=11.3 Hz, 2H), 3.71 (quin, J=8.7 Hz, 1H), 3.52 (br t, J=12.2 Hz, 2H), 3.22-3.10 (m, 6H), 2.30-2.22 (m, 2H), 2.00-1.88 (m, 2H), 1.88-1.78 (m, 2H), 1.77-1.67 (m, 4H).

### 3. Synthesis of 4-(8-((1,3-dimethyl-1H-pyrazol-5-yl)sulfonyl)-8-azaspiro[4.5]decan-2-yl)morpholine

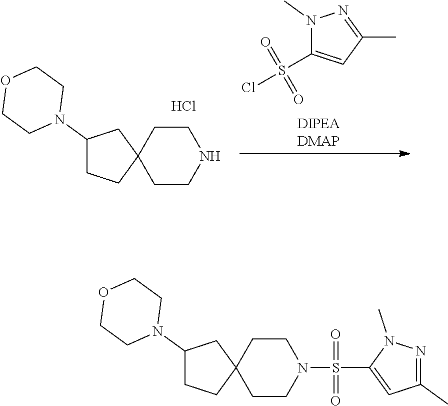 [SMILES: C1CC2(CCN1)CCC(N1CCOCC1)C2 ; Cl ; Cc1cc(S(=O)(=O)Cl)n(C)n1 ; Cc1cc(S(=O)(=O)N2CCC3(CCC(N4CCOCC4)C3)CC2)n(C)n1]

To a solution of 4-(8-azaspiro[4.5]decan-2-yl)morpholine hydrochloride (81 mg, 0.3 mmol) in anhydrous THE (1 mL) was added DIPEA (320 μL, 1.8 mmol) dropwise, DMAP (4 mg, 0.04 mmol) at <5° C. After 5 min, 2,5-dimethylpyrazole-3-sulfonyl chloride (88.3 mg, 0.5 mmol) was added to the cold solution and the reaction was warmed to room temperature and stirred for 30 min. The reaction mixture was quenched with aqueous 2M NaOH solution and the mixture stirred at room temperature for 10 min. The biphasic mixture was loaded onto a silica gel column and purified by chromatography (15-95% 3:1 EtOAc: EtOH in heptane) to give 4-(8-((1,3-dimethyl-1H-pyrazol-5-yl)sulfonyl)-8-azaspiro[4.5]decan-2-yl)morpholine (80 mg, 64%). LCMS m/z=383.1 (M+H)⁺. ¹H-NMR (500 MHz, DMSO-d₆) δ (ppm): 6.59 (s, 1H), 3.92 (s, 3H), 3.53 (br t, J=4.3 Hz, 4H), 3.08-3.03 (m, 4H), 2.49-2.44 (m, 1H), 2.38-2.28 (m, 4H), 2.18 (s, 3H), 1.80-1.74 (m, 1H), 1.71-1.65 (m, 1H), 1.58-1.52 (m, 1H), 1.51-1.44 (m, 4H), 1.43-1.33 (m, 2H), 1.19-1.14 (m, 1H).

### Examples 22a and 22b: (R)-4-(8-((1,3-Dimethyl-1H-pyrazol-5-yl)sulfonyl)-8-azaspiro[4.5]decan-2-yl)morpholine and (S)-4-(8-((1,3-dimethyl-1H-pyrazol-5-yl)sulfonyl)-8-azaspiro[4.5]decan-2-yl)morpholine

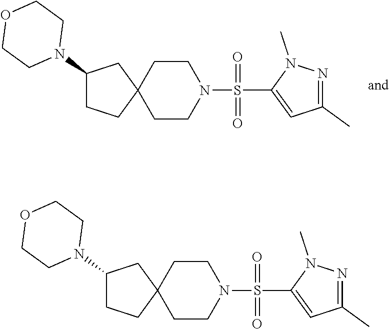 [SMILES: Cc1cc(S(=O)(=O)N2CCC3(CC[C@@H](N4CCOCC4)C3)CC2)n(C)n1 ; Cc1cc(S(=O)(=O)N2CCC3(CC[C@H](N4CCOCC4)C3)CC2)n(C)n1]

### 1. Synthesis of (R)-4-(8-((1,3-dimethyl-1H-pyrazol-5-yl)sulfonyl)-8-azaspiro[4.5]decan-2-yl)morpholine and (S)-4-(8-((1,3-dimethyl-1H-pyrazol-5-yl)sulfonyl)-8-azaspiro[4.5]decan-2-yl)morpholine

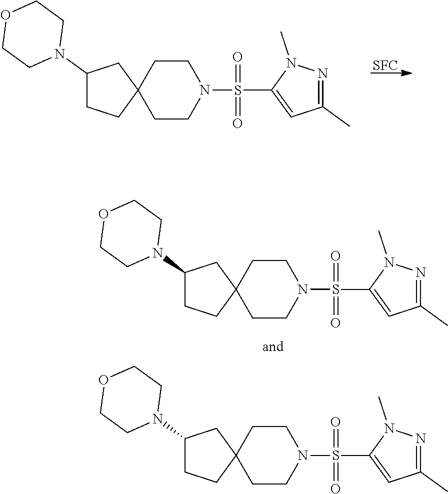

4-(8-((1,3-Dimethyl-1H-pyrazol-5-yl)sulfonyl)-8-azaspiro[4.5]decan-2-yl)morpholine (Example 22, 74 mg, 0.2 mmol) was purified on a Lux Cellulose-4 30×250 mm, 5 μm column eluting with 40% MeOH in CO₂. Flow rate: 100 mL/min; ABPR 120 bar; MBPR 40 psi, column temperature 40° C. to afford two enantiomers of arbitrarily assigned stereochemistry:
Peak 1, (R)-4-(8-((1,3-dimethyl-1H-pyrazol-5-yl)sulfonyl)-8-azaspiro[4.5]decan-2-yl)morpholine (Enantiomer 1 with t_{R}=2.35 min) was obtained as a colorless film (25 mg, 32%). LCMS m/z=383.2 [M+H]⁺. ¹H-NMR (500 MHz, DMSO-d₆) δ (ppm): 6.59 (s, 1H), 3.92 (s, 3H), 3.53 (br s, 4H), 3.09-3.02 (m, 4H), 2.39-2.27 (m, 4H), 2.18 (s, 3H), 1.80-1.73 (m, 1H), 1.71-1.66 (m, 1H), 1.65-1.27 (m, 8H), 1.20-1.12 (m, 1H).
Peak 2, (S)-4-(8-((1,3-Dimethyl-1H-pyrazol-5-yl)sulfonyl)-8-azaspiro[4.5]decan-2-yl)morpholine (Enantiomer 2 with t_{R}=2.68 min) was obtained as a colorless film (25 mg, 33%). LCMS m/z=383.3 [M+H]⁺. ¹H-NMR (500 MHz, DMSO-d₆) δ (ppm): 6.59 (s, 1H), 3.92 (s, 3H), 3.53 (br s, 4H), 3.05 (br s, 4H), 2.37-2.26 (m, 4H), 2.18 (s, 3H), 1.80-1.73 (m, 1H), 1.71-1.65 (m, 1H), 1.59-1.30 (m, 8H), 1.19-1.13 (m, 1H).

### Example 23: 4-(8-((4-(Difluoromethoxy)phenyl)sulfonyl)-8-azaspiro[4.5]decan-2-yl)morpholine

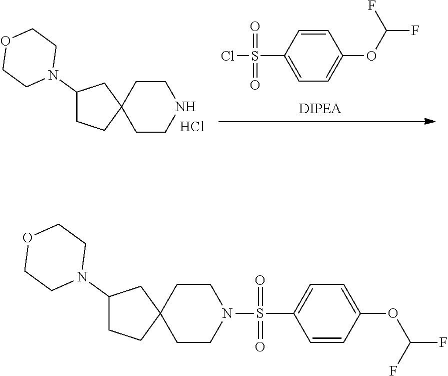 [SMILES: C1CC2(CCN1)CCC(N1CCOCC1)C2 ; Cl ; O=S(=O)(Cl)c1ccc(OC(F)F)cc1 ; O=S(=O)(c1ccc(OC(F)F)cc1)N1CCC2(CCC(N3CCOCC3)C2)CC1]

To a solution of 4-(8-azaspiro[4.5]decan-2-yl)morpholine hydrochloride (130 mg, 0.5 mmol) in DCM (3 mL) was added DIPEA (260 μL, 1.5 mmol) and 4-(difluoromethoxy)benzenesulfonyl chloride (133 mg, 0.6 mmol) at 0° C. and the mixture was stirred at 20° C. for 1.5 h. The reaction mixture was concentrated under reduced pressure and the resulting residue was purified by prep-HPLC (Column: Boston Prime C18 150×30 mm×5 μm; Method: water (0.05% NH₄OH v/v)−MeCN Begin B 34 End B 64; Flow Rate (mL/min) 30 to give 4-(8-((4-(difluoromethoxy)phenyl)sulfonyl)-8-azaspiro[4.5]decan-2-yl)morpholine (98 mg, 46%) as a yellow gum. LCMS m/z=431.2 (M+H)⁺. ¹H-NMR (400 MHz, DMSO-d₆) 7.80 (d, J=8.8 Hz, 2H), 7.62-7.21 (m, 3H), 3.51 (br t, J=4.4 Hz, 4H), 2.96-2.80 (m, 4H), 2.35-2.23 (m, 5H), 1.78-1.03 (m, 10H).

### Example 24: 2-Methyl-3-((2-morpholino-8-azaspiro[4.5]decan-8-yl)sulfonyl)-2,4,6,7-tetrahydropyrano[4,3-c]pyrazole

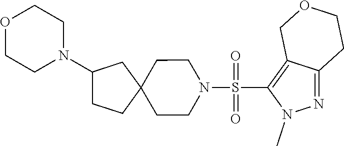 [SMILES: Cn1nc2c(c1S(=O)(=O)N1CCC3(CCC(N4CCOCC4)C3)CC1)COCC2]

### 1. Synthesis of 2-methyl-3-((2-morpholino-8-azaspiro[4.5]decan-8-yl)sulfonyl)-2,4,6,7-tetrahydropyrano[4,3-c]pyrazole

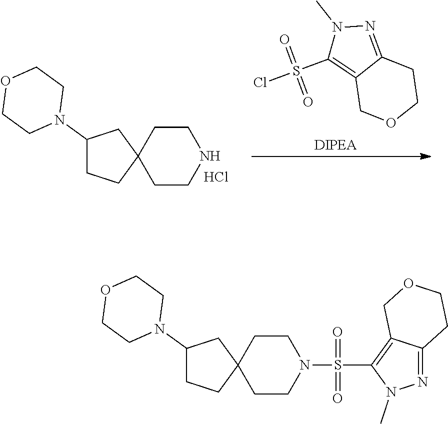 [SMILES: C1CC2(CCN1)CCC(N1CCOCC1)C2 ; Cl ; Cn1nc2c(c1S(=O)(=O)Cl)COCC2 ; Cn1nc2c(c1S(=O)(=O)N1CCC3(CCC(N4CCOCC4)C3)CC1)COCC2]

To a solution of 4-(8-azaspiro[4.5]decan-2-yl)morpholine hydrochloride (Example 22, step 2, 70 mg, 0.3 mmol) in anhydrous THE (1 mL) was added DIPEA (300 μL, 1.7 mmol) dropwise at <5° C. After 5 min, 2-methyl-6,7-dihydro-4H-pyrano[4,3-c]pyrazole-3-sulfonyl chloride (89 mg, 0.4 mmol) was added, the reaction was warmed to room temperature and stirred for 30 min. The heterogeneous reaction mixture was quenched with aqueous 2M NaOH solution and the mixture stirred at room temperature for 10 min. The biphasic mixture was loaded onto a silica gel column and purified with (20-80% 3:1 EtOAc: EtOH in heptane) to afford 2-methyl-3-((2-morpholino-8-azaspiro[4.5]decan-8-yl)sulfonyl)-2,4,6,7-tetrahydropyrano[4,3-c]pyrazole (17 mg, 14%). LCMS m/z=425.3 (M+H)⁺. ¹H-NMR (500 MHz, DMSO-d₆) δ (ppm): 4.63 (s, 2H), 3.94 (s, 3H), 3.84 (t, J=5.8 Hz, 2H), 3.53 (br t, J=4.0 Hz, 4H), 3.08 (br t, J=5.5 Hz, 4H), 2.67 (t, J=5.8 Hz, 2H), 2.48-2.44 (m, 1H), 2.37-2.27 (m, 4H), 1.81-1.73 (m, 1H), 1.69 (dd, J=7.3, 12.8 Hz, 1H), 1.57-1.50 (m, 1H), 1.50-1.44 (m, 4H), 1.43-1.35 (m, 2H), 1.19-1.14 (m, 1H).

### Example 25: 4-(8-((3,5-Dimethylpyridin-2-yl)sulfonyl)-8-azaspiro[4.5]decan-2-yl)morpholine

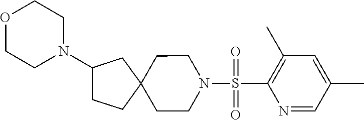 [SMILES: Cc1cnc(S(=O)(=O)N2CCC3(CCC(N4CCOCC4)C3)CC2)c(C)c1]

### 1. Synthesis of 4-(8-((3,5-dimethylpyridin-2-yl)sulfonyl)-8-azaspiro[4.5]decan-2-yl)morpholine

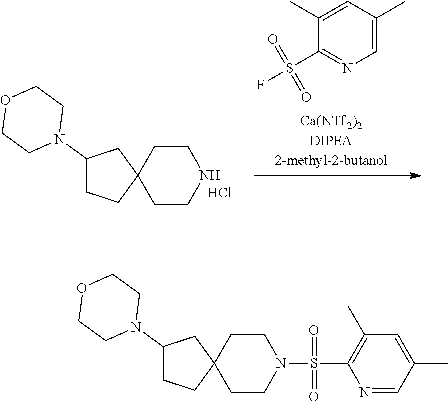 [SMILES: C1CC2(CCN1)CCC(N1CCOCC1)C2 ; Cl ; Cc1cnc(S(=O)(=O)F)c(C)c1 ; Cc1cnc(S(=O)(=O)N2CCC3(CCC(N4CCOCC4)C3)CC2)c(C)c1]

To a solution of 4-(8-azaspiro[4.5]decan-2-yl)morpholine hydrochloride (Example 22, step 2, 154 mg, 0.6 mmol) in anhydrous THE (1.5 mL) and MeOH (0.2 mL) was added DIPEA (300 μL, 1.7 mmol) dropwise at room temperature. The resulting mixture was stirred for 10 min and evaporated under reduced pressure. 2-Methyl-2-butanol (1.5 mL), 3,5-dimethylpyridine-2-sulfonyl fluoride (567 mg, 0.3 mmol) were added followed by the batchwise addition of Ca(NTf₂)₂ (195 mg, 0.3 mmol). the resulting mixture was heated at 60° C. for 19 h. The reaction mixture was brought to room temperature, quenched with aqueous 2 M NaOH and the resulting heterogeneous mixture was loaded onto a silica gel column and purified by column chromatography (25-85% 3:1 EtOAc: EtOH in heptane). The product was further purified by prep HPLC using a Waters XSelect CSH C18, 5 μm, 50 mm×100 mm column with mobile phase water (A) and MeCN (B) and a gradient of 5-60% B (0.2% NH₄OH final v/v % modifier) with flow rate at 80 mL/min, to give 4-(8-((3,5-dimethylpyridin-2-yl)sulfonyl)-8-azaspiro[4.5]decan-2-yl)morpholine as a white solid (30 mg, 24%). LCMS m/z=394.4 (M+H)⁺. ¹H-NMR (500 MHz, DMSO-d₆) δ (ppm): 8.33 (s, 1H), 7.73 (s, 1H), 3.56 (br s, 4H), 3.42-3.36 (m, 4H), 2.56-2.52 (m, 1H), 2.50 (s, 3H), 2.41-2.30 (m, 7H), 1.86-1.79 (m, 1H), 1.79-1.73 (m, 1H), 1.57-1.43 (m, 7H), 1.27-1.21 (m, 1H).

### Example 26: 4-(8-((3-Methoxy-1-methyl-1H-pyrazol-5-yl)sulfonyl)-8-azaspiro[4.5]decan-2-yl)morpholine

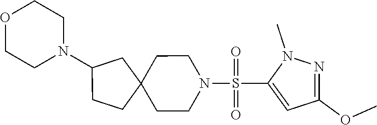 [SMILES: COc1cc(S(=O)(=O)N2CCC3(CCC(N4CCOCC4)C3)CC2)n(C)n1]

### 1. Synthesis of 4-(8-((3-bromo-1-methyl-1H-pyrazol-5-yl)sulfonyl)-8-azaspiro[4.5]decan-2-yl)morpholine

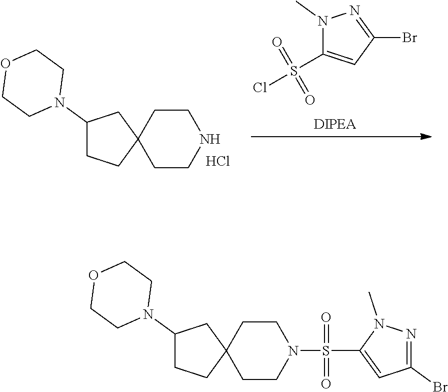 [SMILES: C1CC2(CCN1)CCC(N1CCOCC1)C2 ; Cl ; Cn1nc(Br)cc1S(=O)(=O)Cl ; Cn1nc(Br)cc1S(=O)(=O)N1CCC2(CCC(N3CCOCC3)C2)CC1]

4-(8-((3-Bromo-1-methyl-1H-pyrazol-5-yl)sulfonyl)-8-azaspiro[4.5]decan-2-yl)morpholine was obtained (582 mg, 84%) from 4-(8-azaspiro[4.5]decan-2-yl)morpholine hydrochloride and 5-bromo-2-methyl-pyrazole-3-sulfonyl chloride, following the procedure described in Example 23, step 1. ¹H-NMR (500 MHz, DCM-d₂) δ (ppm): 6.66 (s, 1H), 4.04 (s, 3H), 3.62 (t, J=4.6 Hz, 4H), 3.16-3.09 (m, 4H), 2.55-2.50 (m, 1H), 2.42-2.35 (m, 4H), 1.88-1.82 (m, 1H), 1.75-1.70 (m, 1H), 1.68-1.63 (m, 1H), 1.61-1.56 (m, 2H), 1.55-1.50 (m, 2H), 1.49-1.38 (m, 2H), 1.26-1.21 (m, 1H).

### 2. Synthesis of 4-(8-((3-methoxy-1-methyl-1H-pyrazol-5-yl)sulfonyl)-8-azaspiro[4.5]decan-2-yl)morpholine

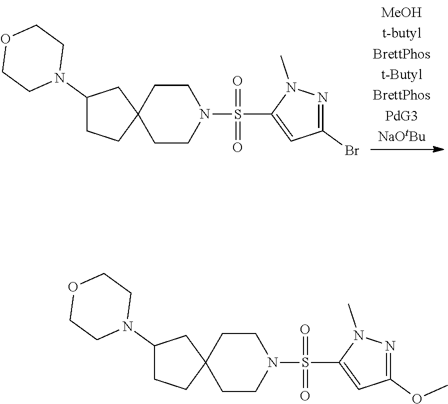 [SMILES: Cn1nc(Br)cc1S(=O)(=O)N1CCC2(CCC(N3CCOCC3)C2)CC1 ; COc1cc(S(=O)(=O)N2CCC3(CCC(N4CCOCC4)C3)CC2)n(C)n1]

A vial containing 4-(8-((3-bromo-1-methyl-1H-pyrazol-5-yl)sulfonyl)-8-azaspiro[4.5]decan-2-yl)morpholine (152 mg, 0.3 mmol), t-Butyl BrettPhos (50 mg, 0.1 mmol), t-Butyl BrettPhos PdG3 (43 mg, 0.01 mmol), and sodium tert-butoxide (176 mg, 1.8 mmol) in anhydrous dioxane (3 mL) was evacuated and backfilled with N₂. Degassed MeOH (400 μL, 9.9 mmol) was added dropwise and resulting mixture was heated at 55° C. for 24 h. The reaction was cooled to room temperature, diluted with water and extracted with DCM (3×). The combined organic layers were dried over anhydrous MgSO₄, filtered and concentrated under reduced pressure. The residue was purified by silica gel column chromatography eluting with (30-90% 3:1 EtOAc: EtOH in heptane). The obtained product was further purified by HPLC using a Waters XSelect CSH C18, 5 μm, 50 mm×100 mm column with mobile phase water (A) and MeCN (B) and a gradient of 5-50% B (0.2% NH₄OH final v/v % modifier) with flow rate at 60 mL/min to give 4-(8-((3-methoxy-1-methyl-1H-pyrazol-5-yl)sulfonyl)-8-azaspiro[4.5]decan-2-yl)morpholine as a colorless film (6 mg, 4%). LCMS m/z=399.3 [M+H]⁺. ¹H-NMR (500 MHz, DCM-d₂) δ (ppm): 6.04 (s, 1H), 3.91 (s, 3H), 3.85 (s, 3H), 3.73-3.54 (m, 4H), 3.15-3.07 (m, 4H), 2.76-2.55 (m, 1H), 2.54-2.22 (m, 4H), 1.90-1.83 (m, 1H), 1.76-1.71 (m, 1H), 1.68-1.64 (m, 1H), 1.61-1.52 (m, 5H), 1.46-1.40 (m, 1H), 1.35-1.20 (m, 1H).

### Example 27: (1R,4R)-5-(8-((4-(Difluoromethoxy)phenyl)sulfonyl)-8-azaspiro[4.5]decan-2-yl)-2-oxa-5-azabicyclo[2.2.1]heptane

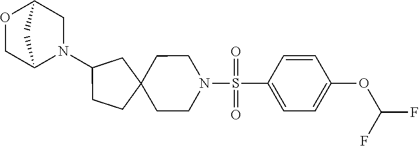 [SMILES: O=S(=O)(c1ccc(OC(F)F)cc1)N1CCC2(CCC(N3C[C@H]4C[C@@H]3CO4)C2)CC1]

### 1. Synthesis of tert-butyl 2-((1R,4R)-2-oxa-5-azabicyclo[2.2.1]heptan-5-yl)-8-azaspiro[4.5]decane-8-carboxylate

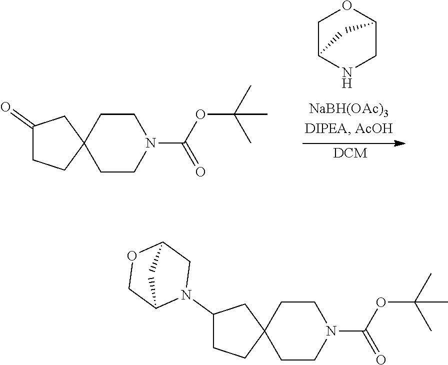 [SMILES: C1O[C@H]2CN[C@@H]1C2 ; CC(C)(C)OC(=O)N1CCC2(CCC(=O)C2)CC1 ; CC(C)(C)OC(=O)N1CCC2(CCC(N3C[C@H]4C[C@@H]3CO4)C2)CC1]

To a solution of tert-butyl 3-oxo-8-azaspiro[4.5]decane-8-carboxylate (247 mg, 1.0 mmol) in anhydrous DCM (3 mL) and acetic acid (200 μL, 3.5 mmol) was added a solution of (1R,4R)-2-oxa-5-azabicyclo[2.2.1]heptane hydrochloride (295 mg, 2.2 mmol) and DIPEA (430 μL, 2.5 mmol) in anhydrous DCM (3 mL). After 15 min, NaBH(OAc)₃ (857 mg, 4.0 mmol) was added in portions, and the reaction mixture was stirred at room temperature for 20 h. The reaction was quenched with aqueous 2M NaOH solution and the mixture was stirred at room temperature for 20 min. The biphasic mixture was extracted with DCM (3×), the combined organic extracts were dried over anhydrous MgSO₄, filtered and evaporated under reduced pressure to give tert-butyl 2-((1R,4R)-2-oxa-5-azabicyclo[2.2.1]heptan-5-yl)-8-azaspiro[4.5]decane-8-carboxylate (307 mg, 94%) that was used without further purification. LCMS m/z=337.3 (M+H)⁺.

### 2. Synthesis of (1R,4R)-5-(8-azaspiro[4.5]decan-2-yl)-2-oxa-5-azabicyclo[2.2.1]heptane hydrochloride

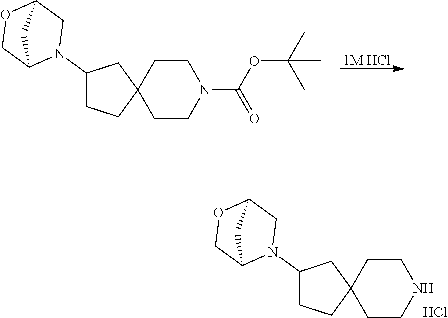

A solution of tert-butyl 2-((1R,4R)-2-oxa-5-azabicyclo[2.2.1]heptan-5-yl)-8-azaspiro[4.5]decane-8-carboxylate (307 mg, 0.9 mmol) in MeOH (0.5 mL) was cooled in an ice water bath and 1M HCl in EtOAc (1 M, 3.5 mL) was added dropwise. Upon complete addition, the reaction mixture was warmed to room temperature and stirred for 5 days. The reaction mixture was concentrated under reduced pressure to give (1R,4R)-5-(8-azaspiro[4.5]decan-2-yl)-2-oxa-5-azabicyclo[2.2.1]heptane hydrochloride which was used without further purification in the next step. LCMS m/z=237.1 (M+H)⁺.

### 3. Synthesis of (1R,4R)-5-(8-((4-(difluoromethoxy)phenyl)sulfonyl)-8-azaspiro[4.5]decan-2-yl)-2-oxa-5-azabicyclo[2.2.1]heptane

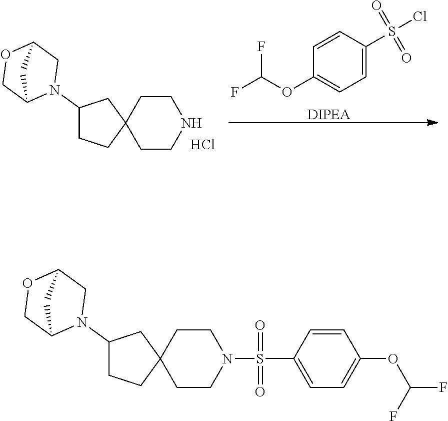

To a solution of (1R,4R)-5-(8-azaspiro[4.5]decan-2-yl)-2-oxa-5-azabicyclo[2.2.1]heptane hydrochloride (124 mg, 0.5 mmol) in anhydrous THE (2 mL) was added DIPEA (500 μL, 2.9 mmol) dropwise at <5° C. After 5 min, 4-(difluoromethoxy)benzenesulfonyl chloride (120 μL, 0.8 mmol) was added, the reaction was warmed to room temperature and stirred for 30 min. The reaction mixture was quenched with aqueous 2 M NaOH solution and the resulting biphasic mixture was stirred at room temperature for 10 min. The biphasic mixture was loaded onto silica gel and purified by column chromatography (15-85% 3:1 EtOAc: EtOH in heptane) The product was further purified by HPLC using a Waters XSelect CSH C18, 5 μm, 50 mm×100 mm column with mobile phase water (A) and MeCN (B) and a gradient of 5-70% B (0.2% NH₄OH final v/v % modifier) with flow rate at 80 mL/min to give (1R,4R)-5-(8-((4-(difluoromethoxy)phenyl)sulfonyl)-8-azaspiro[4.5]decan-2-yl)-2-oxa-5-azabicyclo[2.2.1]heptane as a beige solid (67 mg, 30%). LCMS m/z=443.3 [M+H]⁺. ¹H-NMR (600 MHz, DMSO-d₆) d=7.80 (d, J=9.4 Hz, 2H), 7.61-7.22 (m, 3H), 3.80-3.78 (m, 1H), 2.96-2.84 (m, 5H), 2.76-2.71 (m, 1H), 2.28 (br dd, J=5.1, 9.4 Hz, 1H), 1.63 (br d, J=8.7 Hz, 2H), 1.58-1.53 (m, 2H), 1.52-1.46 (m, 6H), 1.42-1.35 (m, 3H), 1.28 (br dd, J=4.4, 8.0 Hz, 1H), 1.15-1.11 (m, 1H).

### Example 28: 8-((4,6-Dimethylpyridin-3-yl)sulfonyl)-3-(2-oxa-7-azaspiro[4.4]nonan-7-yl)-1-oxa-8-azaspiro[4.5]decane

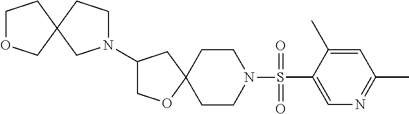 [SMILES: Cc1cc(C)c(S(=O)(=O)N2CCC3(CC2)CC(N2CCC4(CCOC4)C2)CO3)cn1]

### 1. Synthesis of tert-butyl 3-(2-oxa-7-azaspiro[4.4]nonan-7-yl)-1-oxa-8-azaspiro[4.5]decane-8-carboxylate

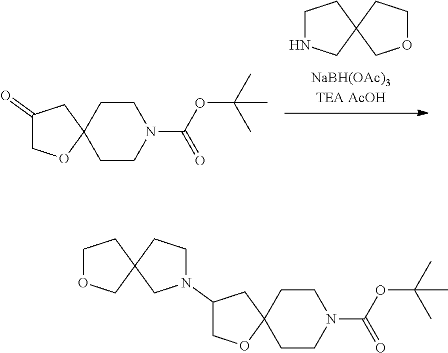 [SMILES: C1CC2(CCOC2)CN1 ; CC(C)(C)OC(=O)N1CCC2(CC1)CC(=O)CO2 ; CC(C)(C)OC(=O)N1CCC2(CC1)CC(N1CCC3(CCOC3)C1)CO2]

TEA (601 μL, 4.3 mmol) was slowly added to a solution of tert-butyl 3-oxo-1-oxa-8-azaspiro[4.5]decane-8-carboxylate (500 mg, 2.0 mmol) and 2-oxa-7-azaspiro[4.4]nonane (299 mg, 2.4 mmol) in DCM (15 mL) at room temperature. After 15 min acetic acid (280 μL, 4.90 mmol) was slowly added followed by NaBH(OAc)₃ (1.7 g, 7.8 mmol) after an additional 30 min. Stirring was continued at room temperature for 1 d saturated aqueous NH₄Cl was added. The phases were separated, and the aqueous Phase was extracted with EtOAc. The combined organic phase was washed with brine, dried over anhydrous Na₂SO₄, filtered and concentrated under reduced pressure. The resulting residue was purified by silica gel chromatography (0-100% EtOH:EtOAc 1:3 in heptane) to give tert-butyl 3-(2-oxa-7-azaspiro[4.4]nonan-7-yl)-1-oxa-8-azaspiro[4.5]decane-8-carboxylate (197 mg, 27%). LCMS m/z=367.2 [M+H]⁺.

### 2. Synthesis of 3-(2-oxa-7-azaspiro[4.4]nonan-7-yl)-1-oxa-8-azaspiro[4.5]decane hydrochloride

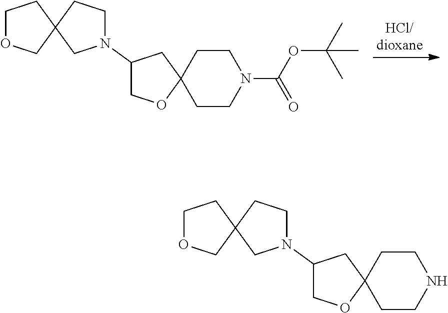 [SMILES: CC(C)(C)OC(=O)N1CCC2(CC1)CC(N1CCC3(CCOC3)C1)CO2 ; C1CC2(CCN1)CC(N1CCC3(CCOC3)C1)CO2]

tert-Butyl 3-(2-oxa-7-azaspiro[4.4]nonan-7-yl)-1-oxa-8-azaspiro[4.5]decane-8-carboxylate (197 mg, 0.5 mmol) was dissolved in EtOAc (4 mL). 4M HCl in dioxane (670 μL, 2.7 mmol) was added and the reaction mixture was stirred at room temperature overnight. The reaction mixture was concentrated under reduced pressure to give 3-(2-oxa-7-azaspiro[4.4]nonan-7-yl)-1-oxa-8-azaspiro[4.5]decane hydrochloride (215 mg). LCMS m/z=267.1 [M+H]⁺.

### 3. Synthesis of 8-((4,6-dimethylpyridin-3-yl)sulfonyl)-3-(2-oxa-7-azaspiro[4.4]nonan-7-yl)-1-oxa-8-azaspiro[4.5]decane

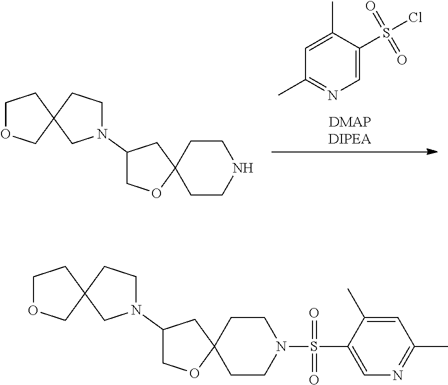 [SMILES: C1CC2(CCN1)CC(N1CCC3(CCOC3)C1)CO2 ; Cc1cc(C)c(S(=O)(=O)Cl)cn1 ; Cc1cc(C)c(S(=O)(=O)N2CCC3(CC2)CC(N2CCC4(CCOC4)C2)CO3)cn1]

To a solution of 3-(2-oxa-7-azaspiro[4.4]nonan-7-yl)-1-oxa-8-azaspiro[4.5]decane hydrochloride (36 mg, 0.1 mmol) in THF (1 mL) was added DMA (83 μL), DMAP (1 mg, 0.01 mmol) and 4,6-dimethylpyridine-3-sulfonyl chloride (29 mg, 0.1 mmol) at room temperature. DCM (1 mL), DMF (0.5 mL) and DIPEA (80 μL, 0.5 mmol) were added and the reaction was stirred for 15 min. The reaction mixture was quenched by addition of saturated aqueous NH₄Cl and brine. The mixture was extracted with EtOAc (2×) and the combined organic extracts were separated and concentrated under reduced pressure. The residue was purified by preparative HPLC (Waters SunFire Prep C18 5 μm OBD 30×100 mm; Method: (A) 95% water//(B) 5% MeCN w/0.1% TFA to 70% (A)/30% (B) over 7.5 min (flow rate: 50 mL/min) to give 8-((4,6-dimethylpyridin-3-yl)sulfonyl)-3-(2-oxa-7-azaspiro[4.4]nonan-7-yl)-1-oxa-8-azaspiro[4.5]decane trifluoroacetate (7.9 mg, 14%) as clear oil. LCMS m/z=436.2 [M+H]⁺.

### Example 29: 8-((1,3-Dimethyl-1H-pyrazol-5-yl)sulfonyl)-3-(2-oxa-7-azaspiro[4.4]nonan-7-yl)-1-oxa-8-azaspiro[4.5]decane

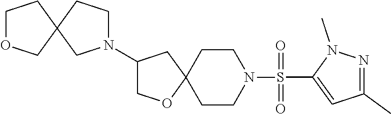 [SMILES: Cc1cc(S(=O)(=O)N2CCC3(CC2)CC(N2CCC4(CCOC4)C2)CO3)n(C)n1]

### 1. Synthesis of 8-((1,3-dimethyl-1H-pyrazol-5-yl)sulfonyl)-3-(2-oxa-7-azaspiro[4.4]nonan-7-yl)-1-oxa-8-azaspiro[4.5]decane

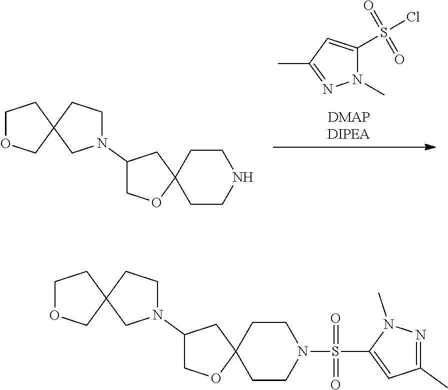 [SMILES: Cc1cc(S(=O)(=O)Cl)n(C)n1 ; C1CC2(CCN1)CC(N1CCC3(CCOC3)C1)CO2 ; Cc1cc(S(=O)(=O)N2CCC3(CC2)CC(N2CCC4(CCOC4)C2)CO3)n(C)n1]

DIPEA (120 μL, 0.7 mmol) followed by DMAP (1 mg, 0.01 mmol) were added to a solution of 3-(2-oxa-7-azaspiro[4.4]nonan-7-yl)-1-oxa-8-azaspiro[4.5]decane hydrochloride (Example 28, step 2, 36 mg, 0.11 mmol) in THF (1 mL). 1,3-Dimethyl-1H-pyrazole-5-sulfonyl chloride (28 mg, 0.1 mmol) was added and the reaction was stirred at room temperature for 1 d. The reaction was diluted with saturated NH₄Cl and brine, extracted with EtOAc (2×). The combined organic extracts were concentrated under reduced pressure. The residue was purified by preparative HPLC (Waters SunFire Prep C18 5 μm OBD 30×100 mm; Method: (A) 95% water//(B) 5% MeCN w/0.1% TFA to 50% (A)/50% (B) over 7.5 min (flow rate: 50 mL/min) to give 8-((1,3-dimethyl-1H-pyrazol-5-yl)sulfonyl)-3-(2-oxa-7-azaspiro[4.4]nonan-7-yl)-1-oxa-8-azaspiro[4.5]decane trifluoroacetate (7.4 mg, 13%) as yellow oil. LCMS m/z=425.3 [M+H]⁺.

### Examples 30a, 30b, 30c, and 30d: (S)-8-((4-(Difluoromethoxy)phenyl)sulfonyl)-3-((R)-2-oxa-7-azaspiro[4.4]nonan-7-yl)-1-oxa-8-azaspiro[4.5]decane, (S)-8-((4-(difluoromethoxy)phenyl)sulfonyl)-3-((S)-2-oxa-7-azaspiro[4.4]nonan-7-yl)-1-oxa-8-azaspiro[4.5]decane, (R)-8-((4-(difluoromethoxy)phenyl)sulfonyl)-3-((R)-2-oxa-7-azaspiro[4.4]nonan-7-yl)-1-oxa-8-azaspiro[4.5]decane and (R)-8-((4-(difluoromethoxy)phenyl)sulfonyl)-3-((S)-2-oxa-7-azaspiro[4.4]nonan-7-yl)-1-oxa-8-azaspiro[4.5]decane

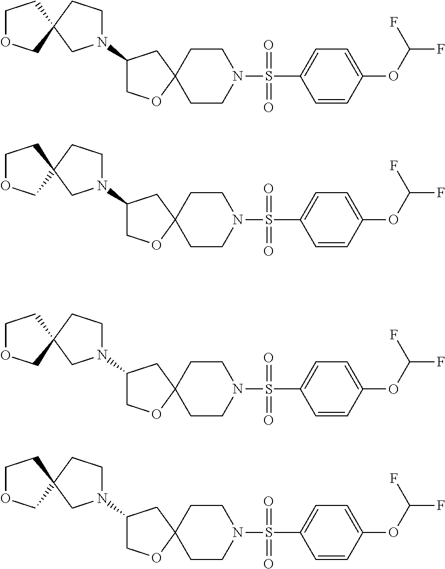

### 1. Synthesis of 8-((4-(difluoromethoxy)phenyl)sulfonyl)-3-(2-oxa-7-azaspiro[4.4]nonan-7-yl)-1-oxa-8-azaspiro[4.5]decane

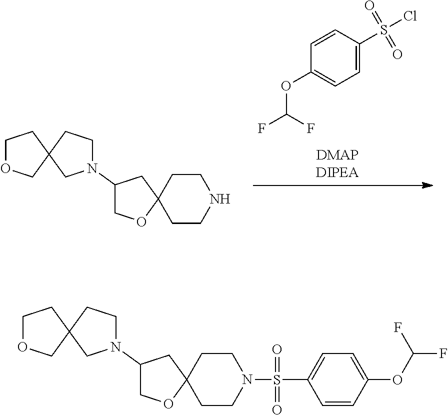 [SMILES: O=S(=O)(Cl)c1ccc(OC(F)F)cc1 ; C1CC2(CCN1)CC(N1CCC3(CCOC3)C1)CO2 ; O=S(=O)(c1ccc(OC(F)F)cc1)N1CCC2(CC1)CC(N1CCC3(CCOC3)C1)CO2]

8-((4-(Difluoromethoxy)phenyl)sulfonyl)-3-(2-oxa-7-azaspiro[4.4]nonan-7-yl)-1-oxa-8-azaspiro[4.5]decane was obtained from 3-(2-oxa-7-azaspiro[4.4]nonan-7-yl)-1-oxa-8-azaspiro[4.5]decane hydrochloride (Example 28, step 2) and 4-(difluoromethoxy)benzenesulfonyl chloride following a similar reaction to that described in Example 28, step 3.

### 2. Separation of 8-((4-(difluoromethoxy)phenyl)sulfonyl)-3-(2-oxa-7-azaspiro[4.4]nonan-7-yl)-1-oxa-8-azaspiro[4.5]decane

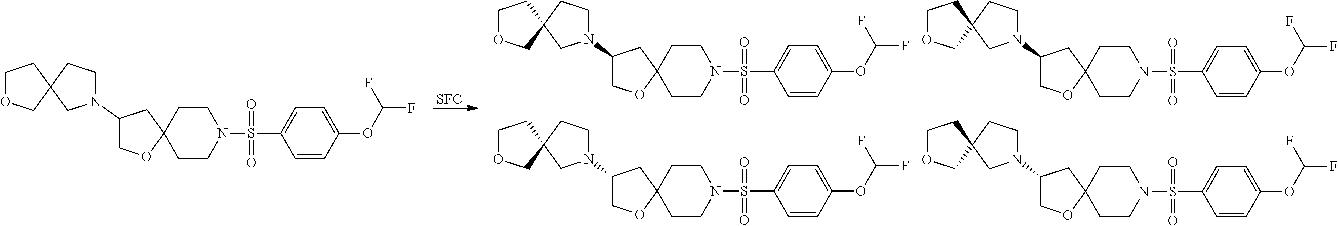

8-((4-(Difluoromethoxy)phenyl)sulfonyl)-3-(2-oxa-7-azaspiro[4.4]nonan-7-yl)-1-oxa-8-azaspiro[4.5]decane was purified by chiral SFC (CHIRALPAK AD-H 30×250 mm, 5 μm; Method: 30% EtOH w/0.1% DEA in CO₂ (flow rate: 100 mL/min, ABPR 120 bar, MBPR 40 psi, column temp 40° C.) to give the following enantiomers of arbitrarily assigned stereochemistry:
Peak 1, Enantiomer 1 (4.1 mg, 3%), R_{f}=4.82 min. LCMS m/z=473.2 [M+H]⁺
Peak 2, Enantiomer 2, (4.4 mg, 3%). R_{f}=5.43 min LCMS m/z=473.2 [M+H]⁺. ¹H-NMR (400 MHz, CD₃OD) δ (ppm): 7.85-7.78 (m, 2H), 7.38-7.32 (m, 2H), 7.21-6.80 (m, 1H), 3.90-3.76 (m, 3H), 3.64-3.51 (m, 3H), 3.41-3.33 (m, 2H), 2.95 (quin, J=7.47 Hz, 1H), 2.80-2.67 (m, 2H), 2.65-2.55 (m, 3H), 2.46 (d, J=9.54 Hz, 1H), 2.01-1.88 (m, 3H), 1.87-1.76 (m, 4H), 1.76-1.61 (m, 3H).
Peak 3, Enantiomer 3 (1.2 mg, 1%), R_{f}=6.27 min. LCMS m/z=473.2 [M+H]⁺
Peak 4, Enantiomer 4 (1.3 mg, 1%), R_{f}=6.50 min. LCMS m/z=473.2 [M+H]⁺

### Example 31: 4-(8-((7-fluoroquinolin-6-yl)sulfonyl)-8-azaspiro[4.5]decan-2-yl)morpholine

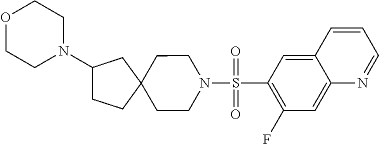 [SMILES: O=S(=O)(c1cc2cccnc2cc1F)N1CCC2(CCC(N3CCOCC3)C2)CC1]

### 1. Synthesis of 7-fluoroquinoline-6-sulfonylfluoride

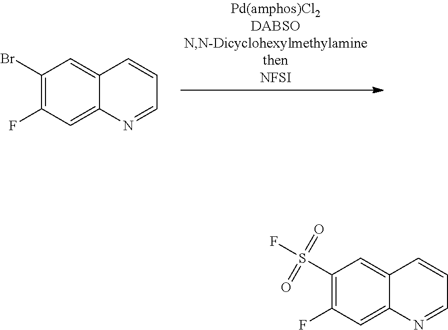 [SMILES: Fc1cc2ncccc2cc1Br ; O=S(=O)(F)c1cc2cccnc2cc1F]

A microwave vial was charged with 6-bromo-7-fluoroquinoline (45 mg, 0.2 mmol), DABSO (48 mg, 0.2 mmol) and Pd(amphos)Cl₂ (7 mg, 0.01 mmol). A solution of N,N-dicyclohexylmethylamine (130 μL, 0.6 mmol) in anhydrous IPA (1.0 mL, 0.2 M) was added under inert atmosphere, the vial was sealed, sparged with N₂ for 5 min and heated at 110° C. under microwave irradiation for 1 h.

The reaction mixture was brought to room temperature NFSI (95 mg, 0.3 mmol) was added and the resulting mixture was stirred for 3 h. The reaction mixture was diluted with EtOAc, washed with water (3 mL), and extracted with EtOAc (5 mL, 2×). The combined organic phase was dried over anhydrous MgSO₄ and concentrated in vacuo to give 7-fluoroquinoline-6-sulfonyl fluoride which was used without further purification in the next step. LCMS m/z=230.0 [M+H]⁺

### 2. Synthesis of 4-(8-((7-fluoroquinolin-6-yl)sulfonyl)-8-azaspiro[4.5]decan-2-yl)morpholine

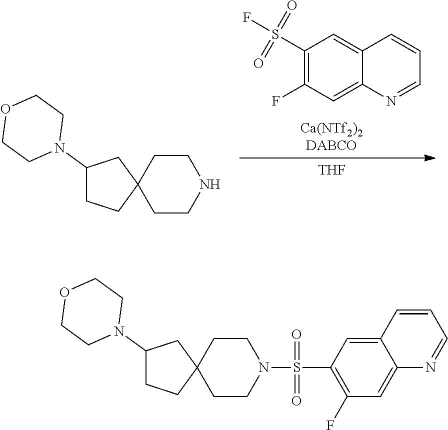 [SMILES: C1CC2(CCN1)CCC(N1CCOCC1)C2 ; O=S(=O)(F)c1cc2cccnc2cc1F ; O=S(=O)(c1cc2cccnc2cc1F)N1CCC2(CCC(N3CCOCC3)C2)CC1 ; "THF"]

To the solution of 7-fluoroquinoline-6-sulfonyl fluoride (23 mg, 0.1 mmol) and 4-(8-azaspiro[4.5]decan-2-yl)morpholine (22 mg, 0.1 mmol) in THE (1.0 mL) was added Ca(NTf₂)₂ (66 mg, 0.1 mmol) and DABCO (17 mg, 0.2 mmol) and the heterogeneous reaction solution was heated at 60° C. for 16 h. The reaction mixture was brought to room temperature, quenched with water and extracted with EtOAc (2 mL, 3×). The combined organic phase was dried over anhydrous MgSO₄ and concentrated in vacuo. The residual material was purified on Waters XSelect CSH Prep C18 column (5 μm OBD 19×100 mm, purification gradient: 5-60%, purification modifier: ammonium hydroxide) to give 4-(8-((7-fluoroquinolin-6-yl)sulfonyl)-8-azaspiro[4.5]decan-2-yl)morpholine (3.2 mg, yield: 7%). LCMS m/z=434.0 (M+H)⁺. R_{f}=1.72 min.

### Example 32: 4-(8-((5-Cyclopropoxypyridin-2-yl)sulfonyl)-8-azaspiro[4.5]decan-2-yl)morpholine

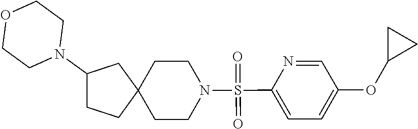 [SMILES: O=S(=O)(c1ccc(OC2CC2)cn1)N1CCC2(CCC(N3CCOCC3)C2)CC1]

### 1. Synthesis of 5-cyclopropoxypyridine-2-sulfonyl fluoride

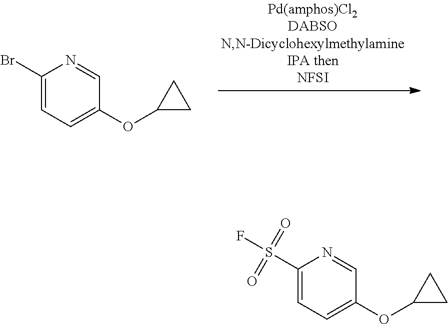 [SMILES: Brc1ccc(OC2CC2)cn1 ; O=S(=O)(F)c1ccc(OC2CC2)cn1]

5-Cyclopropoxypyridine-2-sulfonyl fluoride was obtained from 2-bromo-5-cyclopropoxypyridine following a similar procedure to that described in Example 31, step 1. LCMS m/z=218.0 (M+H)⁺

### 2. Synthesis of 4-(8-((5-cyclopropoxypyridin-2-yl)sulfonyl)-8-azaspiro[4.5]decan-2-yl)morpholine

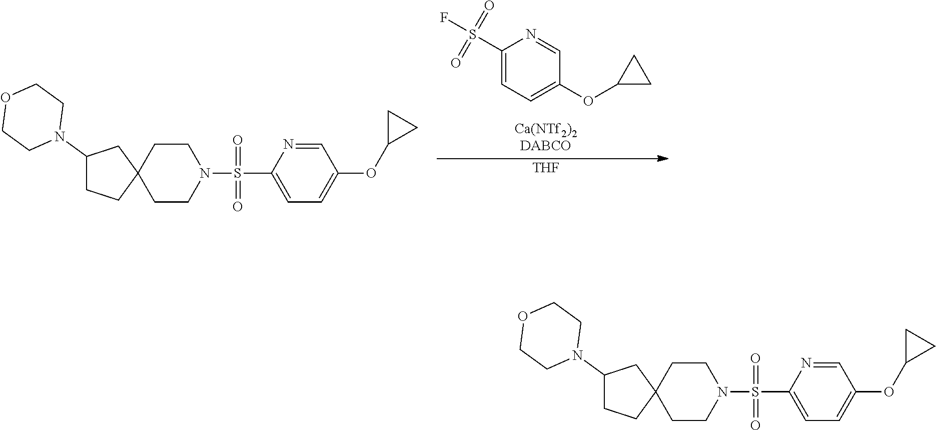 [SMILES: O=S(=O)(c1ccc(OC2CC2)cn1)N1CCC2(CCC(N3CCOCC3)C2)CC1 ; O=S(=O)(F)c1ccc(OC2CC2)cn1 ; O=S(=O)(c1ccc(OC2CC2)cn1)N1CCC2(CCC(N3CCOCC3)C2)CC1 ; "THF"]

4-(8-((5-Cyclopropoxypyridin-2-yl)sulfonyl)-8-azaspiro[4.5]decan-2-yl)morpholine was obtained (11 mg, 27%) from 5-cyclopropoxypyridine-2-sulfonyl fluoride and 4-(8-azaspiro[4.5]decan-2-yl)morpholine following the procedure described in Example 31, step 2. LCMS m/z=422.0 [M+H]⁺. R_{f}=1.80 min.

### Example 33: 4-(8-((3-Fluoro-2-methoxypyridin-4-yl)sulfonyl)-8-azaspiro[4.5]decan-2-yl)morpholine

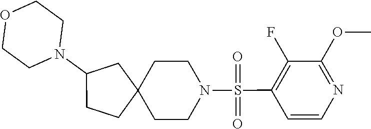 [SMILES: COc1nccc(S(=O)(=O)N2CCC3(CCC(N4CCOCC4)C3)CC2)c1F]

### 1. Synthesis of 3-fluoro-2-methoxypyridine-4-sulfonylfluoride

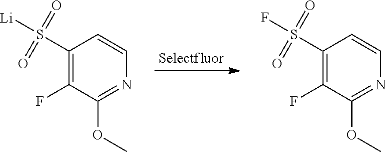

A solution of lithium 3-fluoro-2-methoxypyridine-4-sulfinate (39 mg, 0.2 mmol) and Selectfluor (92 mg, 0.3 mmol) in water (1.0 mL) was stirred at 60° C. for 16 h. The reaction mixture was extracted with EtOAc (2 mL, 3×). The combined organic phase was dried over anhydrous MgSO₄ and concentrated in vacuo to give 3-fluoro-2-methoxypyridine-4-sulfonyl fluoride, which was used without further purification in the next step (assuming 100% yield). LCMS m/z=210.0 [M+H]⁺

### 2. Synthesis of 4-(8-((3-fluoro-2-methoxypyridin-4-yl)sulfonyl)-8-azaspiro[4.5]decan-2-yl)morpholine

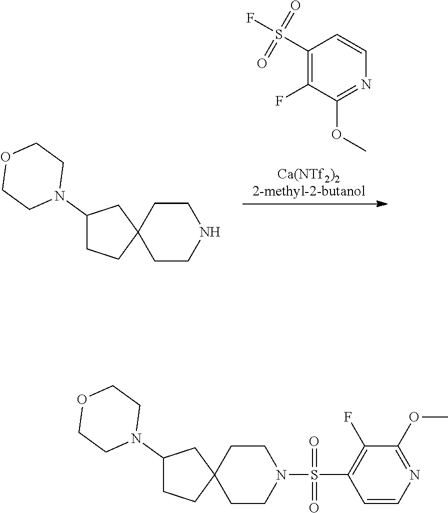 [SMILES: C1CC2(CCN1)CCC(N1CCOCC1)C2 ; COc1nccc(S(=O)(=O)F)c1F ; COc1nccc(S(=O)(=O)N2CCC3(CCC(N4CCOCC4)C3)CC2)c1F]

To a solution of 3-fluoro-2-methoxypyridine-4-sulfonyl fluoride (21 mg, 0.1 mmol) and 4-(8-azaspiro[4.5]decan-2-yl)morpholine (34 mg, 0.2 mmol) in 2-methyl-2-butanol (0.5 mL) was added Ca(NTf₂)₂ (90 mg, 0.2 mmol) and the resulting heterogeneous reaction mixture was heated at 60° C. for 16 h. After cooling to room temperature, the reaction was quenched with water and extracted with EtOAc (2 mL, 3×). The combined organic phases were dried over anhydrous MgSO₄ and concentrated in vacuo. The residual material was purified by prep HPLC using a Waters SunFire Prep C18 column (5 μm OBD 30×100 mm, purification gradient: 5-55%, purification modifier: TFA) to give 4-(8-((3-fluoro-2-methoxypyridin-4-yl)sulfonyl)-8-azaspiro[4.5]decan-2-yl)morpholine (2.3 mg, yield: 6%). LCMS m/z=414.0 (M+H)⁺. R_{f}=1.93 min.

### Example 34: 4-(7-((1-Ethyl-3-methyl-1H-pyrazol-5-yl)sulfonyl)-7-azaspiro[3.5]nonan-2-yl)morpholine

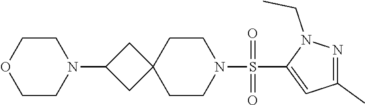 [SMILES: CCn1nc(C)cc1S(=O)(=O)N1CCC2(CC1)CC(N1CCOCC1)C2]

### 1. Synthesis of tert-butyl 2-morpholino-7-azaspiro[3.5]nonane-7-carboxylate

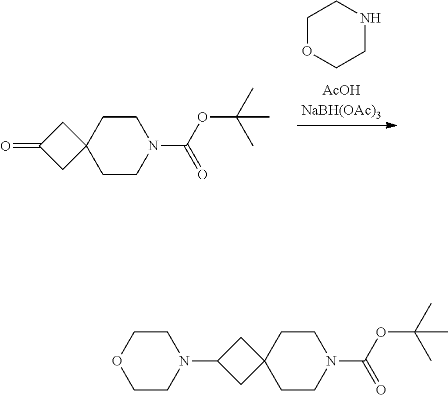 [SMILES: C1COCCN1 ; CC(C)(C)OC(=O)N1CCC2(CC1)CC(=O)C2 ; CC(C)(C)OC(=O)N1CCC2(CC1)CC(N1CCOCC1)C2]

tert-Butyl 2-morpholino-7-azaspiro[3.5]nonane-7-carboxylate was obtained (2.9 g, 96%) from morpholine and tert-butyl 2-oxo-7-azaspiro[3.5]nonane-7-carboxylate, following a similar reaction to that described in Example 21, step 1. LCMS m/z=311.2 [M+H]⁺

### 2. Synthesis of 4-(7-azaspiro[3.5]nonan-2-yl)morpholine hydrochloride

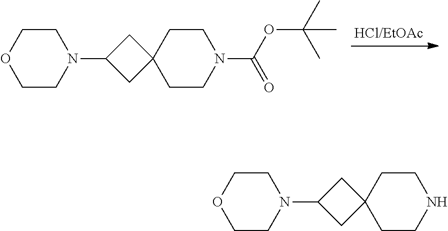 [SMILES: CC(C)(C)OC(=O)N1CCC2(CC1)CC(N1CCOCC1)C2 ; C1CC2(CCN1)CC(N1CCOCC1)C2]

4-(7-Azaspiro[3.5]nonan-2-yl)morpholine hydrochloride was obtained as a pale yellow solid (2.1 g, 93%) from tert-butyl 2-morpholino-7-azaspiro[3.5]nonane-7-carboxylate, following the procedure described in Example 21, step 2. ¹H-NMR (500 MHz, CD₃OD) δ (ppm): 4.11-4.02 (m, 2H), 3.89-3.74 (m, 3H), 3.46-3.37 (m, 2H), 3.21-3.16 (m, 2H), 3.15-3.09 (m, 2H), 2.97 (dt, J=3.1, 12.2 Hz, 2H), 2.43-2.35 (m, 2H), 2.31-2.22 (m, 2H), 1.94-1.86 (m, 4H). LCMS m/z=211.2 (M+H)⁺.

### 3. Synthesis of 4-(7-((I-ethyl-3-methyl-1H-pyrazol-5-yl)sulfonyl)-7-azaspiro[3.5]nonan-2-yl)morpholine

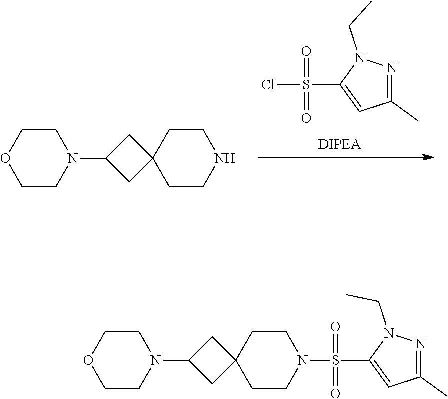 [SMILES: CCn1nc(C)cc1S(=O)(=O)Cl ; C1CC2(CCN1)CC(N1CCOCC1)C2 ; CCn1nc(C)cc1S(=O)(=O)N1CCC2(CC1)CC(N1CCOCC1)C2]

DIPEA (320 μL, 1.8 mmol) was added dropwise to a solution of 4-(7-azaspiro[3.5]nonan-2-yl)morpholine hydrochloride (78 mg, 0.3 mmol) in DCM (1 mL) at <5° C. After 5 min, 2-ethyl-5-methyl-pyrazole-3-sulfonyl chloride (81 mg, 0.4 mmol) was added and the reaction mixture was allowed to warm to room temperature and stirred for 30 min. The reaction mixture was quenched with aqueous 1 M NaOH solution and stirred at room temperature for 10 min. The biphasic mixture was loaded directly onto a silica gel column and purified with (20-80% 3:1 EtOAc:EtOH in heptane) to afford 4-(7-((1-ethyl-3-methyl-1H-pyrazol-5-yl)sulfonyl)-7-azaspiro[3.5]nonan-2-yl)morpholine as a colorless film (65 mg, 51%). LCMS m/z=383.3 (M+H)⁺. ¹H-NMR (500 MHz, DMSO-d₆) δ (ppm): 6.56 (s, 1H), 4.28 (q, J=7.3 Hz, 2H), 3.54 (br t, J=4.3 Hz, 4H), 3.08-3.04 (m, 2H), 2.98-2.94 (m, 2H), 2.65-2.61 (m, 1H), 2.26-2.12 (m, 7H), 1.90-1.84 (m, 2H), 1.64-1.60 (m, 2H), 1.55-1.47 (m, 4H), 1.35-1.31 (m, 3H).

### Example 35: 4-(7-((2-Methyl-6-(trifluoromethyl)pyridin-3-yl)sulfonyl)-7-azaspiro[3.5]nonan-2-yl)morpholine

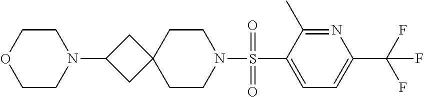 [SMILES: Cc1nc(C(F)(F)F)ccc1S(=O)(=O)N1CCC2(CC1)CC(N1CCOCC1)C2]

4-(7-((2-Methyl-6-(trifluoromethyl)pyridin-3-yl)sulfonyl)-7-azaspiro[3.5]nonan-2-yl)morpholine was obtained (92 mg, 58%) from 4-(7-azaspiro[3.5]nonan-2-yl)morpholine hydrochloride (Example 34, step 2) and 2-methyl-6-(trifluoromethyl)pyridine-3-sulfonyl chloride, following a similar reaction to that described in Example 23. LCMS m/z=434.2 (M+H)⁺. ¹H-NMR (500 MHz, DMSO-d₆) δ (ppm): 8.41 (d, J=7.9 Hz, 1H), 7.96 (d, J=7.9 Hz, 1H), 3.53 (br t, J=4.0 Hz, 4H), 3.17-3.15 (m, 2H), 3.09-3.05 (m, 2H), 2.81 (s, 3H), 2.65-2.58 (m, 1H), 2.30-2.08 (m, 4H), 1.89 (ddd, J=2.4, 7.8, 9.9 Hz, 2H), 1.63-1.59 (m, 2H), 1.54-1.47 (m, 4H).

### Example 36: 4-(7-((2-Methoxy-5-methylpyridin-3-yl)sulfonyl)-7-azaspiro[3.5]nonan-2-yl)morpholine

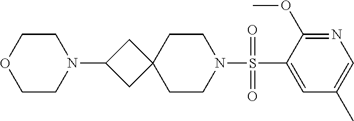 [SMILES: COc1ncc(C)cc1S(=O)(=O)N1CCC2(CC1)CC(N1CCOCC1)C2]

### 1. Synthesis of 4-(7-((2-methoxy-5-methylpyridin-3-yl)sulfonyl)-7-azaspiro[3.5]nonan-2-yl)morpholine

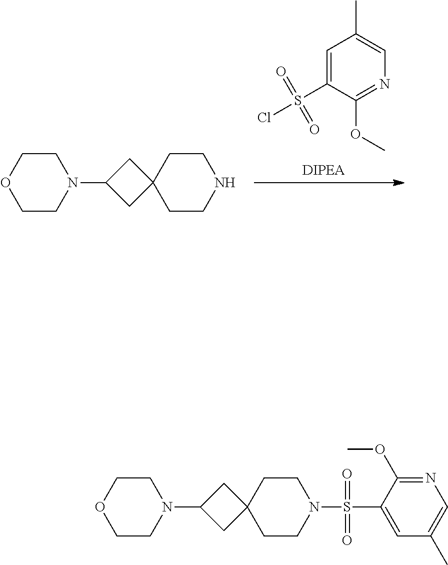 [SMILES: COc1ncc(C)cc1S(=O)(=O)Cl ; C1CC2(CCN1)CC(N1CCOCC1)C2 ; COc1ncc(C)cc1S(=O)(=O)N1CCC2(CC1)CC(N1CCOCC1)C2]

To a solution of 4-(7-azaspiro[3.5]nonan-2-yl)morpholine hydrochloride (Example 34, step 2, 69 mg, 0.3 mmol) in anhydrous DMF (4 mL) was added DIPEA (244 μL, 1.4 mmol). After 5 min, 2-methoxy-5-methyl-pyridine-3-sulfonyl chloride (62 mg, 0.3 mmol) was added and the reaction was stirred at room temperature for 1 h. The reaction was quenched with water and the mixture extracted with EtOAc (3×). The combined organics were washed with saturated aqueous NaHCO₃ solution (2×), dried over anhydrous Na₂SO₄, filtered and concentrated in vacuo. The crude material was purified by silica gel column chromatography (0-25% EtOAc to 3:1 EtOAc:EtOH) to afford 4-(7-((2-methoxy-5-methylpyridin-3-yl)sulfonyl)-7-azaspiro[3.5]nonan-2-yl)morpholine as a yellow oil. LCMS m/z=396.2 (M+H)⁺. ¹H-NMR (400 MHz, CDCl₃) δ (ppm): 8.09 (dd, J=0.8, 2.3 Hz, 1H), 8.00-7.90 (m, 1H), 4.01 (s, 3H), 3.70 (t, J=4.6 Hz, 4H), 3.30-3.20 (m, 2H), 3.20-3.10 (m, 2H), 2.80-2.60 (m, 1H), 2.29 (s, 7H), 2.00-1.90 (m, 2H), 1.70-1.50 (m, 6H).

### Example 37: 4-(7-((3-Cyclopropyl-1-methyl-1H-pyrazol-5-yl)sulfonyl)-7-azaspiro[3.5]nonan-2-yl)morpholine

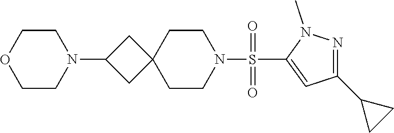 [SMILES: Cn1nc(C2CC2)cc1S(=O)(=O)N1CCC2(CC1)CC(N1CCOCC1)C2]

4-(7-((3-Cyclopropyl-1-methyl-1H-pyrazol-5-yl)sulfonyl)-7-azaspiro[3.5]nonan-2-yl)morpholine was obtained as a yellow oil, from 4-(7-azaspiro[3.5]nonan-2-yl)morpholine hydrochloride and 5-cyclopropyl-2-methyl-pyrazole-3-sulfonyl chloride following the procedure described in Example 36. LCMS m/z=395.3 (M+H)⁺. ¹H-NMR (400 MHz, CDCl₃) δ (ppm): 6.33 (s, 1H), 3.98 (s, 3H), 3.69 (t, 4H, J=4.6 Hz), 3.20-3.10 (m, 2H), 3.10-3.00 (m, 2H), 2.66 (quin, 1H, J=7.8 Hz), 2.29 (br s, 4H), 2.00-1.80 (m, 3H), 1.70-1.50 (m, 6H), 1.00-0.80 (m, 2H), 0.80-0.60 (m, 2H).

### Example 38: 4-(8-((2-Methyl-6-(trifluoromethyl)pyridin-3-yl)sulfonyl)-8-azaspiro[bicyclo[3.2.1]octane-3,1′-cyclobutan]-3′-yl)morpholine

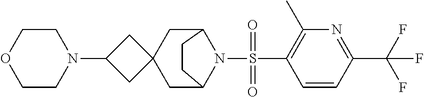 [SMILES: Cc1nc(C(F)(F)F)ccc1S(=O)(=O)N1C2CCC1CC1(CC(N3CCOCC3)C1)C2]

### 1. Synthesis of tert-butyl 3′-morpholino-8-azaspiro[bicyclo[3.2.1]octane-3,1′-cyclobutane]-8-carboxylate

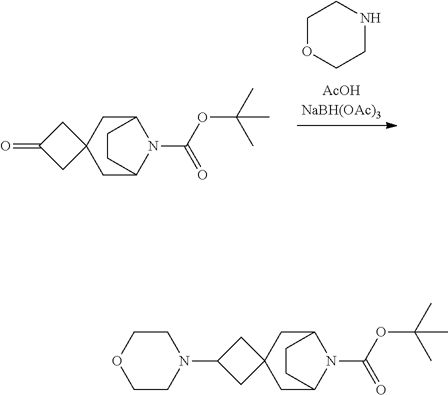 [SMILES: C1COCCN1 ; CC(C)(C)OC(=O)N1C2CCC1CC1(CC(=O)C1)C2 ; CC(C)(C)OC(=O)N1C2CCC1CC1(CC(N3CCOCC3)C1)C2]

tert-Butyl 3′-morpholino-8-azaspiro[bicyclo[3.2.1]octane-3,1′-cyclobutane]-8-carboxylate was obtained (986 mg, crude) from morpholine and tert-butyl 3′-oxo-8-azaspiro[bicyclo[3.2.1]octane-3,1′-cyclobutane]-8-carboxylate, following the procedure described in Example 27, step 1. LCMS m/z=337.2 [M+H]⁺.

### 2. Synthesis of 4-(8-azaspiro[bicyclo[3.2.1]octane-3,1′-cyclobutan]-3′-yl)morpholine

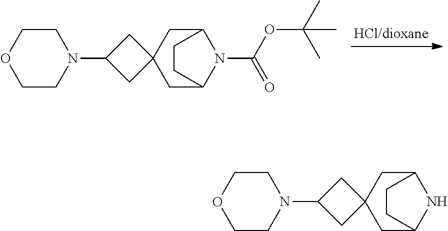 [SMILES: CC(C)(C)OC(=O)N1C2CCC1CC1(CC(N3CCOCC3)C1)C2 ; C1CN(C2CC3(CC4CCC(C3)N4)C2)CCO1]

A solution of tert-butyl 3′-morpholinospiro[8-azabicyclo[3.2.1]octane-3,1′-cyclobutane]-8-carboxylate (986 mg, 2.9 mmol) in MeOH (3 mL) was cooled in an ice water bath, 4M HCl in Dioxane (2.2 mL) was added dropwise and the reaction was allowed to warm to room temperature and stirred for 20 h. The yellow solution was concentrated under reduced pressure and the yellow residue was triturated with a few drops of MeOH in EtOAc. The heterogeneous mixture was filtered to afford 4-(8-azaspiro[bicyclo[3.2.1]octane-3,1′-cyclobutan]-3′-yl)morpholine hydrochloride as a white solid (602 mg, crude) that was used without purification in the next step. LCMS m/z=237.2 (M+H)⁺. ¹H-NMR (500 MHz, CD₃OD) δ (ppm): 4.14-3.96 (m, 4H), 3.94-3.78 (m, 3H), 3.41-3.34 (m, 2H), 3.01-2.90 (m, 2H), 2.82 (ddd, J=4.9, 7.6, 11.9 Hz, 1H), 2.59-2.52 (m, 1H), 2.38-2.32 (m, 1H), 2.29-2.18 (m, 3H), 2.17-2.06 (m, 3H), 2.05-1.94 (m, 3H).

### 3. Synthesis of 4-(8-((2-methyl-6-(trifluoromethyl)pyridin-3-yl)sulfonyl)-8-azaspiro[bicyclo[3.2.1]octane-3,1′-cyclobutan]-3′-yl)morpholine

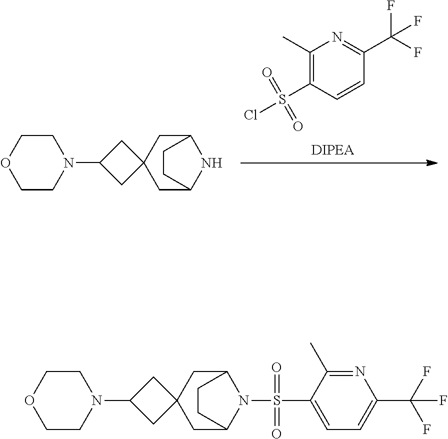 [SMILES: Cc1nc(C(F)(F)F)ccc1S(=O)(=O)Cl ; C1CN(C2CC3(CC4CCC(C3)N4)C2)CCO1 ; Cc1nc(C(F)(F)F)ccc1S(=O)(=O)N1C2CCC1CC1(CC(N3CCOCC3)C1)C2]

To a solution of 4-spiro(8-azabicyclo[3.2.1]octane-3,3′-cyclobutane)-1′-ylmorpholine hydrochloride (61 mg, 0.2 mmol) in anhydrous DCM (0.5 mL) was added DIPEA (171 mg, 1.3 mmol) dropwise at <5° C. After 5 min, 2-methyl-6-(trifluoromethyl)pyridine-3-sulfonylchloride (82 mg, 0.3 mmol) was added and the reaction was allowed to warm to room temperature and was stirred for 30 min. The reaction was quenched with aqueous 1 M NaOH solution, the mixture was stirred at room temperature for 20 min. The biphasic mixture was extracted with DCM (3×) and the combined organic extracts were dried over anhydrous MgSO₄, filtered and concentrated in vacuo. The crude material was purified by HPLC using a Waters XSelect CSH C18, 5 μm, 50 mm×100 mm column with mobile phase water (A) and MeCN (B) and a gradient of 5-60% B (0.2% NH₄OH final v/v % modifier) with flow rate at 60 mL/min to give 4-(8-((2-methyl-6-(trifluoromethyl)pyridin-3-yl)sulfonyl)-8-azaspiro[bicyclo[3.2.1]octane-3,1′-cyclobutan]-3′-yl)morpholine as a colorless film (7.9 mg, 7%). LCMS m/z=460.3 (M+H)⁺. ¹H-NMR (500 MHz, DCM-d₂) d=8.41 (d, J=8.5 Hz, 1H), 7.65 (d, J=7.9 Hz, 1H), 4.22-4.18 (m, 1H), 4.13-4.09 (m, 1H), 3.61 (t, J=4.6 Hz, 4H), 2.89 (s, 3H), 2.68-2.62 (m, 1H), 2.43-2.38 (m, 1H), 2.21 (br d, J=2.4 Hz, 4H), 2.02-1.93 (m, 4H), 1.84-1.75 (m, 6H), 1.68-1.63 (m 1H).

### Example 39: 7-((4-(Difluoromethoxy)phenyl)sulfonyl)-3-morpholino-1-oxa-7-azaspiro[3.5]nonane

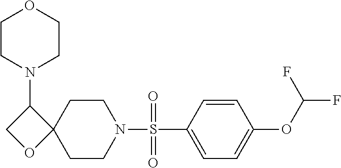 [SMILES: O=S(=O)(c1ccc(OC(F)F)cc1)N1CCC2(CC1)OCC2N1CCOCC1]

### 1. Synthesis of tert-butyl 3-morpholino-1-oxa-7-azaspiro[3.5]nonane-7-carboxylate

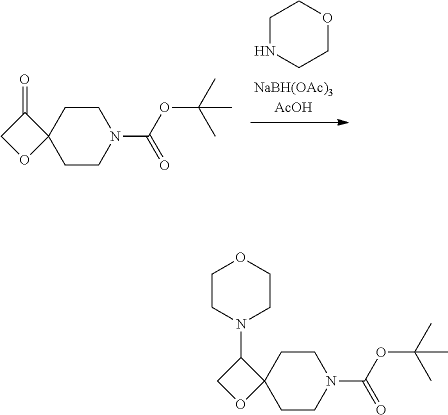 [SMILES: C1COCCN1 ; CC(C)(C)OC(=O)N1CCC2(CC1)OCC2=O ; CC(C)(C)OC(=O)N1CCC2(CC1)OCC2N1CCOCC1]

To a solution of tert-butyl 3-oxo-1-oxa-7-azaspiro[3.5]nonane-7-carboxylate (483 mg, 2.0 mmol) in DCM (15 mL) were added acetic acid (70 μL, 1.2 mmol) and morpholine (500 μL, 5.7 mmol) dropwise at room temperature. After 15 min NaBH(OAc)₃ (1.76 g, 8.29 mmol) was added batchwise and the reaction was stirred at room temperature for 20 h. The reaction was quenched with aqueous saturated NH₄Cl solution then extracted with DCM (3×). The combined organics were washed with brine, dried over anhydrous MgSO₄, filtered and concentrated under reduced pressure, to give tert-butyl 3-morpholino-1-oxa-7-azaspiro[3.5]nonane-7-carboxylate (625 mg, crude) that was used without purification in the next step. LCMS m/z=313.1 (M+H)⁺.

### 2. Synthesis of 3-morpholino-1-oxa-7-azaspiro[3.5]nonane trifluoroacetate

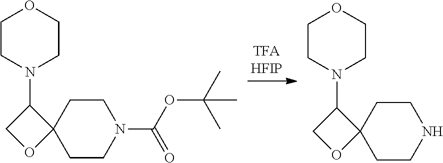 [SMILES: CC(C)(C)OC(=O)N1CCC2(CC1)OCC2N1CCOCC1 ; C1CC2(CCN1)OCC2N1CCOCC1]

To a solution of tert-butyl 3-morpholino-1-oxa-7-azaspiro[3.5]nonane-7-carboxylate (625 mg, 2.0 mmol) in 1,1,1,3,3,3-hexafluoropropan-2-ol (5 mL, 48 mmol) was added TFA (500 μL, 6.5 mmol) dropwise at 0° C. The reaction mixture was allowed to warm to room temperature and stirred for 4 h. The volatiles were removed under reduced pressure to afford 3-morpholino-1-oxa-7-azaspiro[3.5]nonane trifluoroacetate as a brown film, that was used without purification in the next step. LCMS m/z=213.1 (M+H)⁺.

### 3. Synthesis of 7-((4-(difluoromethoxy)phenyl)sulfonyl)-3-morpholino-1-oxa-7-azaspiro[3.5]nonane

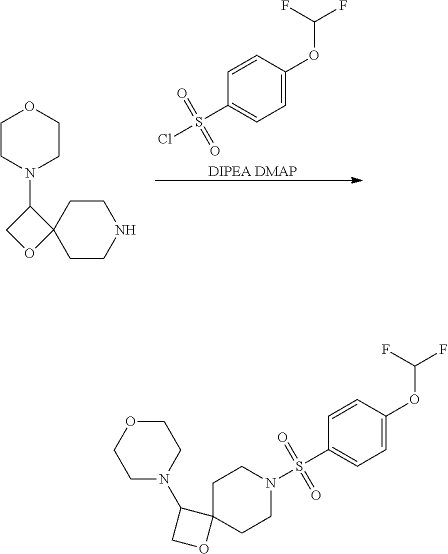

To a solution of 3-morpholino-1-oxa-7-azaspiro[3.5]nonane trifluoroacetate (130 mg, 0.4 mmol) in anhydrous THE (2 mL) was added DIPEA (500 μL, 2.9 mmol) dropwise and DMAP (6 mg, 0.05 mmol) at <5° C. The resulting solution was stirred for 5 min before 4-(difluoromethoxy)benzenesulfonyl chloride (137 mg, 0.6 mmol) was added. The reaction mixture was brought to room temperature and stirred for 30 min. The reaction was quenched with aqueous 1 M NaOH, the mixture was stirred for 10 min, then the biphasic mixture was extracted with EtOAc (3×). The combined organics were washed with saturated aqueous NaHCO₃ solution (2×), dried over anhydrous Na₂SO₄, filtered and concentrated under reduced pressure. The resulting residue was purified by silica gel column chromatography (25-100% EtOAc in heptane) to give 7-((4-(difluoromethoxy)phenyl)sulfonyl)-3-morpholino-1-oxa-7-azaspiro[3.5]nonane as a colorless film that was triturated with MeOH to afford a white solid (10 mg, 6%). LCMS m/z=419.1 (M+H)⁺. ¹H-NMR (500 MHz, DMSO-d₆) δ (ppm): 7.82-7.79 (m, 2H), 7.58-7.27 (m, 3H), 4.21-4.14 (m, 2H), 3.61-3.48 (m, 6H), 2.97 (t, J=7.6 Hz, 1H), 2.37-2.18 (m, 3H), 2.18-2.04 (m, 4H), 1.85 (br dd, J=1.8, 13.4 Hz, 1H), 1.73-1.62 (m, 2H).

### Examples 40 to 43

### 1. Synthesis of 8-((2-methoxy-5-methylpyridin-3-yl)sulfonyl)-8-azaspiro[4.5]decan-2-one

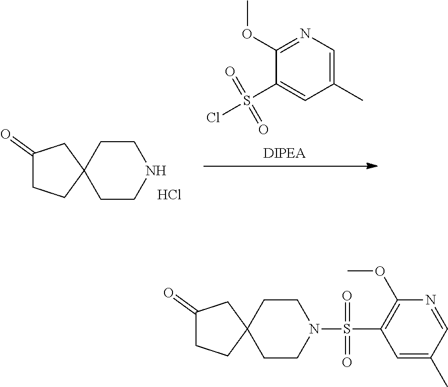 [SMILES: COc1ncc(C)cc1S(=O)(=O)Cl ; O=C1CCC2(CCNCC2)C1 ; Cl ; COc1ncc(C)cc1S(=O)(=O)N1CCC2(CCC(=O)C2)CC1]

To a vial containing 8-azaspiro[4.5]decan-3-one hydrochloride (605 mg, 3.2 mmol) in anhydrous DCM (20 mL) was added DIPEA (2.2 mL, 12.4 mmol) dropwise at <5° C. After 5 min, 2-methoxy-5-methyl-pyridine-3-sulfonyl chloride (825 mg, 3.7 mmol) was added to the cold solution and the reaction was allowed to warm to room temperature. After 30 min, the homogeneous reaction mixture was quenched with aqueous 1 M NaOH solution, the mixture was stirred at room temperature for 10 min. The phases were separated and the aqueous layer was washed with DCM (10 mL×2). The combined organic layer was dried over Na₂SO₄, filtered and concentrated to give an orange oil which was purified by silica gel column chromatography (10-55% EtOAc in heptane) to give 8-((2-methoxy-5-methylpyridin-3-yl)sulfonyl)-8-azaspiro[4.5]decan-2-one (830 mg, 77%). LCMS m/z=338.9 [M+H]⁺. The title compounds were prepared in a single step library on an approximately 85 mg target product scale using the following protocol.

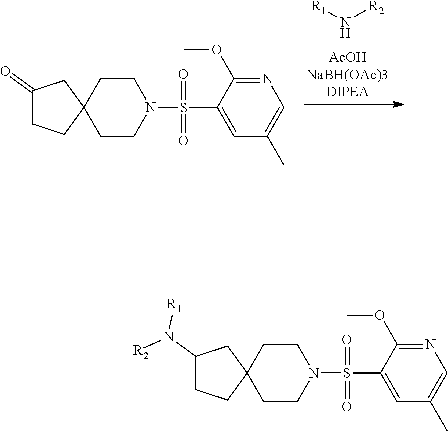

DIPEA (2.0 equiv.) was added to a solution of the appropriate amine (1.5 equiv.) in DCM (1 mL). After 15 min this solution was added to 8-((2-methoxy-5-methylpyridin-3-yl)sulfonyl)-8-azaspiro[4.5]decan-2-one (1.0 equiv.) in DCM (1 mL). After an additional 15 min stirring at room temperature, acetic acid (4.0 equiv.) was added dropwise followed by NaBH(OAc)₃ (8.0 equiv.) and the reaction mixture was stirred at room temperature for overnight. Saturated aqueous NH₄Cl (5 mL) and DCM (5 mL) were added, the aqueous phase was separated and extracted with DCM (3×5 mL). The combined organic layer was washed with water (5 mL) and brine (5 mL), separated, dried over Na₂SO₄, filtered and concentrated. The resulting residue was purified by prep. HPLC (Flow rate: 30 mL/min; Column: Waters XSELECT CSH C18 PREP 19×100 mm, 5 μm; Modifier: 0.2% NH₄OH (v/v) conc.; Method: A % water/B % MeCN linear gradient over 8 min, to give the respective product.

| Example | | HPLC gradient/ |
|---|---|---|
| No. | Name/Structure/Name/Amine | Yield/Data |
| 40 | 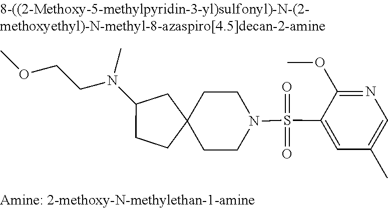 [SMILES: COCCN(C)C1CCC2(CCN(S(=O)(=O)c3cc(C)cnc3OC)CC2)C1] | HPLC gradient: 10-80% B 59.8 mg (71%) LCMS m/z = 412.3 [M + H]⁺. R_{f} = 1.31 min |
| 41 | 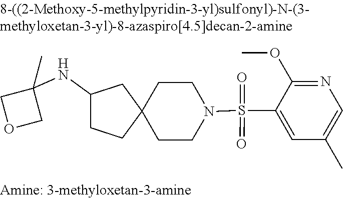 [SMILES: COc1ncc(C)cc1S(=O)(=O)N1CCC2(CCC(NC3(C)COC3)C2)CC1] | HPLC gradient: 5-75% B 4.3 mg (5%) LCMS m/z = 410.3 [M + H]⁺. R_{f} = 1.69 min |
| 42 | 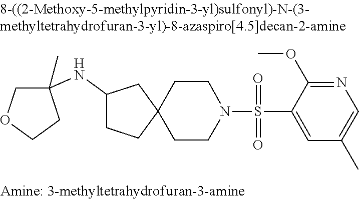 [SMILES: COc1ncc(C)cc1S(=O)(=O)N1CCC2(CCC(NC3(C)CCOC3)C2)CC1] | HPLC gradient: 5-75% B 31.1 mg (36%) LCMS m/z = 424.3 [M + H]⁺. R_{f} = 1.28 min |
| 43 | 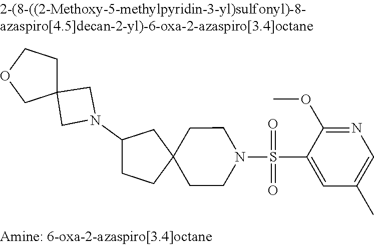 [SMILES: COc1ncc(C)cc1S(=O)(=O)N1CCC2(CCC(N3CC4(CCOC4)C3)C2)CC1] | HPLC gradient: 10-80% B 64.0 mg (72%) LCMS m/z = 436.3 [M + H]⁺. R_{f} = 1.27 min |

### Example 44: 4-Fluoro-3-((3-((2-methoxyethyl)amino)-1-oxa-8-azaspiro[4.5]decan-8-yl)sulfonyl)benzonitrile

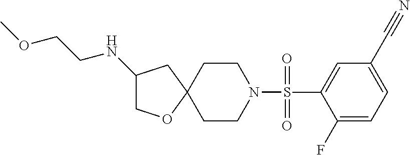 [SMILES: COCCNC1COC2(CCN(S(=O)(=O)c3cc(C#N)ccc3F)CC2)C1]

### 1. Synthesis of 1-oxa-8-azaspiro[4.5]decan-3-one hydrochloride

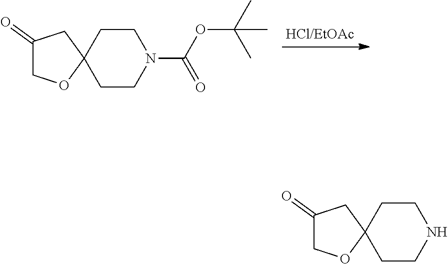 [SMILES: CC(C)(C)OC(=O)N1CCC2(CC1)CC(=O)CO2 ; O=C1COC2(CCNCC2)C1]

tert-Butyl 3-oxo-1-oxa-8-azaspiro[4.5]decane-8-carboxylate (0.5 g, 2.0 mmol) was dissolved in 1M HCl/EtOAc solution (9.8 mL, 9.8 mmol) at 0° C. and the reaction was warmed to room temperature and stirred overnight. The reaction mixture was filtered to afford 1-oxa-8-azaspiro[4.5]decan-3-one hydrochloride as a white solid (300 mg, 99%).

### 2. Synthesis of 4-fluoro-3-((3-oxo-1-oxa-8-azaspiro[4.5]decan-8-yl)sulfonyl)benzonitrile

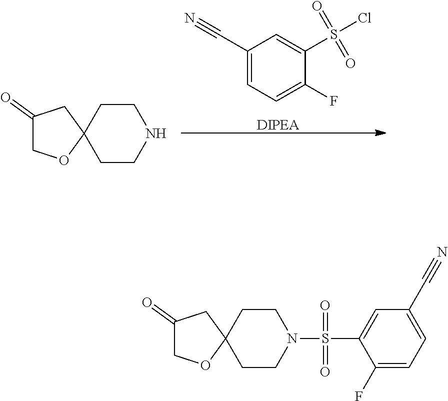

DIPEA (1.1 mL, 6.5 mmol) was added to 1-oxa-8-azaspiro[4.5]decan-3-one hydrochloride (250 mg, 1.3 mmol) in anhydrous DMF (2 mL). After 5 min, 5-cyano-2-fluoro-benzenesulfonyl chloride (287 mg, 1.3 mmol) was added and the reaction was stirred at room temperature for 1 h. The reaction was quenched with water and the mixture was extracted with EtOAc (3×). The combined organics were washed with saturated aqueous NaHCO₃ solution (2×) then dried over anhydrous Na₂SO₄ and concentrated in vacuo. The crude material was purified by silica gel chromatography (0-75% hexanes to EtOAc) to give 4-fluoro-3-((3-oxo-1-oxa-8-azaspiro[4.5]decan-8-yl)sulfonyl)benzonitrile (300 mg, 68%). LCMS m/z=339.0 [M+H]⁺. ¹H-NMR (400 MHz, CDCl₃) δ (ppm): 8.20 (dd, J=2.0, 6.3 Hz, 1H), 7.90 (ddd, J=2.3, 4.5, 8.6 Hz, 1H), 7.39 (t, J=8.9 Hz, 1H), 3.97 (s, 2H), 3.70-3.60 (m, 2H), 3.20-3.10 (m, 2H), 2.39 (s, 2H), 2.00-1.80 (m, 4H).

### 3. Synthesis of 4-fluoro-3-((3-((2-methoxyethyl)amino)-1-oxa-8-azaspiro[4.5]decan-8-yl)sulfonyl)benzonitrile

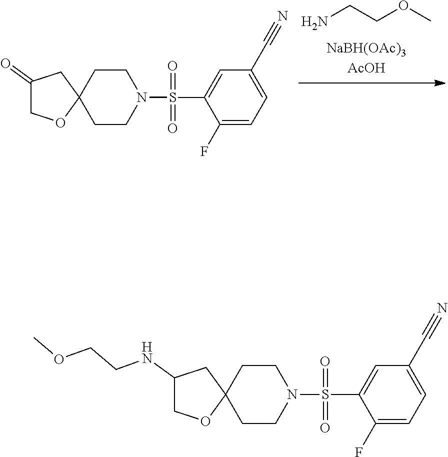

2-Methoxyethanamine hydrochloride (119 mg, 1.1 mmol) was added to a solution of 4-fluoro-3-((3-oxo-1-oxa-8-azaspiro[4.5]decan-8-yl)sulfonyl)benzonitrile (300 mg, 0.9 mmol) in DCM (25 mL) and the solution stirred at room temperature for 15 min. Acetic acid (100 μL, 1.8 mmol) was added dropwise, the reaction was stirred for 30 min, NaBH(OAc)₃ (752 mg, 3.6 mmol) was added and the reaction stirred at room temperature for 3 h. The reaction was quenched with saturated aqueous NH₄Cl and diluted with DCM. The organics were washed with water and brine then concentrated in vacuo. The crude product was purified by silica gel chromatography (0-100% EtOAc to 3:1 EtOAc:EtOH (w/2% NH₃OH)) to afford 4-fluoro-3-((3-((2-methoxyethyl)amino)-1-oxa-8-azaspiro[4.5]decan-8-yl)sulfonyl)benzonitrile (210 mg, 59%). LCMS m/z=398.1 [M+H]⁺. ¹H-NMR (400 MHz, CDCl₃) δ (ppm): 8.18 (dd, 1H, J=2.0, 6.3 Hz), 7.87 (ddd, 1H, J=2.1, 4.4, 8.5 Hz), 7.35 (t, 1H, J=8.8 Hz), 3.93 (dd, 1H, J=6.0, 9.0 Hz), 3.70-3.60 (m, 3H), 3.50-3.40 (m, 3H), 3.36 (s, 3H), 3.10-2.90 (m, 2H), 2.80-2.60 (m, 2H), 2.10-2.00 (m, 1H), 2.00-1.60 (m, 5H).

### Example 45: 3-((3-((1R,4R)-2-oxa-5-azabicyclo[2.2.1]heptan-5-yl)-1-oxa-8-azaspiro[4.5]decan-8-yl)sulfonyl)-4-fluorobenzonitrile

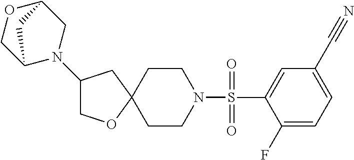 [SMILES: N#Cc1ccc(F)c(S(=O)(=O)N2CCC3(CC2)CC(N2C[C@H]4C[C@@H]2CO4)CO3)c1]

3-((3-((1R,4R)-2-Oxa-5-azabicyclo[2.2.1]heptan-5-yl)-1-oxa-8-azaspiro[4.5]decan-8-yl)sulfonyl)-4-fluorobenzonitrile was obtained (100 mg, 35%) from (1S,4S)-2-oxa-5-azabicyclo[2.2.1]heptane hydrochloride and 4-fluoro-3-((3-oxo-1-oxa-8-azaspiro[4.5]decan-8-yl)sulfonyl)benzonitrile, following the procedure described in Example 44, step 3. LCMS m/z=422.1 [M+H]⁺. ¹H-NMR (400 MHz, CDCl₃) δ (ppm): 8.20-8.10 (m, 1H), 7.86 (ddd, J=1.8, 4.2, 8.3 Hz, 1H), 7.40-7.30 (m, 1H), 4.37 (br s, 1H), 4.00 (br d, 1H, J=7.8 Hz), 3.90-3.70 (m, 1H), 3.70-3.50 (m, 4H), 3.42 (br d, J=19.1 Hz, 1H), 3.31 (dt, J=3.0, 6.4 Hz, 1H), 3.10-2.70 (m, 3H), 2.60-2.30 (m, 1H), 2.10-1.60 (m, 8H).

### Example 46: 3-((3-(2-Oxa-6-azaspiro[3.3]heptan-6-yl)-1-oxa-8-azaspiro[4.5]decan-8-yl)sulfonyl)-4-fluorobenzonitrile

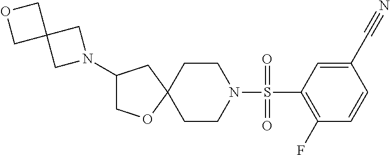 [SMILES: N#Cc1ccc(F)c(S(=O)(=O)N2CCC3(CC2)CC(N2CC4(COC4)C2)CO3)c1]

3-((3-(2-Oxa-6-azaspiro[3.3]heptan-6-yl)-1-oxa-8-azaspiro[4.5]decan-8-yl)sulfonyl)-4-fluorobenzonitrile was obtained (77 mg, 82%) from 2-oxa-6-azaspiro[3.3]heptane hydrochloride and 4-fluoro-3-((3-oxo-1-oxa-8-azaspiro[4.5]decan-8-yl)sulfonyl)benzonitrile, following the procedure described in Example 44, step 3. LCMS m/z=422.1 [M+H]⁺. ¹H-NMR (400 MHz, CDCl₃) δ (ppm): 8.16 (dd, J=2.0, 6.3 Hz, 1H), 7.86 (ddd, J=1.9, 4.3, 8.4 Hz, 1H), 7.34 (t, J=8.8 Hz, 1H), 4.72 (s, 4H), 3.80-3.50 (m, 5H), 3.40-3.20 (m, 4H), 3.00-2.80 (m, 3H), 1.90-1.40 (m, 5H).

### Example 47: 4-Fluoro-3-((3-(piperidin-1-yl)-1-oxa-8-azaspiro[4.5]decan-8-yl)sulfonyl)benzonitrile

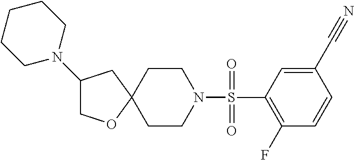 [SMILES: N#Cc1ccc(F)c(S(=O)(=O)N2CCC3(CC2)CC(N2CCCCC2)CO3)c1]

4-Fluoro-3-((3-(piperidin-1-yl)-1-oxa-8-azaspiro[4.5]decan-8-yl)sulfonyl)benzonitrile was obtained (41 mg, 45%) from 4-fluoro-3-((3-oxo-1-oxa-8-azaspiro[4.5]decan-8-yl)sulfonyl)benzonitrile and piperidine hydrochloride, following the procedure described in Example 44, step 3. LCMS m/z=408.1 [M+H]⁺. ¹H-NMR (400 MHz, CDCl₃) δ (ppm): 8.17 (dd, J=2.1, 6.1 Hz, 1H), 7.87 (ddd, J=2.0, 4.3, 8.5 Hz, 1H), 7.35 (t, J=8.8 Hz, 1H), 3.96 (dd, J=7.0, 8.3 Hz, 1H), 3.70-3.50 (m, 3H), 3.10-2.80 (m, 4H), 2.50-2.20 (m, 4H), 2.10-1.90 (m, 2H), 1.90-1.50 (m, 9H).

### Example 48: 3-((3-((4,4-Difluorocyclohexyl)amino)-1-oxa-8-azaspiro[4.5]decan-8-yl)sulfonyl)-4-fluorobenzonitrile

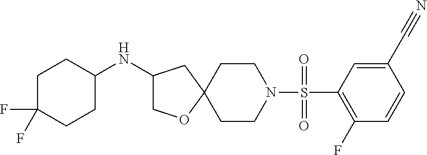 [SMILES: N#Cc1ccc(F)c(S(=O)(=O)N2CCC3(CC2)CC(NC2CCC(F)(F)CC2)CO3)c1]

3-((3-((4,4-Difluorocyclohexyl)amino)-1-oxa-8-azaspiro[4.5]decan-8-yl)sulfonyl)-4-fluorobenzonitrile was obtained (61 mg, 44%) from 4,4-difluorocyclohexanamine hydrochloride and 4-fluoro-3-((3-oxo-1-oxa-8-azaspiro[4.5]decan-8-yl)sulfonyl)benzonitrile, following the procedure described in Example 44, step 3. LCMS m/z=458.1 [M+H]⁺. ¹H-NMR (400 MHz, CDCl₃) δ (ppm): 8.19 (td, J=2.4, 6.1 Hz, 1H), 7.88 (ddd, J=2.0, 4.2, 8.3 Hz, 1H), 7.36 (dt, J=1.6, 8.8 Hz, 1H), 4.00-3.80 (m, 1H), 3.70-3.60 (m, 2H), 3.60-3.40 (m, 1H), 3.20-2.90 (m, 2H), 2.30-1.60 (m, 14H), 1.60-1.40 (m, 2H).

### Example 49: 8-((2-Chloro-4-methylphenyl)sulfonyl)-3-(2-oxa-6-azaspiro[3.3]heptan-6-yl)-1-oxa-8-azaspiro[4.5]decane

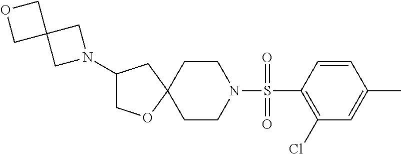 [SMILES: Cc1ccc(S(=O)(=O)N2CCC3(CC2)CC(N2CC4(COC4)C2)CO3)c(Cl)c1]

### 1. Synthesis of 8-((2-chloro-4-methylphenyl)sulfonyl)-1-oxa-8-azaspiro[4.5]decan-3-one

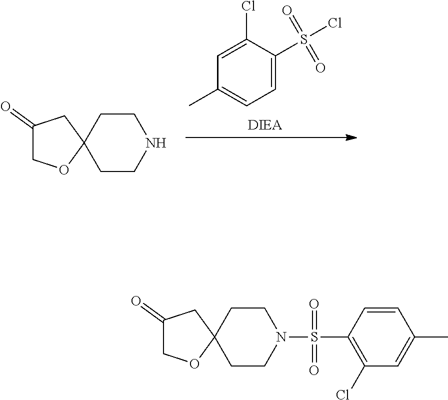 [SMILES: Cc1ccc(S(=O)(=O)Cl)c(Cl)c1 ; O=C1COC2(CCNCC2)C1 ; Cc1ccc(S(=O)(=O)N2CCC3(CC2)CC(=O)CO3)c(Cl)c1]

DIPEA (2.3 mL, 13.0 mmol) was added to a vial containing 1-oxa-8-azaspiro[4.5]decan-3-one hydrochloride (500 mg, 2.6 mmol) in anhydrous DMF (3 mL) the solution stirred for 5 min, then 2-chloro-4-methyl-benzenesulfonyl chloride (587 mg, 2.6 mmol) was added. The reaction was stirred at room temperature for 1 h, then the reaction was quenched with water and the mixture extracted with EtOAc (3×). The combined organics were washed with saturated aqueous NaHCO₃ solution (2×). The organic layer was separated then dried over anhydrous Na₂SO₄ and concentrated in vacuo. The crude material was purified by silica gel chromatography (0-50% EtOAc in Hexanes) to provide 8-((2-chloro-4-methylphenyl)sulfonyl)-1-oxa-8-azaspiro[4.5]decan-3-one. LCMS m/z=344.0 [M+H]⁺.

### 2. Synthesis of 8-((2-chloro-4-methylphenyl)sulfonyl)-3-(2-oxa-6-azaspiro[3.3]heptan-6-yl)-1-oxa-8-azaspiro[4.5]decane

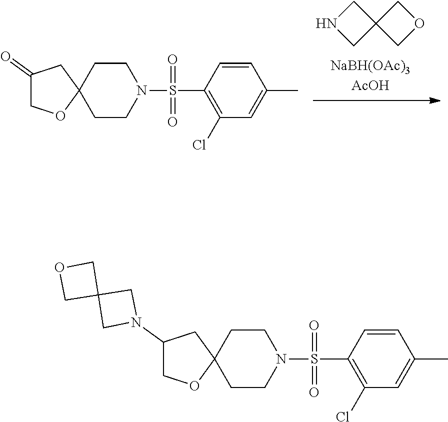 [SMILES: C1NCC12COC2 ; Cc1ccc(S(=O)(=O)N2CCC3(CC2)CC(=O)CO3)c(Cl)c1 ; Cc1ccc(S(=O)(=O)N2CCC3(CC2)CC(N2CC4(COC4)C2)CO3)c(Cl)c1]

2-Oxa-6-azaspiro[3.3]heptane (29 mg, 0.3 mmol) was added to a solution of 8-(2-chloro-4-methyl-phenyl)sulfonyl-1-oxa-8-azaspiro[4.5]decan-3-one (100 mg, 0.3 mmol) in DCM (10 mL) and the solution was stirred at room temperature for 15 min. Acetic acid (40 μL, 0.6 mmol) was added dropwise, followed by NaBH(OAc)₃ (247 mg, 1.16 mmol) and the reaction stirred at room temperature for 3 h. The reaction was quenched with saturated aqueous NH₄Cl and diluted with DCM. The organic solution was washed with water and brine then concentrated in vacuo. The crude product was purified by silica gel column (0-100% EtOAc to 3:1 EtOAc:EtOH (w/2% NH₃OH)) to afford 8-((2-chloro-4-methylphenyl)sulfonyl)-3-(2-oxa-6-azaspiro[3.3]heptan-6-yl)-1-oxa-8-azaspiro[4.5]decane (70 mg, 56%). LCMS m/z=427.1 [M+H]⁺. ¹H-NMR (400 MHz, CDCl₃) δ (ppm): 7.89 (d, J=8.3 Hz, 1H), 7.40-7.30 (m, 1H), 7.16 (d, J=8.0 Hz, 1H), 4.71 (s, 4H), 3.70 (dd, J=5.5, 9.3 Hz, 1H), 3.60-3.40 (m, 3H), 3.40-3.20 (m, 4H), 3.20-3.00 (m, 2H), 3.00-2.80 (m, 1H), 2.39 (s, 3H), 1.90-1.40 (m, 6H).

### Example 50: 1-(8-((2-Chloro-4-methylphenyl)sulfonyl)-1-oxa-8-azaspiro[4.5]decan-3-yl)-3-methylazetidin-3-ol

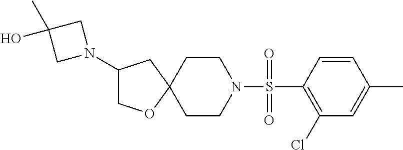 [SMILES: Cc1ccc(S(=O)(=O)N2CCC3(CC2)CC(N2CC(C)(O)C2)CO3)c(Cl)c1]

1-(8-((2-Chloro-4-methylphenyl)sulfonyl)-1-oxa-8-azaspiro[4.5]decan-3-yl)-3-methylazetidin-3-ol was obtained (120 mg, 99%) from 3-methylazetidin-3-ol and 8-(2-chloro-4-methyl-phenyl)sulfonyl-1-oxa-8-azaspiro[4.5]decan-3-one, following the procedure described in Example 49, step 2. LCMS m/z=415.1 [M+H]⁺. ¹H-NMR (400 MHz, CDCl₃) δ (ppm): 7.90 (d, J=8.3 Hz, 1H), 7.33 (d, J=0.8 Hz, 1H), 7.20-7.10 (m, 1H), 3.75 (dd, J=5.5, 9.3 Hz, 1H), 3.70-3.40 (m, 3H), 3.30-3.20 (m, 2H), 3.20-2.90 (m, 5H), 2.40 (s, 3H), 2.00-1.50 (m, 6H), 1.47 (s, 3H).

### Example 51: 8-((2-Chloro-4-methylphenyl)sulfonyl)-N-(tetrahydro-2H-pyran-4-yl)-1-oxa-8-azaspiro[4.5]decan-3-amine

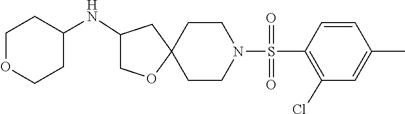 [SMILES: Cc1ccc(S(=O)(=O)N2CCC3(CC2)CC(NC2CCOCC2)CO3)c(Cl)c1]

8-((2-Chloro-4-methylphenyl)sulfonyl)-N-(tetrahydro-2H-pyran-4-yl)-1-oxa-8-azaspiro[4.5]decan-3-amine was obtained (199 mg, 84%) from tetrahydropyran-4-amine and 8-(2-chloro-4-methyl-phenyl)sulfonyl-1-oxa-8-azaspiro[4.5]decan-3-one following the procedure described in Example 49, step 2. LCMS m/z=429.2 [M+H]⁺. ¹H-NMR (400 MHz, CDCl₃) δ (ppm): 7.83 (d, J=8.0 Hz, 1H), 7.26 (s, 1H), 7.20-7.10 (m, 1H), 3.88 (br dd, J=5.6, 8.2 Hz, 3H), 3.60-3.40 (m, 4H), 3.31 (br t, J=11.7 Hz, 2H), 3.20-3.00 (m, 2H), 2.70-2.50 (m, 1H), 2.33 (s, 3H), 2.10-1.80 (m, 1H), 1.70-1.20 (m, 10H).

### Example 52: 1-(8-((2-Chloro-4-methylphenyl)sulfonyl)-1-oxa-8-azaspiro[4.5]decan-3-yl)azetidin-3-ol

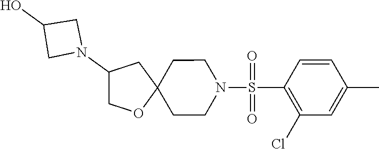 [SMILES: Cc1ccc(S(=O)(=O)N2CCC3(CC2)CC(N2CC(O)C2)CO3)c(Cl)c1]

1-(8-((2-Chloro-4-methylphenyl)sulfonyl)-1-oxa-8-azaspiro[4.5]decan-3-yl)azetidin-3-ol was obtained (110 mg, 94%) from azetidin-3-ol hydrochloride and 8-(2-chloro-4-methyl-phenyl)sulfonyl-1-oxa-8-azaspiro[4.5]decan-3-one, following the procedure described in Example 49, step 2. LCMS m/z=401.2 [M+H]⁺. ¹H-NMR (400 MHz, CDCl₃) δ (ppm): 8.00-7.70 (m, 1H), 7.40-7.30 (m, 1H), 7.17 (d, J=8.0 Hz, 1H), 4.60-4.30 (m, 1H), 3.80-3.70 (m, 1H), 3.60-3.50 (m, 5H), 3.30-3.10 (m, 2H), 3.10-3.00 (m, 1H), 2.90-2.70 (m, 2H), 2.40 (s, 3H), 2.00-1.50 (m, 6H).

### Examples 53a and 53b: (R)-1-(8-((2-Chloro-4-methylphenyl)sulfonyl)-1-oxa-8-azaspiro[4.5]decan-3-yl)azetidin-3-ol and (S)-1-(8-((2-chloro-4-methylphenyl)sulfonyl)-1-oxa-8-azaspiro[4.5]decan-3-yl)azetidin-3-ol

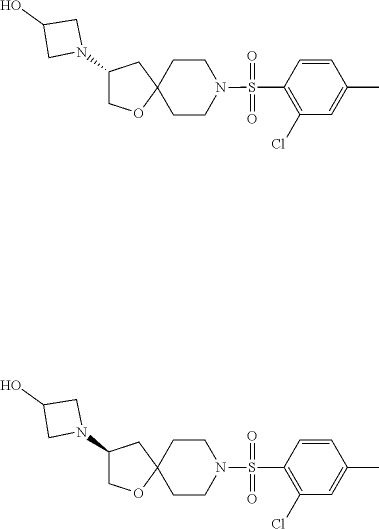 [SMILES: Cc1ccc(S(=O)(=O)N2CCC3(CC2)C[C@@H](N2CC(O)C2)CO3)c(Cl)c1 ; Cc1ccc(S(=O)(=O)N2CCC3(CC2)C[C@H](N2CC(O)C2)CO3)c(Cl)c1]

1-(8-((2-Chloro-4-methylphenyl)sulfonyl)-1-oxa-8-azaspiro[4.5]decan-3-yl)azetidin-3-ol (Example 52) was separated on a CHIRALPAK AD-H 30×250 mm, 5 μm column. Method: 40% MeOH w/0.1% DEA in CO₂ (flow rate: 100 mL/min, ABPR 120 bar, MBPR 40 psi, column temp 40° C.) to give two enantiomers of arbitrarily assigned stereochemistry:
Peak 1, enantiomer 1, (R)-1-(8-((2-chloro-4-methylphenyl)sulfonyl)-1-oxa-8-azaspiro[4.5]decan-3-yl)azetidin-3-ol: LCMS m/z=401.2 [M+H]⁺. R_{f}=2.57 min. ee=100%.
Peak 2, enantiomer 2, (S)-1-(8-((2-chloro-4-methylphenyl)sulfonyl)-1-oxa-8-azaspiro[4.5]decan-3-yl)azetidin-3-ol: LCMS m/z=401.2 [M+H]⁺. R_{f}=3.10 min. ee=100%.

### Example 54: 8-((2-Methyl-4-(trifluoromethoxy)phenyl)sulfonyl)-3-morpholino-1-oxa-8-azaspiro[4.5]decane

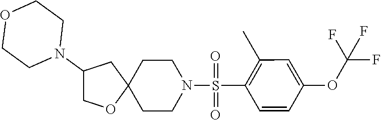 [SMILES: Cc1cc(OC(F)(F)F)ccc1S(=O)(=O)N1CCC2(CC1)CC(N1CCOCC1)CO2]

### 1. Synthesis of 8-((2-methyl-4-(trifluoromethoxy)phenyl)sulfonyl)-1-oxa-8-azaspiro[4.5]decan-3-one

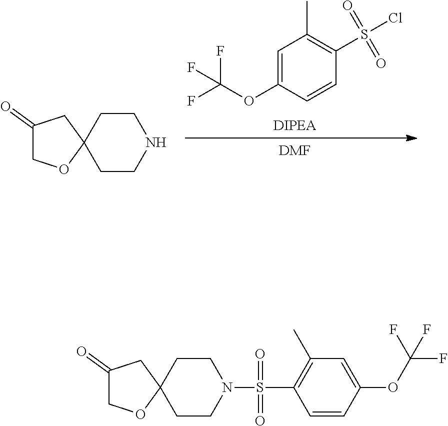 [SMILES: Cc1cc(OC(F)(F)F)ccc1S(=O)(=O)Cl ; O=C1COC2(CCNCC2)C1 ; Cc1cc(OC(F)(F)F)ccc1S(=O)(=O)N1CCC2(CC1)CC(=O)CO2]

DIPEA (800 μL, 4.6 mmol) was added to a vial containing 1-oxa-8-azaspiro[4.5]decan-3-one hydrochloride (800 μL, 0.9 mmol) in anhydrous DMF (2 mL). After 5 min, 2-methyl-4-(trifluoromethoxy)benzenesulfonyl chloride (254 mg, 0.9 mmol) was added and the reaction was stirred at room temperature for 1 h. The reaction was quenched with water and the mixture was extracted with EtOAc (3×). The combined organics were washed with saturated aqueous NaHCO₃ (2×) and the organic layer was dried over anhydrous Na₂SO₄. The crude material was purified by silica gel column (0-40% Hexanes-EtOAc) to afford 8-((2-methyl-4-(trifluoromethoxy)phenyl)sulfonyl)-1-oxa-8-azaspiro[4.5]decan-3-one (358 mg, 98%). LCMS m/z=394.1 [M+H]⁺.

### 2. Synthesis of 8-((2-methyl-4-(trifluoromethoxy)phenyl)sulfonyl)-3-morpholino-1-oxa-8-azaspiro[4.5]decane

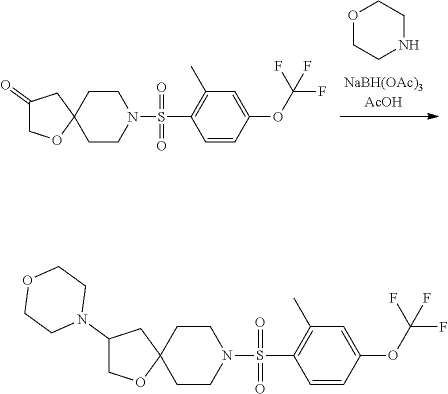 [SMILES: C1COCCN1 ; Cc1cc(OC(F)(F)F)ccc1S(=O)(=O)N1CCC2(CC1)CC(=O)CO2 ; Cc1cc(OC(F)(F)F)ccc1S(=O)(=O)N1CCC2(CC1)CC(N1CCOCC1)CO2]

Morpholine hydrochloride (38 mg, 0.3 mmol) was added to a solution of 8-((2-methyl-4-(trifluoromethoxy)phenyl)sulfonyl)-1-oxa-8-azaspiro[4.5]decan-3-one (100 mg, 0.3 mmol) in DCM (8 mL) and the solution stirred at room temperature for 15 min. Acetic acid (30 μL, 0.5 mmol) was added dropwise, followed by NaBH(OAc)₃ (216 mg, 1.0 mmol) and the reaction stirred at room temperature for 3 h. The reaction was quenched with saturated aqueous NH₄Cl, neutralized with saturated aqueous NaHCO₃ and diluted with DCM. The organics were washed with water and brine, the solvent was removed in vacuo and the crude product purified by silica gel column (0-100% EtOAc to 3:1 EtOAc:EtOH (w/2% NH₃OH)) to give 8-((2-methyl-4-(trifluoromethoxy)phenyl)sulfonyl)-3-morpholino-1-oxa-8-azaspiro[4.5]decane (118 mg, 75%). LCMS m/z=465.1 [M+H]⁺. ¹H-NMR (400 MHz, CDCl₃) δ (ppm): 8.00-7.80 (m, 1H), 7.15 (br d, J=0.8 Hz, 2H), 3.97 (dd, J=6.8, 8.5 Hz, 1H), 3.80-3.60 (m, 5H), 3.60-3.40 (m, 2H), 3.20-2.90 (m, 3H), 2.64 (s, 3H), 2.60-2.30 (m, 4H), 1.97 (dd, J=7.8, 12.3 Hz, 1H), 1.80-1.50 (m, 5H).

### Example 55: 8-((2-Cyclopropylthiazol-5-yl)sulfonyl)-3-(3-methoxyazetidin-1-yl)-1-oxa-8-azaspiro[4.5]decane

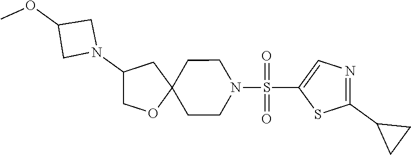 [SMILES: COC1CN(C2COC3(CCN(S(=O)(=O)c4cnc(C5CC5)s4)CC3)C2)C1]

### 1. Synthesis of 8-((2-cyclopropylthiazol-5-yl)sulfonyl)-1-oxa-8-azaspiro[4.5]decan-3-one

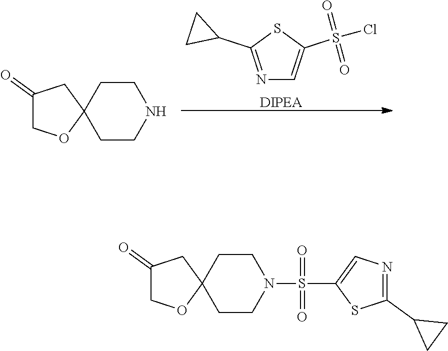

DIPEA (1.9 mL, 11.2 mmol) was added to a vial containing 1-oxa-8-azaspiro[4.5]decan-3-one hydrochloride (428 mg, 2.2 mmol) in DMF (4 mL). After 5 min, 2-cyclopropylthiazole-5-sulfonyl chloride (500 mg, 2.24 mmol) was added and the reaction mixture was stirred at room temperature for 1 h. The reaction was quenched with water and the mixture was extracted with EtOAc (3×). The combined organics were washed with saturated aqueous NaHCO₃ solution (2×). The organic layer was separated and dried over anhydrous Na₂SO₄. The crude material was purified by silica gel column (0-25% EtOAc to 3:1 EtOAc:EtOH) to afford 8-((2-cyclopropylthiazol-5-yl)sulfonyl)-1-oxa-8-azaspiro[4.5]decan-3-one as a yellow solid. (630 mg, 82%). LCMS m/z=343.0 [M+H]⁺. ¹H-NMR (400 MHz, CDCl₃) δ (ppm): 7.94 (s, 1H), 3.97 (s, 2H), 3.60-3.50 (m, 2H), 2.93 (dt, J=3.5, 11.2 Hz, 2H), 2.40-2.20 (m, 3H), 2.00-1.80 (m, 4H), 1.40-1.10 (m, 4H).

### 2. Synthesis of 8-((2-cyclopropylthiazol-5-yl)sulfonyl)-3-(3-methoxyazetidin-1-yl)-1-oxa-8-azaspiro[4.5]decane

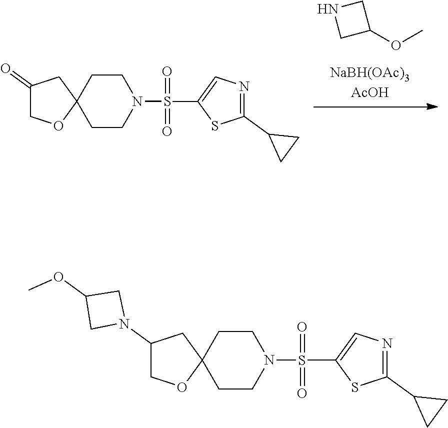

3-Methoxyazetidine hydrochloride (42 mg, 0.3 mmol) was added to a solution of 8-(2-cyclopropylthiazol-5-yl)sulfonyl-1-oxa-8-azaspiro[4.5]decan-3-one (116 mg, 0.3 mmol) in DCM (10 mL) and the solution stirred at room temperature for 15 min. Acetic acid (40 μL, 0.7 mmol) was added dropwise, followed by NaBH(OAc)₃ (287 mg, 1.4 mmol) and the reaction stirred at room temperature for 3 h. The reaction was quenched with saturated aqueous NH₄Cl and diluted with DCM. The organics were washed with water and brine and the solvent was removed in vacuo. The crude product was purified by silica gel column (0-100% EtOAc to 3:1 EtOAc:EtOH (w/2% NH₃OH)) to afford 8-((2-cyclopropylthiazol-5-yl)sulfonyl)-3-(3-methoxyazetidin-1-yl)-1-oxa-8-azaspiro[4.5]decane (140 mg, 95%). LCMS m/z=414.2 [M+H]⁺. ¹H-NMR (400 MHz, CDCl₃) δ (ppm): 7.88 (s, 1H), 3.72 (dd, J=5.4, 9.2 Hz, 1H), 3.58 (dd, J=3.8, 9.0 Hz, 1H), 3.47 (td, J=3.0, 7.5 Hz, 2H), 3.20-3.10 (m, 4H), 3.10-3.00 (m, 1H), 3.00-2.90 (m, 2H), 2.80-2.70 (m, 2H), 2.40-2.20 (m, 1H), 1.80-1.40 (m, 8H), 1.30-1.10 (m, 4H).

### Example 56: 8-((3-Cyclopropyl-1-methyl-1H-pyrazol-5-yl)sulfonyl)-3-(2-oxa-6-azaspiro[3.3]heptan-6-yl)-1-oxa-8-azaspiro[4.5]decane

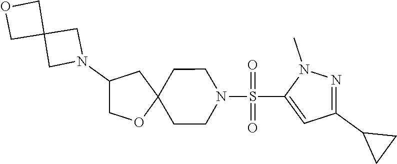 [SMILES: Cn1nc(C2CC2)cc1S(=O)(=O)N1CCC2(CC1)CC(N1CC3(COC3)C1)CO2]

### 1. Synthesis of 8-((3-cyclopropyl-1-methyl-1H-pyrazol-5-yl)sulfonyl)-1-oxa-8-azaspiro[4.5]decan-3-one

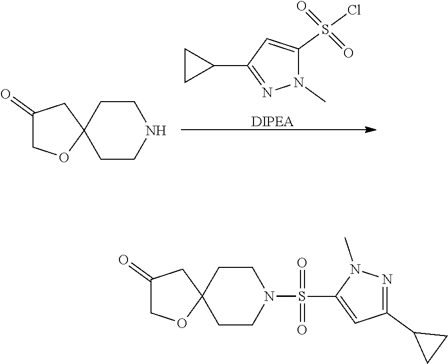

DIPEA (1.6 mL, 9.4 mmol) was added to a vial containing 1-oxa-8-azaspiro[4.5]decan-3-one hydrochloride (360 mg, 1.9 mmol) in DMF (4 mL). After 5 min, 5-cyclopropyl-2-methyl-pyrazole-3-sulfonyl chloride (415 mg, 1.9 mmol) was added and the reaction stirred at room temperature for 1 h. The reaction was quenched with water and the mixture extracted with EtOAc (3×). The combined organics were washed with saturated aqueous NaHCO₃ (2×). The organic layer was separated then dried over Na₂SO₄ and concentrated in vacuo. The crude material was purified by silica gel column (0-25% EtOAc to 3:1 EtOAc:EtOH) to afford 8-((3-cyclopropyl-1-methyl-1H-pyrazol-5-yl)sulfonyl)-1-oxa-8-azaspiro[4.5]decan-3-one (610 mg, 95%). LCMS m/z=340.1 [M+H]⁺.

### 2. Synthesis of 8-((3-cyclopropyl-1-methyl-1H-pyrazol-5-yl)sulfonyl)-3-(2-oxa-6-azaspiro[3.3]heptan-6-yl)-1-oxa-8-azaspiro[4.5]decane

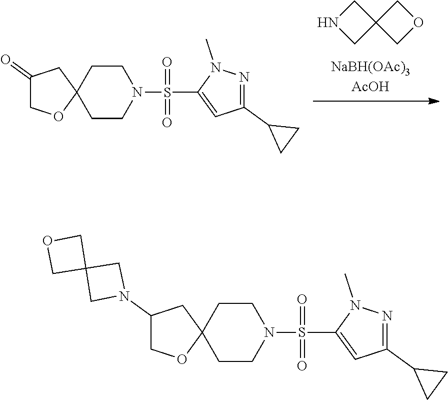 [SMILES: Cn1nc(C2CC2)cc1S(=O)(=O)N1CCC2(CC1)CC(=O)CO2 ; C1NCC12COC2 ; Cn1nc(C2CC2)cc1S(=O)(=O)N1CCC2(CC1)CC(N1CC3(COC3)C1)CO2]

2-Oxa-6-azaspiro[3.3]heptane (25 mg, 0.3 mmol) was added to a solution of 8-(5-cyclopropyl-2-methyl-pyrazol-3-yl)sulfonyl-1-oxa-8-azaspiro[4.5]decan-3-one (84 mg, 0.3 mmol) in DCM (15 mL) and the solution stirred at room temperature for 15 min. Acetic acid (30 mg, 0.5 mmol) was added dropwise, followed by NaBH(OAc)₃ (210 mg, 1.0 mmol) and the reaction stirred at room temperature for 3 h. The reaction was quenched with saturated aqueous NH₄Cl and diluted with DCM. The organics were washed with water and brine and the solvent was removed in vacuo. The crude product was purified by silica gel column (0-100% EtOAc to 3:1 EtOAc:EtOH (w/2% NH₃OH)) to afford 8-((3-cyclopropyl-1-methyl-1H-pyrazol-5-yl)sulfonyl)-3-(2-oxa-6-azaspiro[3.3]heptan-6-yl)-1-oxa-8-azaspiro[4.5]decane (100 mg, 96%). LCMS m/z=423.2 [M+H]⁺. ¹H-NMR (400 MHz, CDCl₃) δ (ppm): 6.25 (s, 1H), 4.70-4.60 (m, 4H), 3.92 (s, 3H), 3.62 (dd, J=5.3, 9.3 Hz, 1H), 3.50-3.40 (m, 3H), 3.30-3.10 (m, 4H), 2.90-2.70 (m, 3H), 1.90-1.70 (m, 2H), 1.70-1.50 (m, 4H), 1.44 (dd, J=3.8, 13.1 Hz, 1H), 0.90-0.80 (m, 2H), 0.70-0.60 (m, 2H).

### Examples 57a and 57b: (R)-8-((3-Cyclopropyl-1-methyl-1H-pyrazol-5-yl)sulfonyl)-3-(2-oxa-6-azaspiro[3.3]heptan-6-yl)-1-oxa-8-azaspiro[4.5]decane and (S)-8-((3-cyclopropyl-1-methyl-1H-pyrazol-5-yl)sulfonyl)-3-(2-oxa-6-azaspiro[3.3]heptan-6-yl)-1-oxa-8-azaspiro[4.5]decane

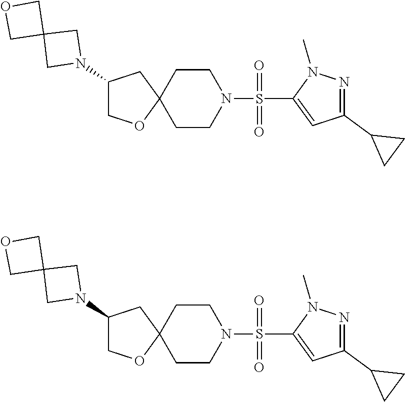 [SMILES: Cn1nc(C2CC2)cc1S(=O)(=O)N1CCC2(CC1)C[C@@H](N1CC3(COC3)C1)CO2 ; Cn1nc(C2CC2)cc1S(=O)(=O)N1CCC2(CC1)C[C@H](N1CC3(COC3)C1)CO2]

8-((3-Cyclopropyl-1-methyl-1H-pyrazol-5-yl)sulfonyl)-3-(2-oxa-6-azaspiro[3.3]heptan-6-yl)-1-oxa-8-azaspiro[4.5]decane (Example 56) was separated on a CHIRALPAK IA 30×250 mm, 5 μm column. Using method: 30% MeOH w/No Modifier in CO₂ (flow rate: 100 mL/min, ABPR 120 bar, MBPR 40 psi, column temp 40 deg ° C.) to give two enantiomers of arbitrarily assigned stereochemistry:
Peak 1, Enantiomer 1, (R)-8-((3-cyclopropyl-1-methyl-1H-pyrazol-5-yl)sulfonyl)-3-(2-oxa-6-azaspiro[3.3]heptan-6-yl)-1-oxa-8-azaspiro[4.5]decane: LCMS m/z=423.2 [M+H]⁺, R_{f}=2.09 min., ee=100%
Peak 2, Enantiomer 2, (S)-8-((3-cyclopropyl-1-methyl-1H-pyrazol-5-yl)sulfonyl)-3-(2-oxa-6-azaspiro[3.3]heptan-6-yl)-1-oxa-8-azaspiro[4.5]decane: LCMS m/z=423.2 [M+H]⁺, R_{f}=2.25 min., ee=82.84%

### Example 58: 8-((3-Cyclopropyl-1-methyl-1H-pyrazol-5-yl)sulfonyl)-3-(3-methoxyazetidin-1-yl)-1-oxa-8-azaspiro[4.5]decane

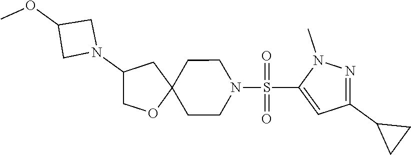 [SMILES: COC1CN(C2COC3(CCN(S(=O)(=O)c4cc(C5CC5)nn4C)CC3)C2)C1]

8-((3-Cyclopropyl-1-methyl-1H-pyrazol-5-yl)sulfonyl)-3-(3-methoxyazetidin-1-yl)-1-oxa-8-azaspiro[4.5]decane was obtained (78 mg, 77%) from 3-methoxyazetidine and 8-(5-cyclopropyl-2-methyl-pyrazol-3-yl)sulfonyl-1-oxa-8-azaspiro[4.5]decan-3-one, following the procedure described in Example 56, step 2. LCMS m/z=411.2 [M+H]⁺. ¹H-NMR (400 MHz, CDCl₃) δ (ppm): 6.32 (s, 1H), 4.10-3.90 (m, 4H), 3.90-3.70 (m, 1H), 3.70-3.40 (m, 5H), 3.24 (s, 3H), 3.10-2.70 (m, 5H), 2.00-1.50 (m, 7H), 1.10-0.80 (m, 2H), 0.80-0.60 (m, 2H).

### Example 59: 8-((3-Cyclopropyl-1-methyl-1H-pyrazol-5-yl)sulfonyl)-N-(2-methoxyethyl)-1-oxa-8-azaspiro[4.5]decan-3-amine

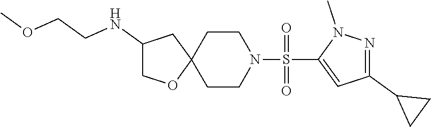 [SMILES: COCCNC1COC2(CCN(S(=O)(=O)c3cc(C4CC4)nn3C)CC2)C1]

8-((3-Cyclopropyl-1-methyl-1H-pyrazol-5-yl)sulfonyl)-N-(2-methoxyethyl)-1-oxa-8-azaspiro[4.5]decan-3-amine was obtained (80 mg, 73%) from 2-methoxyethanamine and 8-(5-cyclopropyl-2-methyl-pyrazol-3-yl)sulfonyl-1-oxa-8-azaspiro[4.5]decan-3-one, following the procedure described in Example 56, step 2. LCMS m/z=399.2 [M+H]⁺. ¹H-NMR (400 MHz, CDCl₃) δ (ppm): 6.32 (s, 1H), 3.98 (s, 3H), 3.91 (dd, J=6.0, 9.0 Hz, 1H), 3.70-3.40 (m, 6H), 3.33 (s, 3H), 2.93 (dq, J=3.5, 11.5 Hz, 2H), 2.80-2.60 (m, 2H), 2.10-1.50 (m, 9H), 1.00-0.80 (m, 2H), 0.80-0.70 (m, 2H).

### Example 60: 8-((3-Cyclopropyl-1-methyl-1H-pyrazol-5-yl)sulfonyl)-N-(2-methoxyethyl)-N-methyl-1-oxa-8-azaspiro[4.5]decan-3-amine

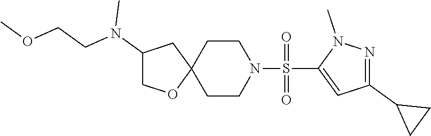 [SMILES: COCCN(C)C1COC2(CCN(S(=O)(=O)c3cc(C4CC4)nn3C)CC2)C1]

### 1. Synthesis of 8-((3-cyclopropyl-1-methyl-H-pyrazol-5-yl)sulfonyl)-N-(2-methoxyethyl)-N-methyl-1-oxa-8-azaspiro[4.5]decan-3-amine

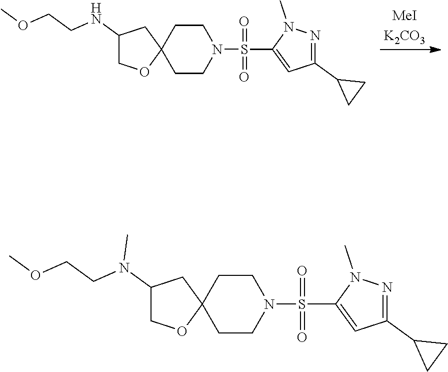 [SMILES: COCCNC1COC2(CCN(S(=O)(=O)c3cc(C4CC4)nn3C)CC2)C1 ; COCCN(C)C1COC2(CCN(S(=O)(=O)c3cc(C4CC4)nn3C)CC2)C1]

8-(5-Cyclopropyl-2-methyl-pyrazol-3-yl)sulfonyl-N-(2-methoxyethyl)-1-oxa-8-azaspiro[4.5]decan-3-amine (70 mg, 0.2 mmol) was dissolved in MeCN (3 mL), K₂CO₃ (49 mg, 0.4 mmol) was added and the mixture was stirred for 30 min. Methyl iodide (10 μL, 0.2 mmol) was added and the reaction stirred overnight. The reaction was diluted with EtOAc and washed with water (3×). The organic phase was concentrated in vacuo and the residue was purified by silica gel column (0-100% EtOAc to 3:1 EtOAc:EtOH (w/2% NH₃OH)) to give 8-((3-cyclopropyl-1-methyl-1H-pyrazol-5-yl)sulfonyl)-N-(2-methoxyethyl)-N-methyl-1-oxa-8-azaspiro[4.5]decan-3-amine (72 mg, 37%). LCMS m/z=413.3 [M+H]⁺. ¹H-NMR (400 MHz, CDCl₃) δ (ppm): 6.26 (s, 1H), 4.00-3.80 (m, 4H), 3.80-3.50 (m, 2H), 3.50-3.30 (m, 4H), 3.28 (s, 3H), 3.00-2.70 (m, 2H), 2.60-2.40 (m, 2H), 2.19 (s, 3H), 1.90-1.50 (m, 7H), 0.90-0.80 (m, 2H), 0.70-0.60 (m, 2H).

### Example 61: 8-((5-Chloro-2-methoxypyridin-3-yl)sulfonyl)-3-(3-methoxyazetidin-1-yl)-1-oxa-8-azaspiro[4.5]decane

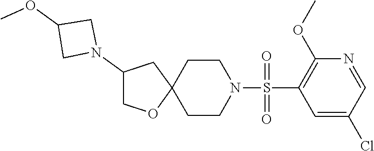 [SMILES: COc1ncc(Cl)cc1S(=O)(=O)N1CCC2(CC1)CC(N1CC(OC)C1)CO2]

### 1. Synthesis of 8-((5-chloro-2-methoxypyridin-3-yl)sulfonyl)-1-oxa-8-azaspiro[4.5]decan-3-one

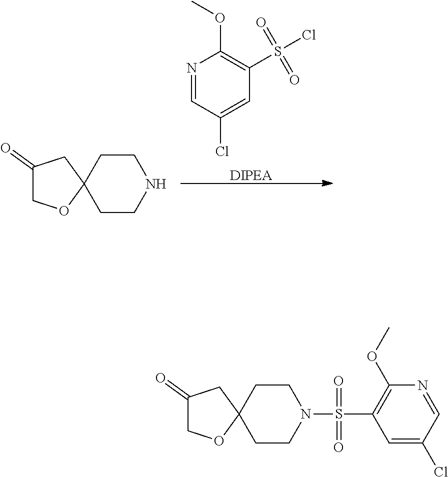

8-((5-Chloro-2-methoxypyridin-3-yl)sulfonyl)-1-oxa-8-azaspiro[4.5]decan-3-one was obtained (120 mg, 40%) from 5-chloro-2-methoxy-pyridine-3-sulfonyl chloride and 1-oxa-8-azaspiro[4.5]decan-3-one hydrochloride, following the procedure described in Example 56, step 1. LCMS m/z=361.1 [M+H]⁺. ¹H-NMR (400 MHz, CDCl₃) δ (ppm): 8.26 (d, J=2.5 Hz, 1H), 8.14 (d, J=2.5 Hz, 1H), 4.05 (s, 3H), 3.98 (s, 2H), 3.66 (td, J=3.5, 12.7 Hz, 2H), 3.30-3.00 (m, 2H), 2.37 (s, 2H), 2.00-1.70 (m, 4H).

### 2. Synthesis of 8-((5-chloro-2-methoxypyridin-3-yl)sulfonyl)-3-(3-methoxyazetidin-1-yl)-1-oxa-8-azaspiro[4.5]decane

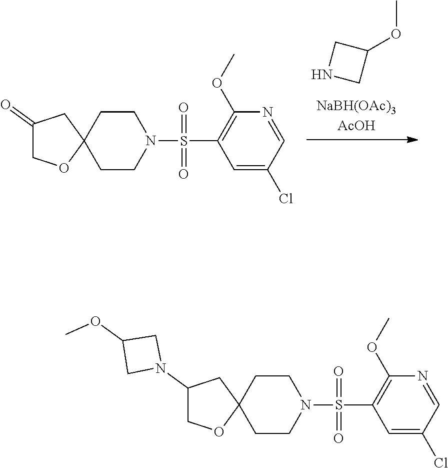

8-((5-Chloro-2-methoxypyridin-3-yl)sulfonyl)-3-(3-methoxyazetidin-1-yl)-1-oxa-8-azaspiro[4.5]decane was obtained (40 mg, 60%) from 3-methoxyazetidine and 8-((5-chloro-2-methoxypyridin-3-yl)sulfonyl)-1-oxa-8-azaspiro[4.5]decan-3-one, following a similar synthesis to that described in Example 56, step 2. LCMS m/z=432.1 [M+H]⁺. ¹H-NMR (400 MHz, CDCl₃) δ (ppm): 8.30-8.20 (m, 1H), 8.20-8.10 (m, 1H), 4.20-4.00 (m, 4H), 3.76 (br dd, J=5.3, 9.3 Hz, 1H), 3.70-3.50 (m, 5H), 3.26 (s, 3H), 3.20-3.00 (m, 3H), 2.87 (td, J=6.4, 19.3 Hz, 2H), 2.00-1.50 (m, 6H).

### Example 62: 8-((5-Chloro-2-methoxypyridin-3-yl)sulfonyl)-3-(2-oxa-6-azaspiro[3.3]heptan-6-yl)-1-oxa-8-azaspiro[4.5]decane

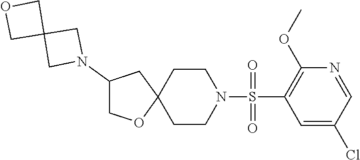 [SMILES: COc1ncc(Cl)cc1S(=O)(=O)N1CCC2(CC1)CC(N1CC3(COC3)C1)CO2]

8-((5-Chloro-2-methoxypyridin-3-yl)sulfonyl)-3-(2-oxa-6-azaspiro[3.3]heptan-6-yl)-1-oxa-8-azaspiro[4.5]decane was obtained (80 mg, 97%) yield from 2-oxa-6-azaspiro[3.3]heptane and 8-((5-chloro-2-methoxypyridin-3-yl)sulfonyl)-1-oxa-8-azaspiro[4.5]decan-3-one, following a similar synthesis to that described in Example 56, step 2. LCMS m/z=444.1 [M+H]⁺. ¹H-NMR (400 MHz, CDCl₃) δ (ppm): 8.16 (d, J=2.5 Hz, 1H), 8.04 (d, J=2.5 Hz, 1H), 4.70-4.60 (m, 4H), 3.95 (s, 3H), 3.63 (dd, J=5.3, 9.3 Hz, 1H), 3.60-3.40 (m, 3H), 3.40-3.20 (m, 4H), 3.10-2.90 (m, 2H), 2.90-2.70 (m, 1H), 1.90-1.50 (m, 5H), 1.43 (dd, J=3.9, 12.9 Hz, 1H).

### Examples 63a and 63b: (R)-8-((5-Chloro-2-methoxypyridin-3-yl)sulfonyl)-3-(2-oxa-6-azaspiro[3.3]heptan-6-yl)-1-oxa-8-azaspiro[4.5]decane and (S)-8-((5-chloro-2-methoxypyridin-3-yl)sulfonyl)-3-(2-oxa-6-azaspiro[3.3]heptan-6-yl)-1-oxa-8-azaspiro[4.5]decane

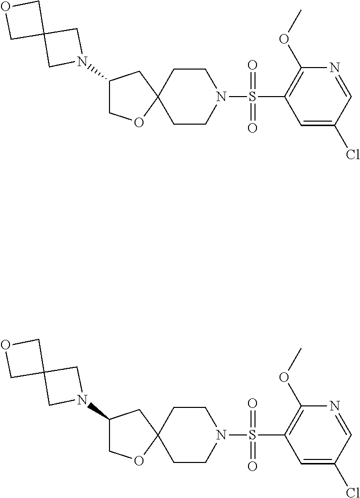 [SMILES: COc1ncc(Cl)cc1S(=O)(=O)N1CCC2(CC1)C[C@@H](N1CC3(COC3)C1)CO2 ; COc1ncc(Cl)cc1S(=O)(=O)N1CCC2(CC1)C[C@H](N1CC3(COC3)C1)CO2]

8-((5-Chloro-2-methoxypyridin-3-yl)sulfonyl)-3-(2-oxa-6-azaspiro[3.3]heptan-6-yl)-1-oxa-8-azaspiro[4.5]decane (Example 62) was separated on a CHIRALPAK AD-H 30×250 mm, 5 μm column. Method: 25% MeOH w/0.1% DEA in CO₂ (flow rate: 100 mL/min, ABPR 120 bar, MBPR 40 psi, column temp 40° C.) to give two enantiomers of arbitrarily assigned stereochemistry:
Peak 1, Enantiomer 1, (R)-8-((5-chloro-2-methoxypyridin-3-yl)sulfonyl)-3-(2-oxa-6-azaspiro[3.3]heptan-6-yl)-1-oxa-8-azaspiro[4.5]decane: LCMS m/z=444.1 [M+H]⁺. R_{f}=2.12 min., ee=100%
Peak 2, Enantiomer 2, (S)-8-((5-chloro-2-methoxypyridin-3-yl)sulfonyl)-3-(2-oxa-6-azaspiro[3.3]heptan-6-yl)-1-oxa-8-azaspiro[4.5]decane: LCMS m/z=444.1 [M+H]⁺. R_{f}=2.33 min., ee=96.9%.

### Example 64: 3-((3-(2-Oxa-6-azaspiro[3.3]heptan-6-yl)-1-oxa-8-azaspiro[4.5]decan-8-yl)sulfonyl)-4-chlorobenzonitrile

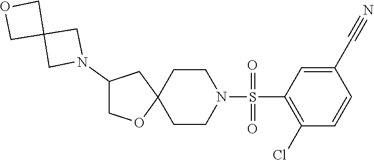 [SMILES: N#Cc1ccc(Cl)c(S(=O)(=O)N2CCC3(CC2)CC(N2CC4(COC4)C2)CO3)c1]

### 1. Synthesis of 4-chloro-3-((3-oxo-1-oxa-8-azaspiro[4.5]decan-8-yl)sulfonyl)benzonitrile

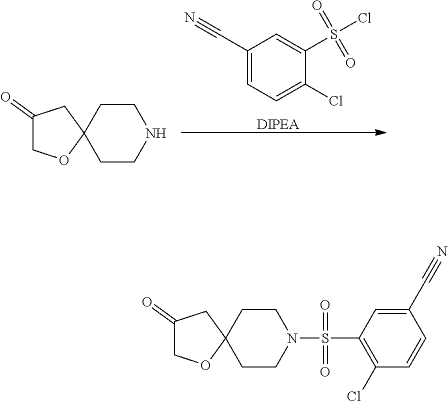

4-Chloro-3-((3-oxo-1-oxa-8-azaspiro[4.5]decan-8-yl)sulfonyl)benzonitrile was obtained (210 mg, 57%) from 2-chloro-5-cyano-benzenesulfonyl chloride and 1-oxa-8-azaspiro[4.5]decan-3-one hydrochloride, following the procedure described in Example 56, step 1. LCMS m/z=355.1 [M+H]⁺. ¹H-NMR (400 MHz, CDCl₃) δ (ppm): 8.38 (d, J=2.0 Hz, 1H), 7.78 (dd, J=2.0, 8.3 Hz, 1H), 7.70-7.60 (m, 1H), 4.01 (s, 2H), 3.80-3.60 (m, 2H), 3.40-3.20 (m, 2H), 2.40 (s, 2H), 2.00-1.80 (m, 4H).

### 2. Synthesis of 3-((3-(2-oxa-6-azaspiro[3.3]heptan-6-yl)-1-oxa-8-azaspiro[4.5]decan-8-yl)sulfonyl)-4-chlorobenzonitrile

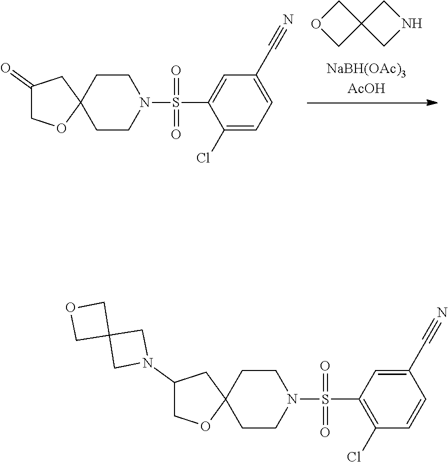

3-((3-(2-Oxa-6-azaspiro[3.3]heptan-6-yl)-1-oxa-8-azaspiro[4.5]decan-8-yl)sulfonyl)-4-chlorobenzonitrile was obtained (68 mg, 79%) from 2-oxa-6-azaspiro[3.3]heptane and 4-chloro-3-((3-oxo-1-oxa-8-azaspiro[4.5]decan-8-yl)sulfonyl)benzonitrile following a similar synthesis to that described in Example 56, step 2. LCMS m/z=438.2 [M+H]⁺. ¹H-NMR (400 MHz, CDCl₃) δ (ppm): 8.32 (d, J=1.8 Hz, 1H), 7.74 (dd, J=2.0, 8.0 Hz, 1H), 7.70-7.50 (m, 1H), 4.80-4.60 (m, 4H), 3.71 (dd, J=5.4, 9.4 Hz, 1H), 3.70-3.50 (m, 3H), 3.40-3.20 (m, 4H), 3.20-3.00 (m, 2H), 3.00-2.90 (m, 1H), 1.94 (br d, J=13.8 Hz, 1H), 1.80-1.60 (m, 4H), 1.52 (dd, J=3.8, 13.1 Hz, 1H).

### Example 65: 8-((2-Methoxy-5-methylpyridin-3-yl)sulfonyl)-N-(2-methoxyethyl)-1-oxa-8-azaspiro[4.5]decan-3-amine

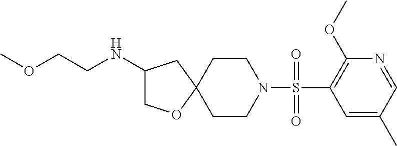 [SMILES: COCCNC1COC2(CCN(S(=O)(=O)c3cc(C)cnc3OC)CC2)C1]

### 1. Synthesis of 8-((2-methoxy-5-methylpyridin-3-yl)sulfonyl)-1-oxa-8-azaspiro[4.5]decan-3-one

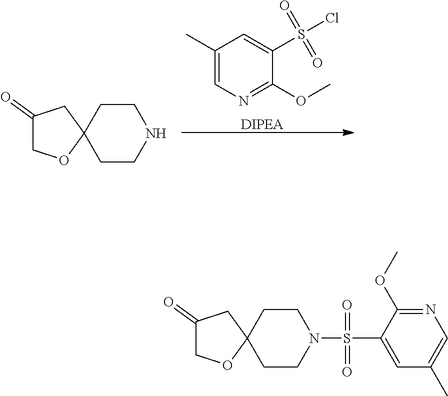

8-((2-Methoxy-5-methylpyridin-3-yl)sulfonyl)-1-oxa-8-azaspiro[4.5]decan-3-one was obtained (260 mg, 73%) from 2-methoxy-5-methyl-pyridine-3-sulfonyl chloride and 1-oxa-8-azaspiro[4.5]decan-3-one hydrochloride, following the procedure described in Example 56, step 1. LCMS m/z=341.1 [M+H]⁺. ¹H-NMR (400 MHz, CDCl₃) δ (ppm): 8.11 (d, J=2.3 Hz, 1H), 7.97 (d, J=2.5 Hz, 1H), 4.02 (s, 3H), 3.96 (s, 2H), 3.61 (td, J=3.8, 12.7 Hz, 2H), 3.30-3.10 (m, 2H), 2.35 (s, 2H), 2.30 (s, 3H), 1.90-1.70 (m, 4H).

### 2. Synthesis of 8-((2-methoxy-5-methylpyridin-3-yl)sulfonyl)-N-(2-methoxyethyl)-1-oxa-8-azaspiro[4.5]decan-3-amine

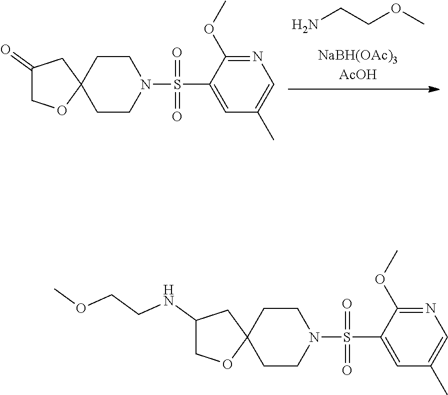

8-((2-Methoxy-5-methylpyridin-3-yl)sulfonyl)-N-(2-methoxyethyl)-1-oxa-8-azaspiro[4.5]decan-3-amine was obtained (85 mg, 72%) from 2-methoxyethanamine and 8-((2-methoxy-5-methylpyridin-3-yl)sulfonyl)-1-oxa-8-azaspiro[4.5]decan-3-one, following a similar synthesis to that described in Example 56, step 2. LCMS m/z=400.2 [M+H]⁺. ¹H-NMR (400 MHz, CDCl₃) δ (ppm): 8.09 (dd, J=0.8, 2.5 Hz, 1H), 7.96 (d, J=2.5 Hz, 1H), 4.01 (s, 3H), 3.92 (dd, J=5.8, 9.0 Hz, 1H), 3.70-3.50 (m, 5H), 3.41 (dd, J=5.8, 7.5 Hz, 1H), 3.35 (s, 3H), 3.10-3.00 (m, 2H), 2.80-2.70 (m, 2H), 2.29 (s, 3H), 2.02 (dd, J=7.5, 12.8 Hz, 1H), 1.90-1.60 (m, 5H), 1.56 (dd, J=5.9, 12.9 Hz, 1H).

### Example 66: 8-((2-Methoxy-5-methylpyridin-3-yl)sulfonyl)-N-(2-methoxyethyl)-N-(methyl-d3)-1-oxa-8-azaspiro[4.5]decan-3-amine

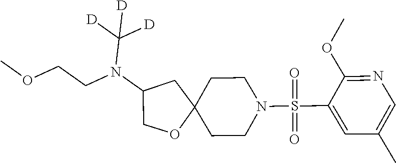

### 1. Synthesis of 8-((2-methoxy-5-methylpyridin-3-yl)sulfonyl)-N-(2-methoxyethyl)-N-(methyl-d3)-1-oxa-8-azaspiro[4.5]decan-3-amine

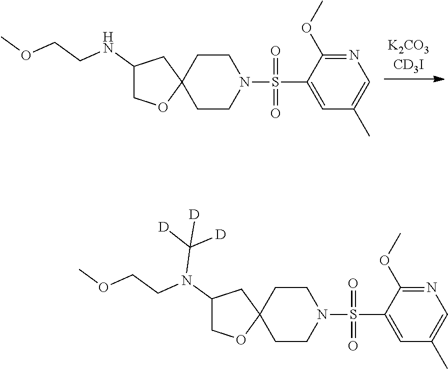

8-((2-Methoxy-5-methylpyridin-3-yl)sulfonyl)-N-(2-methoxyethyl)-1-oxa-8-azaspiro[4.5]decan-3-amine (Example 65, 13 mg, 0.3 mmol) was dissolved in MeCN (3 mL), K₂CO₃ (9 mg, 0.7 mmol) was added and the reaction stirred for 30 min. Trideuterio(iodo)methane (5 mg, 0.03 mmol) was added and the reaction stirred overnight. The reaction was diluted with EtOAc and washed with water (3×). The organic solution was concentrated in vacuo and the crude product was purified by silica gel column chromatography (0-100% EtOAc to 3:1 EtOAc:EtOH (w/2% NH₃OH)) to give 8-((2-methoxy-5-methylpyridin-3-yl)sulfonyl)-N-(2-methoxyethyl)-N-(methyl-d3)-1-oxa-8-azaspiro[4.5]decan-3-amine. LCMS m/z=417.3 [M+H]⁺. ¹H-NMR (400 MHz, CDCl₃) δ (ppm): 8.03 (dd, J=0.9, 2.4 Hz, 1H), 7.90-7.80 (m, 1H), 4.00-3.90 (m, 3H), 3.89 (br dd, J=7.3, 9.0 Hz, 1H), 3.60-3.40 (m, 4H), 3.30-3.20 (m, 3H), 3.10-2.90 (m, 2H), 2.23 (s, 3H), 1.80-1.40 (m, 10H).

### Example 67: 8-((2-Methoxy-5-methylpyridin-3-yl)sulfonyl)-N-(2-methoxyethyl)-N-methyl-1-oxa-8-azaspiro[4.5]decan-3-amine

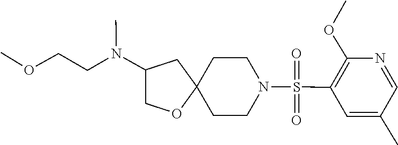

8-((2-Methoxy-5-methylpyridin-3-yl)sulfonyl)-N-(2-methoxyethyl)-N-methyl-1-oxa-8-azaspiro[4.5]decan-3-amine was obtained (27 mg, 60%) from 8-((2-methoxy-5-methylpyridin-3-yl)sulfonyl)-N-(2-methoxyethyl)-1-oxa-8-azaspiro[4.5]decan-3-amine (Example 65) and methyl iodide, following the procedure described in Example 66. LCMS m/z=414.2 [M+H]⁺. ¹H-NMR (400 MHz, CDCl₃) δ (ppm): 8.12 (d, J=2.3 Hz, 1H), 7.98 (d, J=2.5 Hz, 1H), 4.10-4.00 (m, 3H), 3.97 (dd, J=7.0, 9.0 Hz, 1H), 3.70-3.50 (m, 4H), 3.37 (s, 3H), 3.20-3.00 (m, 2H), 2.40-2.30 (m, 6H), 1.99 (br dd, J=8.3, 12.3 Hz, 1H), 1.90-1.50 (m, 9H).

### Examples 68a and 68b: (R)-6-(8-((1,3-dimethyl-1H-pyrazol-5-yl)sulfonyl)-8-azaspiro[4.5]decan-2-yl)-2-oxa-6-azaspiro[3.3]heptane and (S)-6-(8-((1,3-dimethyl-1H-pyrazol-5-yl)sulfonyl)-8-azaspiro[4.5]decan-2-yl)-2-oxa-6-azaspiro[3.3]heptane

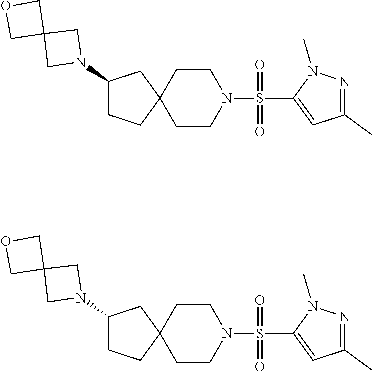 [SMILES: Cc1cc(S(=O)(=O)N2CCC3(CC[C@@H](N4CC5(COC5)C4)C3)CC2)n(C)n1 ; Cc1cc(S(=O)(=O)N2CCC3(CC[C@H](N4CC5(COC5)C4)C3)CC2)n(C)n1]

### 1. Synthesis of 8-((1,3-dimethyl-1H-pyrazol-5-yl)sulfonyl)-8-azaspiro[4.5]decan-2-one

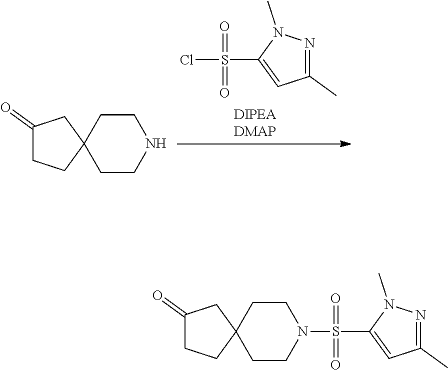 [SMILES: Cc1cc(S(=O)(=O)Cl)n(C)n1 ; O=C1CCC2(CCNCC2)C1 ; Cc1cc(S(=O)(=O)N2CCC3(CCC(=O)C3)CC2)n(C)n1]

A solution of 8-azaspiro[4.5]decan-3-one hydrochloride (2.5 g, 13.1 mmol) in anhydrous DCM (50 mL) was cooled in an ice bath to <5° C., then DIPEA (9 mL 51.7 mmol) and DMAP (123 mg, 1.0 mmol) were added. After 5 min, 2,5-dimethylpyrazole-3-sulfonyl chloride (3.4 g, 17.5 mmol) in anhydrous DCM (50 mL) was added. The reaction mixture was stirred at room temperature for 30 min. The reaction mixture was quenched with aqueous saturated NaHCO₃ solution, stirred for 10 min, and extracted with DCM (3×). The combined organic layers were dried over anhydrous MgSO₄, filtered and concentrated under reduced pressure. The resulting residue was purified by silica gel column eluting with (15-80% EtOAc in heptane) to afford 8-((1,3-dimethyl-1H-pyrazol-5-yl)sulfonyl)-8-azaspiro[4.5]decan-2-one (3.49 g, 81%) as a white solid. LCMS m/z=312.1 (M+H)⁺. ¹H-NMR (500 MHz, DCM-d₂) S (ppm): 6.46 (s, 1H), 4.01 (s, 3H), 3.37-3.32 (m, 2H), 2.95 (ddd, J=4.0, 8.4, 12.1 Hz, 2H), 2.27-2.23 (m, 5H), 2.06 (s, 2H), 1.83 (t, J=7.9 Hz, 2H), 1.71-1.63 (m, 4H).

### 2. Synthesis of 6-(8-((1,3-dimethyl-1H-pyrazol-5-yl)sulfonyl)-8-azaspiro[4.5]decan-2-yl)-2-oxa-6-azaspiro[3.3]heptane

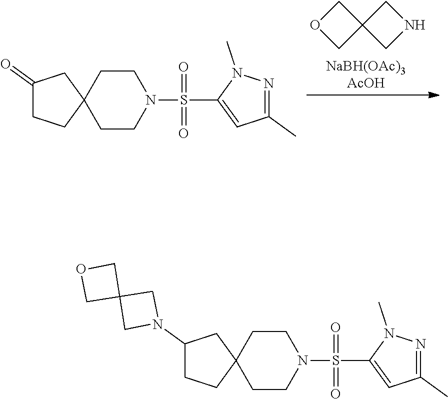 [SMILES: Cc1cc(S(=O)(=O)N2CCC3(CCC(=O)C3)CC2)n(C)n1 ; C1NCC12COC2 ; Cc1cc(S(=O)(=O)N2CCC3(CCC(N4CC5(COC5)C4)C3)CC2)n(C)n1]

A solution of 8-((1,3-dimethyl-1H-pyrazol-5-yl)sulfonyl)-8-azaspiro[4.5]decan-2-one (3.5 g, 11.1 mmol) in anhydrous DCM (40 mL) was cooled to <5° C. After 10 min, acetic acid (400 μL, 7.0 mmol) and a solution of 2-oxa-6-azaspiro[3.3]heptane (2.3 g, 23.2 mmol) in anhydrous DCM (10 mL) were sequentially added dropwise at <5° C. After 20 min, NaBH(OAc)₃ (8.6 g, 40.5 mmol) was added batchwise and the mixture was allowed to warm to room temperature and stirred for 2.5 h. The reaction was quenched with aqueous saturated NaHCO₃ solution After 20 min the biphasic mixture was extracted with DCM (3×). The combined organic extracts were dried over anhydrous MgSO₄, filtered and concentrated under reduced pressure. The resulting residue was purified by silica gel column chromatography (40-90% 3:1 EtOAc: EtOH in heptane) to afford 6-(8-((1,3-dimethyl-1H-pyrazol-5-yl)sulfonyl)-8-azaspiro[4.5]decan-2-yl)-2-oxa-6-azaspiro[3.3]heptane as a colorless oil (3.1 g, 67%). LCMS m/z=395.3 (M+H)⁺. ¹H-NMR (500 MHz, DMSO-d₆) δ (ppm): 6.58 (s, 1H), 4.55 (s, 4H), 3.92 (s, 3H), 3.20-3.10 (m, 4H), 3.06-2.99 (m, 4H), 2.62-2.54 (m, 1H), 2.18 (s, 3H), 1.58-1.51 (m, 3H), 1.46-1.39 (m, 4H), 1.35-1.26 (m, 2H), 1.07 (dd, J=5.2, 13.1 Hz, 1H).

### 3. Synthesis of (R)-6-(8-((1,3-dimethyl-1H-pyrazol-5-yl)sulfonyl)-8-azaspiro[4.5]decan-2-yl)-2-oxa-6-azaspiro[3.3]heptane and (S)-6-(8-((1,3-dimethyl-1H-pyrazol-5-yl)sulfonyl)-8-azaspiro[4.5]decan-2-yl)-2-oxa-6-azaspiro[3.3]heptane

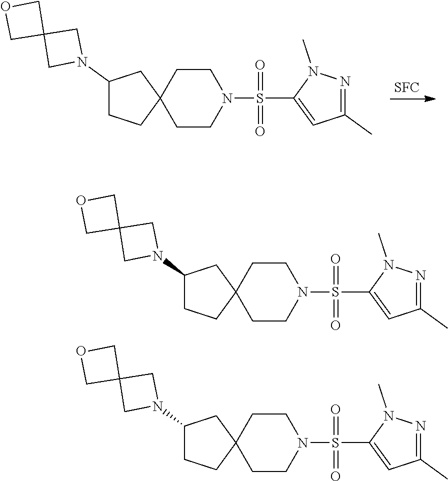

6-(8-((1,3-Dimethyl-1H-pyrazol-5-yl)sulfonyl)-8-azaspiro[4.5]decan-2-yl)-2-oxa-6-azaspiro[3.3]heptane (3.1 g, 7.86 mmol) was purified on a Lux Cellulose 30×250 mm, 5 μm column using 40% MeOH in CO₂. Flow rate: 100 mL/min; ABPR 120 bar; MBPR 40 psi, column temperature 40° C. to afford two enantiomers of arbitrarily assigned stereochemistry:
Peak 1, Enantiomer 1, (R)-6-(8-((1,3-dimethyl-1H-pyrazol-5-yl)sulfonyl)-8-azaspiro[4.5]decan-2-yl)-2-oxa-6-azaspiro[3.3]heptane (E1 with t_{R}=1.86 min) was a colorless film (1.18 g, 36%). LCMS m/z=395.3 [M+H]⁺. ¹H-NMR (400 MHz, DMSO-d₆) δ (ppm): 6.58 (s, 1H), 4.55 (s, 4H), 3.95-3.89 (m, 3H), 3.19-3.11 (m, 4H), 3.07-2.99 (m, 4H), 2.61-2.53 (m, 1H), 2.18 (s, 3H), 1.60-1.50 (m, 3H), 1.48-1.38 (m, 4H), 1.37-1.26 (m, 2H), 1.12-1.03 (m, 1H).
Peak 2, Enantiomer 2, (S)-6-(8-((1,3-dimethyl-1H-pyrazol-5-yl)sulfonyl)-8-azaspiro[4.5]decan-2-yl)-2-oxa-6-azaspiro[3.3]heptane (E2 with t_{R}=2.24 min) was a colorless film (1.24 g, 38%). LCMS m/z=395.3 [M+H]⁺. ¹H-NMR (400 MHz, DMSO-d₆) δ (ppm): 6.58 (s, 1H), 4.55 (s, 4H), 3.92 (s, 3H), 3.16-3.11 (m, 4H), 3.07-2.99 (m, 4H), 2.61-2.53 (m, 1H), 2.18 (s, 3H), 1.58-1.50 (m, 3H), 1.47-1.38 (m, 4H), 1.36-1.25 (m, 2H), 1.11-1.04 (m, 1H).

### Example 69: 8-((4-(Difluoromethoxy)phenyl)sulfonyl)-3-(6-oxa-2-azaspiro[3.4]octan-2-yl)-1-oxa-8-azaspiro[4.5]decane

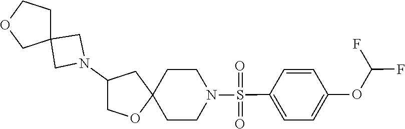 [SMILES: O=S(=O)(c1ccc(OC(F)F)cc1)N1CCC2(CC1)CC(N1CC3(CCOC3)C1)CO2]

### 1. Synthesis of 8-((4-(difluoromethoxy)phenyl)sulfonyl)-1-oxa-8-azaspiro[4.5]decan-3-one

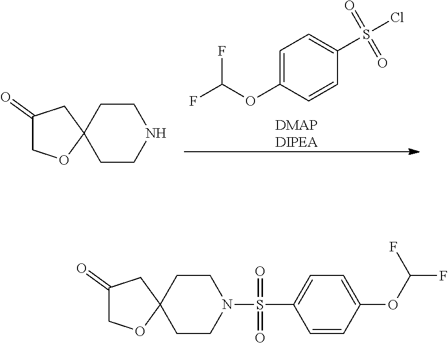

To a vial containing 1-oxa-8-azaspiro[4.5]decan-3-one hydrochloride (1.0 g, 5.2 mmol) in THE (24 mL) was added DIPEA (3.6 mL, 20.9 mmol) dropwise, followed by DMAP (64 mg, 0.5 mmol). After 5 min, 4-(difluoromethoxy)benzenesulfonyl chloride (1.0 mL, 6.3 mmol) was added and the reaction stirred at room temperature for 1 day. The reaction was quenched with saturated NaHCO₃ solution, extracted with EtOAc (3×), the organic layer was separated and washed with brine. The combined organic phase was dried over anhydrous Na₂SO₄, filtered and evaporated. The residue was purified using silica gel chromatography (0-75% EtOAc in heptane) to give 8-((4-(difluoromethoxy)phenyl)sulfonyl)-1-oxa-8-azaspiro[4.5]decan-3-one (1.69 g, 90%). LCMS m/z=362.0 [M+H]⁺. ¹H-NMR (400 MHz, DMSO-d₆) δ (ppm): 7.90-7.74 (m, 2H) 7.64-7.22 (m, 3H) 3.88 (s, 2H) 3.36-3.22 (m, 2H) 2.76-2.61 (m, 2H) 2.38 (s, 2H) 1.89-1.68 (m, 4H).

### 2. Synthesis of 8-((4-(difluoromethoxy)phenyl)sulfonyl)-3-(6-oxa-2-azaspiro[3.4]octan-2-yl)-1-oxa-8-azaspiro[4.5]decane

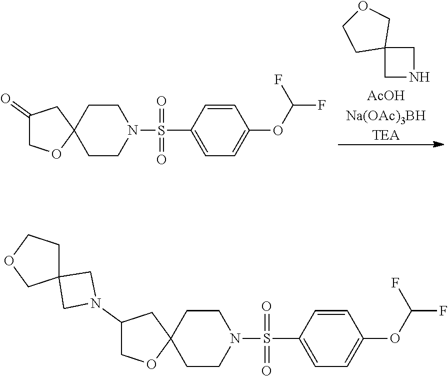

A solution of 8-((4-(difluoromethoxy)phenyl)sulfonyl)-1-oxa-8-azaspiro[4.5]decan-3-one (45 mg, 0.1 mmol) in DCM (1.5 mL) followed by TEA (40 μL, 0.3 mmol) were added to 6-oxa-2-azaspiro[3.4]octane (17 mg, 0.2 mmol) and the solution stirred for 15 min. Acetic acid (18 μL, 0.3 mmol) was added, the solution stirred for 30 min, then NaBH(OAc)₃ (106 mg, 0.5 mmol) was added. The reaction mixture was stirred for id at room temperature, quenched with saturated NH₄Cl solution (2 mL) and extracted with EtOAc (2×). The combined organic solution was concentrated under reduced pressure and the crude residue was purified by preparative HPLC (Waters SunFire Prep C18 5 μm OBD 30×100 mm; Method: (A) 95% water//(B) 5% (MeCN) w/0.1% TFA to 50% (A)/50% (B) over 7.5 min (flow rate: 50 mL/min) to give 8-((4-(difluoromethoxy)phenyl)sulfonyl)-3-(6-oxa-2-azaspiro[3.4]octan-2-yl)-1-oxa-8-azaspiro[4.5]decane (13.1 mg, 18%) as clear oil. LCMS m/z=458.8 [M+H]⁺. ¹H-NMR (500 MHz, DMSO-d₆) δ (ppm): 7.86-7.76 (m, 2H), 7.60-7.28 (m, 3H), 4.16-4.02 (m, 5H), 3.85-3.60 (m, 4H), 3.33-3.19 (m, 2H), 2.66-2.53 (m, 3H), 2.19-2.13 (m, 2H), 2.12-2.05 (m, 2H), 1.83-1.75 (m, 2H), 1.71 (br d, J=13.43 Hz, 1H), 1.61-1.48 (m, 2H).

### Example 70: 8-((4-(Difluoromethoxy)phenyl)sulfonyl)-3-(2-oxa-7-azaspiro[4.4]nonan-7-yl)-1-oxa-8-azaspiro[4.5]decane

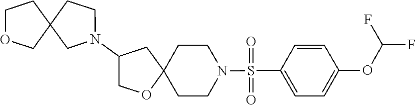 [SMILES: O=S(=O)(c1ccc(OC(F)F)cc1)N1CCC2(CC1)CC(N1CCC3(CCOC3)C1)CO2]

### 1. Synthesis of 8-((4-(difluoromethoxy)phenyl)sulfonyl)-3-(2-oxa-7-azaspiro[4.4]nonan-7-yl)-1-oxa-8-azaspiro[4.5]decane

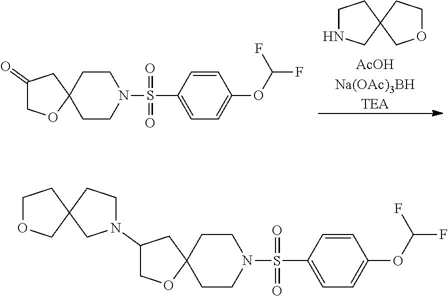 [SMILES: C1CC2(CCOC2)CN1 ; O=C1COC2(CCN(S(=O)(=O)c3ccc(OC(F)F)cc3)CC2)C1 ; "AcOHNa(OAc)3BHTEA" ; O=S(=O)(c1ccc(OC(F)F)cc1)N1CCC2(CC1)CC(N1CCC3(CCOC3)C1)CO2]

8-((4-(Difluoromethoxy)phenyl)sulfonyl)-3-(2-oxa-7-azaspiro[4.4]nonan-7-yl)-1-oxa-8-azaspiro[4.5]decane was prepared from 8-((4-(difluoromethoxy)phenyl)sulfonyl)-1-oxa-8-azaspiro[4.5]decan-3-one and 2-oxa-7-azaspiro[4.4]nonane following the procedure described in Example 69, step 2. The crude product was purified by preparative HPLC (Waters XSelect CSH Prep C18 5 μm OBD 30×100 mm; Method: (A) 95% (water)//(B) 5% (MeCN) w/0.2% NH₄OH to 35% (A)/65% (B) over 7.5 min (flow rate: 50 mL/min) (0.5 mg, 1%) as clear oil. LCMS m/z=472.8 [M+H]⁺, R_{f}=0.52 min.

### Example 71: 8-((4-(Difluoromethoxy)phenyl)sulfonyl)-3-((3aR,6aS)-tetrahydro-1H-furo[3,4-c]pyrrol-5(3H)-yl)-1-oxa-8-azaspiro[4.5]decane

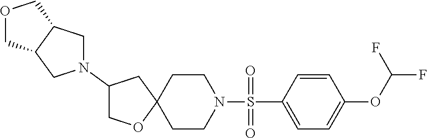 [SMILES: O=S(=O)(c1ccc(OC(F)F)cc1)N1CCC2(CC1)CC(N1C[C@H]3COC[C@H]3C1)CO2]

### 1. Synthesis of 8-((4-(difluoromethoxy)phenyl)sulfonyl)-3-((3aR,6aS)-tetrahydro-1H-furo[3,4-c]pyrrol-5(3H)-yl)-1-oxa-8-azaspiro[4.5]decane

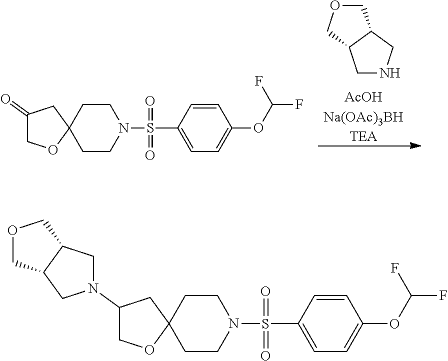 [SMILES: C1NC[C@H]2COC[C@@H]12 ; O=C1COC2(CCN(S(=O)(=O)c3ccc(OC(F)F)cc3)CC2)C1 ; "AcOHNa(OAc)3BHTEA" ; O=S(=O)(c1ccc(OC(F)F)cc1)N1CCC2(CC1)CC(N1C[C@H]3COC[C@H]3C1)CO2]

8-((4-(Difluoromethoxy)phenyl)sulfonyl)-3-((3aR,6aS)-tetrahydro-1H-furo[3,4-c]pyrrol-5(3H)-yl)-1-oxa-8-azaspiro[4.5]decane was obtained from 8-((4-(difluoromethoxy)phenyl)sulfonyl)-1-oxa-8-azaspiro[4.5]decan-3-one and (3aR,6aS)-hexahydro-1H-furo[3,4-c]pyrrole following the procedure described in Example 69, step 2. The crude was purified by preparative HPLC (Waters XSelect CSH Prep C18 5 μm OBD 30×100 mm; Method: (A) 95% (water)//(B) 5% (MeCN) w/0.2% NH₄OH to 40% (A)/60% (B) over 7.5 min (flow rate: 50 mL/min) to give 8-((4-(difluoromethoxy)phenyl)sulfonyl)-3-((3aR,6aS)-tetrahydro-1H-furo[3,4-c]pyrrol-5(3H)-yl)-1-oxa-8-azaspiro[4.5]decane (14.9 mg, 26%) as clear oil. LCMS m/z=458.8 [M+H]⁺, R_{f}=1.91 min.

### Examples 72a, 72b, 72c, and 72d: (S)-8-((1,3-Dimethyl-1H-pyrazol-5-yl)sulfonyl)-3-((R)-2-oxa-7-azaspiro[4.4]nonan-7-yl)-1-oxa-8-azaspiro[4.5]decane, (S)-8-((1,3-dimethyl-1H-pyrazol-5-yl)sulfonyl)-3-((S)-2-oxa-7-azaspiro[4.4]nonan-7-yl)-1-oxa-8-azaspiro[4.5]decane, (R)-8-((1,3-dimethyl-1H-pyrazol-5-yl)sulfonyl)-3-((R)-2-oxa-7-azaspiro[4.4]nonan-7-yl)-1-oxa-8-azaspiro[4.5]decane and (S)-8-((1,3-dimethyl-1H-pyrazol-5-yl)sulfonyl)-3-((S)-2-oxa-7-azaspiro[4.4]nonan-7-yl)-1-oxa-8-azaspiro[4.5]decane

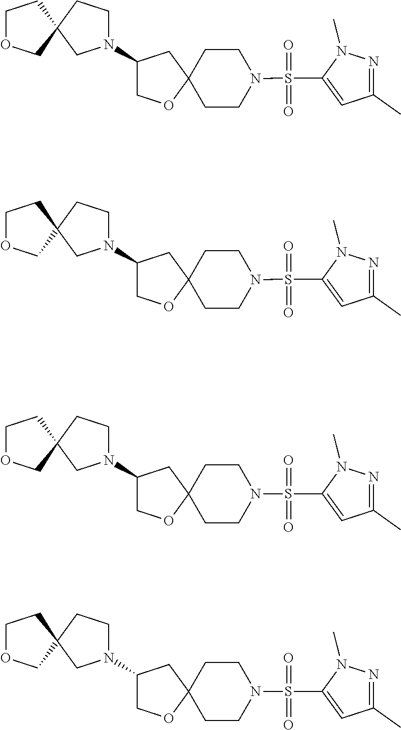

### 1. Synthesis of 8-((1,3-dimethyl-1H-pyrazol-5-yl)sulfonyl)-1-oxa-8-azaspiro[4.5]decan-3-one

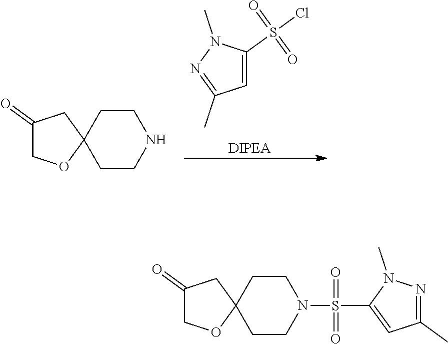 [SMILES: O=C1COC2(CCNCC2)C1 ; Cc1cc(S(=O)(=O)Cl)n(C)n1 ; "DIPEA" ; Cc1cc(S(=O)(=O)N2CCC3(CC2)CC(=O)CO3)n(C)n1]

To a vial containing 1-oxa-8-azaspiro[4.5]decan-3-one hydrochloride (397 mg, 2.1 mmol) in anhydrous DMF (7.5 mL) was added DIPEA (1.8 mL, 10.4 mmol). After 5 min, 1,3-dimethyl-1H-pyrazole-5-sulfonyl chloride (403 mg, 2.1 mmol) was added and the reaction was stirred at room temperature for 2.5 h. The reaction was quenched by addition of saturated NaHCO₃ solution, extracted with EtOAc, and washed with brine. The organic layer was dried over anhydrous Na₂SO₄, filtered and evaporated under reduced pressure. The residue was purified by silica gel chromatography (0-100% EtOAc in heptanes) to give 8-((1,3-dimethyl-1H-pyrazol-5-yl)sulfonyl)-1-oxa-8-azaspiro[4.5]decan-3-one (572.3 mg, 88%). LCMS m/z=314.0 [M+H]⁺. ¹H-NMR (400 MHz, CD₃OD) δ (ppm): 6.57 (s, 1H) 4.00 (s, 3H) 3.96 (s, 2H) 3.55-3.47 (m, 2H) 3.08 (td, J=11.6, 3.3 Hz, 2H) 2.41 (s, 2H) 2.25 (s, 3H) 1.98-1.91 (m, 2H) 1.89-1.80 (m, 2H).

### 2. Synthesis of 8-((1,3-dimethyl-1H-pyrazol-5-yl)sulfonyl)-3-(2-oxa-7-azaspiro[4.4]nonan-7-yl)-1-oxa-8-azaspiro[4.5]decane

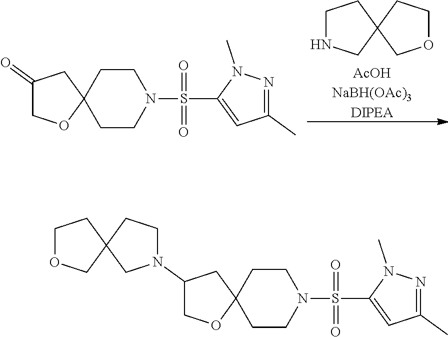

Hunig's base (110 μL, 0.6 mmol) was added slowly to a solution of 8-((1,3-dimethyl-1H-pyrazol-5-yl)sulfonyl)-1-oxa-8-azaspiro[4.5]decan-3-one (200 mg, 0.6 mmol) and 2-oxa-7-azaspiro[4.4]nonane (146 mg, 1.2 mmol) in DCM (4 mL). After 15 min, acetic acid (110 μL, 1.9 mmol) was added and the solution stirred for 30 min. NaBH(OAc)₃ (541 mg, 2.6 mmol) was added and the reaction was stirred at room temperature for 1 d. The reaction was quenched with saturated NaHCO₃ solution, extracted with EtOAc, washed with brine, dried over anhydrous Na₂SO₄, filtered and evaporated. The residue was purified by silica gel chromatography (0-100% EtOH:EtOAc (2% NH₄OH) 1:3 in EtOAc) to give 8-((1,3-dimethyl-1H-pyrazol-5-yl)sulfonyl)-3-(2-oxa-7-azaspiro[4.4]nonan-7-yl)-1-oxa-8-azaspiro[4.5]decane.

### 3. Separation of 8-((1,3-dimethyl-1H-pyrazol-5-yl)sulfonyl)-3-(2-oxa-7-azaspiro[4.4]nonan-7-yl)-1-oxa-8-azaspiro[4.5]decane

8-((1,3-Dimethyl-1H-pyrazol-5-yl)sulfonyl)-3-(2-oxa-7-azaspiro[4.4]nonan-7-yl)-1-oxa-8-azaspiro[4.5]decane was further purified by chiral SFC (CHIRALPAK AD-H 30×250 mm, 5 μm; Method: 20% EtOH w/0.1% DEA in CO₂ (flow rate: 100 mL/min, ABPR 120 bar, MBPR 40 psi, column temp 40° C.) to give 4 enantiomers of arbitrarily assigned stereochemistry:
Peak 1, Enantiomer 1, (S)-8-((1,3-dimethyl-1H-pyrazol-5-yl)sulfonyl)-3-((R)-2-oxa-7-azaspiro[4.4]nonan-7-yl)-1-oxa-8-azaspiro[4.5]decane (35.1 mg, 12%): R_{f}=2.97 min LCMS m/z=425.3 [M+H]⁺. ¹H-NMR (400 MHz, CD₃OD) δ (ppm): 6.54 (s, 1H) 3.99 (s, 3H) 3.92 (dd, J=8.8, 6.5 Hz, 1H) 3.88-3.78 (m, 2H) 3.67-3.61 (m, 2H) 3.54 (d, J=8.0 Hz, 1H) 3.49-3.41 (m, 2H) 3.03-2.92 (m, 3H) 2.71-2.49 (m, 4H) 2.25 (s, 3H) 2.05-1.87 (m, 3H) 1.86-1.73 (m, 5H) 1.72-1.63 (m, 2H).
Peak 2, Enantiomer 2, (S)-8-((1,3-dimethyl-1H-pyrazol-5-yl)sulfonyl)-3-((S)-2-oxa-7-azaspiro[4.4]nonan-7-yl)-1-oxa-8-azaspiro[4.5]decane (33.2 mg, 11%): R_{f}=3.26 min. LCMS m/z=425.3 [M+H]⁺.
Peak 3, Enantiomer 3, (R)-8-((1,3-dimethyl-1H-pyrazol-5-yl)sulfonyl)-3-((R)-2-oxa-7-azaspiro[4.4]nonan-7-yl)-1-oxa-8-azaspiro[4.5]decane (18.2 mg, 6%): R_{f}=3.42 min. LCMS m/z=425.3 [M+H]⁺.
Peak 4, Enantiomer 4, (S)-8-((1,3-dimethyl-1H-pyrazol-5-yl)sulfonyl)-3-((S)-2-oxa-7-azaspiro[4.4]nonan-7-yl)-1-oxa-8-azaspiro[4.5]decane (24 mg, 8%): R_{f}=3.76 min. LCMS m/z=425.3 [M+H]⁺.

### Example 73: 4-(7-((4-(Difluoromethoxy)phenyl)sulfonyl)-7-azaspiro[3.5]nonan-2-yl)morpholine

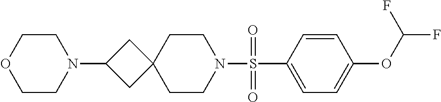 [SMILES: O=S(=O)(c1ccc(OC(F)F)cc1)N1CCC2(CC1)CC(N1CCOCC1)C2]

### 1. Synthesis of 7-((4-(difluoromethoxy)phenyl)sulfonyl)-7-azaspiro[3.5]nonan-2-one

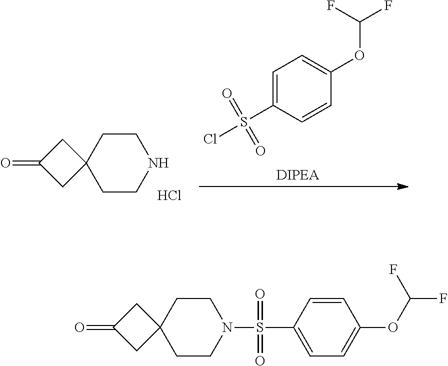

To a vial containing 7-azaspiro[3.5]nonan-2-one hydrochloride (522 mg, 3.0 mmol) in anhydrous THE (10 mL) was added DIPEA (1.2 g, 9.2 mmol) dropwise at <5° C. After 5 min, 4-(difluoromethoxy)benzenesulfonyl chloride (1.1 g, 4.4 mmol) was added to the cold solution. The reaction was warmed to room temperature and stirred for 30 min. The reaction mixture was quenched with aqueous 1 M NaOH solution and stirred for 10 min. The biphasic mixture was loaded onto a silica gel column and purified with (10-50% EtOAc in heptane) to afford 7-((4-(difluoromethoxy)phenyl)sulfonyl)-7-azaspiro[3.5]nonan-2-one as a white solid (977 mg, 95%) which was used without further purification in the next step. LCMS m/z=346.1 (M+H)⁺. ¹H-NMR (500 MHz, DMSO-d₆) δ (ppm): 7.84-7.79 (m, 2H), 7.60-7.27 (m, 3H), 2.96-2.89 (m, 4H), 2.72 (s, 4H), 1.78-1.70 (m, 4H).

### 2. Synthesis of 4-(7-((4-(difluoromethoxy)phenyl)sulfonyl)-7-azaspiro[3.5]nonan-2-yl)morpholine

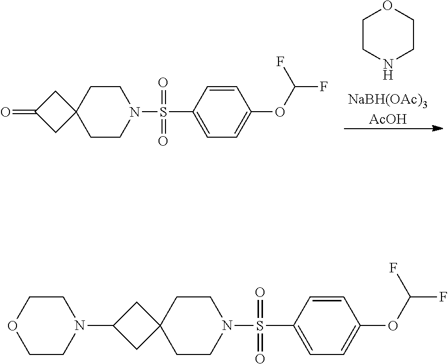 [SMILES: O=C1CC2(CCN(S(=O)(=O)c3ccc(OC(F)F)cc3)CC2)C1 ; C1COCCN1 ; O=S(=O)(c1ccc(OC(F)F)cc1)N1CCC2(CC1)CC(N1CCOCC1)C2 ; "NaBH(OAc)3AcOH"]

To a vial containing 7-((4-(difluoromethoxy)phenyl)sulfonyl)-7-azaspiro[3.5]nonan-2-one (84 mg, 0.2 mmol) in anhydrous DCM (2 mL) was added AcOH (10 μL, 0.2 mmol), then morpholine (50 μL, 0.6 mmol) dropwise at room temperature. After 15 min, NaBH(OAc)₃ (212 mg, 1.0 mmol) was added in portions to the reaction mixture. After 6.5 h, the reaction was quenched with aqueous 1 M NaOH solution, stirred for 20 min, and extracted with DCM (3×).

The combined organic layer was dried over anhydrous MgSO₄, filtered and concentrated under reduced pressure. The resulting residue was purified by silica gel column chromatography (10-70% 3:1 EtOAc:EtOH in heptane) to afford 4-(7-((4-(difluoromethoxy)phenyl)sulfonyl)-7-azaspiro[3.5]nonan-2-yl)morpholine as a colorless film (72 mg, 67%). LCMS m/z=417.2 (M+H)⁺. ¹H-NMR (500 MHz, DCM-d₂) δ (ppm): 7.77-7.73 (m, 2H), 7.26 (d, J=8.5 Hz, 2H), 6.66 (t, J=72.9 Hz, 1H), 4.25-3.83 (m, 1H), 3.71-3.57 (m, 3H), 3.41-3.03 (m, 1H), 3.03-2.76 (m, 5H), 2.74-2.40 (m, 2H), 2.34-2.17 (m, 3H), 1.93-1.82 (m, 2H), 1.74-1.64 (m, 3H), 1.63-1.58 (m, 1H).

### Example 74: 4-(7-((1,3-Dimethyl-1H-pyrazol-5-yl)sulfonyl)-7-azaspiro[3.5]nonan-2-yl)morpholine

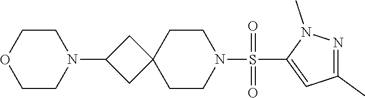 [SMILES: Cc1cc(S(=O)(=O)N2CCC3(CC2)CC(N2CCOCC2)C3)n(C)n1]

4-(7-((1,3-Dimethyl-1H-pyrazol-5-yl)sulfonyl)-7-azaspiro[3.5]nonan-2-yl)morpholine was prepared following the two step procedure described for Example 73 starting with 7-azaspiro[3.5]nonan-2-one hydrochloride and 1,3-dimethyl-1H-pyrazole-5-sulfonyl chloride to afford 7-((1,3-dimethyl-1H-pyrazol-5-yl)sulfonyl)-7-azaspiro[3.5]nonan-2-one as a white solid. (201 mg, 67%) LCMS m/z=298.0 (M+H)⁺. ¹H-NMR (500 MHz, CD₃OD) δ (ppm): 6.56 (s, 1H), 4.00 (s, 3H), 3.22-3.19 (m, 4H), 2.81 (s, 4H), 2.24 (s, 3H), 1.87-1.84 (m, 4H).

Step 2: Reaction of this compound with morpholine yielded 4-(7-((1,3-dimethyl-1H-pyrazol-5-yl)sulfonyl)-7-azaspiro[3.5]nonan-2-yl)morpholine as a peach oil (56 mg, 43%). LCMS m/z=369.3 (M+H)⁺. ¹H-NMR (600 MHz, DMSO-d₆) d=6.57 (s, 1H), 3.91 (s, 3H), 3.54 (br s, 1H), 3.35-3.31 (m, 1H), 3.05 (br t, J=5.1 Hz, 2H), 3.00-2.94 (m, 2H), 2.18 (s, 6H), 1.87 (br t, J=9.4 Hz, 3H), 1.64-1.59 (m, 3H), 1.56-1.47 (m, 6H).

### Example 75: 6-(7-((6-Methoxy-2-methylpyridin-3-yl)sulfonyl)-7-azaspiro[3.5]nonan-2-yl)-2-oxa-6-azaspiro[3.3]heptane

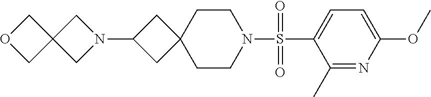 [SMILES: COc1ccc(S(=O)(=O)N2CCC3(CC2)CC(N2CC4(COC4)C2)C3)c(C)n1]

### 1. Synthesis of 7-((6-methoxy-2-methylpyridin-3-yl)sulfonyl)-7-azaspiro[3.5]nonan-2-one

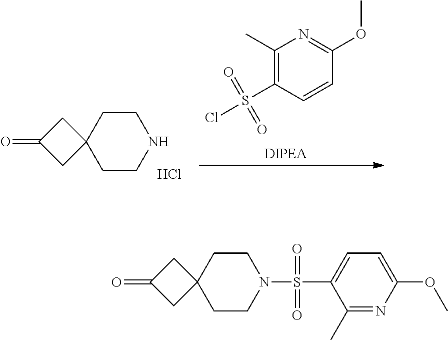

To a vial containing 7-azaspiro[3.5]nonan-2-one hydrochloride (375 mg, 2.1 mmol) in anhydrous THE (5 mL) was added DIPEA (890 mg, 6.9 mmol) dropwise at <5° C. After 5 min, 6-methoxy-2-methyl-pyridine-3-sulfonyl chloride (571 mg, 2.6 mmol) was added. The reaction was brought to room temperature stirred for 30 min and quenched by slow addition of aqueous 1 M NaOH solution and stirred for another 10 min. The biphasic mixture was directly loaded onto silica gel and purified by column chromatography (10-50% EtOAc in heptane) to afford 7-((6-methoxy-2-methylpyridin-3-yl)sulfonyl)-7-azaspiro[3.5]nonan-2-one as a white solid (595 mg, 86%) that was used without further purification in the next step. LCMS m/z=325.0 (M+H)⁺. ¹H-NMR (500 MHz, DCM-d₂) δ (ppm): 8.04 (d, J=9.2 Hz, 1H), 6.69 (d, J=8.5 Hz, 1H), 3.99 (s, 3H), 3.16-3.13 (m, 4H), 2.77 (s, 4H), 2.76 (s, 3H), 1.82-1.79 (m, 4H).

### 2. Synthesis of 6-(7-((6-methoxy-2-methylpyridin-3-yl)sulfonyl)-7-azaspiro[3.5]nonan-2-yl)-2-oxa-6-azaspiro[3.3]heptane

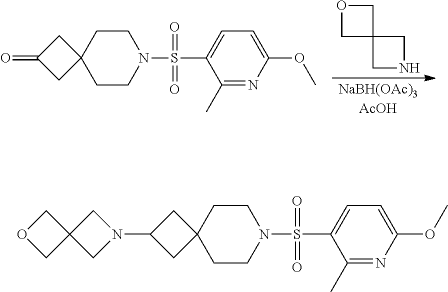 [SMILES: COc1ccc(S(=O)(=O)N2CCC3(CC2)CC(=O)C3)c(C)n1 ; C1NCC12COC2 ; COc1ccc(S(=O)(=O)N2CCC3(CC2)CC(N2CC4(COC4)C2)C3)c(C)n1 ; "NaBH(OAc)3AcOH"]

To a vial containing 7-((6-methoxy-2-methylpyridin-3-yl)sulfonyl)-7-azaspiro[3.5]nonan-2-one (76 mg, 0.2 mmol) in anhydrous DCM (2 mL) was added 2-oxa-6-azaspiro[3.3]heptane (47 mg, 0.5 mmol) followed by AcOH (10 μL, 0.2 mmol) dropwise at room temperature. After 15 min, NaBH(OAc)₃ (156 mg, 0.7 mmol) was added. After 1.5 h, the reaction mixture was quenched with aqueous 1 M NaOH, stirred for 10 min, and extracted with DCM (3×). The organic layer was dried over anhydrous MgSO₄. After filtration and concentration under reduced pressure, the residue was loaded onto a silica gel column and purified (25-100% 3:1 EtOAc:EtOH in heptane) to afford 6-(7-((6-methoxy-2-methylpyridin-3-yl)sulfonyl)-7-azaspiro[3.5]nonan-2-yl)-2-oxa-6-azaspiro[3.3]heptane as a colorless film (26 mg, 26%). LCMS m/z=408.3 (M+H)⁺. ¹H-NMR (500 MHz, DCM-d₂) δ (ppm): 7.96 (d, J=8.5 Hz, 1H), 6.63 (d, J=8.5 Hz, 1H), 4.64 (s, 4H), 3.96 (s, 3H), 3.19 (s, 4H), 3.05-3.02 (m, 2H), 3.00-2.97 (m, 2H), 2.91 (quin, J=7.3 Hz, 1H), 2.70 (s, 3H), 1.80-1.75 (m, 2H), 1.59 (q, J=5.7 Hz, 4H), 1.48-1.44 (m, 2H).

### Example 76: 6-(7-((2-Methyl-6-(trifluoromethyl)pyridin-3-yl)sulfonyl)-7-azaspiro[3.5]nonan-2-yl)-2-oxa-6-azaspiro[3.3]heptane

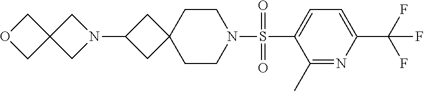 [SMILES: Cc1nc(C(F)(F)F)ccc1S(=O)(=O)N1CCC2(CC1)CC(N1CC3(COC3)C1)C2]

6-(7-((2-Methyl-6-(trifluoromethyl)pyridin-3-yl)sulfonyl)-7-azaspiro[3.5]nonan-2-yl)-2-oxa-6-azaspiro[3.3]heptane was prepared following the two step procedure described for Example 75 starting with 7-azaspiro[3.5]nonan-2-one hydrochloride and 2-methyl-6-(trifluoromethyl)pyridine-3-sulfonyl chloride to afford 7-((2-methyl-6-(trifluoromethyl)pyridin-3-yl)sulfonyl)-7-azaspiro[3.5]nonan-2-one (2.2 g, 82%). LCMS m/z=363.2 (M+H)⁺. ¹H-NMR (500 MHz, DCM-d₂) δ (ppm): 8.36 (d, J=8.2 Hz, 1H), 7.69 (d, J=8.2 Hz, 1H), 3.27-3.23 (m, 4H), 2.89 (s, 3H), 2.80 (s, 4H), 1.86-1.82 (m, 4H).

Step 2: Reaction of this compound with 2-oxa-6-azaspiro[3.3]heptane yielded the title compound as a white solid (1.73 g, 60%). LCMS m/z=446.2 (M+H)⁺. ¹H-NMR (500 MHz, DMSO-d₆) δ (ppm): 8.40 (d, J=7.9 Hz, 1H), 7.95 (d, J=8.2 Hz, 1H), 4.55 (s, 4H), 3.14 (s, 4H), 3.12-3.09 (m, 2H), 3.08-3.04 (m, 2H), 2.92 (quin, J=7.3 Hz, 1H), 2.80 (s, 3H), 1.76-1.71 (m, 2H), 1.56-1.52 (m, 4H), 1.45-1.40 (m, 2H).

### Example 77: 6-(7-((6-Chloro-2-methylpyridin-3-yl)sulfonyl)-7-azaspiro[3.5]nonan-2-yl)-2-oxa-6-azaspiro[3.3]heptane

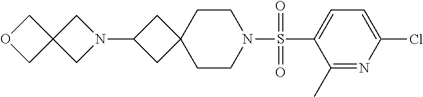 [SMILES: Cc1nc(Cl)ccc1S(=O)(=O)N1CCC2(CC1)CC(N1CC3(COC3)C1)C2]

6-(7-((6-Chloro-2-methylpyridin-3-yl)sulfonyl)-7-azaspiro[3.5]nonan-2-yl)-2-oxa-6-azaspiro[3.3]heptane was prepared following the two step procedure described for Example 75 starting with 7-azaspiro[3.5]nonan-2-one hydrochloride and 6-chloro-2-methylpyridine-3-sulfonyl chloride to afford 7-((6-chloro-2-methylpyridin-3-yl)sulfonyl)-7-azaspiro[3.5]nonan-2-one as a white solid (274 mg, 81%). LCMS m/z=329.0 (M+H)⁺. ¹H-NMR (500 MHz, CD₃OD) δ (ppm): 8.20 (d, J=8.2 Hz, 1H), 7.48 (d, J=8.2 Hz, 1H), 3.27-3.23 (m, 4H), 2.84-2.81 (m, 4H), 2.81-2.78 (m, 3H), 1.87-1.82 (m, 4H).

Step 2: Reaction of this compound with 2-oxa-6-azaspiro[3.3]heptane yielded the title compound as a white solid (32.5 mg, 35%). LCMS m/z=412.2 (M+H)⁺. ¹H-NMR (500 MHz, DMSO-d₆) δ (ppm): 8.14 (d, J=8.2 Hz, 1H), 7.58 (d, J=8.5 Hz, 1H), 4.55 (s, 4H), 3.14 (s, 4H), 3.06-3.02 (m, 2H), 3.01-2.97 (m, 2H), 2.96-2.88 (m, 1H), 2.70 (s, 3H), 1.76-1.69 (m, 2H), 1.56-1.50 (m, 4H), 1.45-1.39 (m, 2H).

### Example 78: 6-(7-((4-Methyl-6-(trifluoromethyl)pyridin-3-yl)sulfonyl)-7-azaspiro[3.5]nonan-2-yl)-2-oxa-6-azaspiro[3.3]heptane

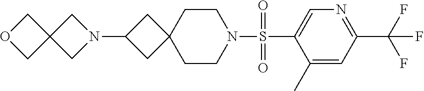 [SMILES: Cc1cc(C(F)(F)F)ncc1S(=O)(=O)N1CCC2(CC1)CC(N1CC3(COC3)C1)C2]

6-(7-((4-Methyl-6-(trifluoromethyl)pyridin-3-yl)sulfonyl)-7-azaspiro[3.5]nonan-2-yl)-2-oxa-6-azaspiro[3.3]heptane was prepared following the two step procedure described for Example 75 starting with 7-azaspiro[3.5]nonan-2-one hydrochloride and 4-methyl-6-(trifluoromethyl)pyridine-3-sulfonyl chloride to afford 7-((4-methyl-6-(trifluoromethyl)pyridin-3-yl)sulfonyl)-7-azaspiro[3.5]nonan-2-one as a white solid (285 mg, 75%). LCMS m/z=363.2 (M+H)⁺. ¹H-NMR (500 MHz, DCM-d₂) δ (ppm): 9.06 (s, 1H), 7.67 (s, 1H), 3.28-3.25 (m, 4H), 2.80 (s, 4H), 2.72 (s, 3H), 1.86-1.83 (m, 4H).

Step 2: Reaction of this compound with 2-oxa-6-azaspiro[3.3]heptane yielded the title compound as a colorless film (11 mg, 8%). LCMS m/z=446.2 (M+H)⁺. ¹H-NMR (500 MHz, DMSO-d₆) δ (ppm): 8.99 (s, 1H), 8.10 (s, 1H), 4.56 (s, 4H), 3.32-3.30 (m, 1H), 3.18-3.10 (m, 5H), 3.09-3.04 (m, 2H), 3.00-2.86 (m, 1H), 2.67 (s, 3H), 1.77-1.70 (m, 2H), 1.57-1.50 (m, 4H), 1.46-1.36 (m, 2H).

### Example 79: 6-(7-((1-Methyl-3-(trifluoromethyl)-1H-pyrazol-5-yl)sulfonyl)-7-azaspiro[3.5]nonan-2-yl)-2-oxa-6-azaspiro[3.3]heptane

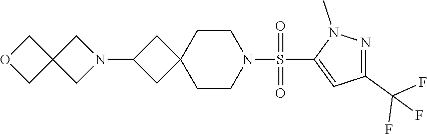 [SMILES: Cn1nc(C(F)(F)F)cc1S(=O)(=O)N1CCC2(CC1)CC(N1CC3(COC3)C1)C2]

6-(7-((1-Methyl-3-(trifluoromethyl)-1H-pyrazol-5-yl)sulfonyl)-7-azaspiro[3.5]nonan-2-yl)-2-oxa-6-azaspiro[3.3]heptane was prepared following the two step procedure described for Example 75 starting with 7-azaspiro[3.5]nonan-2-one hydrochloride and 2-methyl-5-(trifluoromethyl)pyrazole-3-sulfonyl chloride (307 mg, 1.2 mmol) to afford 7-((1-methyl-3-(trifluoromethyl)-1H-pyrazol-5-yl)sulfonyl)-7-azaspiro[3.5]nonan-2-one as white solid (206 mg, 58%). ¹H-NMR (500 MHz, DCM-d₂) δ (ppm): 6.95 (s, 1H), 4.15 (s, 3H), 3.25-3.21 (m, 4H), 2.80 (s, 4H), 1.89-1.86 (m, 4H).

Step 2: Reaction of this compound with 2-oxa-6-azaspiro[3.3]heptane yielded the title compound as a colorless film (12 mg, 9%). LCMS m/z=435.3 (M+H)⁺. ¹H-NMR (500 MHz, DMSO-d₆) δ (ppm): 7.39-7.37 (m, 1H), 4.56 (s, 4H), 4.12-4.09 (m, 4H), 3.16-3.14 (m, 4H), 3.11 (br dd, J=4.6, 5.5 Hz, 2H), 3.09-3.04 (m, 2H), 1.76-1.72 (m, 2H), 1.59-1.56 (m, 4H), 1.45-1.41 (m, 2H).

### Example 80: 6-(7-((3-Cyclopropyl-1-methyl-1H-pyrazol-5-yl)sulfonyl)-7-azaspiro[3.5]nonan-2-yl)-2-oxa-6-azaspiro[3.3]heptane

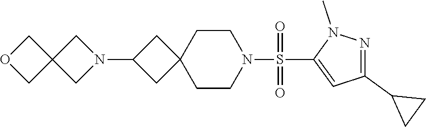 [SMILES: Cn1nc(C2CC2)cc1S(=O)(=O)N1CCC2(CC1)CC(N1CC3(COC3)C1)C2]

6-(7-((3-Cyclopropyl-1-methyl-1H-pyrazol-5-yl)sulfonyl)-7-azaspiro[3.5]nonan-2-yl)-2-oxa-6-azaspiro[3.3]heptane was prepared following the two step procedure described for Example 75 starting with 7-azaspiro[3.5]nonan-2-one hydrochloride and 5-cyclopropyl-2-methyl-pyrazole-3-sulfonylchloride to afford 7-((3-cyclopropyl-1-methyl-1H-pyrazol-5-yl)sulfonyl)-7-azaspiro[3.5]nonan-2-one as a white solid (195 mg, 87%). LCMS m/z=324.1 [M+H]⁺. ¹H-NMR (500 MHz, DCM-d₂) δ (ppm): 6.35 (s, 1H), 3.97 (s, 3H), 3.16-3.12 (m, 4H), 2.77 (s, 4H), 1.91-1.86 (m, 1H), 1.86-1.83 (m, 4H), 0.94-0.90 (m, 2H), 0.73-0.69 (m, 2H).

Step 2: Reaction of this compound with 2-oxa-6-azaspiro[3.3]heptane yielded the title compound as a colorless film (31 mg, 23%). LCMS m/z=407.3 (M+H)⁺. ^{v}(500 MHz, DMSO-d₆) δ (ppm): 6.51 (s, 1H), 4.56 (s, 1H), 3.89 (s, 3H), 3.32-3.29 (m, 3H), 3.22-3.10 (m, 4H), 3.01-2.97 (m, 2H), 2.95-2.88 (m, 3H), 1.91-1.86 (m, 1H), 1.74-1.68 (m, 2H), 1.58-1.53 (m, 4H), 1.45-1.39 (m, 2H), 0.89-0.85 (m, 2H), 0.70-0.67 (m, 2H).

### Example 81: (1R,4R)-5-(7-((6-Methoxy-2-methylpyridin-3-yl)sulfonyl)-7-azaspiro[3.5]nonan-2-yl)-2-oxa-5-azabicyclo[2.2.1]heptane

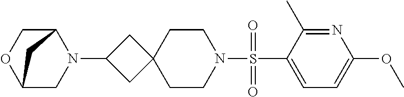 [SMILES: COc1ccc(S(=O)(=O)N2CCC3(CC2)CC(N2C[C@H]4C[C@@H]2CO4)C3)c(C)n1]

### 1. Synthesis of (1R,4R)-5-(7-((6-methoxy-2-methylpyridin-3-yl)sulfonyl)-7-azaspiro[3.5]nonan-2-yl)-2-oxa-5-azabicyclo[2.2.1]heptane

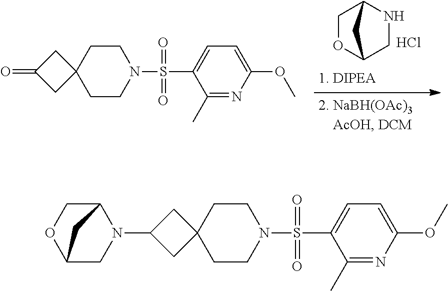

To a vial containing (1R,4R)-2-oxa-5-azabicyclo[2.2.1]heptane hydrochloride (78 mg, 0.6 mmol) in anhydrous DCM (1 mL) was added DIPEA (100 μL, 0.6 mmol) dropwise at room temperature. After 15 min, AcOH (50 μL, 0.9 mmol) and 7-((6-methoxy-2-methylpyridin-3-yl)sulfonyl)-7-azaspiro[3.5]nonan-2-one (Example 75, step 1) (87 mg, 0.3 mmol) were added dropwise. After 30 min, NaBH(OAc)₃ (200 mg, 1.0 mmol) was added in portions to the reaction mixture and the resulting mixture was stirred for 2.5 h. The reaction was quenched with aqueous 1 M sodium NaOH and stirred for an additional 10 min, then extracted with DCM (3×). The combined organic layer was dried over anhydrous MgSO₄, filtered and concentrated under reduced pressure. The residue was loaded onto silica gel and purified by column chromatography (25-100% 3:1 EtOAc:EtOH in heptane) to afford (1R,4R)-5-(7-((6-methoxy-2-methylpyridin-3-yl)sulfonyl)-7-azaspiro[3.5]nonan-2-yl)-2-oxa-5-azabicyclo[2.2.1]heptane as a colorless film (78 mg, 68%). LCMS m/z=408.3 (M+H)⁺. ¹H-NMR (500 MHz, DMSO-d₆) δ (ppm): 7.99 (d, J=9.2 Hz, 1H), 6.82 (d, J=8.5 Hz, 1H), 4.30 (br s, 1H), 3.92 (s, 3H), 3.73-3.65 (m, 1H), 3.48-3.41 (m, 1H), 3.37-3.32 (m, 1H), 3.16-3.08 (m, 1H), 3.02-2.96 (m, 2H), 2.95-2.90 (m, 2H), 2.66 (s, 3H), 2.64-2.56 (m, 1H), 2.38-2.31 (m, 1H), 1.92-1.85 (m, 1H), 1.84-1.77 (m, 1H), 1.66-1.61 (m, 1H), 1.60-1.56 (m, 2H), 1.56-1.52 (m, 2H), 1.52-1.43 (m, 3H).

### Example 82: (1R,4R)-5-(7-((1-methyl-3-(trifluoromethyl)-1H-pyrazol-5-yl)sulfonyl)-7-azaspiro[3.5]nonan-2-yl)-2-oxa-5-azabicyclo[2.2.1]heptane

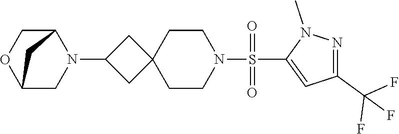 [SMILES: Cn1nc(C(F)(F)F)cc1S(=O)(=O)N1CCC2(CC1)CC(N1C[C@H]3C[C@@H]1CO3)C2]

(1R,4R)-5-(7-((1-Methyl-3-(trifluoromethyl)-1H-pyrazol-5-yl)sulfonyl)-7-azaspiro[3.5]nonan-2-yl)-2-oxa-5-azabicyclo[2.2.1]heptane was prepared from (1R,4R)-2-oxa-5-azabicyclo[2.2.1]heptane hydrochloride and 7-((1-methyl-3-(trifluoromethyl)-1H-pyrazol-5-yl)sulfonyl)-7-azaspiro[3.5]nonan-2-one (Example 79, step 1) using the method described for Example 81 to afford the title compound (32 mg, 26%). LCMS m/z=435.2 (M+H)⁺. ¹H-NMR (500 MHz, DMSO-d₆) δ (ppm): 7.38 (s, 1H), 4.30 (s, 1H), 4.10 (s, 3H), 3.69 (d, J=6.7 Hz, 1H), 3.44 (br d, J=7.3 Hz, 1H), 3.37-3.34 (m, 1H), 3.18-3.12 (m, 3H), 3.10-3.05 (m, 2H), 2.62-2.58 (m, 1H), 2.37-2.34 (m, 1H), 1.95-1.91 (m, 1H), 1.88-1.83 (m, 1H), 1.65-1.57 (m, 5H), 1.54-1.47 (m, 3H).

### Example 83: 7-((1-Methyl-3-(trifluoromethyl)-1H-pyrazol-5-yl)sulfonyl)-N-(tetrahydro-2H-pyran-4-yl)-7-azaspiro[3.5]nonan-2-amine

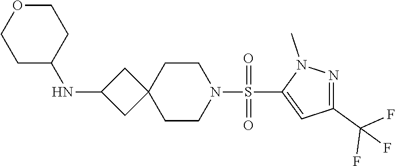 [SMILES: Cn1nc(C(F)(F)F)cc1S(=O)(=O)N1CCC2(CC1)CC(NC1CCOCC1)C2]

7-((1-Methyl-3-(trifluoromethyl)-1H-pyrazol-5-yl)sulfonyl)-N-(tetrahydro-2H-pyran-4-yl)-7-azaspiro[3.5]nonan-2-amine was prepared from tetrahydropyran-4-amine and 7-((1-methyl-3-(trifluoromethyl)-1H-pyrazol-5-yl)sulfonyl)-7-azaspiro[3.5]nonan-2-one (Example 79, step 1) using the method described for Example 81 to afford the title compound. LCMS m/z=437.1 [M+H]⁺. ¹H-NMR (CDCl₃, 500 MHz) δ (ppm): 6.88 (s, 1H), 4.12 (s, 3H), 3.92 (br d, J=11.3 Hz, 2H), 3.40-3.20 (m, 3H), 3.20-3.10 (m, 2H), 3.10-3.00 (m, 2H), 2.70-2.60 (m, 1H), 2.16 (br t, J=9.8 Hz, 2H), 1.80-1.60 (m, 7H), 1.48 (br t, J=10.1 Hz, 2H), 1.40-1.30 (m, 2H).

### Example 84: 1-(8-((1,3-dimethyl-1H-pyrazol-5-yl)sulfonyl)-8-azaspiro[4.5]decan-2-yl)-4-methylpiperidin-4-ol

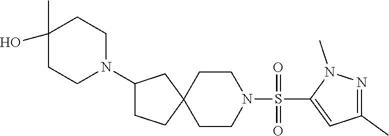 [SMILES: Cc1cc(S(=O)(=O)N2CCC3(CCC(N4CCC(C)(O)CC4)C3)CC2)n(C)n1]

### 1. Synthesis of 1-(8-((1,3-dimethyl-1H-pyrazol-5-yl)sulfonyl)-8-azaspiro[4.5]decan-2-yl)-4-methylpiperidin-4-ol

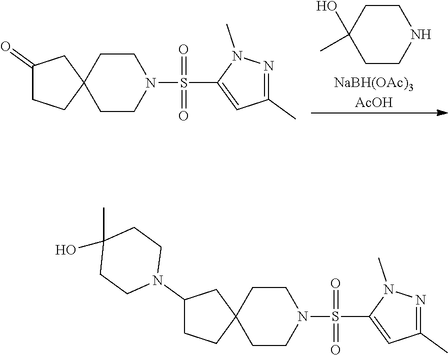

To a vial containing 8-((1,3-dimethyl-1H-pyrazol-5-yl)sulfonyl)-8-azaspiro[4.5]decan-2-one (75 mg, 242 umol) in DCM (2 mL) was added AcOH (0.02 mL, 349 umol) then 4-methylpiperidin-4-ol (57 mg, 491 umol) carefully dropwise at 23° C. After 15 minutes, NaBH(OAc)₃ (203 mg, 960 umol) was carefully added in portions to the reaction mixture. Upon complete addition of NaBH(OAc)₃, the reaction was stirred at 23° C. and monitored with LCMS. After 6.5 hours, the reaction was carefully quenched with slow addition of aq. 1 M NaOH solution. The mixture was stirred at 23° C. for 20 minutes, then the biphasic mixture was extracted with DCM (3×). The organic extractions were pooled then dried over MgSO₄. After filtration and concentration under reduced pressure, the residue was loaded onto a silica gel column and purified with (40-100% 3:1 EtOAc:ethanol in heptane.) Fractions containing product were pooled then concentrated under reduced pressure to afford a colorless film that was dissolved in DMSO and few drops of water then filtered. The homogeneous solution was submitted for mass directed reverse phase HPLC purification. Liquid chromatography was performed using a Waters XSelect CSH C18, 5 m, 50 mm×100 mm column with mobile phase H₂O (A) and MeCN (B) and a gradient of 5-65% B (0.2% NH₄OH final v/v % modifier) with flow rate at 50 mL/min. Fractions containing desired product were pooled then concentrated under reduced pressure to afford a yellow oil as 1-(8-((1,3-dimethyl-1H-pyrazol-5-yl)sulfonyl)-8-azaspiro[4.5]decan-2-yl)-4-methylpiperidin-4-ol (34 mg, 32%). LCMS m/z=411.3 (M+H)⁺. ¹H NMR (600 MHz, DMSO-d₆) δ (ppm)=6.58 (s, 1H), 3.92 (s, 3H), 3.10-3.01 (m, 3H), 2.49-2.25 (m, 2H), 2.18 (s, 3H), 1.79 (br d, J=6.5 Hz, 1H), 1.75-1.69 (m, 1H), 1.57-1.53 (m, 1H), 1.50-1.33 (m, 14H), 1.19 (br d, J=10.9 Hz, 1H), 1.07 (s, 4H).

### Example 85a and 85b: (R)-3-((2-(2-oxa-6-azaspiro[3.3]heptan-6-yl)-8-azaspiro[4.5]decan-8-yl)sulfonyl)-5-fluorobenzonitrile OR (S)-3-((2-(2-oxa-6-azaspiro[3.3]heptan-6-yl)-8-azaspiro[4.5]decan-8-yl)sulfonyl)-5-fluorobenzonitrile

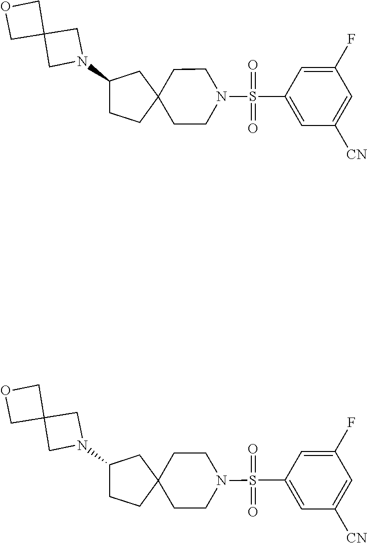 [SMILES: N#Cc1cc(F)cc(S(=O)(=O)N2CCC3(CC[C@@H](N4CC5(COC5)C4)C3)CC2)c1 ; N#Cc1cc(F)cc(S(=O)(=O)N2CCC3(CC[C@H](N4CC5(COC5)C4)C3)CC2)c1]

### 1. Synthesis of 3-fluoro-5-((2-oxo-8-azaspiro[4.5]decan-8-yl)sulfonyl)benzonitrile

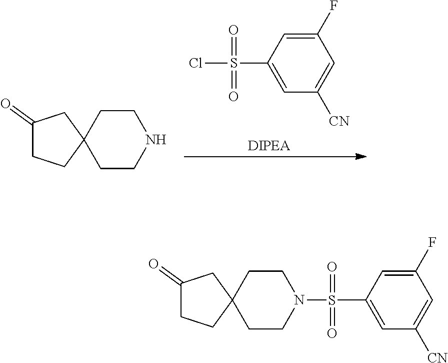 [SMILES: O=C1CCC2(CCNCC2)C1 ; N#Cc1cc(F)cc(S(=O)(=O)Cl)c1 ; "DIPEA" ; N#Cc1cc(F)cc(S(=O)(=O)N2CCC3(CCC(=O)C3)CC2)c1]

To a flask containing 1-oxa-8-azaspiro[4.5]decan-3-one hydrochloride (3 g, 16 mmol) in DCM (60 mL) was added DIPEA (8.5 mL, 48.8 mmol) dropwise. After 5 min, 3-cyano-5-fluoro-benzenesulfonyl chloride (4.13 g, 18.8 mmol) was added, and the reaction stirred at RT for 30 min. The reaction was quenched with sat. NaHCO₃ solution then extracted with DCM (3×). The organic layers were pooled then washed with brine. The combined organic phase was dried over Na₂SO₄, filtered, and evaporated. The residue was purified using silica gel chromatography (0-75% EtOAc in heptane) to give 3-fluoro-5-((2-oxo-8-azaspiro[4.5]decan-8-yl)sulfonyl)benzonitrile (4.9 g, 91%). ¹H NMR (500 MHz, DCM-d₂) δ (ppm)=7.87 (s, 1H), 7.74-7.70 (m, 1H), 7.66-7.61 (m, 1H), 3.34-3.27 (m, 2H), 2.89-2.81 (m, 2H), 2.22 (t, J=7.9 Hz, 2H), 2.02-1.99 (m, 2H), 1.79 (t, J=7.9 Hz, 2H), 1.73-1.64 (m, 4H).

### 2. Synthesis of (R)-3-((2-(2-oxa-6-azaspiro[3.3]heptan-6-yl)-8-azaspiro[4.5]decan-8-yl)sulfonyl)-5-fluorobenzonitrile AND (S)-3-((2-(2-oxa-6-azaspiro[3.3]heptan-6-yl)-8-azaspiro[4.5]decan-8-yl)sulfonyl)-5-fluorobenzonitrile

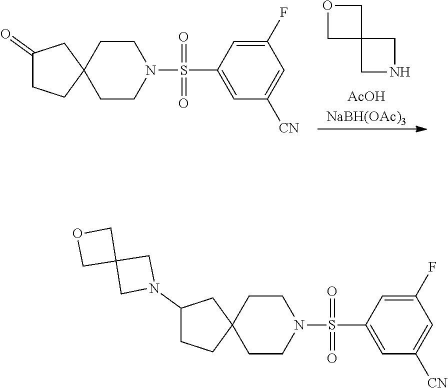

A flask containing 3-fluoro-5-((2-oxo-8-azaspiro[4.5]decan-8-yl)sulfonyl)benzonitrile (4.9 g, 14.6 mmol) in DCM (50 mL) was cooled in ice water bath to <5° C. then AcOH (0.8 mL, 14 mmol) and 2-oxa-6-azaspiro[3.3]heptane (3.0 g, 30 mmol) were added carefully dropwise at <5° C. After 15 minutes, NaBH(OAc)₃ (10.5 g, 49.4 mmol) was carefully added in portions to the cold reaction mixture. Upon complete addition, the reaction was warmed to 23° C. and monitored with LCMS. After 1 hour, the reaction was carefully quenched with slow addition of aq. 1 M NaOH solution. The mixture was stirred at 23° C. for 20 minutes, then the biphasic mixture was extracted with DCM (3×). The organic extractions were pooled then dried over MgSO₄. After filtration and concentration under reduced pressure, the residue was loaded onto a silica gel column and purified with (35-95% 3:1 EtOAc:ethanol in heptane.) The desired fractions were pooled then concentrated under reduced pressure to afford a white solid as (R)-3-((2-(2-oxa-6-azaspiro[3.3]heptan-6-yl)-8-azaspiro[4.5]decan-8-yl)sulfonyl)-5-fluorobenzonitrile AND (S)-3-((2-(2-oxa-6-azaspiro[3.3]heptan-6-yl)-8-azaspiro[4.5]decan-8-yl)sulfonyl)-5-fluorobenzonitrile (4.6 g, 71%) that was submitted for chiral SFC purification. LCMS m/z=420.4 (M+H)⁺. ¹H NMR (500 MHz, DMSO-d₆) δ (ppm)=8.27 (br d, J=8.2 Hz, 1H), 8.06 (s, 1H), 7.97-7.94 (m, 1H), 4.53 (s, 4H), 3.11 (br s, 4H), 2.99-2.91 (m, 4H), 2.59-2.52 (m, 1H), 1.55-1.49 (m, 3H), 1.43-1.33 (m, 4H), 1.31-1.23 (m, 2H), 1.01 (dd, J=5.0, 13.3 Hz, 1H).

### 3. Synthesis of (R)-3-((2-(2-oxa-6-azaspiro[3.3]heptan-6-yl)-8-azaspiro[4.5]decan-8-yl)sulfonyl)-5-fluorobenzonitrile OR (S)-3-((2-(2-oxa-6-azaspiro[3.3]heptan-6-yl)-8-azaspiro[4.5]decan-8-yl)sulfonyl)-5-fluorobenzonitrile

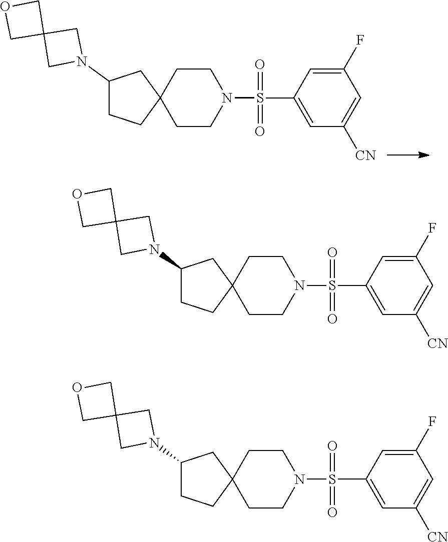 [SMILES: N#Cc1cc(F)cc(S(=O)(=O)N2CCC3(CCC(N4CC5(COC5)C4)C3)CC2)c1 ; N#Cc1cc(F)cc(S(=O)(=O)N2CCC3(CC[C@@H](N4CC5(COC5)C4)C3)CC2)c1 ; N#Cc1cc(F)cc(S(=O)(=O)N2CCC3(CC[C@H](N4CC5(COC5)C4)C3)CC2)c1]

(R)-3-((2-(2-oxa-6-azaspiro[3.3]heptan-6-yl)-8-azaspiro[4.5]decan-8-yl)sulfonyl)-5-fluorobenzonitrile AND (S)-3-((2-(2-oxa-6-azaspiro[3.3]heptan-6-yl)-8-azaspiro[4.5]decan-8-yl)sulfonyl)-5-fluorobenzonitrile (4.6 g, 10.9 mmol) was dissolved in methanol (12 mL) and DCM (8 mL) then purified on a Lux Cellulose-4 LC 30×250 mm, 5 μm column using 40% methanol. Flow rate: 100 mL/min; ABPR 120 bar; MBPR 40 psi, column temperature 40° C. to afford the following compounds that were concentrated to dryness then lyophilized:
Peak 1 or Example 85a was a white solid arbitrarily assigned (R)-3-((2-(2-oxa-6-azaspiro[3.3]heptan-6-yl)-8-azaspiro[4.5]decan-8-yl)sulfonyl)-5-fluorobenzonitrile (2.1 g, 45%). LCMS m/z=420.3 (M+H)⁺. ¹H NMR (500 MHz, DMSO-d₆) δ (ppm)=8.30-8.25 (m, 1H), 8.07 (s, 1H), 7.98-7.94 (m, 1H), 4.53 (s, 4H), 3.13-3.09 (m, 4H), 3.00-2.92 (m, 4H), 2.58-2.53 (m, 1H), 1.54-1.48 (m, 3H), 1.43-1.34 (m, 4H), 1.30-1.23 (m, 2H), 1.01 (dd, J=5.2, 13.1 Hz, 1H).
Peak 2 or Example 85b was a white solid arbitrarily assigned (S)-3-((2-(2-oxa-6-azaspiro[3.3]heptan-6-yl)-8-azaspiro[4.5]decan-8-yl)sulfonyl)-5-fluorobenzonitrile (2.1 g, 45% yield). LCMS m/z=420.3 (M+H)⁺. ¹H NMR (500 MHz, DMSO-d₆) δ (ppm)=8.30-8.25 (m, 1H), 8.07 (s, 1H), 7.97-7.93 (m, 1H), 4.54 (s, 4H), 3.11 (br s, 4H), 3.00-2.92 (m, 4H), 2.60-2.55 (m, 1H), 1.54-1.49 (m, 3H), 1.43-1.33 (m, 4H), 1.30-1.23 (m, 2H), 1.01 (dd, J=5.2, 13.1 Hz, 1H).

### Example 86a and 86b: (1R,4R)-5-((R)-8-((4-(difluoromethoxy)phenyl)sulfonyl)-8-azaspiro[4.5]decan-2-yl)-2-oxa-5-azabicyclo[2.2.1]heptane and (1R,4R)-5-((S)-8-((4-(difluoromethoxy)phenyl)sulfonyl)-8-azaspiro[4.5]decan-2-yl)-2-oxa-5-azabicyclo[2.2.1]heptane

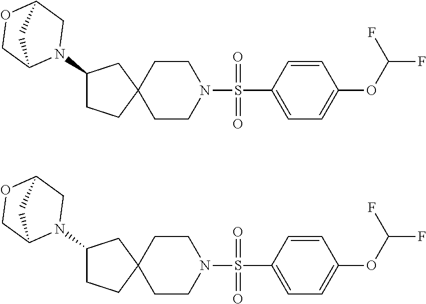 [SMILES: O=S(=O)(c1ccc(OC(F)F)cc1)N1CCC2(CC[C@@H](N3C[C@H]4C[C@@H]3CO4)C2)CC1 ; O=S(=O)(c1ccc(OC(F)F)cc1)N1CCC2(CC[C@H](N3C[C@H]4C[C@@H]3CO4)C2)CC1]

### 1. Synthesis of 8-((4-(difluoromethoxy)phenyl)sulfonyl)-8-azaspiro[4.5]decan-2-one

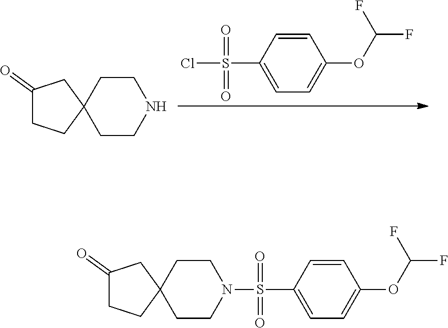 [SMILES: O=C1CCC2(CCNCC2)C1 ; O=S(=O)(Cl)c1ccc(OC(F)F)cc1 ; O=C1CCC2(CCN(S(=O)(=O)c3ccc(OC(F)F)cc3)CC2)C1]

To a flask containing 8-azaspiro[4.5]decan-3-one hydrochloride (2.8 g, 15 mmol) in DCM (30 mL) was added DIPEA (10.5 mL, 60 mmol) carefully dropwise at <5° C. After 15 minutes, 4-(difluoromethoxy)benzenesulfonyl chloride (4.3 g, 17.6 mmol) was added carefully to the cold solution. Upon complete addition of sulfonyl chloride, the reaction was warmed to 23° C. and monitored with LCMS. After 30 minutes, the reaction was carefully quenched with slow addition of aq. sat. NaHCO₃ solution. The mixture was stirred at 23° C. for 20 minutes, then the biphasic mixture was extracted with DCM (3×). The organic extractions were pooled then dried over MgSO₄. After filtration and concentration under reduced pressure, the residue was loaded onto a silica gel column and purified with (25-75% EtOAc in heptane.) The desired fractions were pooled then concentrated under reduced pressure to afford a white solid as 8-((4-(difluoromethoxy)phenyl)sulfonyl)-8-azaspiro[4.5]decan-2-one (4.9 g, 92%). LCMS m/z=360.1 (M+H)⁺. ¹H NMR (500 MHz, DCM-d₂) δ (ppm)=7.79-7.75 (m, 2H), 7.28 (d, J=8.5 Hz, 2H), 6.67 (t, J=72.9 Hz, 1H), 3.30-3.22 (m, 2H), 2.75 (ddd, J=3.4, 8.8, 12.0 Hz, 2H), 2.21 (t, J=7.9 Hz, 2H), 1.98 (s, 2H), 1.77 (t, J=8.1 Hz, 2H), 1.72-1.61 (m, 4H).

### 2. Synthesis of (1R,4R)-5-((R)-8-((4-(difluoromethoxy)phenyl)sulfonyl)-8-azaspiro[4.5]decan-2-yl)-2-oxa-5-azabicyclo[2.2.1]heptane AND (1R,4R)-5-((S)-8-((4-(difluoromethoxy)phenyl)sulfonyl)-8-azaspiro[4.5]decan-2-yl)-2-oxa-5-azabicyclo[2.2.1]heptane

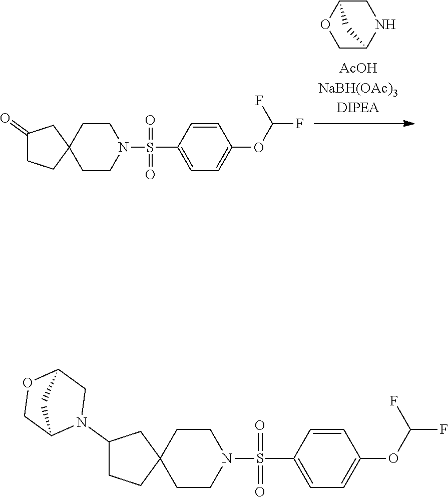 [SMILES: C1O[C@H]2CN[C@@H]1C2 ; O=C1CCC2(CCN(S(=O)(=O)c3ccc(OC(F)F)cc3)CC2)C1 ; O=S(=O)(c1ccc(OC(F)F)cc1)N1CCC2(CCC(N3C[C@H]4C[C@@H]3CO4)C2)CC1]

To a flask containing 8-((4-(difluoromethoxy)phenyl)sulfonyl)-8-azaspiro[4.5]decan-2-one (4.9 g, 13.6 mmol) in DCM (40 mL) was added AcOH (3 mL, 52 mmol) then a solution of (1R,4R)-2-oxa-5-azabicyclo[2.2.1]heptane hydrochloride (3.69 g, 27 mmol) and DIPEA (7.5 mL, 43 mmol) in DCM (20 mL) was carefully dropwise at 23° C. After 15 minutes, NaBH(OAc)₃ (12 g, 57 mmol) was carefully added in portions to the reaction mixture. Upon complete addition of NaBH(OAc)₃, the reaction was stirred at 23° C. and monitored with LCMS. After 3 hours, the reaction was carefully quenched with slow addition of aq. 1 M NaOH solution. The mixture was stirred at 23° C. for 45 minutes, then the biphasic mixture was extracted with DCM (3×). The organic extractions were pooled then dried over MgSO₄. After filtration and concentration under reduced pressure, the residue was loaded onto a silica gel column and purified with (20-80% 3:1 EtOAc:ethanol in heptane.) The desired fractions were pooled then concentrated under reduced pressure to afford a sticky white wax as (1R,4R)-5-((R)-8-((4-(difluoromethoxy)phenyl)sulfonyl)-8-azaspiro[4.5]decan-2-yl)-2-oxa-5-azabicyclo[2.2.1]heptane and (1R,4R)-5-((S)-8-((4-(difluoromethoxy)phenyl)sulfonyl)-8-azaspiro[4.5]decan-2-yl)-2-oxa-5-azabicyclo[2.2.1]heptane (5 g, 83%) that was submitted for chiral SFC purification. LCMS m/z=443.2 (M+H)⁺. ¹H NMR (500 MHz, DMSO-d₆) δ (ppm)=7.83-7.78 (m, 2H), 7.59-7.28 (m, 3H), 4.25 (s, 1H), 3.81-3.78 (m, 1H), 3.45-3.40 (m, 2H), 2.97-2.82 (m, 5H), 2.73 (ddd, J=1.5, 9.8, 15.0 Hz, 1H), 2.27 (dd, J=4.3, 9.8 Hz, 1H), 1.75-1.60 (m, 2H), 1.58-1.45 (m, 6H), 1.42-1.34 (m, 2H), 1.30-1.22 (m, 1H), 1.16-1.09 (m, 1H).

### 3. Synthesis of (1R,4R)-5-((R)-8-((4-(difluoromethoxy)phenyl)sulfonyl)-8-azaspiro[4.5]decan-2-yl)-2-oxa-5-azabicyclo[2.2.1]heptane OR (1R,4R)-5-((S)-8-((4-(difluoromethoxy)phenyl)sulfonyl)-8-azaspiro[4.5]decan-2-yl)-2-oxa-5-azabicyclo[2.2.1]heptane

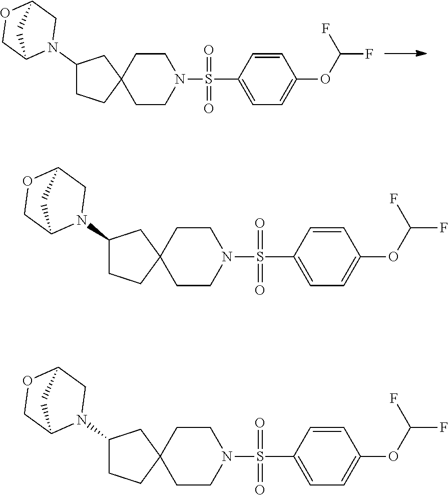 [SMILES: O=S(=O)(c1ccc(OC(F)F)cc1)N1CCC2(CCC(N3C[C@H]4C[C@@H]3CO4)C2)CC1 ; O=S(=O)(c1ccc(OC(F)F)cc1)N1CCC2(CC[C@@H](N3C[C@H]4C[C@@H]3CO4)C2)CC1 ; O=S(=O)(c1ccc(OC(F)F)cc1)N1CCC2(CC[C@H](N3C[C@H]4C[C@@H]3CO4)C2)CC1]

(1R,4R)-5-((R)-8-((4-(difluoromethoxy)phenyl)sulfonyl)-8-azaspiro[4.5]decan-2-yl)-2-oxa-5-azabicyclo[2.2.1]heptane and (1R,4R)-5-((S)-8-((4-(difluoromethoxy)phenyl)sulfonyl)-8-azaspiro[4.5]decan-2-yl)-2-oxa-5-azabicyclo[2.2.1]heptane (5 g, 11 mmol) was dissolved in methanol (50 mL) then purified on a Lux Cellulose-4 30×250 mm, 5 μm column using 45% methanol in CO₂. Flow rate: 100 mL/min; ABPR 120 bar; MBPR 40 psi, column temperature 40° C. to afford the following compounds that were concentrated to dryness then lyophilized:
Peak 1 or Example 86a (E1 with R_{f}=2.31 min) was an off-white solid arbitrarily assigned (1R,4R)-5-((R)-8-((4-(difluoromethoxy)phenyl)sulfonyl)-8-azaspiro[4.5]decan-2-yl)-2-oxa-5-azabicyclo[2.2.1]heptane (1.9 g, 36%). LCMS m/z=443.2 (M+H)⁺. ¹H NMR (500 MHz, DMSO-d₆) δ (ppm)=7.80 (d, J=8.9 Hz, 2H), 7.59-7.27 (m, 3H), 4.25 (br s, 1H), 3.79 (br d, J=7.0 Hz, 1H), 3.45-3.37 (m, 2H), 2.99-2.90 (m, 2H), 2.85 (br s, 3H), 2.75 (br d, J=9.2 Hz, 1H), 2.36-2.23 (m, 1H), 1.78-1.69 (m, 1H), 1.68-1.60 (m, 1H), 1.59-1.44 (m, 6H), 1.42-1.33 (m, 2H), 1.31-1.24 (m, 1H), 1.18-1.09 (m, 1H).
Peak 2 or Example 86b (E2 with R_{f}=3.98 min) was an off-white solid arbitrarily assigned (1R,4R)-5-((S)-8-((4-(difluoromethoxy)phenyl)sulfonyl)-8-azaspiro[4.5]decan-2-yl)-2-oxa-5-azabicyclo[2.2.1]heptane (2.4 g, 46%). LCMS m/z=443.2 (M+H)⁺. ¹H NMR (500 MHz, DMSO-d₆) δ (ppm)=7.80 (d, J=8.9 Hz, 2H), 7.60-7.28 (m, 3H), 4.25 (br s, 1H), 3.79 (br d, J=7.3 Hz, 1H), 3.47-3.37 (m, 2H), 2.96-2.82 (m, 5H), 2.76-2.68 (m, 1H), 2.30-2.23 (m, 1H), 1.68-1.59 (m, 2H), 1.59-1.46 (m, 6H), 1.42-1.34 (m, 2H), 1.30-1.23 (m, 1H), 1.15-1.09 (m, 1H).

### Example 87: (1S,4S)-5-(8-((1,3-dimethyl-1H-pyrazol-5-yl)sulfonyl)-8-azaspiro[4.5]decan-2-yl)-2-oxa-5-azabicyclo[2.2.1]heptane

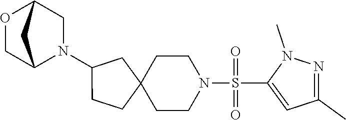 [SMILES: Cc1cc(S(=O)(=O)N2CCC3(CCC(N4C[C@@H]5C[C@H]4CO5)C3)CC2)n(C)n1]

### 1. Synthesis of (1S,4S)-5-(8-((1,3-dimethyl-1H-pyrazol-5-yl)sulfonyl)-8-azaspiro[4.5]decan-2-yl)-2-oxa-5-azabicyclo[2.2.1]heptane

(1S,4S)-5-(8-((1,3-dimethyl-1H-pyrazol-5-yl)sulfonyl)-8-azaspiro[4.5]decan-2-yl)-2-oxa-5-azabicyclo[2.2.1]heptane can be synthesized similar to method used to make Example 21 but starting with (1S,4S)-2-oxa-5-azabicyclo[2.2.1]heptane hydrochloride. (1S,4S)-5-(8-((1,3-dimethyl-1H-pyrazol-5-yl)sulfonyl)-8-azaspiro[4.5]decan-2-yl)-2-oxa-5-azabicyclo[2.2.1]heptane (33 mg, 20%). LCMS m/z=395.3 (M+H)⁺. ¹H NMR (500 MHz, DMSO-d₆) δ (ppm)=6.59 (s, 1H), 4.27 (br s, 1H), 3.92 (s, 3H), 3.82 (d, J=7.9 Hz, 1H), 3.45 (br d, J=7.3 Hz, 2H), 3.08-3.02 (m, 4H), 2.94-2.86 (m, 1H), 2.81-2.74 (m, 1H), 2.36-2.27 (m, 1H), 2.18 (s, 3H), 1.70-1.62 (m, 2H), 1.59-1.44 (m, 7H), 1.44-1.34 (m, 2H), 1.23-1.16 (m, 1H).

### Example 88: (1R,4R)-5-(8-((1,3-dimethyl-1H-pyrazol-5-yl)sulfonyl)-8-azaspiro[4.5]decan-2-yl)-2-oxa-5-azabicyclo[2.2.1]heptane

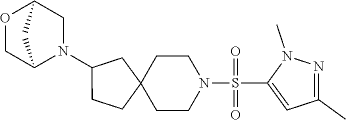 [SMILES: Cc1cc(S(=O)(=O)N2CCC3(CCC(N4C[C@H]5C[C@@H]4CO5)C3)CC2)n(C)n1]

### 1. Synthesis of (1R,4R)-5-(8-((1,3-dimethyl-1H-pyrazol-5-yl)sulfonyl)-8-azaspiro[4.5]decan-2-yl)-2-oxa-5-azabicyclo[2.2.1]heptane

(1R,4R)-5-(8-((1,3-dimethyl-1H-pyrazol-5-yl)sulfonyl)-8-azaspiro[4.5]decan-2-yl)-2-oxa-5-azabicyclo[2.2.1]heptane can be synthesized similar to method used to make Example 21 but starting with (1R,4R)-2-oxa-5-azabicyclo[2.2.1]heptane hydrochloride. (1R,4R)-5-(8-((1,3-dimethyl-1H-pyrazol-5-yl)sulfonyl)-8-azaspiro[4.5]decan-2-yl)-2-oxa-5-azabicyclo[2.2.1]heptane (49 mg, 26%). LCMS m/z=395.3 (M+H)⁺. ¹H NMR (600 MHz, DMSO-d₆) δ (ppm)=6.58 (s, 1H), 3.92 (s, 3H), 3.82 (d, J=8.0 Hz, 1H), 3.09-3.03 (m, 3H), 2.93-2.88 (m, 1H), 2.77 (dd, J=9.4, 15.3 Hz, 1H), 2.31 (d, J=10.2 Hz, 1H), 2.18 (s, 3H), 1.68-1.63 (m, 3H), 1.59-1.52 (m, 4H), 1.52-1.47 (m, 5H), 1.46-1.33 (m, 3H), 1.22-1.19 (m, 1H).

### Example 89: (1S,4S)-5-(8-((4-(difluoromethoxy)phenyl)sulfonyl)-8-azaspiro[4.5]decan-2-yl)-2-oxa-5-azabicyclo[2.2.1]heptane

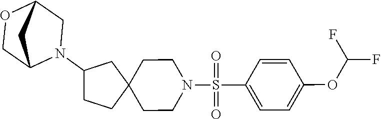 [SMILES: O=S(=O)(c1ccc(OC(F)F)cc1)N1CCC2(CCC(N3C[C@@H]4C[C@H]3CO4)C2)CC1]

### 1. Synthesis of (1S,4S)-5-(8-((4-(difluoromethoxy)phenyl)sulfonyl)-8-azaspiro[4.5]decan-2-yl)-2-oxa-5-azabicyclo[2.2.1]heptane

(1S,4S)-5-(8-((1,3-dimethyl-1H-pyrazol-5-yl)sulfonyl)-8-azaspiro[4.5]decan-2-yl)-2-oxa-5-azabicyclo[2.2.1]heptane can be synthesized similar to method used to make Example 21 but starting with 4-(difluoromethoxy)benzenesulfonyl chloride. (1S,4S)-5-(8-((4-(difluoromethoxy)phenyl)sulfonyl)-8-azaspiro[4.5]decan-2-yl)-2-oxa-5-azabicyclo[2.2.1]heptane (87 mg, 44%). LCMS m/z=443.3 (M+H)⁺. ¹H NMR (600 MHz, DMSO-d₆) δ (ppm)=7.80 (d, J=8.7 Hz, 2H), 7.58-7.27 (m, 3H), 3.81-3.78 (m, 1H), 2.96-2.84 (m, 5H), 2.73 (dd, J=9.8, 17.1 Hz, 1H), 2.29-2.26 (m, 1H), 1.63 (br d, J=9.4 Hz, 2H), 1.58-1.53 (m, 2H), 1.52-1.46 (m, 6H), 1.42-1.35 (m, 3H), 1.28 (br dd, J=4.0, 7.6 Hz, 1H), 1.15-1.11 (m, 1H).

### Example 90a and 90b: (1S,4S)-5-((R)-8-((4-(difluoromethoxy)phenyl)sulfonyl)-8-azaspiro[4.5]decan-2-yl)-2-oxa-5-azabicyclo[2.2.1]heptane OR (1S,4S)-5-((S)-8-((4-(difluoromethoxy)phenyl)sulfonyl)-8-azaspiro[4.5]decan-2-yl)-2-oxa-5-azabicyclo[2.2.1]heptane

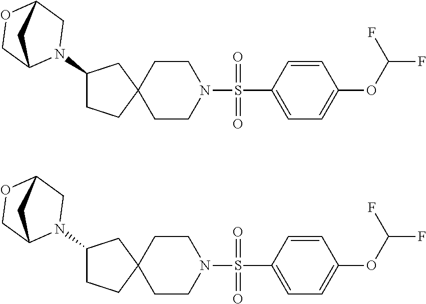 [SMILES: O=S(=O)(c1ccc(OC(F)F)cc1)N1CCC2(CC[C@@H](N3C[C@@H]4C[C@H]3CO4)C2)CC1 ; O=S(=O)(c1ccc(OC(F)F)cc1)N1CCC2(CC[C@H](N3C[C@@H]4C[C@H]3CO4)C2)CC1]

### 1. Synthesis of (1S,4S)-5-((R)-8-((4-(difluoromethoxy)phenyl)sulfonyl)-8-azaspiro[4.5]decan-2-yl)-2-oxa-5-azabicyclo[2.2.1]heptane OR (1S,4S)-5-((S)-8-((4-(difluoromethoxy)phenyl)sulfonyl)-8-azaspiro[4.5]decan-2-yl)-2-oxa-5-azabicyclo[2.2.1]heptane

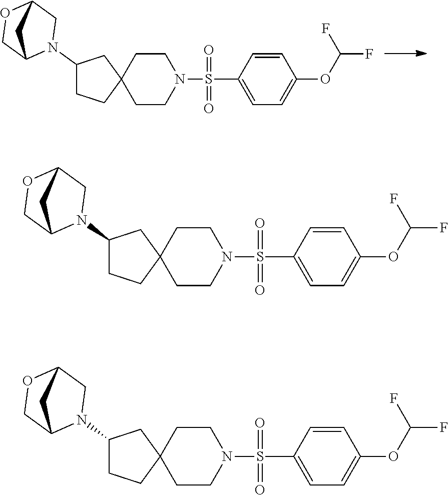 [SMILES: O=S(=O)(c1ccc(OC(F)F)cc1)N1CCC2(CCC(N3C[C@@H]4C[C@H]3CO4)C2)CC1 ; O=S(=O)(c1ccc(OC(F)F)cc1)N1CCC2(CC[C@@H](N3C[C@@H]4C[C@H]3CO4)C2)CC1 ; O=S(=O)(c1ccc(OC(F)F)cc1)N1CCC2(CC[C@H](N3C[C@@H]4C[C@H]3CO4)C2)CC1]

(1S,4S)-5-(8-((4-(difluoromethoxy)phenyl)sulfonyl)-8-azaspiro[4.5]decan-2-yl)-2-oxa-5-azabicyclo[2.2.1]heptane (80 mg, 181 umol) was dissolved in methanol (8 mL) then purified on a Chiralpak AD-H 30×250 mm, 5 μm column using 45% methanol with 0.1% DEA in CO₂. Flow rate: 100 mL/min; ABPR 120 bar; MBPR 40 psi, column temperature 40° C. to afford the following compounds that were concentrated to dryness then lyophilized:
Peak 1 or Example 90a (E1 with R_{f}=2.43 min) was a white solid arbitrarily assigned (1S,4S)-5-[(3R)-8-[4-(difluoromethoxy)phenyl]sulfonyl-8-azaspiro[4.5]decan-3-yl]-2-oxa-5-azabicyclo[2.2.1]heptane (26 mg, 31%). LCMS m/z=443.3 (M+H)⁺. ¹H NMR (500 MHz, DMSO-d₆) δ (ppm)=7.80 (d, J=8.9 Hz, 2H), 7.59-7.29 (m, 3H), 4.25 (br s, 1H), 3.79 (br d, J=7.3 Hz, 1H), 3.41 (br d, J=11.9 Hz, 2H), 2.97-2.90 (m, 2H), 2.88-2.80 (m, 3H), 2.77-2.73 (m, 1H), 2.30-2.23 (m, 1H), 1.76-1.69 (m, 1H), 1.65-1.61 (m, 1H), 1.57-1.45 (m, 6H), 1.41-1.34 (m, 2H), 1.31-1.25 (m, 1H), 1.18-1.11 (m, 1H).
Peak 2 or Example 90b (E2 with R_{f}=2.62 min) was a white solid arbitrarily assigned (1S,4S)-5-[(3S)-8-[4-(difluoromethoxy)phenyl]sulfonyl-8-azaspiro[4.5]decan-3-yl]-2-oxa-5-azabicyclo[2.2.1]heptane (34 mg, 40%). LCMS m/z=443.2 (M+H)⁺. ¹H NMR (500 MHz, DMSO-d₆) δ (ppm)=7.80 (d, J=8.9 Hz, 2H), 7.60-7.28 (m, 3H), 4.25 (s, 1H), 3.79 (d, J=7.6 Hz, 1H), 3.42 (br d, J=5.8 Hz, 2H), 2.94-2.83 (m, 5H), 2.72 (br d, J=9.8 Hz, 1H), 2.29-2.23 (m, 1H), 1.66-1.60 (m, 2H), 1.57-1.46 (m, 6H), 1.41-1.32 (m, 2H), 1.31-1.25 (m, 1H), 1.14-1.11 (m, 1H).

### Example 91: 7-((1,3-dimethyl-1H-pyrazol-5-yl)sulfonyl)-N-(2-methoxyethyl)-N-methyl-7-azaspiro[3.5]nonan-2-amine

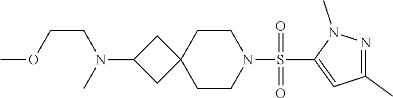

### 1. Synthesis of 7-((1,3-dimethyl-1H-pyrazol-5-yl)sulfonyl)-7-azaspiro[3.5]nonan-2-one

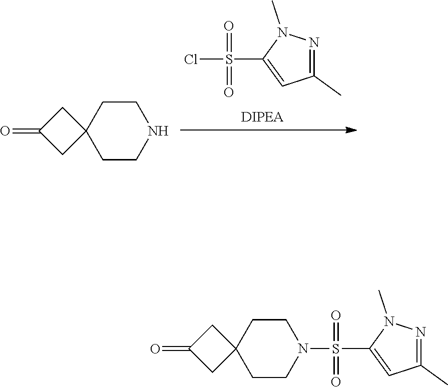 [SMILES: Cc1cc(S(=O)(=O)Cl)n(C)n1 ; O=C1CC2(CCNCC2)C1 ; Cc1cc(S(=O)(=O)N2CCC3(CC2)CC(=O)C3)n(C)n1]

To a vial containing 7-azaspiro[3.5]nonan-2-one hydrochloride (177 mg, 1.01 mmol) in THE (2 mL) was added DIPEA (0.54 mL, 3.1 mmol) carefully dropwise at <5° C. After 5 minutes, 2,5-dimethylpyrazole-3-sulfonyl chloride (222 mg, 1.14 mmol) was added carefully to the cold solution. Upon complete addition of sulfonyl chloride, the reaction was warmed to 23° C. and monitored with LCMS. After 30 minutes, the reaction mixture was carefully quenched with slow addition of aq. 1 M NaOH solution. The mixture was stirred at 23° C. for 10 minutes, then the biphasic mixture was loaded onto a silica gel column and purified with (15-80% EtOAc in heptane.) Fractions containing product were pooled then concentrated under reduced pressure to afford a white solid as 7-((1,3-dimethyl-1H-pyrazol-5-yl)sulfonyl)-7-azaspiro[3.5]nonan-2-one (201 mg, 67%) that was used without further purification. LCMS m/z=298.0 (M+H)⁺. ¹H NMR (500 MHz, METHANOL-d₄) δ (ppm)=6.56 (s, 1H), 4.00 (s, 3H), 3.22-3.19 (m, 4H), 2.81 (s, 4H), 2.24 (s, 3H), 1.87-1.84 (m, 4H).

### 2. Synthesis of 7-((1,3-dimethyl-1H-pyrazol-5-yl)sulfonyl)-N-(2-methoxyethyl)-N-methyl-7-azaspiro[3.5]nonan-2-amine

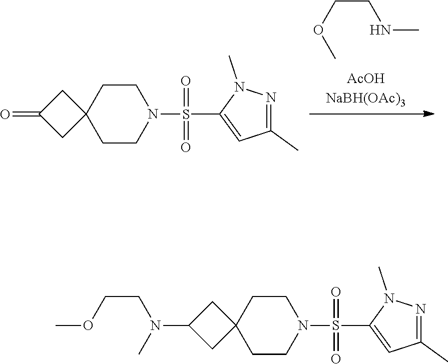

To a vial containing 7-((1,3-dimethyl-1H-pyrazol-5-yl)sulfonyl)-7-azaspiro[3.5]nonan-2-one (100 mg, 336 umol) in DCM (2 mL) was added AcOH (0.02 mL, 349 umol) then 2-methoxy-N-methyl-ethanamine (57 mg, 644 umol) carefully dropwise at 23° C. After 15 minutes, NaBH(OAc)₃ (279 mg, 1.32 mmol) was carefully added in portions to the reaction mixture. Upon complete addition of NaBH(OAc)₃, the reaction was stirred at 23° C. and monitored with LCMS. After 2 hours, the reaction was carefully quenched with slow addition of aq. 1 M NaOH solution. The mixture was stirred at 23° C. for 20 minutes, then the biphasic mixture was extracted three times with DCM. The organic extractions were pooled then dried over MgSO₄. After filtration and concentration under reduced pressure, the residue was loaded onto a silica gel column and purified with (20-90% 3:1 EtOAc:ethanol in heptane.) Fractions containing product were pooled then concentrated under reduced pressure to afford a colorless film that was dissolved in DMSO and few drops of water then filtered. The homogeneous solution was submitted for mass directed reverse phase HPLC purification. Liquid chromatography was performed using a Waters XSelect CSH C18, 5 m, 50 mm×100 mm column with mobile phase H₂O (A) and MeCN (B) and a gradient of 5-60% B (0.2% NH₄OH final v/v % modifier) with flow rate at 60 mL/min. Fractions containing desired product were pooled then concentrated under reduced pressure to afford a clear oil as 7-((1,3-dimethyl-1H-pyrazol-5-yl)sulfonyl)-N-(2-methoxyethyl)-N-methyl-7-azaspiro[3.5]nonan-2-amine (39 mg, 30% yield). LCMS m/z=371.3 (M+H)⁺. ¹H NMR (500 MHz, DMSO-d₆) δ (ppm)=6.59 (s, 1H), 3.92 (s, 3H), 3.75-3.32 (m, 5H), 3.24 (br s, 2H), 3.07-3.03 (m, 2H), 2.99-2.94 (m, 2H), 2.80-2.61 (m, 1H), 2.47-2.21 (m, 2H), 2.18 (s, 3H), 2.13-1.83 (m, 4H), 1.63-1.59 (m, 2H), 1.59-1.53 (m, 2H), 1.50-1.34 (m, 1H).

### Example 92: 6-(7-((1,3-dimethyl-1H-pyrazol-5-yl)sulfonyl)-7-azaspiro[3.5]nonan-2-yl)-2-oxa-6-azaspiro[3.3]heptane

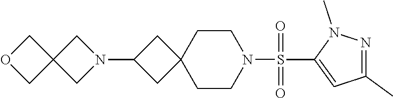 [SMILES: Cc1cc(S(=O)(=O)N2CCC3(CC2)CC(N2CC4(COC4)C2)C3)n(C)n1]

### 1. Synthesis of 6-(7-((1,3-dimethyl-1H-pyrazol-5-yl)sulfonyl)-7-azaspiro[3.5]nonan-2-yl)-2-oxa-6-azaspiro[3.3]heptane

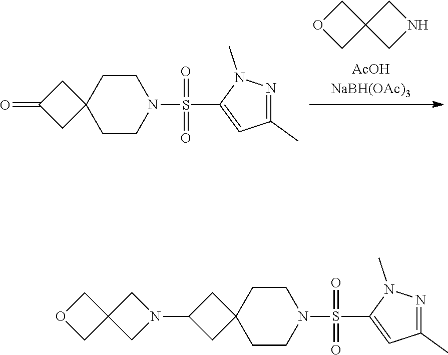 [SMILES: Cc1cc(S(=O)(=O)N2CCC3(CC2)CC(=O)C3)n(C)n1 ; C1NCC12COC2 ; Cc1cc(S(=O)(=O)N2CCC3(CC2)CC(N2CC4(COC4)C2)C3)n(C)n1]

To a vial containing 7-((1,3-dimethyl-1H-pyrazol-5-yl)sulfonyl)-7-azaspiro[3.5]nonan-2-one (91 mg, 306 umol) in DCM (1.5 mL) was added AcOH (0.02 mL, 349 umol) then 2-oxa-6-azaspiro[3.3]heptane (62 mg, 623 umol) carefully dropwise at 23° C. After 15 minutes, NaBH(OAc)₃ (238 mg, 1.12 mmol) was carefully added in portions to the reaction mixture. Upon complete addition of NaBH(OAc)₃, the reaction was stirred at 23° C. and monitored with LCMS. After 2 hours, the reaction was carefully quenched with slow addition of aq. 1 M NaOH solution. The mixture was stirred at 23° C. for 20 minutes, then the biphasic mixture was extracted three times with DCM. The organic extractions were pooled then dried over MgSO₄. After filtration and concentration under reduced pressure, the residue was dissolved in DMSO and few drops of water then filtered. The homogeneous solution was submitted for mass directed reverse phase HPLC purification. Liquid chromatography was performed using a Waters XSelect CSH C18, 5 m, 50 mm×100 mm column with mobile phase H₂O (A) and MeCN (B) and a gradient of 5-40% B (0.2% NH₄OH final v/v % modifier) with flow rate at 60 mL/min. Fractions containing desired product were pooled then concentrated under reduced pressure to afford a colorless film as 6-(7-((1,3-dimethyl-1H-pyrazol-5-yl)sulfonyl)-7-azaspiro[3.5]nonan-2-yl)-2-oxa-6-azaspiro[3.3]heptane (13 mg, 11%). LCMS m/z=381.3 (M+H)⁺. ¹H NMR (500 MHz, METHANOL-d₄) δ (ppm)=6.53 (s, 1H), 4.71 (s, 4H), 3.98 (s, 3H), 3.35-3.33 (m, 4H), 3.14-3.10 (m, 2H), 3.10-3.03 (m, 3H), 2.24 (s, 3H), 1.91-1.85 (m, 2H), 1.71-1.66 (m, 2H), 1.64-1.60 (m, 2H), 1.56-1.50 (m, 2H).

### Example 93: 6-(7-((2-methoxy-5-methylpyridin-3-yl)sulfonyl)-7-azaspiro[3.5]nonan-2-yl)-2-oxa-6-azaspiro[3.3]heptane

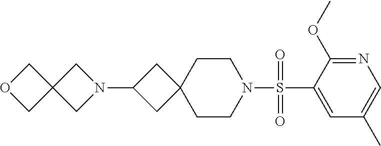 [SMILES: COc1ncc(C)cc1S(=O)(=O)N1CCC2(CC1)CC(N1CC3(COC3)C1)C2]

### 1. Synthesis of 7-((2-methoxy-5-methylpyridin-3-yl)sulfonyl)-7-azaspiro[3.5]nonan-2-one

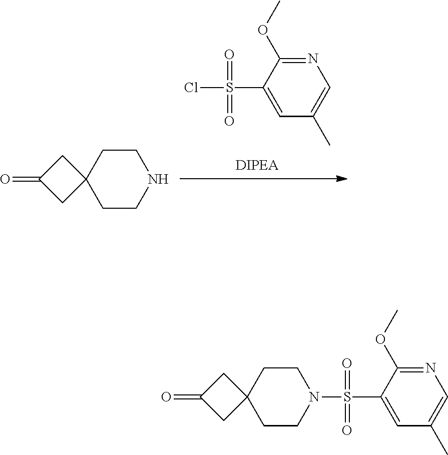 [SMILES: COc1ncc(C)cc1S(=O)(=O)Cl ; O=C1CC2(CCNCC2)C1 ; COc1ncc(C)cc1S(=O)(=O)N1CCC2(CC1)CC(=O)C2]

To a vial containing 7-azaspiro[3.5]nonan-2-one hydrochloride (183 mg, 1.04 mmol) in DCM (4 mL) was added DIPEA (0.73 mL, 4.2 mmol) carefully dropwise at <5° C. After 5 minutes, 2-methoxy-5-methyl-pyridine-3-sulfonyl chloride (243 mg, 1.10 mmol) was added carefully to the cold solution. Upon complete addition of sulfonyl chloride solution, the reaction was warmed to 23° C. and monitored with LCMS. After 30 minutes, the reaction was carefully quenched with slow addition of aq. 1 M NaOH solution. The mixture was stirred at 23° C. for 20 minutes, then the biphasic mixture was extracted three times with DCM. The organic extractions were pooled then dried over MgSO₄. After filtration and concentration under reduced pressure, the residue was loaded onto a silica gel column and purified with (15-65% EtOAc in heptane.) The desired fractions were pooled then concentrated under reduced pressure to afford a white solid as 7-((2-methoxy-5-methylpyridin-3-yl)sulfonyl)-7-azaspiro[3.5]nonan-2-one (337 mg, 99%) that was used without further purification. LCMS m/z=325.1 (M+H)⁺. ¹H NMR (400 MHz, DCM-d₂) δ (ppm)=8.16-8.12 (m, 1H), 8.04-7.99 (m, 1H), 4.04 (s, 3H), 3.29-3.22 (m, 4H), 2.76 (s, 4H), 2.31 (s, 3H), 1.82-1.77 (m, 4H).

### 2. Synthesis of 6-(7-((2-methoxy-5-methylpyridin-3-yl)sulfonyl)-7-azaspiro[3.5]nonan-2-yl)-2-oxa-6-azaspiro[3.3]heptane

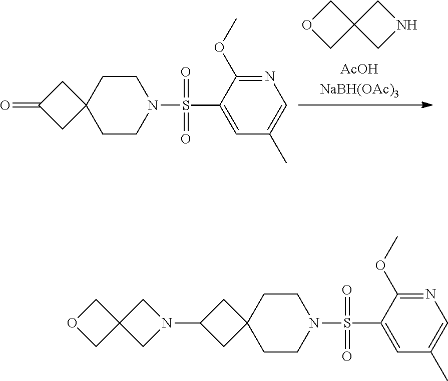 [SMILES: COc1ncc(C)cc1S(=O)(=O)N1CCC2(CC1)CC(=O)C2 ; C1NCC12COC2 ; COc1ncc(C)cc1S(=O)(=O)N1CCC2(CC1)CC(N1CC3(COC3)C1)C2]

To a vial containing 7-((2-methoxy-5-methylpyridin-3-yl)sulfonyl)-7-azaspiro[3.5]nonan-2-one (62 mg, 192 umol) in DCM (2 mL) was added AcOH (0.01 mL, 201 umol) then 2-oxa-6-azaspiro[3.3]heptane (40 mg, 406 umol) carefully dropwise at 23° C. After 15 minutes, NaBH(OAc)₃ (163 mg, 771 umol) was carefully added in portions to the reaction mixture. Upon complete addition of NaBH(OAc)₃, the reaction was stirred at 23° C. and monitored with LCMS. After 19 hours, the reaction was quenched with slow addition of aq. 1 M NaOH solution. The mixture was stirred at 23° C. for 20 minutes, then the biphasic mixture was extracted three times with DCM. The organic extractions were pooled then dried over MgSO₄. After filtration and concentration under reduced pressure, the residue was dissolved in DMSO and few drops of water then filtered. The homogeneous solution was submitted for mass directed reverse phase HPLC purification. Liquid chromatography was performed using a Waters XSelect CSH C18, 5 μm, 50 mm×100 mm column with mobile phase H₂O (A) and MeCN (B) and a gradient of 5-50% B (0.2% NH₄OH final v/v % modifier) with flow rate at 60 mL/min. Fractions containing desired product were pooled then concentrated under reduced pressure to afford a colorless film as 6-(7-((2-methoxy-5-methylpyridin-3-yl)sulfonyl)-7-azaspiro[3.5]nonan-2-yl)-2-oxa-6-azaspiro[3.3]heptane (8 mg, 10%). LCMS m/z=408.3 (M+H)⁺. ¹H NMR (400 MHz, DCM-d₂) δ (ppm)=8.10 (dd, J=0.8, 2.3 Hz, 1H), 7.97-7.90 (m, 1H), 4.65 (s, 4H), 4.03-3.96 (m, 3H), 3.25 (s, 4H), 3.18-3.13 (m, 2H), 3.11-3.03 (m, 2H), 2.96 (quin, J=7.4 Hz, 1H), 2.29 (s, 3H), 1.85-1.76 (m, 2H), 1.63-1.54 (m, 4H), 1.54-1.45 (m, 2H).

### Example 94: (1R,4R)-5-(7-((6-chloro-2-methylpyridin-3-yl)sulfonyl)-7-azaspiro[3.5]nonan-2-yl)-2-oxa-5-azabicyclo[2.2.1]heptane

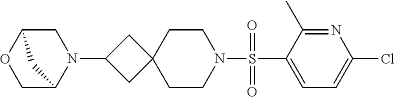 [SMILES: Cc1nc(Cl)ccc1S(=O)(=O)N1CCC2(CC1)CC(N1C[C@H]3C[C@@H]1CO3)C2]

### 1. Synthesis of 7-((6-chloro-2-methylpyridin-3-yl)sulfonyl)-7-azaspiro[3.5]nonan-2-one

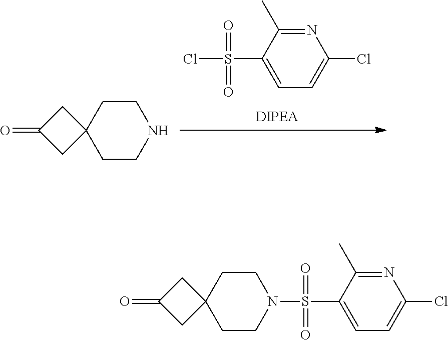 [SMILES: Cc1nc(Cl)ccc1S(=O)(=O)Cl ; O=C1CC2(CCNCC2)C1 ; Cc1nc(Cl)ccc1S(=O)(=O)N1CCC2(CC1)CC(=O)C2]

To a vial containing 7-azaspiro[3.5]nonan-2-one hydrochloride (180 mg, 1.02 mmol) in DCM (4 mL) was added DIPEA (0.7 mL, 4.02 mmol) carefully dropwise at <5° C. After 5 minutes, 6-chloro-2-methyl-pyridine-3-sulfonyl chloride (300 mg, 1.33 mmol) was added carefully to the cold solution. Upon complete addition of sulfonyl chloride solution, the reaction was warmed to 23° C. and monitored with LCMS. After 30 minutes, the reaction was carefully quenched with slow addition of aq. 1 M NaOH solution. The mixture was stirred at 23° C. for 20 minutes, then the biphasic mixture was extracted three times with DCM. The organic extractions were pooled then dried over MgSO₄. After filtration and concentration under reduced pressure, the residue was loaded onto a silica gel column and purified with (25-80% EtOAc in heptane.) The desired fractions were pooled then concentrated under reduced pressure to afford a white solid as 7-((6-chloro-2-methylpyridin-3-yl)sulfonyl)-7-azaspiro[3.5]nonan-2-one (274 mg, 81%) that was used without further purification. LCMS m/z=329.0 (M+H)⁺. ¹H NMR (500 MHz, Methanol-d₄) δ (ppm)=8.20 (d, J=8.2 Hz, 1H), 7.48 (d, J=8.2 Hz, 1H), 3.27-3.23 (m, 4H), 2.84-2.81 (m, 4H), 2.81-2.78 (m, 3H), 1.87-1.82 (m, 4H).

### 2. Synthesis of (1R,4R)-5-(7-((6-chloro-2-methylpyridin-3-yl)sulfonyl)-7-azaspiro[3.5]nonan-2-yl)-2-oxa-5-azabicyclo[2.2.1]heptane

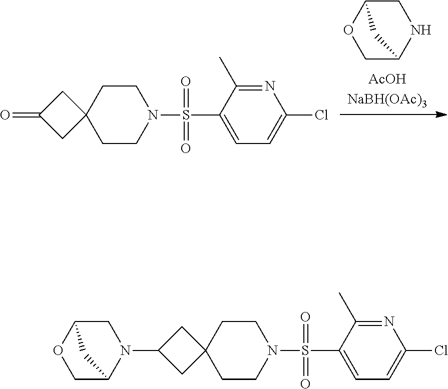 [SMILES: Cc1nc(Cl)ccc1S(=O)(=O)N1CCC2(CC1)CC(=O)C2 ; C1O[C@H]2CN[C@@H]1C2 ; Cc1nc(Cl)ccc1S(=O)(=O)N1CCC2(CC1)CC(N1C[C@H]3C[C@@H]1CO3)C2]

To a vial containing 7-((6-chloro-2-methylpyridin-3-yl)sulfonyl)-7-azaspiro[3.5]nonan-2-one (80 mg, 244 umol) in DCM (2 mL) was added AcOH (0.08 mL mg, 1.40 mmol) then a solution of (1R,4R)-2-oxa-5-azabicyclo[2.2.1]heptane hydrochloride (67 mg, 497 umol) and DIPEA (0.15 mL, 861 umol) in DCM was carefully dropwise at 23° C. After 15 minutes, NaBH(OAc)₃ (222 mg, 1.05 mmol) was carefully added in portions to the reaction mixture. Upon complete addition of NaBH(OAc)₃, the reaction was stirred at 23° C. and monitored with LCMS. After 2 hours, the reaction was carefully quenched with slow addition of aq. 1 M NaOH solution. The mixture was stirred at 23° C. for 20 minutes, then the biphasic mixture was extracted three times with DCM. The organic extractions were pooled then dried over MgSO₄. After filtration and concentration under reduced pressure, the residue was dissolved in DMSO and few drops of water then filtered. The homogeneous solution was submitted for mass directed reverse phase HPLC purification. Liquid chromatography was performed using a Waters XSelect CSH C18, 5 m, 50 mm×100 mm column with mobile phase H₂O (A) and MeCN (B) and a gradient of 5-60% B (0.2% NH₄OH final v/v % modifier) with flow rate at 60 mL/min. Fractions containing desired product were pooled then concentrated under reduced pressure to afford a colorless film as (1R,4R)-5-(7-((6-chloro-2-methylpyridin-3-yl)sulfonyl)-7-azaspiro[3.5]nonan-2-yl)-2-oxa-5-azabicyclo[2.2.1]heptane (25 mg, 23%). LCMS m/z=412.2 (M+H)⁺. ¹H NMR (500 MHz, DMSO-d₆) δ (ppm)=8.15 (d, J=8.5 Hz, 1H), 7.58 (d, J=8.5 Hz, 1H), 4.30 (s, 1H), 3.68 (d, J=7.3 Hz, 1H), 3.43 (dd, J=1.7, 7.2 Hz, 1H), 3.34 (s, 1H), 3.18-3.11 (m, 1H), 3.10-3.05 (m, 2H), 3.03-2.98 (m, 2H), 2.71 (s, 3H), 2.59 (dd, J=1.4, 9.9 Hz, 1H), 2.34 (d, J=10.1 Hz, 1H), 1.94-1.88 (m, 1H), 1.87-1.80 (m, 1H), 1.65-1.61 (m, 1H), 1.60-1.52 (m, 4H), 1.52-1.45 (m, 3H).

### Example 95: 1-(7-((2-methyl-6-(trifluoromethyl)pyridin-3-yl)sulfonyl)-7-azaspiro[3.5]nonan-2-yl)azetidin-3-ol

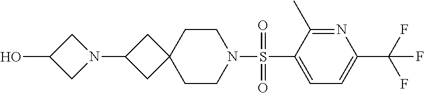 [SMILES: Cc1nc(C(F)(F)F)ccc1S(=O)(=O)N1CCC2(CC1)CC(N1CC(O)C1)C2]

### 1. Synthesis of 7-((6-chloro-2-methylpyridin-3-yl)sulfonyl)-7-azaspiro[3.5]nonan-2-one

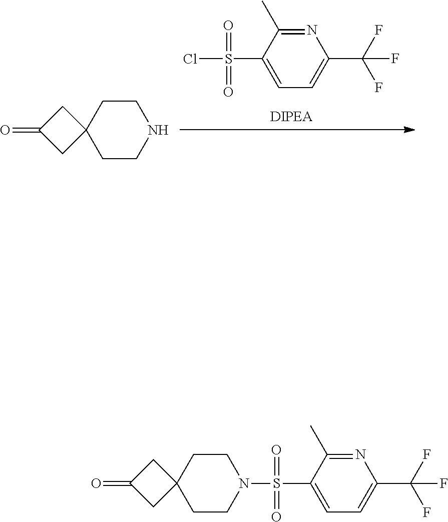 [SMILES: Cc1nc(C(F)(F)F)ccc1S(=O)(=O)Cl ; O=C1CC2(CCNCC2)C1 ; Cc1nc(C(F)(F)F)ccc1S(=O)(=O)N1CCC2(CC1)CC(=O)C2]

To a vial containing 7-azaspiro[3.5]nonan-2-one hydrochloride (531 mg, 3.02 mmol) in DCM (10 mL) was added DIPEA (2 mL, 11.51 mmol) carefully dropwise at RT. After 10 minutes, 2-methyl-6-(trifluoromethyl)pyridine-3-sulfonyl chloride (789 mg, 3.04 mmol) was added carefully to the mixture. Upon complete addition of sulfonyl chloride, the reaction was maintained at 23° C. and monitored with LCMS. After 1 hour, the reaction mixture was carefully quenched with slow addition of sat., aq. NaHCO₃. The heterogeneous mixture was extracted three times with DCM. The organic extractions were pooled then dried over MgSO₄. After filtration and concentration under reduced pressure, the residue was loaded onto a silica gel column and purified with (5-60% EtOAc in heptane.) The desired fractions were pooled then concentrated under reduced pressure to afford a white solid as 7-((6-chloro-2-methylpyridin-3-yl)sulfonyl)-7-azaspiro[3.5]nonan-2-one (925 mg, 84%) that was used without further purification. LCMS m/z=363.0 (M+H)⁺. ¹H NMR (500 MHz, DMSO-d₆) δ (ppm)=8.45 (d, J=8.2 Hz, 1H), 7.97 (d, J=7.9 Hz, 1H), 3.25-3.17 (m, 4H), 2.83 (s, 3H), 2.80 (s, 4H), 1.79-1.73 (m, 4H).

### 2. Synthesis of 1-(7-((2-methyl-6-(trifluoromethyl)pyridin-3-yl)sulfonyl)-7-azaspiro[3.5]nonan-2-yl)azetidin-3-ol

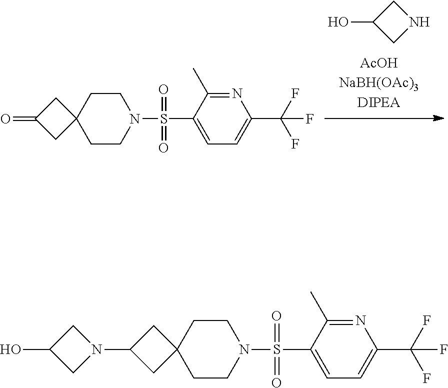 [SMILES: OC1CNC1 ; Cc1nc(C(F)(F)F)ccc1S(=O)(=O)N1CCC2(CC1)CC(=O)C2 ; Cc1nc(C(F)(F)F)ccc1S(=O)(=O)N1CCC2(CC1)CC(N1CC(O)C1)C2]

A vial containing azetidin-3-ol hydrochloride (66 mg, 601 umol) in methanol (2 mL) was cooled in an ice water bath, then DIPEA (0.2 mL, 1.15 mmol) was added carefully to free base the starting material. After 20 minutes, 7-((6-chloro-2-methylpyridin-3-yl)sulfonyl)-7-azaspiro[3.5]nonan-2-one (102 mg, 282 umol) and AcOH (0.08 mL, 1.40 mmol) were carefully added to the cooled mixture. After 15 minutes, NaBH(OAc)₃ (345 mg, 1.63 mmol) was added carefully in portions to the cooled reaction solution. Upon complete addition of NaBH(OAc)₃, the reaction was maintained at <5° C. and monitored with LCMS. After 1.5 hours, the reaction was carefully quenched with slow addition of aq. sat. NaHCO₃ solution. The mixture was stirred at 23° C. for 30 minutes, then the mixture was extracted three times with DCM. The organic extractions were pooled then dried over MgSO₄. After filtration and concentration under reduced pressure, the residue was loaded onto a silica gel column and purified with (40-100% 3:1 EtOAc:ethanol in heptane.) The desired fractions were pooled then concentrated under reduced pressure to afford a colorless film that was dissolved in DMSO and few drops of water then filtered. The homogeneous solution was submitted for mass directed reverse phase HPLC purification. Liquid chromatography was performed using a Waters XSelect CSH C18, 5 m, 30 mm×100 mm column with mobile phase H₂O (A) and MeCN (B) and a gradient of 5-50% B (0.2% NH₄OH final v/v % modifier) with flow rate at 30 mL/min. The desired fractions were pooled then concentrated under reduced pressure to afford a colorless film as 1-(7-((2-methyl-6-(trifluoromethyl)pyridin-3-yl)sulfonyl)-7-azaspiro[3.5]nonan-2-yl)azetidin-3-ol (8 mg, 6%). LCMS m/z=420.1 (M+H)⁺. ¹H NMR (500 MHz, DCM-d₂) δ (ppm)=8.41-8.35 (m, 1H), 7.75-7.69 (m, 1H), 4.64-4.27 (m, 1H), 3.75-3.37 (m, 2H), 3.23-3.10 (m, 4H), 2.90 (s, 3H), 2.12-1.75 (m, 4H), 1.72-1.68 (m, 2H), 1.61-1.43 (m, 6H).

### Example 96: 3-((2-(2-oxa-6-azaspiro[3.3]heptan-6-yl)-7-azaspiro[3.5]nonan-7-yl)sulfonyl)-5-fluorobenzonitrile

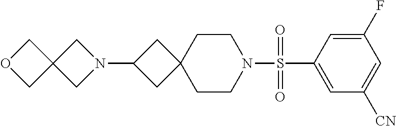 [SMILES: N#Cc1cc(F)cc(S(=O)(=O)N2CCC3(CC2)CC(N2CC4(COC4)C2)C3)c1]

### 1. Synthesis of 3-fluoro-5-((2-oxo-7-azaspiro[3.5]nonan-7-yl)sulfonyl)benzonitrile

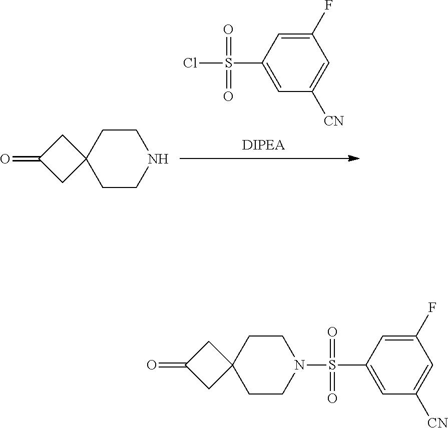 [SMILES: N#Cc1cc(F)cc(S(=O)(=O)Cl)c1 ; O=C1CC2(CCNCC2)C1 ; N#Cc1cc(F)cc(S(=O)(=O)N2CCC3(CC2)CC(=O)C3)c1]

To a vial containing 7-azaspiro[3.5]nonan-2-one hydrochloride (188 mg, 1.07 mmol) in DCM (4 mL) was added DIPEA (0.6 mL, 3.44 mmol) carefully dropwise at <5° C. After 5 minutes, 3-cyano-5-fluoro-benzenesulfonyl chloride (341 mg, 1.55 mmol) was added carefully to the cold solution. Upon complete addition of sulfonyl chloride, the reaction was warmed to 23° C. and monitored with LCMS. After 30 minutes, the reaction was carefully quenched with slow addition of aq. 1 M NaOH solution. The mixture was stirred at 23° C. for 20 minutes, then the biphasic mixture was extracted three times with DCM. The organic extractions were pooled then dried over MgSO₄. After filtration and concentration under reduced pressure, the residue was loaded onto a silica gel column and purified with (20-80% EtOAc in heptane.) The desired fractions were pooled then concentrated under reduced pressure to afford a white solid as 3-fluoro-5-((2-oxo-7-azaspiro[3.5]nonan-7-yl)sulfonyl)benzonitrile (267 mg, 78%) that was used without further purification. ¹H NMR (500 MHz, DCM-d₂) δ (ppm)=7.87 (s, 1H), 7.74-7.70 (m, 1H), 7.65-7.61 (m, 1H), 3.10-3.07 (m, 4H), 2.74 (s, 4H), 1.88-1.85 (m, 4H).

### 2. Synthesis of 3-((2-(2-oxa-6-azaspiro[3.3]heptan-6-yl)-7-azaspiro[3.5]nonan-7-yl)sulfonyl)-5-fluorobenzonitrile

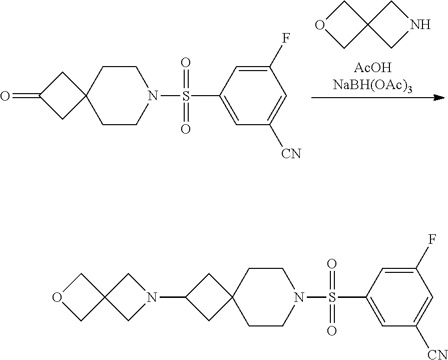

To a vial containing 3-fluoro-5-((2-oxo-7-azaspiro[3.5]nonan-7-yl)sulfonyl)benzonitrile (111 mg, 345 umol) in DCM (2 mL) was added AcOH (0.02 mL, 349 umol) then 2-oxa-6-azaspiro[3.3]heptane (72 mg, 728 umol) carefully dropwise at 23° C. After 15 minutes, NaBH(OAc)₃ (278 mg, 1.31 mmol) was carefully added in portions to the reaction mixture. Upon complete addition of NaBH(OAc)₃, the reaction was stirred at 23° C. and monitored with LCMS. After 1 hour, the reaction was carefully quenched with slow addition of aq. 1 M NaOH solution. The mixture was stirred at 23° C. for 20 minutes, then the biphasic mixture was extracted three times with DCM. The organic extractions were pooled then dried over MgSO₄. After filtration and concentration under reduced pressure, the residue was dissolved in DMSO and few drops of water then filtered. The homogeneous solution was submitted for mass directed reverse phase HPLC purification. Liquid chromatography was performed using a Waters XSelect CSH C18, 5 m, 50 mm×100 mm column with mobile phase H₂O (A) and MeCN (B) and a gradient of 5-55% B (0.2% NH₄OH final v/v % modifier) with flow rate at 60 mL/min. Fractions containing desired product were pooled then concentrated under reduced pressure to afford a white solid as 3-((2-(2-oxa-6-azaspiro[3.3]heptan-6-yl)-7-azaspiro[3.5]nonan-7-yl)sulfonyl)-5-fluorobenzonitrile (32 mg, 22%). LCMS m/z=406.2 (M+H)⁺. ¹H NMR (500 MHz, DMSO-d₆) δ (ppm)=8.27 (br d, J=8.5 Hz, 1H), 8.06 (s, 1H), 7.95 (dd, J=1.5, 7.9 Hz, 1H), 4.54 (s, 4H), 3.12 (s, 4H), 2.95-2.86 (m, 5H), 1.69-1.63 (m, 2H), 1.53 (br t, J=4.3 Hz, 4H), 1.39-1.33 (m, 2H).

### Example 97: 3-((2-((1R,4R)-2-oxa-5-azabicyclo[2.2.1]heptan-5-yl)-8-azaspiro[4.5]decan-8-yl)sulfonyl)-5-fluorobenzonitrile

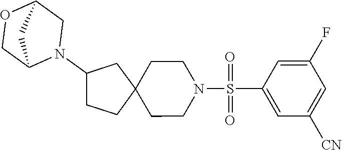 [SMILES: N#Cc1cc(F)cc(S(=O)(=O)N2CCC3(CCC(N4C[C@H]5C[C@@H]4CO5)C3)CC2)c1]

### 1. Synthesis of 3-((2-((1R,4R)-2-oxa-5-azabicyclo[2.2.1]heptan-5-yl)-8-azaspiro[4.5]decan-8-yl)sulfonyl)-5-fluorobenzonitrile

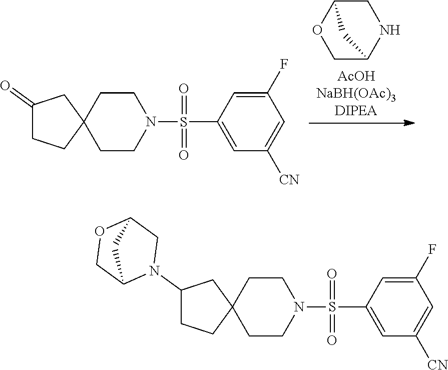 [SMILES: C1O[C@H]2CN[C@@H]1C2 ; N#Cc1cc(F)cc(S(=O)(=O)N2CCC3(CCC(=O)C3)CC2)c1 ; N#Cc1cc(F)cc(S(=O)(=O)N2CCC3(CCC(N4C[C@H]5C[C@@H]4CO5)C3)CC2)c1]

To a vial containing 3-fluoro-5-[(3-oxo-8-azaspiro[4.5]decan-8-yl)sulfonyl]benzonitrile (99 mg, 294 umol) in DCM (5 mL) was added AcOH (0.08 mL 1.4 mmol) then a solution of (1R,4R)-2-oxa-5-azabicyclo[2.2.1]heptane hydrochloride (92 mg, 681 umol) and DIPEA (0.2 mL, 1.15 mmol) in DCM carefully dropwise at 23° C. After 15 minutes, NaBH(OAc)₃ (311 mg, 1.47 mmol) was carefully added in portions to the reaction mixture. Upon complete addition of NaBH(OAc)₃, the reaction was stirred at 23° C. and monitored with LCMS. After 1 hour, the reaction was carefully quenched with slow addition of aq. 1 M NaOH solution. The mixture was stirred at 23° C. for 20 minutes, then the biphasic mixture was extracted three times with DCM. The organic extractions were pooled then dried over MgSO₄. After filtration and concentration under reduced pressure, the residue was loaded onto a silica gel column and purified with (45-100% 3:1 EtOAc:ethanol in heptane.) The desired fractions were pooled then concentrated under reduced pressure to afford a colorless film as 3-((2-((1R,4R)-2-oxa-5-azabicyclo[2.2.1]heptan-5-yl)-8-azaspiro[4.5]decan-8-yl)sulfonyl)-5-fluorobenzonitrile (49 mg, 38%). LCMS m/z=420.3 (M+H)⁺. ¹H NMR (500 MHz, DMSO-d₆) δ (ppm)=8.27 (br d, J=8.2 Hz, 1H), 8.08 (s, 1H), 7.97 (br d, J=7.6 Hz, 1H), 4.25 (br s, 1H), 3.80 (br d, J=7.0 Hz, 1H), 3.46-3.40 (m, 2H), 3.03-2.93 (m, 4H), 2.92-2.83 (m, 1H), 2.79-2.69 (m, 1H), 2.28 (br d, J=9.5 Hz, 1H), 1.76-1.61 (m, 2H), 1.55-1.25 (m, 9H), 1.19-1.11 (m, 1H).

### Example 98: 3-fluoro-5-((3-(((tetrahydro-2H-pyran-4-yl)methyl)amino)-1-oxa-8-azaspiro[4.5]decan-8-yl)sulfonyl)benzonitrile

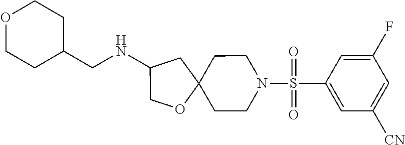 [SMILES: N#Cc1cc(F)cc(S(=O)(=O)N2CCC3(CC2)CC(NCC2CCOCC2)CO3)c1]

### 1. Synthesis of 3-fluoro-5-((3-oxo-1-oxa-8-azaspiro[4.5]decan-8-yl)sulfonyl)benzonitrile

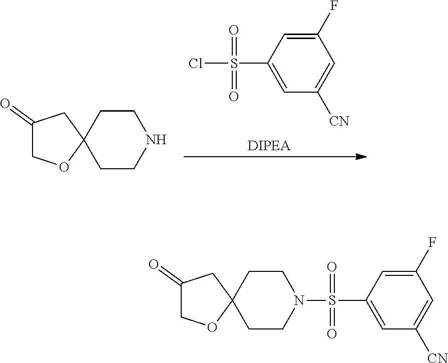 [SMILES: O=C1COC2(CCNCC2)C1 ; N#Cc1cc(F)cc(S(=O)(=O)Cl)c1 ; "DIPEA" ; N#Cc1cc(F)cc(S(=O)(=O)N2CCC3(CC2)CC(=O)CO3)c1]

To a vial containing 1-oxa-8-azaspiro[4.5]decan-3-one hydrochloride (139 mg, 897 umol) in DCM (3 mL) was added DIPEA (0.7 mL, 4.02 mmol) carefully dropwise at <5° C. After 5 minutes, 3-cyano-5-fluoro-benzenesulfonyl chloride (293 mg, 1.33 mmol) was added carefully to the cold solution. Upon complete addition of sulfonyl chloride, the reaction was warmed to 23° C. and monitored with LCMS. After 30 minutes, the reaction was carefully quenched with slow addition of aq. 1 M NaOH solution. The mixture was stirred at 23° C. for 20 minutes, then the biphasic mixture was extracted three times with DCM. The organic extractions were pooled then dried over MgSO₄. After filtration and concentration under reduced pressure, the residue was loaded onto a silica gel column and purified with (20-80% EtOAc in heptane.) The desired fractions were pooled then concentrated under reduced pressure to afford a sticky white foam as 3-fluoro-5-((3-oxo-1-oxa-8-azaspiro[4.5]decan-8-yl)sulfonyl)benzonitrile (169 mg, 56%) that was used without further purification. ¹H NMR (500 MHz, DCM-d₂) δ (ppm)=7.88 (s, 1H), 7.73 (td, J=1.8, 7.6 Hz, 1H), 7.63 (dd, J=1.2, 7.6 Hz, 1H), 3.90 (s, 2H), 3.57-3.53 (m, 2H), 2.86 (dt, J=3.4, 11.4 Hz, 2H), 2.34 (s, 2H), 1.93-1.84 (m, 4H).

### 2. Synthesis of 3-fluoro-5-((3-(((tetrahydro-2H-pyran-4-yl)methyl)amino)-1-oxa-8-azaspiro[4.5]decan-8-yl)sulfonyl)benzonitrile

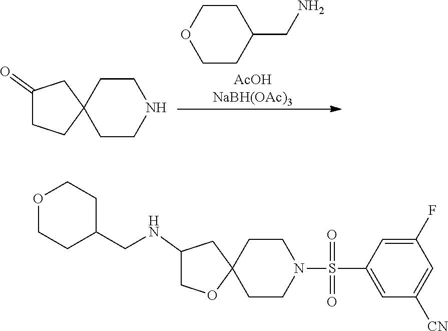

To a vial containing 3-fluoro-5-((3-oxo-1-oxa-8-azaspiro[4.5]decan-8-yl)sulfonyl)benzonitrile (81 mg, 239 umol) in DCM (3 mL) was added AcOH (0.02 mL, 349 umol) then tetrahydropyran-4-ylmethanamine (60 mg, 521 umol) carefully dropwise at 23° C. After 15 minutes, NaBH(OAc)₃ (200 mg, 943 umol) was carefully added in portions to the reaction mixture. Upon complete addition of NaBH(OAc)₃, the reaction was stirred at 23° C. and monitored with LCMS. After 18 hours, the reaction was carefully quenched with slow addition of aq. 1 M NaOH solution. The mixture was stirred at 23° C. for 20 minutes, then the biphasic mixture was extracted three times with DCM. The organic extractions were pooled then dried over MgSO₄. After filtration and concentration under reduced pressure, the residue was loaded onto a silica gel column and purified with (35-90% 3:1 EtOAc:ethanol in heptane.) The desired fractions were pooled then concentrated under reduced pressure to afford a colorless film as 3-fluoro-5-((3-(((tetrahydro-2H-pyran-4-yl)methyl)amino)-1-oxa-8-azaspiro[4.5]decan-8-yl)sulfonyl)benzonitrile (42 mg, 38%). LCMS m/z=438.3 (M+H)⁺. ¹H NMR (500 MHz, DMSO-d₆) δ (ppm)=8.27 (br d, J=8.5 Hz, 1H), 8.10 (s, 1H), 7.98 (br d, J=7.6 Hz, 1H), 3.80 (br dd, J=3.4, 11.3 Hz, 2H), 3.74 (dd, J=6.1, 8.5 Hz, 1H), 3.39-3.35 (m, 1H), 3.31-3.15 (m, 6H), 2.78-2.69 (m, 2H), 2.34-2.24 (m, 2H), 1.88 (s, 1H), 1.80-1.75 (m, 1H), 1.72-1.64 (m, 1H), 1.59-1.49 (m, 5H), 1.44 (dd, J=5.6, 12.7 Hz, 1H), 1.07 (br s, 2H).

### Example 99: 1-(8-((6-methoxy-2-methylpyridin-3-yl)sulfonyl)-8-azaspiro[4.5]decan-2-yl)azetidin-3-ol

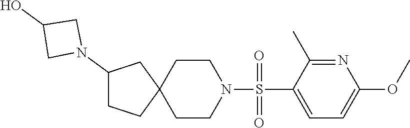 [SMILES: COc1ccc(S(=O)(=O)N2CCC3(CCC(N4CC(O)C4)C3)CC2)c(C)n1]

### 1. Synthesis of 8-((6-methoxy-2-methylpyridin-3-yl)sulfonyl)-8-azaspiro[4.5]decan-2-one

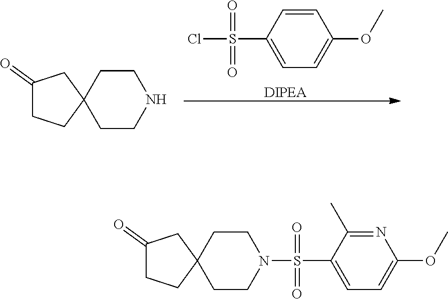

To a flask containing 8-azaspiro[4.5]decan-3-one hydrochloride (1.0 g, 5.3 mmol) in DCM (35 mL) was added DIPEA (3.6 mL, 21 mmol) carefully dropwise at <5° C. After 5 minutes, 6-methoxy-2-methyl-pyridine-3-sulfonyl chloride (1.4 g, 6.1 mmol) was added carefully to the cold solution. Upon complete addition of sulfonyl chloride, the reaction was warmed to 23° C. and monitored with LCMS. After 30 minutes, the reaction mixture was carefully quenched with slow addition of aq. 1 M NaOH solution. The mixture was stirred at 23° C. for 10 minutes, then the phases of the biphasic mixture were separated. The aq. phase was washed with DCM (10 mL×2), and the combined organic phase was dried over Na₂SO₄, filtered, and concentrated to give crude material. The crude was loaded onto a silica gel column and purified with (10-55% EtOAc in heptane.) The desired fractions were pooled then concentrated under reduced pressure to afford a white solid as 8-((6-methoxy-2-methylpyridin-3-yl)sulfonyl)-8-azaspiro[4.5]decan-2-one (1.7 g, 97%). LCMS m/z=338.9 (M+H)⁺.

### 2. Synthesis of 1-(8-((6-methoxy-2-methylpyridin-3-yl)sulfonyl)-8-azaspiro[4.5]decan-2-yl)azetidin-3-ol

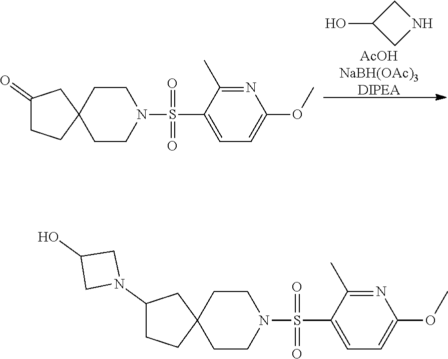

A vial containing azetidin-3-ol hydrochloride (24 mg, 323 umol) in methanol (2 mL) was cooled in an ice water bath, then DIPEA (0.15 mL, 652 umol) was added carefully to free base the starting material. After 20 minutes, 8-((6-methoxy-2-methylpyridin-3-yl)sulfonyl)-8-azaspiro[4.5]decan-2-one (73 mg, 215 umol) and AcOH (0.05 mL, 862 umol) were carefully added to the cooled mixture. After 15 minutes, NaBH(OAc)₃ (365 mg, 1.73 mmol) was added carefully in portions to the cooled reaction solution. Upon complete addition of NaBH(OAc)₃, the reaction was maintained at <5° C. and monitored with LCMS. After 18 hours, the reaction was carefully quenched with slow addition of aq. 1 M NaOH solution. The mixture was stirred at 23° C. for 20 minutes, then the biphasic mixture was extracted three times with DCM. The organic extractions were pooled then dried over MgSO₄. After filtration and concentration under reduced pressure, the residue was dissolved in DMSO and few drops of water then filtered. The homogeneous solution was submitted for mass directed reverse phase HPLC purification. Liquid chromatography was performed using a Waters XSelect CSH C18, 5 m, 50 mm×100 mm column with mobile phase H₂O (A) and MeCN (B) and a gradient of 5-65% B (0.2% NH₄OH final v/v % modifier) with flow rate at 60 mL/min. Fractions containing desired product were pooled then concentrated under reduced pressure to afford a white solid as 1-(8-((6-methoxy-2-methylpyridin-3-yl)sulfonyl)-8-azaspiro[4.5]decan-2-yl)azetidin-3-ol (56 mg, 66%). LCMS m/z=396.2 (M+H)⁺. ¹H NMR (600 MHz, DMSO-d₆) δ (ppm)=7.96 (d, J=8.7 Hz, 1H), 6.78 (d, J=8.7 Hz, 1H), 3.88 (s, 3H), 3.21-3.15 (m, 2H), 3.08-2.98 (m, 2H), 2.97-2.89 (m, 2H), 2.63 (s, 3H), 2.46 (td, J=1.8, 3.6 Hz, 3H), 1.72-1.55 (m, 2H), 1.51-1.40 (m, 4H), 1.40-1.31 (m, 5H), 1.12-1.07 (m, 1H).

### Example 100: 8-((6-methoxy-2-methylpyridin-3-yl)sulfonyl)-N-(2-methoxyethyl)-N-methyl-8-azaspiro[4.5]decan-2-amine

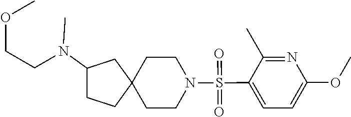 [SMILES: COCCN(C)C1CCC2(CCN(S(=O)(=O)c3ccc(OC)nc3C)CC2)C1]

### 1. Synthesis of 8-((6-methoxy-2-methylpyridin-3-yl)sulfonyl)-N-(2-methoxyethyl)-N-methyl-8-azaspiro[4.5]decan-2-amine

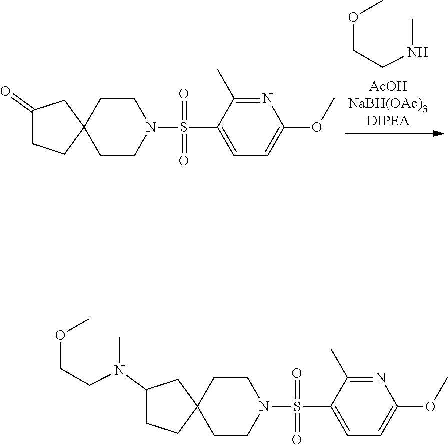

8-((6-methoxy-2-methylpyridin-3-yl)sulfonyl)-N-(2-methoxyethyl)-N-methyl-8-azaspiro[4.5]decan-2-amine was prepared in a manner similar to Example 99, step 2 using 2-methoxy-N-methylethan-1-amine (0.03 mL, 323 umol) to afford a colorless film as 8-((6-methoxy-2-methylpyridin-3-yl)sulfonyl)-N-(2-methoxyethyl)-N-methyl-8-azaspiro[4.5]decan-2-amine (50 mg, 57%). LCMS m/z=412.3 (M+H)⁺. ¹H NMR (600 MHz, DMSO-d₆) δ (ppm)=8.01 (d, J=9.1 Hz, 1H), 6.82 (d, J=8.7 Hz, 1H), 3.50-3.30 (m, 4H), 3.25 (s, 3H), 3.10-2.99 (m, 5H), 2.67 (s, 3H), 2.54 (s, 2H), 2.43-2.32 (m, 2H), 1.90-1.75 (m, 2H), 1.59-1.52 (m, 2H), 1.51-1.34 (m, 7H), 1.32-1.23 (m, 1H).

### Example 101: 6-(8-((5-(trifluoromethyl)furan-3-yl)sulfonyl)-8-azaspiro[4.5]decan-2-yl)-2-oxa-6-azaspiro[3.3]heptane

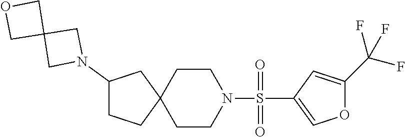 [SMILES: O=S(=O)(c1coc(C(F)(F)F)c1)N1CCC2(CCC(N3CC4(COC4)C3)C2)CC1]

### 1. Synthesis of benzyl 2-oxo-8-azaspiro[4.5]decane-8-carboxylate

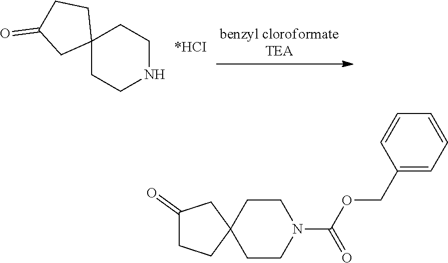 [SMILES: O=C1CCC2(CCNCC2)C1 ; O=C1CCC2(CCN(C(=O)OCc3ccccc3)CC2)C1]

To a flask containing 8-azaspiro[4.5]decan-3-one hydrochloride (10 g, 53 mmol) suspended in DCM (200 ml) under argon was added TEA (8.4 mL, 61 mmol), then benzyl chloroformate (8.3 mL, 58 mmol) was added dropwise to the reaction mixture with stirring at 0° C. The mixture was stirred at room temperature under argon atmosphere, then the reaction mixture was washed with brine (2×50 mL), dried over Na₂SO₄, filtered and concentrated in vacuo. The yellow oil was identified as benzyl 2-oxo-8-azaspiro[4.5]decane-8-carboxylate (14 g, 88%) that was used without further purification. ¹H NMR (400 MHz, CDCl₃) δ (ppm)=7.41-7.34 (m, 5H), 5.14 (s, 2H), 3.77-3.64 (m, 1H), 3.35-3.25 (m, 2H), 2.31 (br t, J=7.9 Hz, 2H), 2.18 (s, 2H), 1.88 (br t, J=7.9 Hz, 2H), 1.55 (br s, 4H).

### 2. Synthesis of benzyl 2-(2-oxa-6-azaspiro[3.3]heptan-6-yl)-8-azaspiro[4.5]decane-8-carboxylate

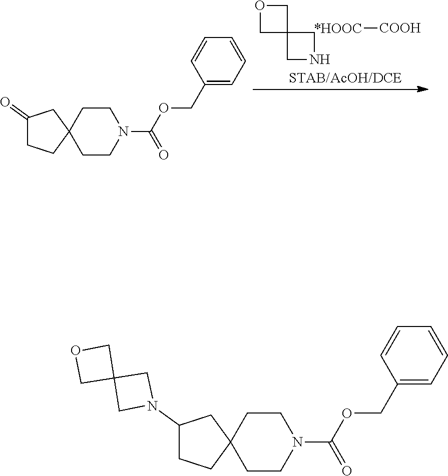

To a solution of benzyl 2-oxo-8-azaspiro[4.5]decane-8-carboxylate (14 g, 49 mmol) and 2-oxa-6-azaspiro[3.3]heptane oxalate salt (11 g, 59 mmol) in DCE (250 mL) was added AcOH (2.8 mL, 49 mmol) and STAB (21 g, 97 mmol). The resulting mixture was stirred at room temperature for 72 hours. The reaction mixture was concentrated under reduced pressure, then NaHCO₃ solution was added carefully to pH 8-9. After extracting with ethyl acetate (100 mL×3), the combined organic layers were washed with water (50 mL) then brine (50 mL). After drying over Na₂SO₄, filtration and concentration in vacuo, the yellow oil was identified as benzyl 2-(2-oxa-6-azaspiro[3.3]heptan-6-yl)-8-azaspiro[4.5]decane-8-carboxylate (11 g, 59%) that was used without further purification. LCMS m/z=371.2 (M+H)⁺.

### 3. Synthesis of 6-(8-Azaspiro[4.5]Decan-2-Yl)-2-Oxa-6-Azaspiro[3.3]Heptane

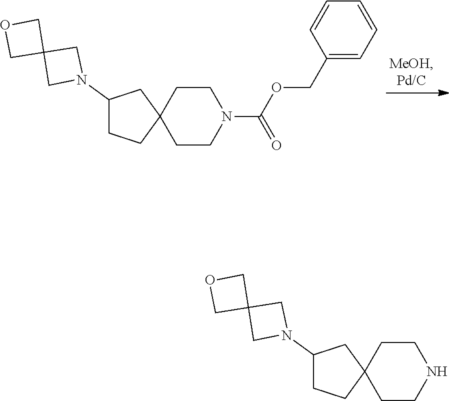 [SMILES: O=C(OCc1ccccc1)N1CCC2(CCC(N3CC4(COC4)C3)C2)CC1 ; C1CC2(CCN1)CCC(N1CC3(COC3)C1)C2]

To a solution of benzyl 2-(2-oxa-6-azaspiro[3.3]heptan-6-yl)-8-azaspiro[4.5]decane-8-carboxylate (11 g, 30 mmol) in MeOH (200 mL) was added 10% Pd/C (16 g, 15 mmol). The reaction mixture was stirred at room temperature under hydrogen atmosphere. After 4 hours, nitrogen was bubbled through the reaction mixture then carefully filtered through a celite plug. The filtrate was concentrated under reduced pressure. The residue was purified by flash column chromatography (Interchim, SiO₂ (40 g), EtOAc/Methanol with Methanol from 0˜95%, flow rate=60 mL/min, Rt=24 min.) to give afford a white solid as 6-(8-azaspiro[4.5]decan-2-yl)-2-oxa-6-azaspiro[3.3]heptane (6 g, 51%). LCMS m/z=237.2 (M+H)⁺.

### 4. Synthesis of 6-(8-((5-(trifluoromethyl)furan-3-yl)sulfonyl)-8-azaspiro[4.5]decan-2-yl)-2-oxa-6-azaspiro[3.3]heptane

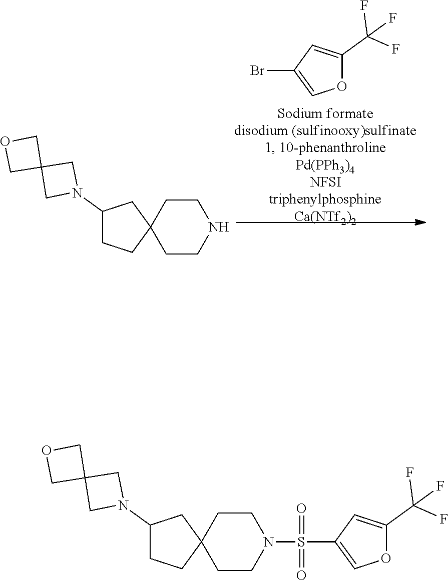 [SMILES: FC(F)(F)c1cc(Br)co1 ; C1CC2(CCN1)CCC(N1CC3(COC3)C1)C2 ; O=S(=O)(c1coc(C(F)(F)F)c1)N1CCC2(CCC(N3CC4(COC4)C3)C2)CC1]

4-Bromo-2-(trifluoromethyl)furan (0.25 g, 1.2 mmol) was suspended in DMSO (2 mL) under argon, then sodium formate (87 mg, 1.3 mmol), disodium (sulfinooxy)sulfinate (782 mg, 2.33 mmol), 1,10-phenanthroline (31 mg, 174 umol), triphenylphosphine (305 mg, 1.2 mmol), and Pd(PPh₃)₄ (134 mg, 116 umol) were added. The mixture was heated at 60° C. After 14 hours, the reaction mixture was cooled to room temperature, NFSI (550 mg, 1.74 mmol) was added. After 14 hours, the suspension was diluted with water (5 mL) then extracted with EtOAc (3×10 mL). The combined organic layers were washed with brine (2×mL) then dried over Na₂SO₄. After filtration and concentration under reduced pressure, the crude sulfonyl fluoride was dissolved in THE (2 mL). 6-(8-azaspiro[4.5]decan-2-yl)-2-oxa-6-azaspiro[3.3]heptane (60 mg, 1.2 mmol), Ca(NTf₂)₂ (1.05 g, 1.74 mmol), and DABCO (0.26 mL, 2.33 mmol) were added, then the mixture was stirred at 60° C. under argon atmosphere. After 14 hours, the reaction was diluted with water (5 mL) and extracted with EtOAc (3×10 mL). The combined organic layers were washed with brine (2×10 mL) then dried over Na₂SO₄. After filtration and concentration under reduced pressure, the residue was purified by HPLC (Column: XBridge BEH C18 5 μm 130A; 35-35-55% 0-1-6 min H₂O/ACN/0.1% NH₄OH, flow: 30 ml/min) to give 6-(8-((5-(trifluoromethyl)furan-3-yl)sulfonyl)-8-azaspiro[4.5]decan-2-yl)-2-oxa-6-azaspiro[3.3]heptane (1.3 mg, 0.26%). LCMS m/z=435.0 (M+H)⁺. LCMS R_{f}=2.75 mins.

### Example 102: 6-(8-((7-fluoroquinolin-6-yl)sulfonyl)-8-azaspiro[4.5]decan-2-yl)-2-oxa-6-azaspiro[3.3]heptane

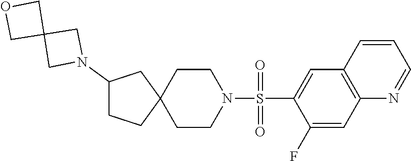 [SMILES: O=S(=O)(c1cc2cccnc2cc1F)N1CCC2(CCC(N3CC4(COC4)C3)C2)CC1]

### 1. Synthesis of 6-(8-((7-fluoroquinolin-6-yl)sulfonyl)-8-azaspiro[4.5]decan-2-yl)-2-oxa-6-azaspiro[3.3]heptane

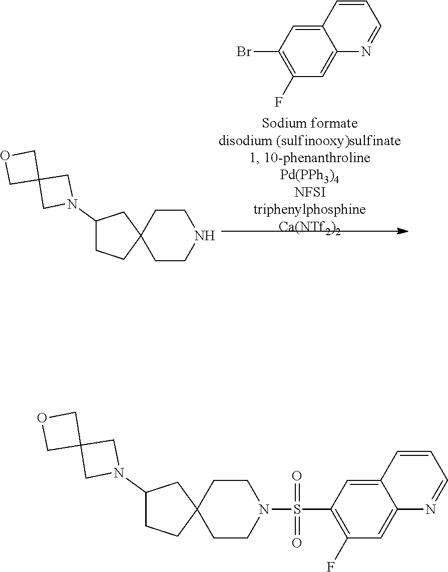 [SMILES: Fc1cc2ncccc2cc1Br ; C1CC2(CCN1)CCC(N1CC3(COC3)C1)C2 ; O=S(=O)(c1cc2cccnc2cc1F)N1CCC2(CCC(N3CC4(COC4)C3)C2)CC1]

Example 102 was prepared in a similar manner to Example 101, step 4 starting with 6-bromo-7-fluoro-quinoline to afford 6-(8-((7-fluoroquinolin-6-yl)sulfonyl)-8-azaspiro[4.5]decan-2-yl)-2-oxa-6-azaspiro[3.3]heptane (28 mg, 29%). LCMS m/z=446.2 (M+H)⁺. LCMS Rf (2 min)=0.86.

### Examples 103-111

The title compounds were prepared in a single step library on an approximately 25 mg target product scale using the following protocol.

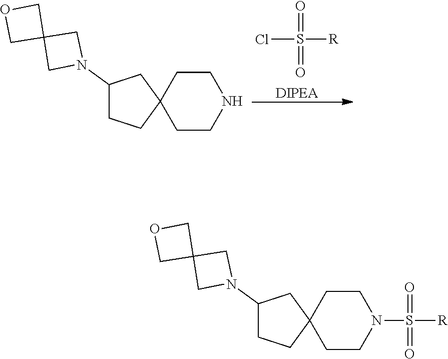

The appropriate sulfonyl chloride (1.1 equiv.) was added to a solution of 6-(8-azaspiro[4.5]decan-2-yl)-2-oxa-6-azaspiro[3.3]heptane (1.0 equiv.) and DIPEA (4.5 equiv.+1.1 equiv. per each acid equiv. for sulfonyl chloride building block salts) in dry MeCN (0.7 mL), and the reaction mixture was stirred at room temperature for 24 h. The solvent was evaporated in vacuo, and the residue was dissolved in DMSO (0.2 mL) and purified by prep. HPLC (Column: YMC Actus Trial C18 20×100 mm, 5 μm; Method water—MeOH—NH₃0.1% as a mobile phase) to afford pure product.

| Example No. | Name/Structure/Sulfonyl Chloride | Amount/Yield/Data |
|---|---|---|
| 103 | 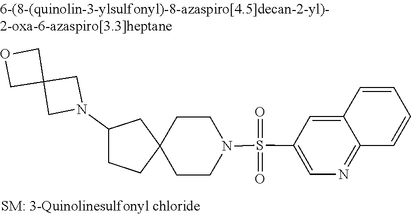 [SMILES: O=S(=O)(c1cnc2ccccc2c1)N1CCC2(CCC(N3CC4(COC4)C3)C2)CC1] | Amount: 2.2 mg Yield: 9% LCMS m/z = 428.2 (M + H)⁺. LCMS R_{f}(6 min) = 1.82 mins. |
| 104 | 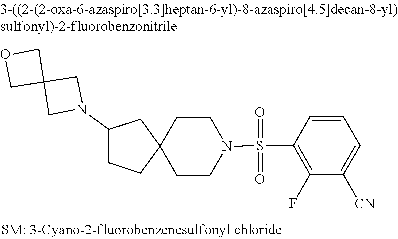 [SMILES: N#Cc1cccc(S(=O)(=O)N2CCC3(CCC(N4CC5(COC5)C4)C3)CC2)c1F] | Amount: 1.8 mg Yield: 7% LCMS m/z = 420.2 (M + H)⁺. LCMS R_{f}(2 min) = 0.61 mins. |
| 105 | 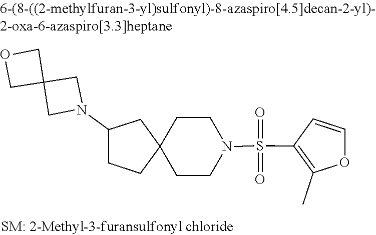 [SMILES: Cc1occc1S(=O)(=O)N1CCC2(CCC(N3CC4(COC4)C3)C2)CC1] | Amount: 3.4 mg Yield: 14% LCMS m/z = 381.2 (M + H)⁺. LCMS R_{f}(6 min) = 1.54 mins. |
| 106 | 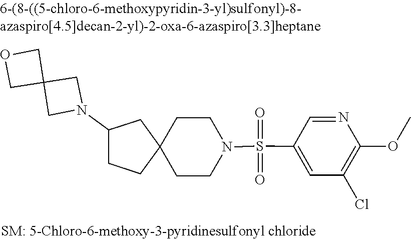 [SMILES: COc1ncc(S(=O)(=O)N2CCC3(CCC(N4CC5(COC5)C4)C3)CC2)cc1Cl] | Amount: 3.6 mg Yield: 14% LCMS m/z = 442.0 (M + H)⁺. LCMS R_{f}(2 min) = 0.78 mins. |
| 107 | 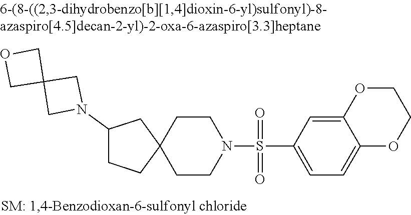 [SMILES: O=S(=O)(c1ccc2c(c1)OCCO2)N1CCC2(CCC(N3CC4(COC4)C3)C2)CC1] | Amount: 1.6 mg Yield: 6% LCMS m/z = 435.1 (M + H)⁺. LCMS R_{f}(2 min) = 0.89 mins. |
| 108 | 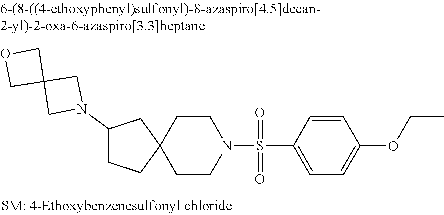 [SMILES: CCOc1ccc(S(=O)(=O)N2CCC3(CCC(N4CC5(COC5)C4)C3)CC2)cc1] | Amount: mg Yield: % LCMS m/z = 421.2 (M + H)⁺. LCMS R_{f}(2 min) = 0.96 mins. |
| 109 | 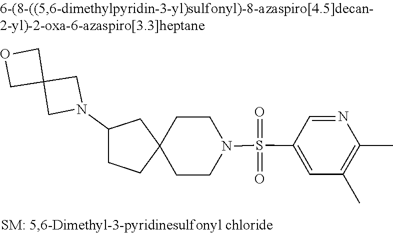 [SMILES: Cc1cc(S(=O)(=O)N2CCC3(CCC(N4CC5(COC5)C4)C3)CC2)cnc1C] | Amount: 4.4 mg Yield: 18% LCMS m/z = 406.2 (M + H)⁺. LCMS R_{f}(2 min) = 0.84 mins. |
| 110 | 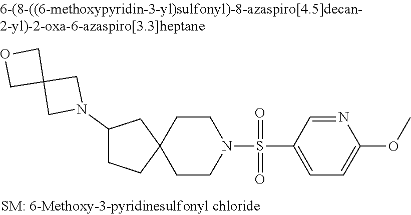 [SMILES: COc1ccc(S(=O)(=O)N2CCC3(CCC(N4CC5(COC5)C4)C3)CC2)cn1] | Amount: 6.4 mg Yield: 26% LCMS m/z = 408.1 (M + H)⁺. LCMS R_{f}(2 min) = 0.86 mins. |
| 111 | 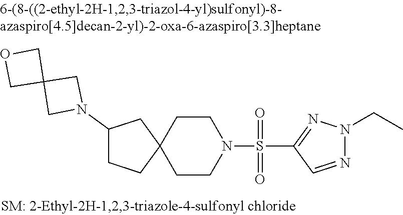 [SMILES: CCn1ncc(S(=O)(=O)N2CCC3(CCC(N4CC5(COC5)C4)C3)CC2)n1] | Amount: 4.7 mg Yield: 19% LCMS m/z = 396.1 (M + H)⁺. LCMS R_{f}(2 min) = 0.82 mins. |

### Examples 112-114

The title compounds were prepared in a single step library on an approximately 60 mg target product scale using the following protocol.

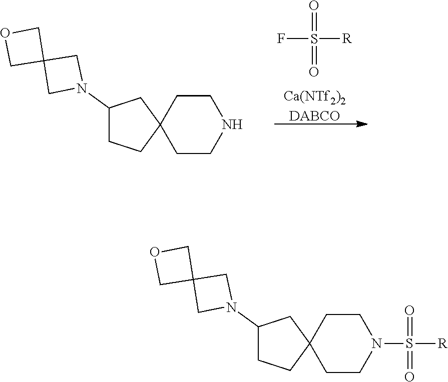

The appropriate sulfonyl fluoride (1.0 equiv.) was added to a solution of 6-(8-azaspiro[4.5]decan-2-yl)-2-oxa-6-azaspiro[3.3]heptane (1.0 equiv.), Ca(NTf₂)₂ (1.1 equiv.), and DABCO (1.5 equiv.) in dry THF. The reaction mixture was stirred at 60° C. for 16 h. The solvent was evaporated in vacuo, and the residue was dissolved in DMSO (0.5 mL) and purified by prep. HPLC (Column: YMC Actus Trial C18 20×100 mm, 5 μm; Method water—MeOH—NH₃ 0.1% as a mobile phase) to afford pure product.

| Example No. | Name/Structure/Sulfonyl Fluoride | Amount/Yield/Data |
|---|---|---|
| 112 | 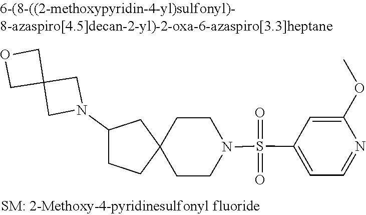 [SMILES: COc1cc(S(=O)(=O)N2CCC3(CCC(N4CC5(COC5)C4)C3)CC2)ccn1] | Amount: 10.6 mg Yield: 18% LCMS m/z = 408.2 (M + H)⁺ LCMS R_{f}(2 min) = 0.67 mins. |
| 113 | 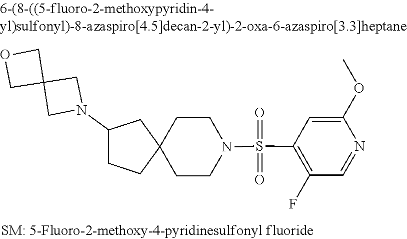 [SMILES: COc1cc(S(=O)(=O)N2CCC3(CCC(N4CC5(COC5)C4)C3)CC2)c(F)cn1] | Amount: 6.3 mg Yield: 11% LCMS m/z = 426.2 (M + H)⁺. LCMS R_{f}(2 min) = 0.91 mins. |
| 114 | 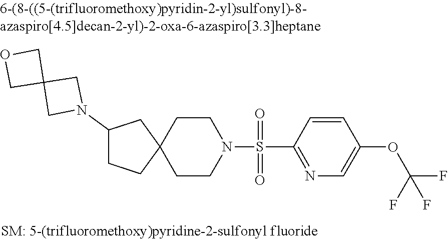 [SMILES: O=S(=O)(c1ccc(OC(F)(F)F)cn1)N1CCC2(CCC(N3CC4(COC4)C3)C2)CC1] | Amount: 1 mg Yield: 2% LCMS m/z = 462.2 (M + H)⁺. LCMS R_{f}(2 min) = 0.89 mins. |

### Example 114

The title compound was prepared in a single step library on an approximately 60 mg target product scale using the following protocol.

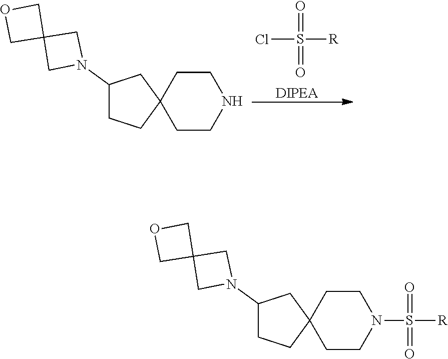

The appropriate sulfonyl chloride (1.1 equiv.) was added to a solution of 3-(2-oxa-6-azaspiro[3.3]heptan-6-yl)-1-oxa-8-azaspiro[4.5]decane as bis(trifluoroacetic acid) salt (1.0 equiv.) and DIPEA (6.0 equiv.) in dry ACN (1.2 mL), and the reaction mixture was stirred at room temperature for 24 h. The solids were filtered off, and the filtrate was concentrated under reduced pressure. The resulting residue was dissolved in DMSO (0.5 mL) then purified by prep HPLC (Waters SunFire C18 19×100 5 mm column; gradient mixture H₂O-MeOH-TFA 0.10% as a mobile phase or YMC Actus Trial C18 20×100 mm, 5 μm column; gradient mixture H₂O-MeOH-Ammonia 0.1% as a mobile phase) at an appropriate gradient to afford the desired product.

| Example No. | Name/Structure/Sulfonyl Chloride | Amount/Yield/Data |
|---|---|---|
| 114 | 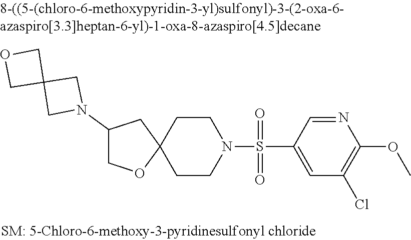 [SMILES: COc1ncc(S(=O)(=O)N2CCC3(CC2)CC(N2CC4(COC4)C2)CO3)cc1Cl] | Amount: 10.5 mg Yield: 17% LCMS m/z = 444.0 (M + H)⁺. LCMS R_{f}(2 min) = 0.94 mins. |

### Example 115

The title compound was prepared in a single step library on an approximately 60 mg target product scale using the following protocol.

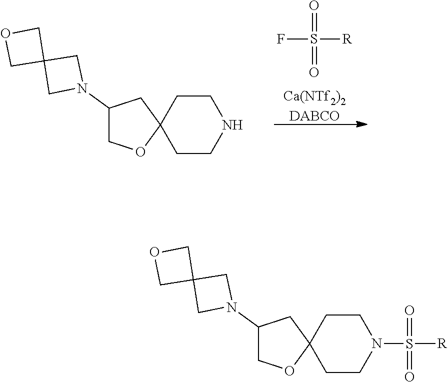

The appropriate sulfonyl fluoride (1.0 equiv.) was added to a solution of 3-(2-oxa-6-azaspiro[3.3]heptan-6-yl)-1-oxa-8-azaspiro[4.5]decane as bis(trifluoroacetic acid) salt (1.6 equiv.), Ca(NTf₂)₂ (1.2 equiv.), and DABCO (5 equiv.) in dry THF. The reaction mixture was stirred at 60° C. for 16 h. The solvent was evaporated in vacuo, and the residue was dissolved in DMSO (0.5 mL) and purified by prep. HPLC (Column: YMC Actus Trial C18 20×100 mm, 5 μm; Method water—MeOH—NH₃ 0.1% as a mobile phase) to afford pure product.

| Example No. | Name/Structure/Sulfonyl Fluoride | Amount/Yield/Data |
|---|---|---|
| 115 | 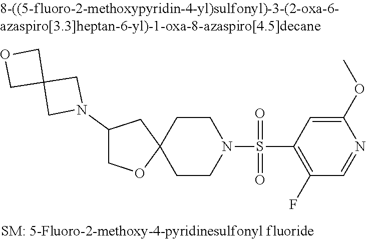 [SMILES: COc1cc(S(=O)(=O)N2CCC3(CC2)CC(N2CC4(COC4)C2)CO3)c(F)cn1] | Amount: 3 mg Yield: 5% LCMS m/z = 428.2 (M + H)⁺. LCMS R_{f}(2 min) = 0.83 mins. |

### Example 116: 8-((2,4-dimethylphenyl)sulfonyl)-N-(2-methoxyethyl)-1-oxa-8-azaspiro[4.5]decan-3-amine

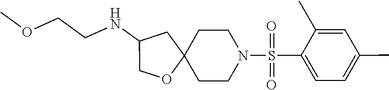

### 1. Synthesis of 8-((2,4-dimethylphenyl)sulfonyl)-1-oxa-8-azaspiro[4.5]decan-3-one

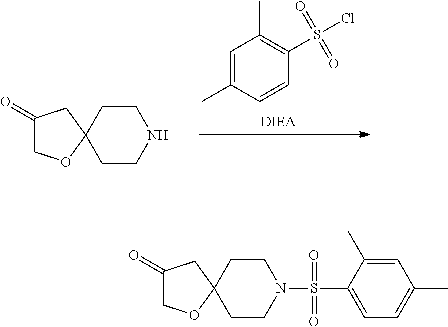 [SMILES: Cc1ccc(S(=O)(=O)Cl)c(C)c1 ; O=C1COC2(CCNCC2)C1 ; Cc1ccc(S(=O)(=O)N2CCC3(CC2)CC(=O)CO3)c(C)c1]

The synthesis was performed in a similar manner to that described in Example 56 step 1, using 1-oxa-8-azaspiro[4.5]decan-3-one hydrochloride and 2,4-dimethylbenzenesulfonyl chloride (260 mg, 73%). LCMS m/z=324.0 [M+H]⁺, ¹H NMR (CHLOROFORM-d, 400 MHz) δ (ppm) 7.80 (d, 1H, J=8.5 Hz), 7.1-7.2 (m, 2H), 3.98 (s, 2H), 3.3-3.6 (m, 2H), 3.0-3.2 (m, 2H), 2.60 (s, 3H), 2.38 (d, 5H, J=10.5 Hz), 1.8-1.9 (m, 4H).

### 2. Synthesis of 8-((2,4-dimethylphenyl)sulfonyl)-N-(2-methoxyethyl)-1-oxa-8-azaspiro[4.5]decan-3-amine

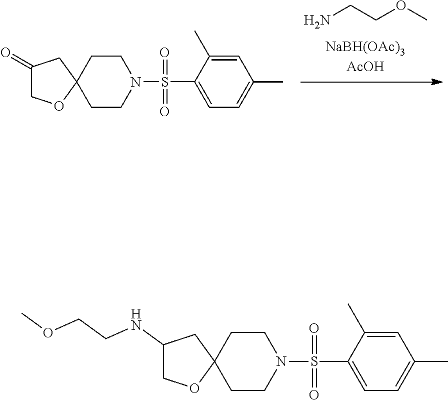

8-((2,4-dimethylphenyl)sulfonyl)-N-(2-methoxyethyl)-1-oxa-8-azaspiro[4.5]decan-3-amine was obtained (100 mg, 84%) from 2-methoxyethanamine and 8-((2,4-dimethylphenyl)sulfonyl)-1-oxa-8-azaspiro[4.5]decan-3-one, following a similar synthesis to that described in Example 56, step 2. LCMS m/z=382.2 [M+H]⁺, ¹H NMR (CHLOROFORM-d, 400 MHz) δ (ppm) 7.75 (d, 1H, J=8.8 Hz), 7.0-7.2 (m, 2H), 3.92 (dd, 1H, J=6.0, 9.0 Hz), 3.70 (q, 1H, J=7.0 Hz), 3.57 (dd, 1H, J=5.5, 9.0 Hz), 3.4-3.5 (m, 5H), 3.34 (s, 3H), 3.0-3.1 (m, 2H), 2.6-2.8 (m, 2H), 2.57 (s, 3H), 2.36 (s, 3H), 2.0-2.1 (m, 2H), 1.7-1.9 (m, 2H), 1.55 (dd, 1H, J=6.0, 12.8 Hz).

### Example 117: 8-((2-methoxy-5-methylpyridin-3-yl)sulfonyl)-3-(2-oxa-6-azaspiro[3.3]heptan-6-yl)-1-oxa-8-azaspiro[4.5]decane

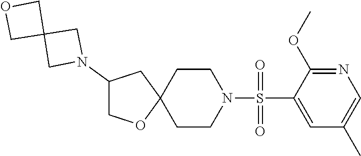 [SMILES: COc1ncc(C)cc1S(=O)(=O)N1CCC2(CC1)CC(N1CC3(COC3)C1)CO2]

8-((2-methoxy-5-methylpyridin-3-yl)sulfonyl)-3-(2-oxa-6-azaspiro[3.3]heptan-6-yl)-1-oxa-8-azaspiro[4.5]decane was obtained (150 mg, 80%) yield from 2-oxa-6-azaspiro[3.3]heptane and 8-((2-methoxy-5-methylpyridin-3-yl)sulfonyl)-1-oxa-8-azaspiro[4.5]decan-3-one, following a similar synthesis to that described in Example 56, step 2. LCMS m/z=424.2 [M+H]⁺, ¹H NMR (CHLOROFORM-d, 400 MHz) δ (ppm) 8.05 (dd, 1H, J=0.8, 2.3 Hz), 7.90 (d, 1H, J=2.5 Hz), 4.6-4.7 (m, 4H), 3.96 (s, 3H), 3.6-3.7 (m, 1H), 3.4-3.5 (m, 3H), 3.2-3.3 (m, 4H), 2.9-3.1 (m, 2H), 2.86 (tdd, 1H, J=3.9, 5.3, 7.3 Hz), 2.25 (s, 3H), 1.84 (br d, 1H, J=13.6 Hz), 1.5-1.7 (m, 4H), 1.45 (dd, 1H, J=4.0, 13.1 Hz).

### Example 118: rac-8-((3,5-difluorophenyl)sulfonyl)-3-(2-oxa-6-azaspiro[3.3]heptan-6-yl)-1-oxa-8-azaspiro[4.5]decane

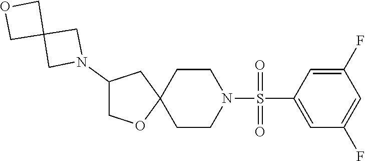 [SMILES: O=S(=O)(c1cc(F)cc(F)c1)N1CCC2(CC1)CC(N1CC3(COC3)C1)CO2]

### 1. Synthesis of rac-8-((2,4-dimethylphenyl)sulfonyl)-1-oxa-8-azaspiro[4.5]decan-3-one

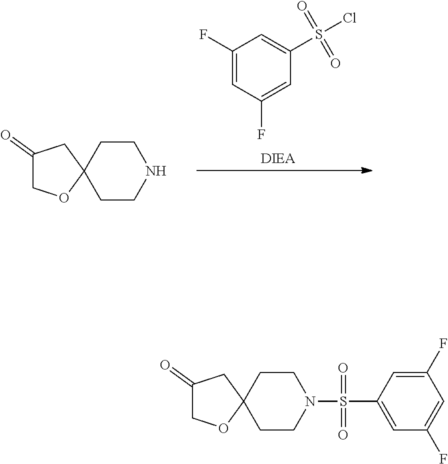

The synthesis was performed in a similar manner to that described in Example 56 step 1, using 1-oxa-8-azaspiro[4.5]decan-3-one hydrochloride and 2 3,5-difluorobenzenesulfonyl chloride (92 mg, 74%). LCMS m/z=332.0 [M+H]⁺, ¹H NMR (CHLOROFORM-d, 400 MHz) δ (ppm) 7.3-7.3 (m, 2H), 7.09 (tt, 1H, J=2.4, 8.4 Hz), 3.95 (s, 2H), 3.5-3.7 (m, 2H), 2.88 (dt, 2H, J=3.9, 11.2 Hz), 2.37 (s, 2H), 1.8-2.0 (m, 4H).

### 2. Synthesis of rac-8-((3,5-difluorophenyl)sulfonyl)-3-(2-oxa-6-azaspiro[3.3]heptan-6-yl)-1-oxa-8-azaspiro[4.5]decane

The synthesis was performed in a similar manner to that described in Example 56 step 2, using rac-8-((2,4-dimethylphenyl)sulfonyl)-1-oxa-8-azaspiro[4.5]decan-3-one and 2-oxa-6-azaspiro[3.3]heptane (92 mg, 74%). LCMS m/z=415.2 [M+H]⁺, R_{f}=0.56 min (Column: Waters ACQUITY UPLC BEH C18 2.1×30 mm, 1.7 μm, Modifier: Trifluoroacetic acid 0.1% (v/v) conc.
Method: 95% H₂O/5% MeCN (initial conditions) linear gradient to 5% H₂O/95% MeCN at 1.0 min, HOLD 5% H₂O/95% MeCN to 1.3 min. Flow rate, 0.7 mL/min).

### Examples 119a and 119b: (S)-8-((3,5-difluorophenyl)sulfonyl)-3-(2-oxa-6-azaspiro[3.3]heptan-6-yl)-1-oxa-8-azaspiro[4.5]decane and (R)-8-((3,5-difluorophenyl)sulfonyl)-3-(2-oxa-6-azaspiro[3.3]heptan-6-yl)-1-oxa-8-azaspiro[4.5]decane

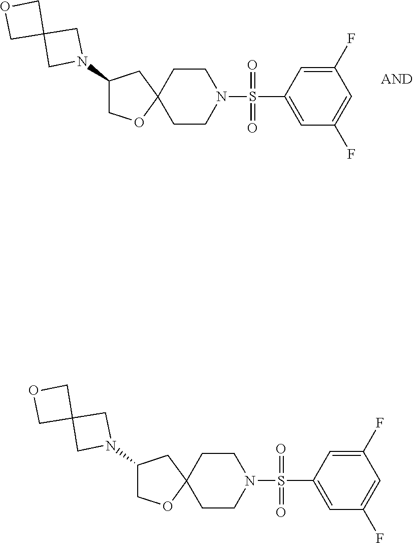 [SMILES: O=S(=O)(c1cc(F)cc(F)c1)N1CCC2(CC1)C[C@H](N1CC3(COC3)C1)CO2 ; O=S(=O)(c1cc(F)cc(F)c1)N1CCC2(CC1)C[C@@H](N1CC3(COC3)C1)CO2]

70 mg of rac-8-((3,5-difluorophenyl)sulfonyl)-3-(2-oxa-6-azaspiro[3.3]heptan-6-yl)-1-oxa-8-azaspiro[4.5]decane was dissolved in MeOH and purified on a CHIRALPAK AD-H 30×250 mm, 5 μm column using 20% methanol with 0.1% DEA in C₀₂. Flow rate: 100 mL/min; ABPR 120 bar; MBPR 40 psi, column temperature 40° C. to afford the following compounds that were concentrated to dryness then lyophilized:
Peak 1 or Example 119a was a white solid arbitrarily assigned as (S)-8-((3,5-difluorophenyl)sulfonyl)-3-(2-oxa-6-azaspiro[3.3]heptan-6-yl)-1-oxa-8-azaspiro[4.5]decane (20.1 mg). LCMS m/z=415.2 (M+H)⁺. R_{f}=1.99 min.
Peak 2 or Example 119b was a white solid arbitrarily assigned as (R)-8-((3,5-difluorophenyl)sulfonyl)-3-(2-oxa-6-azaspiro[3.3]heptan-6-yl)-1-oxa-8-azaspiro[4.5]decane (23.5 mg). LCMS m/z=415.2 (M+H)⁺. R_{f}=2.16 min.

### Example 120: 7-((3-cyclopropyl-1-methyl-1H-pyrazol-5-yl)sulfonyl)-N-(tetrahydro-2H-pyran-4-yl)-7-azaspiro[3.5]nonan-2-amine

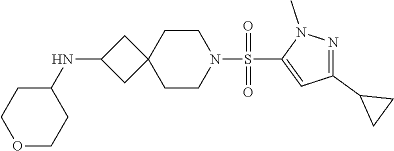 [SMILES: Cn1nc(C2CC2)cc1S(=O)(=O)N1CCC2(CC1)CC(NC1CCOCC1)C2]

### 1. Synthesis of 7-((3-cyclopropyl-1-methyl-1H-pyrazol-5-yl)sulfonyl)-7-azaspiro[3.5]nonan-2-one

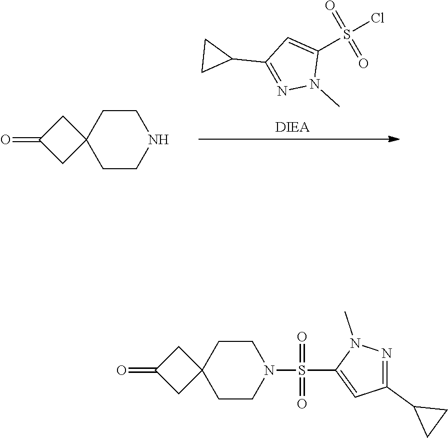

The synthesis was performed in a similar manner to that described in Example 93 step 1, using 7-azaspiro[3.5]nonan-2-one and 3-cyclopropyl-1-methyl-1H-pyrazole-5-sulfonyl chloride (194 mg, 87%). LCMS m/z=324.0 [M+H]⁺, ¹H NMR (CHLOROFORM-d, 400 MHz) δ (ppm) 7.80 (d, 1H, J=8.5 Hz), 7.1-7.2 (m, 2H), 3.98 (s, 2H), 3.3-3.6 (m, 2H), 3.0-3.2 (m, 2H), 2.60 (s, 3H), 2.38 (d, 5H, J=10.5 Hz), 1.8-1.9 (m, 4H).

### 2. Synthesis of 7-((3-cyclopropyl-1-methyl-1H-pyrazol-5-yl)sulfonyl)-N-(tetrahydro-2H-pyran-4-yl)-7-azaspiro[3.5]nonan-2-amine

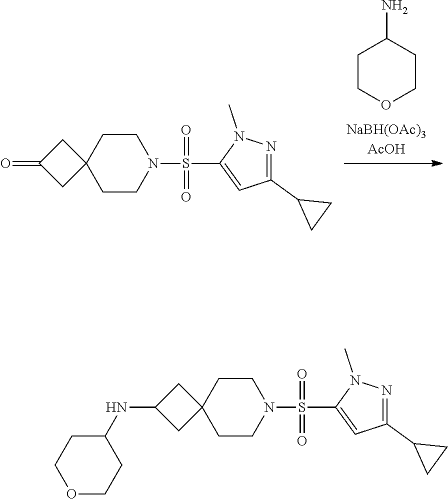 [SMILES: Cn1nc(C2CC2)cc1S(=O)(=O)N1CCC2(CC1)CC(=O)C2 ; NC1CCOCC1 ; Cn1nc(C2CC2)cc1S(=O)(=O)N1CCC2(CC1)CC(NC1CCOCC1)C2 ; "NaBH(OAc)3AcOH"]

The synthesis was performed in a similar manner to that described in Example 93 step 2, using 7-((3-cyclopropyl-1-methyl-1H-pyrazol-5-yl)sulfonyl)-7-azaspiro[3.5]nonan-2-one and tetrahydro-2H-pyran-4-amine (94 mg, 78%). LCMS m/z=409.3 [M+H]⁺, ¹H NMR (CHLOROFORM-d, 500 MHz) δ (ppm) 6.34 (s, 1H), 4.00 (s, 3H), 3.95 (br d, 2H, J=11.3 Hz), 3.3-3.4 (m, 3H), 3.1-3.2 (m, 2H), 3.0-3.1 (m, 2H), 2.6-2.7 (m, 1H), 2.16 (ddd, 2H, J=2.4, 7.8, 9.9 Hz), 1.8-1.9 (m, 1H), 1.6-1.8 (m, 6H), 1.4-1.5 (m, 4H), 0.9-1.0 (m, 4H), 0.7-0.8 (m, 4H).

### Example 121: 6-(7-((6-(difluoromethoxy)-2-methylpyridin-3-yl)sulfonyl)-7-azaspiro[3.5]nonan-2-yl)-2-oxa-6-azaspiro[3.3]heptane

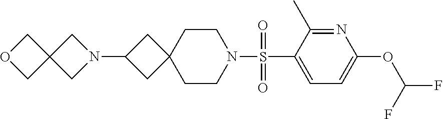 [SMILES: Cc1nc(OC(F)F)ccc1S(=O)(=O)N1CCC2(CC1)CC(N1CC3(COC3)C1)C2]

### 1. Synthesis of 7-((6-(difluoromethoxy)-2-methylpyridin-3-yl)sulfonyl)-7-azaspiro[3.5]nonan-2-one

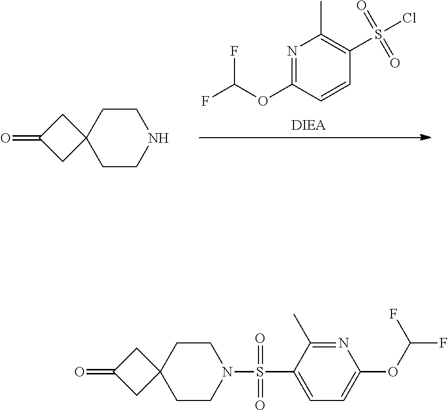

The synthesis was performed in a similar manner to that described in Example 91 step 1, using 7-azaspiro[3.5]nonan-2-one and 6-(difluoromethoxy)-2-methylpyridine-3-sulfonyl chloride (90 mg, 31%). LCMS m/z=360.9 [M+H]⁺, TH NMR (400 MHz, CDCl₃) δ (ppm) 8.21 (d, J=8.4 Hz, 1H), 7.76-7.33 (m, 1H), 6.84 (d, J=8.4 Hz, 1H), 3.24-3.18 (m, 4H), 2.83-2.80 (m, 4H), 2.78-2.73 (m, 3H), 1.87-1.82 (m, 4H).

### 2. Synthesis of 6-(7-((6-(difluoromethoxy)-2-methylpyridin-3-yl)sulfonyl)-7-azaspiro[3.5]nonan-2-yl)-2-oxa-6-azaspiro[3.3]heptane

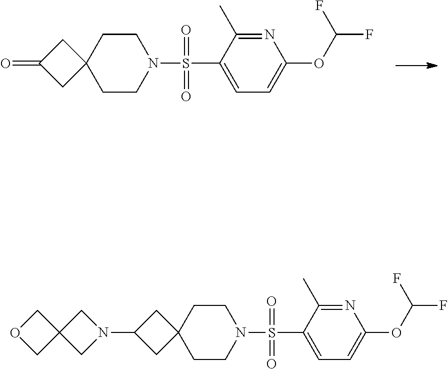 [SMILES: Cc1nc(OC(F)F)ccc1S(=O)(=O)N1CCC2(CC1)CC(=O)C2 ; Cc1nc(OC(F)F)ccc1S(=O)(=O)N1CCC2(CC1)CC(N1CC3(COC3)C1)C2]

The synthesis was performed in a similar manner to that described in Example 91 step 2, using 7-((6-(difluoromethoxy)-2-methylpyridin-3-yl)sulfonyl)-7-azaspiro[3.5]nonan-2-one and 2-oxa-6-azaspiro[3.3]heptane (88 mg, 28%). LCMS m/z=444.1 [M+H]⁺, TH NMR: (400 MHz, MeOD) δ (ppm) 8.25 (d, J=8.0 Hz, 1H), 7.88-7.41 (m, 1H), 6.97 (d, J=8.0 Hz, 1H), 4.72 (s, 4H), 3.35 (s, 4H), 3.18-3.13 (m, 2H), 3.12-3.05 (m, 3H), 2.75 (s, 3H), 1.96-1.87 (m, 2H), 1.71-1.64 (m, 2H), 1.64-1.60 (m, 2H), 1.59-1.51 (m, 2H).

### Example 122: 6-(7-((3-(difluoromethyl)-1-methyl-1H-pyrazol-5-yl)sulfonyl)-7-azaspiro[3.5]nonan-2-yl)-2-oxa-6-azaspiro[3.3]heptane

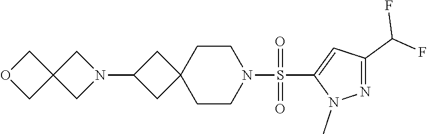 [SMILES: Cn1nc(C(F)F)cc1S(=O)(=O)N1CCC2(CC1)CC(N1CC3(COC3)C1)C2]

### 1. Synthesis of 6-(7-((3-(difluoromethyl)-1-methyl-H-pyrazol-5-yl)sulfonyl)-7-azaspiro[3.5]nonan-2-yl)-2-oxa-6-azaspiro[3.3]heptane

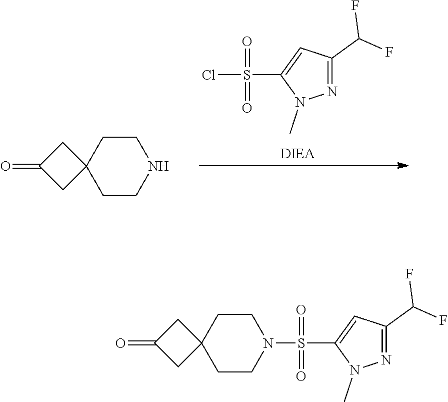 [SMILES: Cn1nc(C(F)F)cc1S(=O)(=O)Cl ; O=C1CC2(CCNCC2)C1 ; Cn1nc(C(F)F)cc1S(=O)(=O)N1CCC2(CC1)CC(=O)C2]

To a solution of 7-azaspiro[3.5]nonan-2-one (150 mg, 853.94 umol, Hydrochloride) and DIPEA (331.09 mg, 2.56 mmol, 446.21 uL) in DCM (6 mL) was added 7-[5-(difluoromethyl)-2-methyl-pyrazol-3-yl]sulfonyl-7-azaspiro[3.5]nonan-2-one (240 mg, 719.96 umol) at 0-5° C. The reaction mixture was stirred at 15° C. for 2 h. The reaction was diluted with water (15 mL), extracted with DCM (25 mL×3). The combined organic phase was washed with brine (15 mL×2), dried over anhydrous sodium sulfate, filtered and concentrated in vacuum. The crude material was purified by flash column (EtOAc in petroleum ether=10%˜40%) to give the desired compound (240 mg, 84%) as a colorless oil. LCMS m/z=334.1 [M+H]⁺. ¹H NMR (400 MHz, CDCl₃) δ (ppm) 6.88 (s, 1H), 6.81-6.52 (m, 1H), 4.16-4.09 (m, 1H), 4.12 (s, 2H), 3.33-3.19 (m, 4H), 2.83 (s, 4H), 1.99-1.81 (m, 4H).

### 2. Synthesis of 6-(7-((3-(difluoromethyl)-1-methyl-H-pyrazol-5-yl)sulfonyl)-7-azaspiro[3.5]nonan-2-yl)-2-oxa-6-azaspiro[3.3]heptane

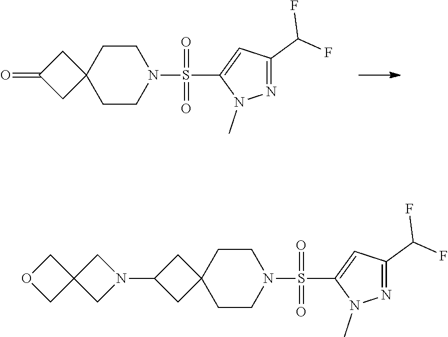 [SMILES: Cn1nc(C(F)F)cc1S(=O)(=O)N1CCC2(CC1)CC(=O)C2 ; Cn1nc(C(F)F)cc1S(=O)(=O)N1CCC2(CC1)CC(N1CC3(COC3)C1)C2]

The synthesis was performed in a similar manner to that described in Example 91 step 2, using 6-(7-((3-(difluoromethyl)-1-methyl-1H-pyrazol-5-yl)sulfonyl)-7-azaspiro[3.5]nonan-2-yl)-2-oxa-6-azaspiro[3.3]heptane and 2-oxa-6-azaspiro[3.3]heptane (150 mg, 59%). LCMS m/z=417.2 [M+H]⁺. ¹H NMR (400 MHz, CDCl₃) δ (ppm) 6.84 (s, 1H), 6.80-6.51 (m, 1H), 4.71 (s, 4H), 4.10 (s, 3H), 3.26 (s, 4H), 3.18-3.05 (m, 4H), 3.00-2.90 (m, 1H), 1.89-1.77 (m, 2H), 1.69-1.64 (m, 4H), 1.58-1.47 (m, 2H).

### Example 123: 6-(7-((1-cyclopropyl-3-(difluoromethyl)-1H-pyrazol-5-yl)sulfonyl)-7-azaspiro[3.5]nonan-2-yl)-2-oxa-6-azaspiro[3.3]heptane

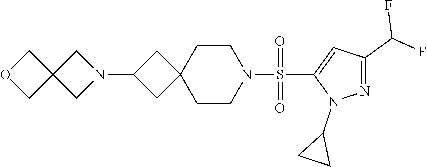 [SMILES: O=S(=O)(c1cc(C(F)F)nn1C1CC1)N1CCC2(CC1)CC(N1CC3(COC3)C1)C2]

### 1. Synthesis of 1-cyclopropyl-1H-pyrazole-3-carbaldehyde

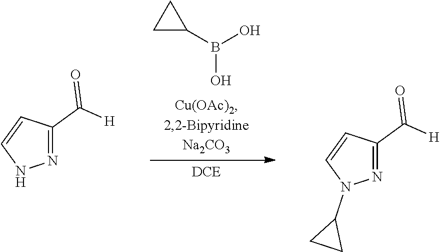 [SMILES: O=Cc1cc[nH]n1 ; OB(O)C1CC1 ; O=Cc1ccn(C2CC2)n1]

To a stirred solution of 1H-pyrazole-3-carbaldehyde (5 g, 52.04 mmol), cyclopropylboronic acid (8.94 g, 104.07 mmol) and Na₂CO₃ (11.03 g, 104.07 mmol) in DCE (150 mL) was stirred at 70° C. for 30 min, and the mixture was added a solution of 2-(2-pyridyl)pyridine (8.13 g, 52.04 mmol) and Cu(OAc)₂ (9.45 g, 52.04 mmol) in DCE (50 mL) at 15° C. under air. Then the reaction was heated to 70° C. for 4 h. The reaction was cooled to 15° C. and 5 mL AcOH was added. The reaction mixture was concentrated in vacuo and the residue diluted with H₂O (100 mL) and extracted with EtOAc (100 mL×3). The organic layer was washed with 1M HCl and dried over Na₂SO₄, filtered and concentrated in vacuo. The residue was purified by column chromatography (SiO₂, Petroleum ether/Ethyl acetate=100/0 to 85/15) to give the desired compound (2.55 g, 32%) as a yellow oil. R_{f}=0.46, ¹H NMR (400 MHz, CDCl₃) δ (ppm) 9.93 (s, 1H), 7.50 (d, J=2.4 Hz, 1H), 6.75 (d, J=2.4 Hz, 1H), 3.75-3.59 (m, 1H), 1.23-1.18 (m, 2H), 1.12-1.06 (m, 2H).

### 2. Synthesis of 1-cyclopropyl-3-(difluoromethyl)-1H-pyrazole

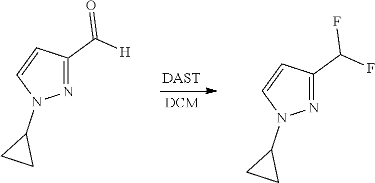

To a solution of 1-cyclopropyl-1H-pyrazole-3-carbaldehyde (5 g, 33.05 mmol, 90% purity) in DCM (180 mL) was added DAST (15.98 g, 99.15 mmol, 13.10 mL) at −30° C. The mixture was stirred at 20° C. for 12 hr. The reaction mixture was quenched by saturated NH₄Cl (50 mL) at 15° C., and then diluted with H₂O (50 mL) and extracted with DCM (200 mL×3). The combined organic layers were dried over Na₂SO₄, filtered, and concentrated under reduced pressure to give a residue. The residue was purified by column chromatography (SiO₂, Petroleum ether/Ethyl acetate=100/1 to 83/17) to give the desired compound (4.9 g, 84% yield) as colorless oil. LCMS m/z=159.1 [M+H]⁺. ¹H NMR (400 MHz, CDCl₃) δ (ppm) 7.47 (d, J=2.4 Hz, 1H), 6.65 (t, J=55.2 Hz, 1H), 6.43 (d, J=1.2 Hz, 1H), 3.64-3.57 (m, 1H), 1.16-1.10 (m, 2H), 1.08-1.01 (m, 2H).

### 3. Synthesis of 5-bromo-1-cyclopropyl-3-(difluoromethyl)-1H-pyrazole

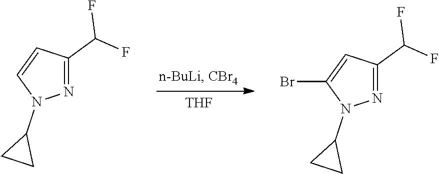

To a solution of 1-cyclopropyl-3-(difluoromethyl)-1H-pyrazole (4.9 g, 27.89 mmol, 90% purity) in THE (50 mL) was added n-BuLi (2.5 M, 16.73 mL, 1.5 eq) at −70° C. over 0.5 hr. CBr₄ (12.02 g, 36.25 mmol, 1.3 eq) in THE (2 mL) was carefully added dropwise to the reaction mixture. The mixture was stirred at 25° C. for 1.5 hr under N₂. The reaction mixture was quenched with a saturated solution of NH₄Cl (30 mL) at 0° C., and allowed to warm to 25° C. The reaction mixture was diluted with H₂O (50 mL) and extracted with EtOAc (100 mL×3). The combined organic layers were dried over Na₂SO₄, filtered, and concentrated under reduced pressure to give a residue, which was purified by column chromatography (SiO₂, Petroleum ether/Ethyl acetate=100/1 to 80/20) to give the desired compound (2.5 g, 34%) as yellow oil. LCMS: m/z=236.7 [M+H]⁺. ¹H NMR (400 MHz, CDCl₃) δ (ppm) 6.73-6.41 (m, 2H), 3.52-3.46 (m Hz, 1H), 1.23-1.19 (m, 2H), 1.14-1.08 (m, 2H).

### 4. Synthesis of 5-(benzylthio)-1-cyclopropyl-3-(difluoromethyl)-1H-pyrazole

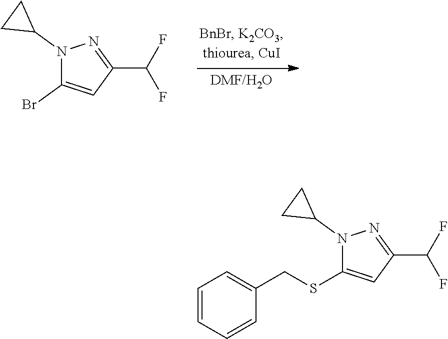 [SMILES: FC(F)c1cc(Br)n(C2CC2)n1 ; "BnBr, K2CO3,thiourea, CuI" ; "DMF/H2O" ; FC(F)c1cc(SCc2ccccc2)n(C2CC2)n1]

To a solution of benzyl bromide (4.87 g, 28.48 mmol, 3.38 mL), K₂CO₃ (13.12 g, 94.92 mmol), CuI (180.77 mg, 949.19 umol) and thiourea (2.89 g, 37.97 mmol) in DMF (50 mL) and H₂O (2 mL). 5-bromo-1-cyclopropyl-3-(difluoromethyl)-1H-pyrazole (2.5 g, 9.49 mmol) was added and the mixture was stirred at 100° C. for 12 hr. The reaction mixture was diluted with H₂O (100 mL) and extracted with EtOAc (100 mL×3). The combined organic layers were washed with brine (200 mL), dried over Na₂SO₄, filtered and concentrated under reduced pressure to give a residue, which was purified by flash silica gel chromatography (ISCO®; 20 g SepaFlash® Silica Flash Column, Eluent of 0˜10% Ethyl acetate/Petroleum ether gradient @25 mL/min) to give the desired compound (290 mg, 10%) as a yellow oil. LCMS m/z=281.9[M+H]⁺. ¹H NMR (400 MHz, CDCl₃) δ (ppm) 7.31-7.27 (m, 3H), 7.21-7.17 (m, 2H), 6.72-6.41 (m, 2H), 4.02 (s, 2H), 3.47-3.39 (m, 1H), 1.14-1.08 (m, 2H), 0.96-0.91 (m, 2H).

### 5. Synthesis of 1-cyclopropyl-3-(difluoromethyl)-1H-pyrazole-5-sulfonyl chloride

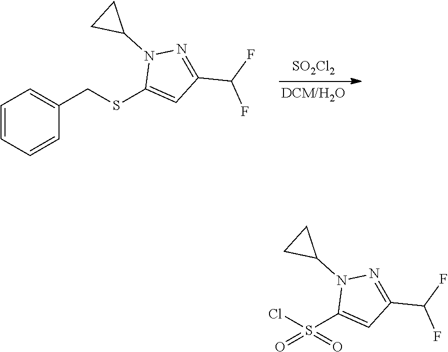

To a solution of 5-(benzylthio)-1-cyclopropyl-3-(difluoromethyl)-1H-pyrazole (290 mg, 931.03 umol, 90% purity) in DCM (10 mL) and H₂O (1 mL) was added dropwise SO₂Cl₂ (879.62 mg, 6.52 mmol, 651.57 uL) in DCM (0.5 mL). The mixture was stirred at 0° C. for 1 hr. The reaction mixture was diluted with H₂O (20 mL) and extracted with EtOAc (20 mL×3). The combined organic layers were dried over Na₂SO₄, filtered, and concentrated under reduced pressure to give the desired compound (230 mg, crude) as yellowish oil which was used without purification.

### 6. Synthesis of 7-((I-cyclopropyl-3-(difluoromethyl)-1H-pyrazol-5-yl)sulfonyl)-7-azaspiro[3.5]nonan-2-one

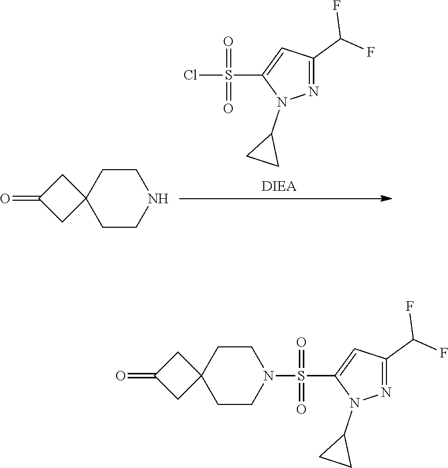 [SMILES: O=S(=O)(Cl)c1cc(C(F)F)nn1C1CC1 ; O=C1CC2(CCNCC2)C1 ; O=C1CC2(CCN(S(=O)(=O)c3cc(C(F)F)nn3C3CC3)CC2)C1]

The synthesis was performed in a similar manner to that described in Example 122, step 1, using 7-azaspiro[3.5]nonan-2-one and 1-cyclopropyl-3-(difluoromethyl)-1H-pyrazole-5-sulfonyl chloride (10 mg, 7%). LCMS m/z=360.0 [M+H]⁺. ¹H NMR (500 MHz, CHLOROFORM-d) δ (ppm) 6.81 (s, 1H), 6.56 (t, J=68.5 Hz, 1H), 4.11-3.98 (m, 1H), 3.30-3.16 (m, 4H), 2.77 (s, 4H), 1.86-1.76 (m, 4H), 1.34-1.27 (m, 2H), 1.08-0.98 (m, 2H).

### 7. Synthesis of 6-(7-((I-cyclopropyl-3-(difluoromethyl)-H-pyrazol-5-yl)sulfonyl)-7-azaspiro[3.5]nonan-2-yl)-2-oxa-6-azaspiro[3.3]heptane

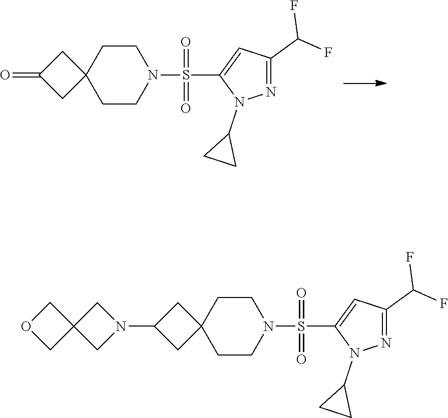 [SMILES: O=C1CC2(CCN(S(=O)(=O)c3cc(C(F)F)nn3C3CC3)CC2)C1 ; O=S(=O)(c1cc(C(F)F)nn1C1CC1)N1CCC2(CC1)CC(N1CC3(COC3)C1)C2]

The synthesis was performed in a similar manner to that described in Example 91 step 2, using 7-((1-cyclopropyl-3-(difluoromethyl)-1H-pyrazol-5-yl)sulfonyl)-7-azaspiro[3.5]nonan-2-one and 2-oxa-6-azaspiro[3.3]heptane (7 mg, 78%). LCMS m/z=443.2 [M+H]⁺. ¹H NMR (400 MHz, MeOD) δ (ppm) 8.56-8.28 (m, 0.5H), 6.95 (s, 1H), 6.72 (t, J=54.8 Hz, 1H), 4.76 (s, 4H), 4.27-4.13 (m, 1H), 4.02-3.83 (m, 4H), 3.63-3.50 (m, 1H), 3.29-3.13 (m, 4H), 2.18-2.06 (m, 2H), 1.83-1.62 (m, 6H), 1.36-1.29 (m, 2H), 1.14-1.03 (m, 2H).

### Example 124: 6-(8-((3-methoxy-1-methyl-1H-pyrazol-5-yl)sulfonyl)-8-azaspiro[4.5]decan-2-yl)-2-oxa-6-azaspiro[3.3]heptane

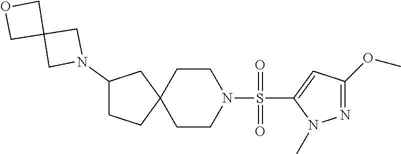 [SMILES: COc1cc(S(=O)(=O)N2CCC3(CCC(N4CC5(COC5)C4)C3)CC2)n(C)n1]

### 1. Synthesis of 3-methoxy-1-methyl-1H-pyrazole

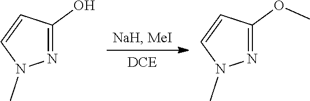

To a solution of 1-methyl-1H-pyrazol-3-ol (10 g, 101.93 mmol) in THE (300 mL) was added NaH (6.12 g, 152.90 mmol, 60% purity) at 0° C. After 30 min, Mel (36.17 g, 254.83 mmol, 15.86 mL) was added the mixture was stirred at 25° C. for 16 h under N₂. The mixture was quenched with H₂O (20 mL) and extracted with DCM (20 mL×2). The combined organic phase was washed with brine (20 mL×2), dried over Na₂SO₄ and filtered. The filtrate was concentrated under reduced pressure and the residue was purified by flash column (EtOAc in Petroleum ether from 0% to 30%) to give the desired compound (4.30 g, 37%) as colorless oil. ¹H NMR (500 MHz, CDCl₃), δ (ppm) 7.10 (d, J=2.5 Hz, 1H), 5.59 (d, J=2.5 Hz, 1H), 3.86 (s, 3H), 3.72 (s, 3H).

### 2. Synthesis of lithium 3-methoxy-1-methyl-1H-pyrazole-5-sulfinate

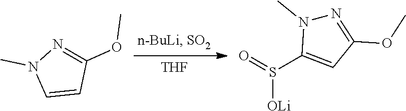

To a solution of 3-methoxy-1-methyl-1H-pyrazole (500 mg, 4.46 mmol) in THE (10 mL) was added n-BuLi (2.5 M, 2.68 mL) dropwise over 5 min at −65° C. under N₂. The mixture was then stirred at 0° C. for 1 h and cooled to −70° C. Excess SO₂ was bubbled into THE (2 mL) and then added into the mixture through a syringe slowly over 3 min, while maintaining the temperature below −65° C. The reaction was stirred at −65° C. for 1 h and was then allowed to warm to 25° C. The mixture was concentrated and the residue was triturated in petroleum ether (60 mL) and filtered. The filter cake was dried in vacuum to give the desired compound (900 mg, crude) as yellow solid. ¹H NMR (400 MHz, DMSO-d₆) δ (ppm) 5.45 (s, 1H), 3.71 (s, 3H), 3.67 (s, 3H).

### 3. Synthesis of 8-((3-methoxy-1-methyl-1H-pyrazol-5-yl)sulfonyl)-8-azaspiro[4.5]decan-2-one

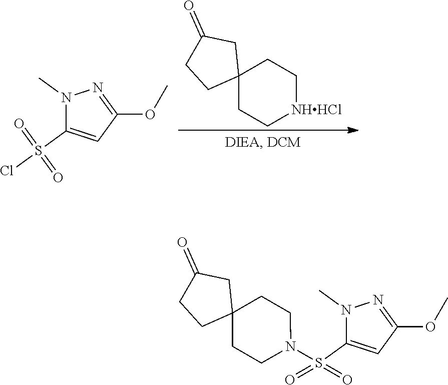

To a solution of 8-azaspiro[4.5]decan-2-one hydrochloride (375.21 mg, 1.98 mmol, HCl salt), DIEA (766.95 mg, 5.93 mmol, 1.03 mL) in DCM (6 mL) was added lithium 3-methoxy-1-methyl-1H-pyrazole-5-sulfinate (500 mg, 2.37 mmol) at 0˜5° C. Then the mixture was stirred at 25° C. for 1 h. The combined mixture was concentrated in vacuum. The crude was purified by flash column (MeOH in DCM from 0%˜6%) to give the desired compound (700 mg, 88%) as a yellow oil. LCMS m/z=328.1 [M+H]⁺. TH NMR (400 MHz, CDCl₃) δ (ppm) 6.05 (s, 1H), 3.93 (s, 3H), 3.88 (s, 3H), 3.40-3.34 (m, 2H), 3.02-2.96 (m, 2H), 2.29 (t, J=8.0 Hz, 2H), 2.10 (s, 2H), 1.84 (t, J=8.0 Hz, 2H), 1.72-1.66 (m, 4H).

### 4. Synthesis of 6-(8-((3-methoxy-1-methyl-1H-pyrazol-5-yl)sulfonyl)-8-azaspiro[4.5]decan-2-yl)-2-oxa-6-azaspiro[3.3]heptane

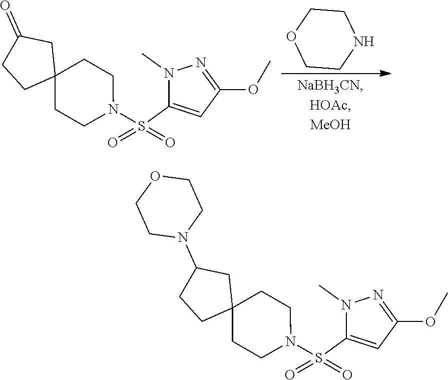

A solution of 8-((3-methoxy-1-methyl-1H-pyrazol-5-yl)sulfonyl)-8-azaspiro[4.5]decan-2-one 300 mg, 916.31 μmol) and morpholine (119.74 mg, 1.37 mmol, 120.23 μL) in MeOH (10 mL) was adjusted pH 5-6 by HOAc at 25-30° C. and was stirred at 25-30° C. for 1 h. NaBH₃CN (172.75 mg, 2.75 mmol) was added and the mixture was stirred at 25-30° C. for 1 h. The mixture was quenched with water (20 mL) and adjusted pH 7-8 by saturated aqueous NaHCO₃ (30 mL). The reaction mixture was extracted with DCM (50 mL×2), the combined organic layers were washed with brine (50 mL), dried over Na₂SO₄, filtered, and concentrated under reduced pressure. The residue was purified by prep-HPLC (Boston Green ODS 150*30 mm*5 μm, Condition: water (NH₄HCO₃)−ACN, Flow Rate (ml/min) 25) to give the desired compound (150 mg, 40%) as a colorless oil. LCMS m/z=399.4 [M+H]⁺. TH NMR (400 MHz, CDCl₃) δ (ppm) 6.01 (s, 1H), 3.89 (s, 3H), 3.84 (s, 3H), 3.67-3.65 (m, 4H), 3.10-3.08 (m, 4H), 2.54-2.46 (m, 1H), 2.40 (br s, 4H), 1.87-1.82 (m, 1H), 1.74-1.69 (m, 1H), 1.64-1.56 (m, 1H), 1.55-1.48 (m, 4H), 1.47-1.37 (m, 2H) 1.25-1.20 (m, 1H).

### Example 125: 6-(8-((3-(difluoromethyl)-1-methyl-1H-pyrazol-5-yl)sulfonyl)-8-azaspiro[4.5]decan-2-yl)-2-oxa-6-azaspiro[3.3]heptane

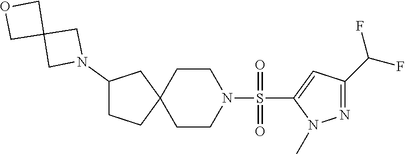 [SMILES: Cn1nc(C(F)F)cc1S(=O)(=O)N1CCC2(CCC(N3CC4(COC4)C3)C2)CC1]

### 1. Synthesis of 3-(difluoromethyl)-1-methyl-1H-pyrazole

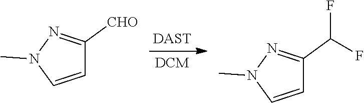

To a −20° C. solution of 1-methyl-1H-pyrazole-3-carbaldehyde (7 g, 63.57 mmol) in DCM (100 mL) was added DAST (30.74 g, 190.71 mmol, 25.20 mL) dropwise over 5 min and the reaction mixture was then stirred for 2 h at 15° C. The mixture was quenched with saturated aqueous NaHCO₃ (40 mL), extracted with DCM (30 mL×2). The combined organic phase was washed with brine (80 mL×2), dried over anhydrous sodium sulfate, filtered, and concentrated in vacuum to give the desired compound (6.5 g, crude) as a brown oil. ¹H NMR (400 MHz, CDCl₃) δ (ppm) 7.38 (d, J=2.0 Hz, 1H), 6.67 (t, J=55.2 Hz, 1H), 6.48-6.42 (m, 1H), 3.92 (s, 3H).

### 2. Synthesis of lithium 3-(difluoromethyl)-1-methyl-1H-pyrazole-5-sulfinate

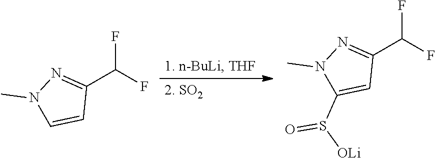

To a −40˜−50° C. solution of 3-(difluoromethyl)-1-methyl-1H-pyrazole (3 g, 22.71 mmol) in THF (60 mL) was added n-BuLi (2.5 M, 14.53 mL) dropwise over 10 min under nitrogen and the mixture was then stirred at −40˜−50° C. for 1 h. Excess SO₂ was bubbled into a solution THF (10 mL) for 10 min and then added into the above solution at −50° C. The mixture was concentrated in vacuum to give the desired compound (4.5 g, crude) as brown solid. ¹H NMR (400 MHz, DMSO-d₆) δ (ppm) 6.87 (t, J=55.2 Hz, 1H), 6.29 (s, 1H), 3.91 (s, 3H).

### 3. Synthesis of 3-(difluoromethyl)-1-methyl-1H-pyrazole-5-sulfonyl chloride

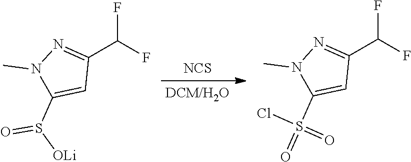

To a 0-5° C. solution of lithium 3-(difluoromethyl)-1-methyl-1H-pyrazole-5-sulfinate (4.5 g, 22.27 mmol) in DCM (35 mL) and water (35 mL) was added NCS (4.46 g, 33.40 mmol) under nitrogen and the mixture was then stirred at 0-5° C. for 1 h. The mixture was diluted with water (10 mL), extracted with DCM (15 mL×3). The combined organic phase was dried over anhydrous sodium sulfate, filtered, and concentrated in vacuum. The crude was purified by flash column (EtOAc in petroleum ether=0%˜10%) to give the desired compound (1.26 g, 24%) as yellow oil. H NMR (400 MHz, CDCl₃) δ (ppm) 7.22 (s, 1H), 6.69 (t, J=54.4 Hz, 1H), 4.24 (s, 3H).

### 4. Synthesis of 8-((3-(difluoromethyl)-1-methyl-H-pyrazol-5-yl)sulfonyl)-8-azaspiro[4.5]decan-2-one

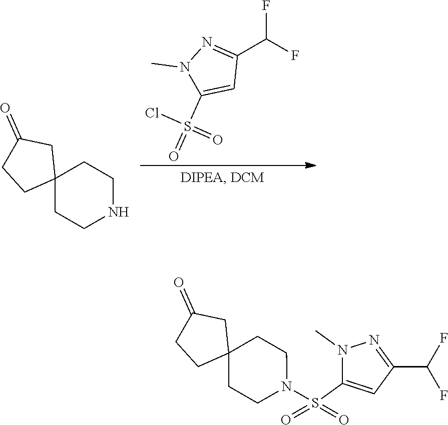 [SMILES: O=C1CCC2(CCNCC2)C1 ; Cn1nc(C(F)F)cc1S(=O)(=O)Cl ; Cn1nc(C(F)F)cc1S(=O)(=O)N1CCC2(CCC(=O)C2)CC1]

To a 0-5° C. solution of 8-azaspiro[4.5]decan-2-one (240 mg, 1.27 mmol, HCl salt) and DIPEA (490.58 mg, 3.80 mmol, 661.16 μL) in DCM (15 mL) was added 3-(difluoromethyl)-1-methyl-1H-pyrazole-5-sulfonyl chloride (320.98 mg, 1.39 mmol) and the reaction mixture was stirred at 15° C. for 1 h. The mixture was concentrated in vacuum. The residue was purified by flash column (MeOH in DCM=0%˜3%) to give the desired compound (220 mg, 50%) as a yellow oil. LCMS m/z=348.0 [M+H]⁺. ¹H NMR (500 MHz, CDCl₃) δ (ppm) 6.87 (s, 1H), 6.767 (t, J=55.0 Hz, 1H), 4.12 (s, 3H), 3.46-3.38 (m, 2H), 3.10-3.03 (m, 2H), 2.30 (t, J=8.0 Hz, 2H), 2.12 (s, 2H), 1.87 (t, J=8.0 Hz, 2H), 1.74-1.66 (m, 4H).

### 5. Synthesis 6-(8-((3-(difluoromethyl)-1-methyl-1H-pyrazol-5-yl)sulfonyl)-8-azaspiro[4.5]decan-2-yl)-2-oxa-6-azaspiro[3.3]heptane

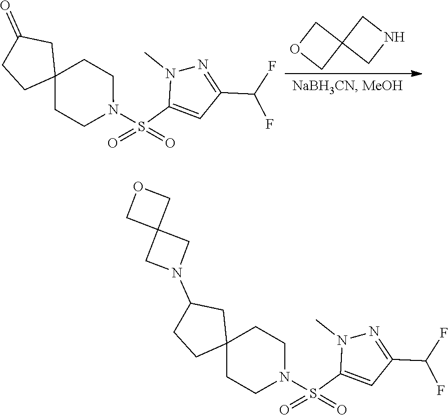

To a solution of 8-((3-(difluoromethyl)-1-methyl-1H-pyrazol-5-yl)sulfonyl)-8-azaspiro[4.5]decan-2-one (170 mg, 489.38 μmol) and 2-oxa-6-azaspiro[3.3]heptane (53.36 mg, 538.32 μmol) in MeOH (8 mL) was added acetic acid (58.77 mg, 978.76 μmol, 56.03 μL) and the resulting mixture was stirred for 1 h at 15° C., followed by NaBH₃CN (92.26 mg, 1.47 mmol). The mixture stirred at 15° C. for 2 h. The mixture was concentrated in vacuo and the residue was purified by pre-HPLC (Column: Welch Xtimate C18 150*30 mm*5 μm, Condition: water (10 mM NH₄HCO₃)−ACN, 32%˜61%, Flow Rate (mL/min): 25) to give the title compound (92.53 mg, 44%) as a yellow oil. LCMS m/z=431.2 [M+H]⁺. ¹H NMR (400 MHz, CDCl₃) δ (ppm) 6.85 (s, 1H), 6.67 (t, J=54.8 Hz, 1H), 4.71 (s, 4H), 4.10 (s, 3H), 3.26 (s, 4H), 3.20-3.11 (m, 4H), 2.66-2.56 (m, 1H), 1.71-1.62 (m, 4H), 1.55-1.50 (m, 3H), 1.45-1.36 (m, 2H), 1.16-1.12 (m, 1H).

### Example 126: 4-((2-(2-oxa-6-azaspiro[3.3]heptan-6-yl)-8-azaspiro[4.5]decan-8-yl)sulfonyl)-3-chlorobenzonitrile

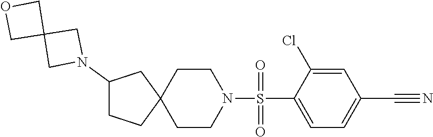 [SMILES: N#Cc1ccc(S(=O)(=O)N2CCC3(CCC(N4CC5(COC5)C4)C3)CC2)c(Cl)c1]

### 1. Synthesis of tert-butyl 2-(2-oxa-6-azaspiro[3.3]heptan-6-yl)-8-azaspiro[4.5]decane-8-carboxylate

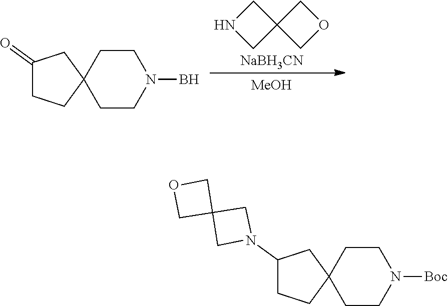

To a solution of tert-butyl 3-oxo-8-azaspiro[4.5]decane-8-carboxylate (100 mg, 394.73 umol) in MeOH (20 mL) was added 2-oxa-6-azaspiro[3.3]heptane; oxalic acid (113.80 mg, 394.73 umol). Sodium cyanoborohydride (74.42 mg, 1.18 mmol) was added and the mixture was stirred at 25° C. for 12 h. Water (50 ml) was added and the mixture was extracted with EtOH (3×50 mL). The combined organics were dried over Na₂SO₃. The mixture was filtered and concentrated under vacuum to give the crude product which was not purified further.

### 2. Synthesis of tert-butyl 2-(2-oxa-6-azaspiro[3.3]heptan-6-yl)-8-azaspiro[4.5]decane-8-carboxylate

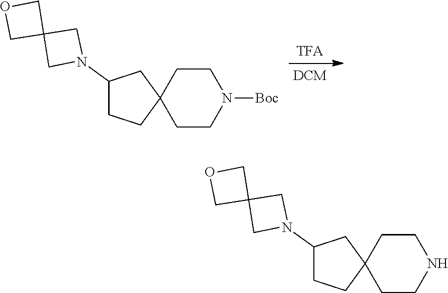

To a solution of tert-butyl 3-(2-oxa-6-azaspiro[3.3]heptan-6-yl)-8-azaspiro[4.5]decane-8-carboxylate (100 mg, 297.21 umol) in DCM (2 mL) was added TFA (67.77 mg, 594.41 umol, 45.52 uL) at 25° C. Then the mixture was stirred at 25° C. for 3 h. DIPEA (0.3 mL) was added and the mixture was filtered and concentrated under vacuum to give 6-(8-azaspiro[4.5]decan-3-yl)-2-oxa-6-azaspiro[3.3]heptane (120 mg, crude, Trifluoroacetate) as colorless oil.

### 3. Synthesis of 4-((2-(2-oxa-6-azaspiro[3.3]heptan-6-yl)-8-azaspiro[4.5]decan-8-yl)sulfonyl)-3-chlorobenzonitrile

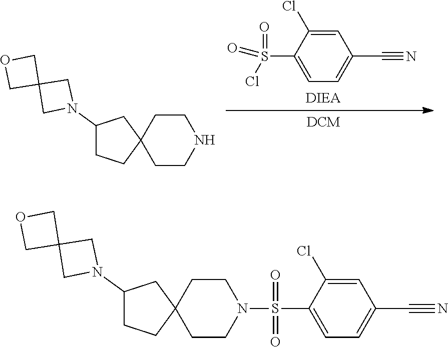 [SMILES: C1CC2(CCN1)CCC(N1CC3(COC3)C1)C2 ; N#Cc1ccc(S(=O)(=O)Cl)c(Cl)c1 ; N#Cc1ccc(S(=O)(=O)N2CCC3(CCC(N4CC5(COC5)C4)C3)CC2)c(Cl)c1]

To a solution of 6-(8-azaspiro[4.5]decan-2-yl)-2-oxa-6-azaspiro[3.3]heptane (50 mg, 211 mmol) in DCM (5 mL) was added DIPEA at 25° C. Then 2-chloro-4-cyanobenzenesulfonyl chloride (49.9 mg, 211 mmol) was added into the mixture at 25° C. The mixture was stirred at 25° C. for 0.5 h. The reaction was filtered to give the crude reaction mixture. The residue was dissolved in DMSO (0.5 mL) and purified by prep. HPLC (Column: YMC Actus Trial C18 20×100 mm, 5 μm; Method water—MeOH—NH₃ 0.1% as a mobile phase) to afford the desired product (24 mg, 26%). LCMS m/z=436.0 [M+H]⁺, R_{f}=1.80 min.

### Example 127: 5-((2-(2-oxa-6-azaspiro[3.3]heptan-6-yl)-8-azaspiro[4.5]decan-8-yl)sulfonyl)-2-fluorobenzonitrile

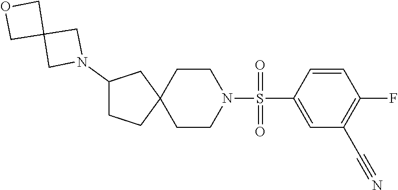 [SMILES: N#Cc1cc(S(=O)(=O)N2CCC3(CCC(N4CC5(COC5)C4)C3)CC2)ccc1F]

The synthesis was performed in a similar manner to that described in Example 126, step 3, using 6-(8-azaspiro[4.5]decan-2-yl)-2-oxa-6-azaspiro[3.3]heptane and 3-cyano-4-fluorobenzenesulfonyl chloride (15 mg, 17%). LCMS m/z=420.1 [M+H]⁺, R_{f}=1.72 min (Column: Waters ACQUITY UPLC BEH C18 2.1×30 mm, 1.7 μm, Modifier: Trifluoroacetic acid 0.1% (v/v) conc. Method: 95% H₂O/5% MeCN (initial conditions) linear gradient to 5% H₂O/95% MeCN at 1.0 min, HOLD 5% H₂O/95% MeCN to 1.3 min. Flow rate, 0.7 mL/min).

### Example 128: 5-((2-(2-oxa-6-azaspiro[3.3]heptan-6-yl)-8-azaspiro[4.5]decan-8-yl)sulfonyl)-2-methylbenzonitrile

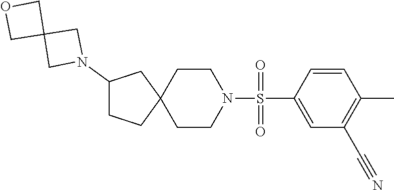 [SMILES: Cc1ccc(S(=O)(=O)N2CCC3(CCC(N4CC5(COC5)C4)C3)CC2)cc1C#N]

The synthesis was performed in a similar manner to that described in Example 126, step 3, using 6-(8-azaspiro[4.5]decan-2-yl)-2-oxa-6-azaspiro[3.3]heptane and 3-cyano-4-methylbenzenesulfonyl chloride (21 mg, 24%). LCMS m/z=416.1 [M+H]⁺, R_{f}=1.75 min (Column: Waters ACQUITY UPLC BEH C18 2.1×30 mm, 1.7 μm, Modifier: Trifluoroacetic acid 0.1% (v/v) conc. Method: 95% H₂O/5% MeCN (initial conditions) linear gradient to 5% H₂O/95% MeCN at 1.0 min, HOLD 5% H₂O/95% MeCN to 1.3 min. Flow rate, 0.7 mL/min).

### Example 129: 6-(8-((2-fluoro-4-methoxyphenyl)sulfonyl)-8-azaspiro[4.5]decan-2-yl)-2-oxa-6-azaspiro[3.3]heptane

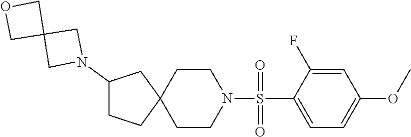 [SMILES: COc1ccc(S(=O)(=O)N2CCC3(CCC(N4CC5(COC5)C4)C3)CC2)c(F)c1]

The synthesis was performed in a similar manner to that described in Example 126 step 3, using 6-(8-azaspiro[4.5]decan-2-yl)-2-oxa-6-azaspiro[3.3]heptane and 2-fluoro-4-methoxybenzenesulfonyl chloride (17 mg, 19%). LCMS m/z=425.1 [M+H]⁺, R_{f}=1.77 min. (Column: Waters ACQUITY UPLC BEH C18 2.1×30 mm, 1.7 μm, Modifier: Trifluoroacetic acid 0.1% (v/v) conc. Method: 95% H₂O/5% MeCN (initial conditions) linear gradient to 5% H₂O/95% MeCN at 1.0 min, HOLD 5% H₂O/95% MeCN to 1.3 min. Flow rate, 0.7 mL/min).

### Example 130: 6-(8-((6-isopropoxypyridin-3-yl)sulfonyl)-8-azaspiro[4.5]decan-2-yl)-2-oxa-6-azaspiro[3.3]heptane

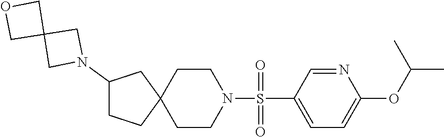 [SMILES: CC(C)Oc1ccc(S(=O)(=O)N2CCC3(CCC(N4CC5(COC5)C4)C3)CC2)cn1]

The synthesis was performed in a similar manner to that described in Example 112 using 6-(8-azaspiro[4.5]decan-2-yl)-2-oxa-6-azaspiro[3.3]heptane and 6-isopropoxypyridine-3-sulfonyl fluoride (5 mg, 8%). LCMS m/z=436.1 [M+H]⁺, R_{f}=1.84 min (Column: Waters ACQUITY UPLC BEH C18 2.1×30 mm, 1.7 μm, Modifier: Trifluoroacetic acid 0.1% (v/v) conc. Method: 95% H₂O/5% MeCN (initial conditions) linear gradient to 5% H₂O/95% MeCN at 1.0 min, HOLD 5% H₂O/95% MeCN to 1.3 min. Flow rate, 0.7 mL/min).

### Example 131: 6-(8-(pyrazolo[1,5-a]pyridin-7-ylsulfonyl)-8-azaspiro[4.5]decan-2-yl)-2-oxa-6-azaspiro[3.3]heptane

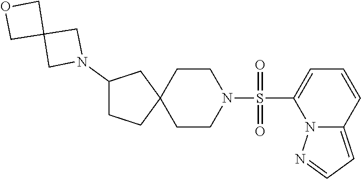 [SMILES: O=S(=O)(c1cccc2ccnn12)N1CCC2(CCC(N3CC4(COC4)C3)C2)CC1]

The synthesis was performed in a similar manner to that described in Example 112 using 6-(8-azaspiro[4.5]decan-2-yl)-2-oxa-6-azaspiro[3.3]heptane and pyrazolo[1,5-a]pyridine-6-sulfonyl fluoride (14 mg, 22%). LCMS m/z=417.1 [M+H]⁺, R_{f}=1.55 min (Column: Waters ACQUITY UPLC BEH C18 2.1×30 mm, 1.7 μm, Modifier: Trifluoroacetic acid 0.1% (v/v) conc.
Method: 95% H₂O/5% MeCN (initial conditions) linear gradient to 5% H₂O/95% MeCN at 1.0 min, HOLD 5% H₂O/95% MeCN to 1.3 min. Flow rate, 0.7 mL/min).

### Example 132: 6-(8-((5-fluoroquinolin-3-yl)sulfonyl)-8-azaspiro[4.5]decan-2-yl)-2-oxa-6-azaspiro[3.3]heptane

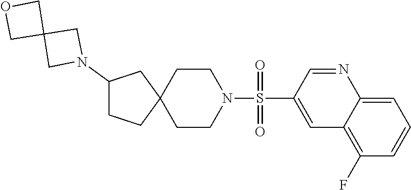 [SMILES: O=S(=O)(c1cnc2cccc(F)c2c1)N1CCC2(CCC(N3CC4(COC4)C3)C2)CC1]

The synthesis was performed in a similar manner to that described in Example 112 using 6-(8-azaspiro[4.5]decan-2-yl)-2-oxa-6-azaspiro[3.3]heptane and 5-fluoroquinoline-3-sulfonyl fluoride (5 mg, 9%). LCMS m/z=446.3 [M+H]⁺, R_{f}=1.65 min (Column: Waters ACQUITY UPLC BEH C18 2.1×30 mm, 1.7 μm, Modifier: Trifluoroacetic acid 0.1% (v/v) conc. Method: 95% H₂O/5% MeCN (initial conditions) linear gradient to 5% H₂O/95% MeCN at 1.0 min, HOLD 5% H₂O/95% MeCN to 1.3 min. Flow rate, 0.7 mL/min).

### Example 133: 6-(8-((3-fluoroquinolin-6-yl)sulfonyl)-8-azaspiro[4.5]decan-2-yl)-2-oxa-6-azaspiro[3.3]heptane

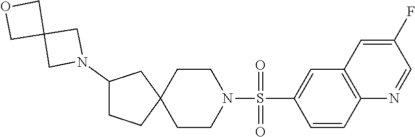 [SMILES: O=S(=O)(c1ccc2ncc(F)cc2c1)N1CCC2(CCC(N3CC4(COC4)C3)C2)CC1]

The synthesis was performed in a similar manner to that described in Example 122 using 6-(8-azaspiro[4.5]decan-2-yl)-2-oxa-6-azaspiro[3.3]heptane and 3-fluoroquinoline-6-sulfonyl fluoride (27 mg, 44%). LCMS m/z=446.3 [M+H]⁺, R_{f}=1.62 min (Column: Waters ACQUITY UPLC BEH C18 2.1×30 mm, 1.7 μm, Modifier: Trifluoroacetic acid 0.1% (v/v) conc.
Method: 95% H₂O/5% MeCN (initial conditions) linear gradient to 5% H₂O/95% MeCN at 1.0 min, HOLD 5% H₂O/95% MeCN to 1.3 min. Flow rate, 0.7 mL/min).

### Example 134: 6-(8-((4-cyclopropoxyphenyl)sulfonyl)-8-azaspiro[4.5]decan-2-yl)-2-oxa-6-azaspiro[3.3]heptane

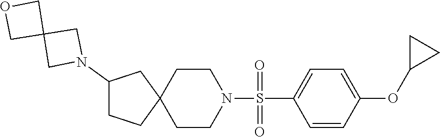 [SMILES: O=S(=O)(c1ccc(OC2CC2)cc1)N1CCC2(CCC(N3CC4(COC4)C3)C2)CC1]

The synthesis was performed in a similar manner to that described in Example 112 using 6-(8-azaspiro[4.5]decan-2-yl)-2-oxa-6-azaspiro[3.3]heptane and 4-cyclopropoxybenzenesulfonyl fluoride (8 mg, 12%). LCMS m/z=433.1 [M+H]⁺, R_{f}=1.79 min (Column: Waters ACQUITY UPLC BEH C18 2.1×30 mm, 1.7 μm, Modifier: Trifluoroacetic acid 0.1% (v/v) conc. Method: 95% H₂O/5% MeCN (initial conditions) linear gradient to 5% H₂O/95% MeCN at 1.0 min, HOLD 5% H₂O/95% MeCN to 1.3 min. Flow rate, 0.7 mL/min).

### Example 135: 6-(8-(benzofuran-6-ylsulfonyl)-8-azaspiro[4.5]decan-2-yl)-2-oxa-6-azaspiro[3.3]heptane

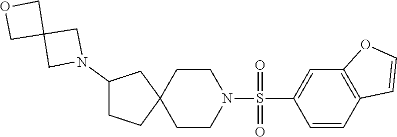 [SMILES: O=S(=O)(c1ccc2ccoc2c1)N1CCC2(CCC(N3CC4(COC4)C3)C2)CC1]

The synthesis was performed in a similar manner to that described in Example 112 using 6-(8-azaspiro[4.5]decan-2-yl)-2-oxa-6-azaspiro[3.3]heptane and 4-cyclopropoxybenzenesulfonyl fluoride (10 mg, 15%). LCMS m/z=419.1 [M+H]⁺, R_{f}=1.54 min (Column: Waters ACQUITY UPLC BEH C18 2.1×30 mm, 1.7 μm, Modifier: Trifluoroacetic acid 0.1% (v/v) conc. Method: 95% H₂O/5% MeCN (initial conditions) linear gradient to 5% H₂O/95% MeCN at 1.0 min, HOLD 5% H₂O/95% MeCN to 1.3 min. Flow rate, 0.7 mL/min).

### Example 136: 8-((2-fluoro-4-methoxyphenyl)sulfonyl)-N-methyl-N-(2-oxaspiro[3.3]heptan-6-yl)-1-oxa-8-azaspiro[4.5]decan-3-amine

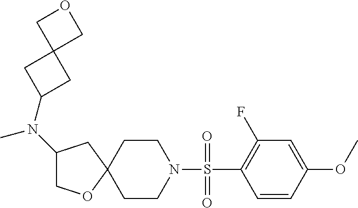

### 1. Synthesis of 8-((2-fluoro-4-methoxyphenyl)sulfonyl)-1-oxa-8-azaspiro[4.5]decan-3-one

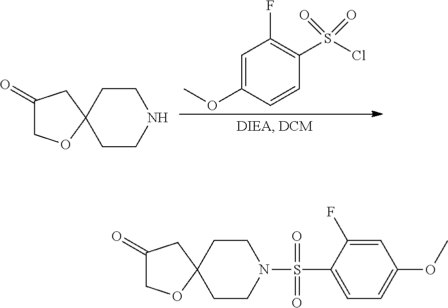

The synthesis was performed in a similar manner to that described in Example 98 step 1, using 1-oxa-8-azaspiro[4.5]decan-3-one hydrochloride and 2-fluoro-4-methoxybenzenesulfonyl chloride (980 mg, 74%). LCMS m/z=344.0 [M+H]⁺, ¹H NMR (500 MHz, CHLOROFORM-d) δ (ppm) 7.76 (t, J=8.5 Hz, 1H), 6.80-6.70 (m, 2H), 3.96 (s, 2H), 3.88 (s, 3H), 3.61-3.55 (m, 2H), 3.08-3.00 (m, 2H), 2.35 (s, 2H), 1.92-1.81 (m, 4H).

### 2. Synthesis of 8-((2-fluoro-4-methoxyphenyl)sulfonyl)-N-methyl-N-(2-oxaspiro[3.3]heptan-6-yl)-1-oxa-8-azaspiro[4.5]decan-3-amine

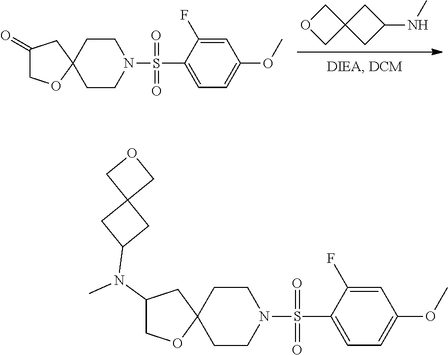

The synthesis was performed in a similar manner to that described in Example 126 step 1 using 8-((2-fluoro-4-methoxyphenyl)sulfonyl)-1-oxa-8-azaspiro[4.5]decan-3-one and N-Methyl-2-oxaspiro[3.3]heptan-6-amine (25 mg, 35%). LCMS m/z=455.2 [M+H]⁺, ¹H NMR (400 MHz, CDCl₃) δ (ppm) 7.74-7.70 (m, 1H), 6.76-6.73 (m, 1H), 6.70-6.67 (m, 1H), 4.69 (s, 2H), 4.55 (s, 2H), 3.86 (s, 3H), 3.84-3.79 (m, 1H), 3.61-3.48 (m, 3H), 3.08-3.00 (m, 1H), 2.92-2.83 (m, 2H), 2.66-2.58 (m, 1H), 2.41-2.30 (m, 2H), 2.03 (s, 3H), 2.00-1.94 (m, 1H), 1.86-1.73 (m, 3H), 1.71-1.63 (m, 2H), 1.61-1.55 (m, 2H).

### Example 137: 8-((2-methyl-6-(trifluoromethyl)pyridin-3-yl)sulfonyl)-N-(2-oxaspiro[3.3]heptan-6-yl)-1-oxa-8-azaspiro[4.5]decan-3-amine

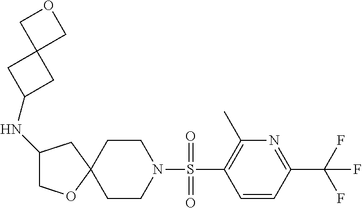 [SMILES: Cc1nc(C(F)(F)F)ccc1S(=O)(=O)N1CCC2(CC1)CC(NC1CC3(COC3)C1)CO2]

### 1. Synthesis of 8-((2-methyl-6-(trifluoromethyl)pyridin-3-yl)sulfonyl)-1-oxa-8-azaspiro[4.5]decan-3-one

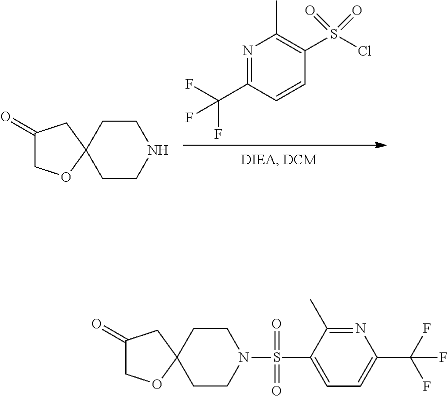 [SMILES: O=C1COC2(CCNCC2)C1 ; Cc1nc(C(F)(F)F)ccc1S(=O)(=O)Cl ; Cc1nc(C(F)(F)F)ccc1S(=O)(=O)N1CCC2(CC1)CC(=O)CO2]

To a solution of 1-oxa-8-azaspiro[4.5]decan-3-one hydrochloride (100 mg, 521.77 umol, HCl) and DIPEA (202.30 mg, 1.57 mmol, 272.64 uL) in DCM (6 mL) was added 2-methyl-6-(trifluoromethyl)pyridine-3-sulfonyl chloride (142.24 mg, 547.86 umol) at 0° C. under N₂.

The mixture was stirred at 25° C. for 3 h. The mixture was diluted with water (20 mL), extracted with DCM (30 mL×2). The combined organic phase was washed with brine (30 mL), dried over anhydrous sodium sulfate, filtered, and concentrated to give the desired compound (170 mg, crude) as a yellow oil. LCMS m/z=379.1 [M+H]⁺. ¹H NMR (500 MHz, CHLOROFORM-d) δ (ppm) 8.36 (d, J=8.0 Hz, 1H), 7.68 (d, J=8.0 Hz, 1H), 3.99 (s, 2H), 3.67-3.58 (m, 2H), 3.29-3.19 (m, 2H), 2.92 (s, 3H), 2.39 (s, 2H), 1.94-1.85 (m, 4H).

### 2. Synthesis of 8-((2-methyl-6-(trifluoromethyl)pyridin-3-yl)sulfonyl)-N-(2-oxaspiro[3.3]heptan-6-yl)-1-oxa-8-azaspiro[4.5]decan-3-amine

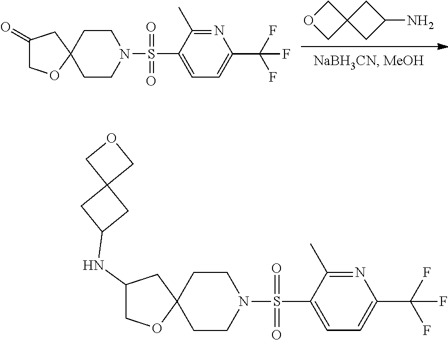

A solution of 8-((2-methyl-6-(trifluoromethyl)pyridin-3-yl)sulfonyl)-1-oxa-8-azaspiro[4.5]decan-3-one (170 mg, 449.30 umol) and 2-oxaspiro[3.3]heptan-6-amine (168.06 mg, 1.12 mmol, HCl) in MeOH (5 mL) was adjusted pH=5-6 by Acetic acid at 25° C. and was stirred at 25° C. for 1 h. NaBH₃CN (141.17 mg, 2.25 mmol) was added at 25° C., then the mixture was stirred at 25° C. for 16 h. LCMS showed the desired product mass was observed. The mixture was concentrated and diluted with water (10 mL), extracted with DCM (30 mL×2). The combined organic phase was washed with brine (30 mL), dried over anhydrous sodium sulfate, filtered, and concentrated. The residue was purified by flash silica gel chromatography (EtOAc in petroleum ether=from 0% to 70%) to give crude product (130 mg, 61% yield) as white oil. The product was dissolved in ACN (1 mL) and purified by prep-HPLC (Column: Welch Xtimate C18 150*25 mm*5 μm; Condition: water (NH₄HCO₃)−ACN; Begin B: 29; End B: 58; Flow Rate: 25 mL/min) to give the title compound (30 mg, 50% yield) as a yellow solid. LCMS m/z=476.1 [M+H]⁺. ¹H NMR (400 MHz, CHLOROFORM-d) δ (ppm) 8.33 (d, J=8.0 Hz, 1H), 7.65 (d, J=8.0 Hz, 1H), 4.70 (s, 2H), 4.59 (s, 2H), 3.92-3.84 (m, 1H), 3.59-3.46 (m, 3H), 3.39-3.31 (m, 1H), 3.15-3.02 (m, 3H), 2.91 (s, 3H), 2.61-2.47 (m, 2H), 2.02-1.96 (m, 1H), 1.89-1.78 (m, 4H), 1.71-1.66 (m, 2H), 1.53-1.47 (m, 1H).

### Example 138: 8-((2-methyl-6-(trifluoromethyl)pyridin-3-yl)sulfonyl)-N-(tetrahydro-2H-pyran-4-yl)-1-oxa-8-azaspiro[4.5]decan-3-amine

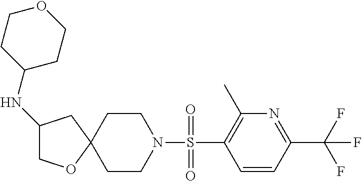 [SMILES: Cc1nc(C(F)(F)F)ccc1S(=O)(=O)N1CCC2(CC1)CC(NC1CCOCC1)CO2]

The synthesis was performed in a similar manner to that described in Example 137 step 2 using 8-((2-fluoro-4-methoxyphenyl)sulfonyl)-1-oxa-8-azaspiro[4.5]decan-3-one and tetrahydro-2H-pyran-4-amine (67 mg, 20%). LCMS m/z=464.2 [M+H]⁺. ¹H NMR (400 MHz, CD₃OD) δ (ppm) 8.43 (d, J 8.0 Hz, 1H), 7.83 (d, J 8.0 Hz, 1H), 3.99-3.96 (m, 1H), 3.93-3.90 (m, 2H), 3.64-3.55 (m, 1H), 3.53-3.43 (m, 3H), 3.41-3.38 (m, 2H), 3.23-3.09 (m, 2H), 2.87 (s, 3H), 2.73-2.63 (m, 1H), 2.15-2.11 (m, 1H), 1.89-1.77 (m, 4H), 1.77-1.60 (m, 2H), 1.56-1.53 (m, 1H), 1.44-1.28 (m, 2H).

### Example 139: 8-((1-methyl-3-(trifluoromethyl)-1H-pyrazol-5-yl)sulfonyl)-N-(2-oxaspiro[3.3]heptan-6-yl)-1-oxa-8-azaspiro[4.5]decan-3-amine

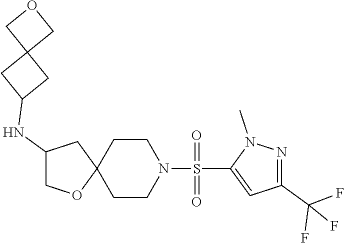 [SMILES: Cn1nc(C(F)(F)F)cc1S(=O)(=O)N1CCC2(CC1)CC(NC1CC3(COC3)C1)CO2]

### 1. Synthesis of 8-((I-methyl-3-(trifluoromethyl)-1H-pyrazol-5-yl)sulfonyl)-1-oxa-8-azaspiro[4.5]decan-3-one

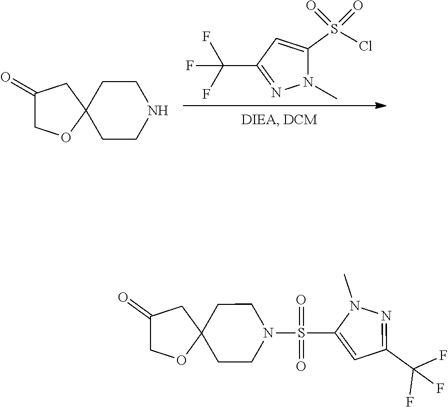 [SMILES: O=C1COC2(CCNCC2)C1 ; Cn1nc(C(F)(F)F)cc1S(=O)(=O)Cl ; Cn1nc(C(F)(F)F)cc1S(=O)(=O)N1CCC2(CC1)CC(=O)CO2]

The synthesis was performed in a similar manner to that described in Example 137 step 1 using 1-oxa-8-azaspiro[4.5]decan-3-one and 1-methyl-3-(trifluoromethyl)-1H-pyrazole-5-sulfonyl chloride (370 mg, 73%). LCMS m/z=368.1 [M+H]⁺. ¹H NMR (500 MHz, CDCl₃) δ (ppm) 6.92 (s, 1H), 4.16 (s, 3H), 3.99 (s, 2H), 3.70-3.65 (m, 2H), 3.15-3.08 (m, 2H), 2.39 (s, 2H), 1.97-1.87 (m, 4H).

### 2. Synthesis of 8-((I-methyl-3-(trifluoromethyl)-H-pyrazol-5-yl)sulfonyl)-N-(2-oxaspiro[3.3]heptan-6-yl)-1-oxa-8-azaspiro[4.5]decan-3-amine

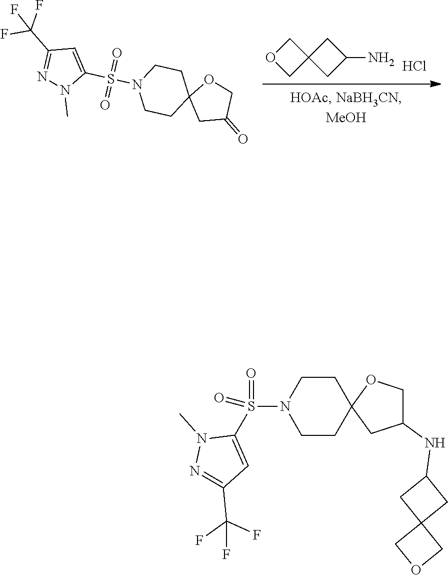 [SMILES: Cn1nc(C(F)(F)F)cc1S(=O)(=O)N1CCC2(CC1)CC(=O)CO2 ; NC1CC2(COC2)C1 ; Cl ; Cn1nc(C(F)(F)F)cc1S(=O)(=O)N1CCC2(CC1)CC(NC1CC3(COC3)C1)CO2]

The synthesis was performed in a similar manner to that described in Example 137 step 2 using 8-((1-methyl-3-(trifluoromethyl)-1H-pyrazol-5-yl)sulfonyl)-1-oxa-8-azaspiro[4.5]decan-3-one and 2-oxaspiro[3.3]heptan-6-amine (150 mg, 28%). LCMS m/z=465.1 [M+H]⁺. ¹H NMR (400 MHz, CDCl₃) δ (ppm) 6.89 (s, 1H), 4.69 (s, 2H), 4.58 (s, 2H), 4.13 (s, 3H), 3.90-3.84 (m, 1H), 3.63-3.55 (m, 2H), 3.51-3.46 (m, 1H), 3.39-3.31 (m, 1H), 3.10-2.93 (m, 3H), 2.59-2.49 (m, 2H), 2.00-1.94 (m, 1H), 1.92-1.78 (m, 4H), 1.73-1.63 (m, 2H), 1.53-1.47 (m, 1H).

### Example 140: N-methyl-8-((4-methyl-2-(trifluoromethyl)pyrimidin-5-yl)sulfonyl)-N-(2-oxaspiro[3.3]heptan-6-yl)-1-oxa-8-azaspiro[4.5]decan-3-amine

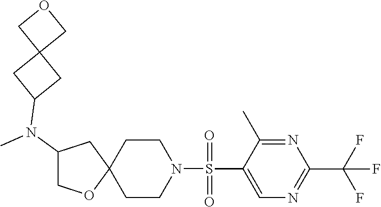 [SMILES: Cc1nc(C(F)(F)F)ncc1S(=O)(=O)N1CCC2(CC1)CC(N(C)C1CC3(COC3)C1)CO2]

### 1. Synthesis of 4-bromo-2-(trifluoromethyl)pyrimidin-5-amine

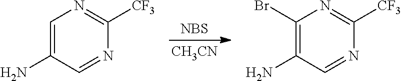

To a solution of 2-(trifluoromethyl)pyrimidin-5-amine (3 g, 18.39 mmol) in CH₃CN (60 mL) was added NBS (3.93 g, 22.07 mmol). The mixture was stirred at room temperature for 16 h under nitrogen. The acetonitrile was evaporated, and the residue partitioned in water (100 mL) and ethyl acetate (100 mL), the layers were separated, and the aqueous layer was extracted with ethyl acetate (50 mL×2). The combined organic layers were washed with brine (50 mL), dried over anhydrous sodium sulfate, filtered, and concentrated. The residue was purified by silica gel chromatography eluted with 0-30% ethyl acetate in heptane to give the desired compound (2.6 g, 58%) as a yellow solid. LCMS m/z=243.9 [M+H]⁺. ¹H NMR (400 MHz, CDCl₃) δ (ppm) 8.18 (s, 1H), 4.58 (br s, 2H).

### 2. Synthesis of 4-methyl-2-(trifluoromethyl)pyrimidin-5-amine

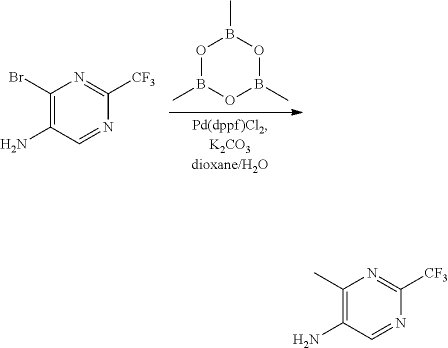

To a solution of 4-bromo-2-(trifluoromethyl)pyrimidin-5-amine (2.55 g, 10.54 mmol) and 2,4,6-trimethyl-1,3,5,2,4,6-trioxatriborinane (7.94 g, 31.61 mmol, 8.84 mL) in dioxane (30 mL) and H₂O (3 mL) was added K₂CO₃ (2.91 g, 21.07 mmol) and Pd(dppf)Cl₂ (616.82 mg, 842.99 umol). The reaction mixture was stirred at 100° C. for 2 h under nitrogen. The mixture was diluted with water (15 mL), extracted with EtOAc (15 mL×3). The combined organic phase was washed with brine (40 mL×1), dried over anhydrous sodium sulfate, filtered, and concentrated in vacuo. The crude was purified by flash column (EtOAc in petroleum ether=5%˜22%) to give the desired compound (1.4 g, 75%) as a yellow solid. LCMS m/z=178.1 [M+H]⁺. ¹H NMR (400 MHz, CDCl₃) δ (ppm) 8.13 (s, 1H), 4.00 (br s, 2H), 2.46 (s, 3H).

### 3. Synthesis of 5-bromo-4-methyl-2-(trifluoromethyl)pyrimidine

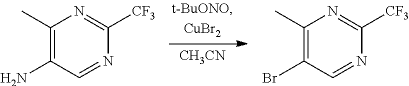

To a solution of 4-methyl-2-(trifluoromethyl)pyrimidin-5-amine (1 g, 5.65 mmol) in CH₃CN (30 mL) was added CuBr₂ (1.89 g, 8.47 mmol, 396.54 uL) and t-BuONO (873.27 mg, 8.47 mmol, 990.10 uL) in sequence under N₂. The reaction mixture was stirred at 15° C. for 30 min. The mixture was concentrated in vacuo and the residue was purified by flash column (EtOAc in petroleum ether=2%˜10%) to give the desired product (900 mg, 66%) as a colorless oil. ¹H NMR (400 MHz, CDCl₃) δ ppm 8.85 (s, 1H), 2.74 (s, 3H).

### 4. Synthesis of 5-(benzylthio)-4-methyl-2-(trifluoromethyl)pyrimidine

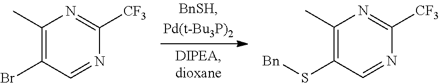 [SMILES: Cc1nc(C(F)(F)F)ncc1Br ; Cc1nc(C(F)(F)F)ncc1SCc1ccccc1]

To a solution of 5-bromo-4-methyl-2-(trifluoromethyl)pyrimidine (600 mg, 2.49 mmol) and BnSH (556.58 mg, 4.48 mmol, 526.06 uL) in dioxane (15 mL) was added DIPEA (965.27 mg, 7.47 mmol, 1.30 mL) and Pd(t-Bu₃P)₂ (190.84 mg, 373.43 umol). The reaction mixture was stirred at 100° C. for 12 h under nitrogen atmosphere. The mixture was diluted with water (15 mL), extracted with EtOAc (10 mL×3). The combined organic phase was washed with brine (30 mL), dried over anhydrous sodium sulfate, filtered, and concentrated in vacuum. The crude was purified by flash column (EtOAc in petroleum ether=0˜8%) to give the desired product (0.67 g, 95%) as a yellow solid. LCMS m/z=285.0 [M+H]⁺. ¹H NMR (400 MHz, CDCl₃) δ (ppm) 8.50 (s, 1H), 7.36-7.29 (m, 5H), 4.23 (s, 2H), 2.58 (s, 3H).

### 5. Synthesis of 4-methyl-2-(trifluoromethyl)pyrimidine-5-sulfonyl chloride

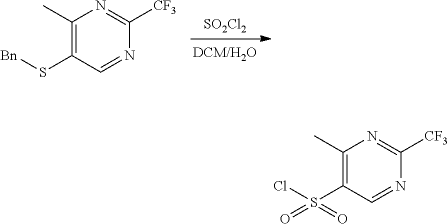

To a solution of 5-(benzylthio)-4-methyl-2-(trifluoromethyl)pyrimidine (550 mg, 1.93 mmol) in DCM (5 mL) and H₂O (1 mL) was added a solution of SO₂Cl₂ (1.83 g, 13.54 mmol, 1.10 mL) in DCM (1 mL) dropwise over 5 minutes at 0˜5° C. The mixture was stirred at 0-5° C. for 15 h. TLC (DCM/MeOH=10/1) showed the starting material was consumed completely and a new spot was observed. The mixture was diluted with water (10 mL), extracted with EtOAc (15 mL×4). The combined organic phase was dried over anhydrous sodium sulfate and concentrated in vacuo to give the desired product (350 mg, crude) as a yellowish oil. ¹H NMR (400 MHz, CDCl3) δ (ppm) 9.36 (s, 1H), 3.10 (s, 3H).

### 6. Synthesis of 8-((4-methyl-2-(trifluoromethyl)pyrimidin-5-yl)sulfonyl)-1-oxa-8-azaspiro[4.5]decan-3-one

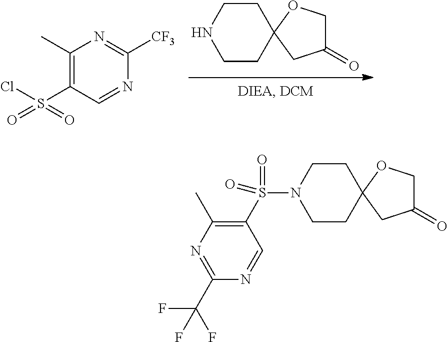 [SMILES: Cc1nc(C(F)(F)F)ncc1S(=O)(=O)Cl ; O=C1COC2(CCNCC2)C1 ; Cc1nc(C(F)(F)F)ncc1S(=O)(=O)N1CCC2(CC1)CC(=O)CO2]

To a solution of 1-oxa-8-azaspiro[4.5]decan-3-one (300 mg, 1.12 mmol, TFA salt) and DIPEA (433.69 mg, 3.36 mmol, 584.48 uL) in DCM (5 mL) was added 4-methyl-2-(trifluoromethyl) pyrimidine-5-sulfonyl chloride (349.82 mg, 1.34 mmol) at 0° C. The mixture was stirred at 20° C. for 3 h. The mixture was diluted with water (10 mL), extracted with DCM (15 mL×3). The combined organic phase was washed with brine (20 mL×1), dried over anhydrous sodium sulfate, and concentrated in vacuo. The crude was purified by flash silica gel chromatography (MeOH in DCM from 6% to 9%) to give the desired product (376 mg, 89%) as a yellowish solid. LCMS m/z=380.1 [M+H]⁺. ¹H NMR (400 MHz, CDCl₃) δ (ppm) 9.17 (s, 1H), 3.99 (s, 2H), 3.72-3.66 (m, 2H), 3.27-3.20 (m, 2H), 2.93 (s, 3H), 2.39 (s, 2H), 1.96-1.85 (m, 4H).

### 7. Synthesis of 8-((4-methyl-2-(trifluoromethyl)pyrimidin-5-yl)sulfonyl)-N-(2-oxaspiro[3.3]heptan-6-yl)-1-oxa-8-azaspiro[4.5]decan-3-amine

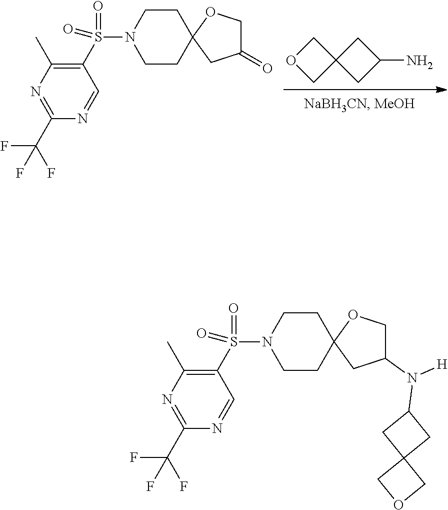

To a solution of 8-((4-methyl-2-(trifluoromethyl)pyrimidin-5-yl)sulfonyl)-1-oxa-8-azaspiro[4.5]decan-3-one (350 mg, 922.62 umol) and 2-oxaspiro[3.3]heptan-6-amine (151.84 mg, 1.01 mmol, HCl salt) in MeOH (3 mL) was added acetic acid (110.81 mg, 1.85 mmol, 105.63 uL) and NaBH₃CN (173.94 mg, 2.77 mmol). The mixture was stirred at 20° C. for 3 h. The mixture was purified by prep-HPLC (Column: Welch Xtimate C18 150*25 mm*5 μm, Condition: water (NH₄HCO₃)−ACN, 30%˜60%, Flow Rate (ml/min): 25) to give to give the desired compound (200 mg, 45%) as a white solid. LCMS m/z=477.1 [M+H]⁺. ¹H NMR (400 MHz, CDCl₃) δ (ppm) 9.14 (s, 1H), 4.69 (s, 2H), 4.58 (s, 2H), 3.88-3.85 (m, 1H), 3.64-3.59 (m, 2H), 3.52-3.49 (m, 1H), 3.31-3.26 (m, 1H), 3.12-3.05 (m, 3H), 2.91 (s, 3H), 2.55-2.53 (m, 2H), 1.97-1.95 (m, 1H), 1.87-1.81 (m, 4H), 1.72-1.69 (m, 2H), 1.53-1.49 (m, 1H).

### 8. Synthesis of N-methyl-8-((4-methyl-2-(trifluoromethyl)pyrimidin-5-yl)sulfonyl)-N-(2-oxaspiro[3.3]heptan-6-yl)-1-oxa-8-azaspiro[4.5]decan-3-amine

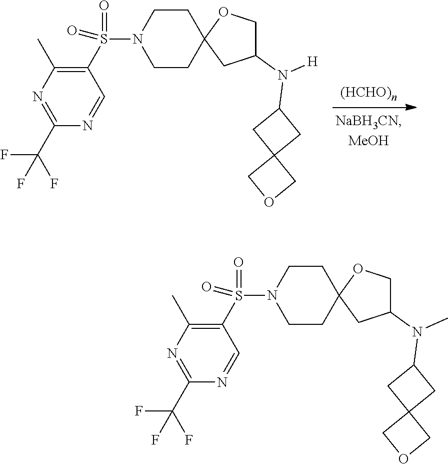 [SMILES: Cc1nc(C(F)(F)F)ncc1S(=O)(=O)N1CCC2(CC1)CC(NC1CC3(COC3)C1)CO2 ; Cc1nc(C(F)(F)F)ncc1S(=O)(=O)N1CCC2(CC1)CC(N(C)C1CC3(COC3)C1)CO2]

To a solution of 8-((4-methyl-2-(trifluoromethyl)pyrimidin-5-yl)sulfonyl)-N-(2-oxaspiro[3.3]heptan-6-yl)-1-oxa-8-azaspiro[4.5]decan-3-amine (80 mg, 167.89 umol) and (CH₂O)ₙ(241.66 mg, 201.46 umol, 274.62 uL) was added acetic acid (2.02 mg, 33.58 umol, 1.92 uL) and NaBH₃CN (31.65 mg, 503.66 umol). The mixture was stirred at 20° C. for 5 h. The mixture was purified by prep-HPLC (Column: Welch Xtimate C18 150*25 mm*5 μm, Condition: water (NH₄HCO₃)−ACN, 34%˜60%, Flow Rate (ml/min): 25) to give to give the title compound (40 mg, 49% yield) as a white solid. LCMS m/z=491.1 [M+H]⁺. ¹H NMR (400 MHz, CDCl₃) δ (ppm) 9.13 (s, 1H), 4.68 (s, 2H), 4.55 (s, 2H), 3.86-3.81 (m, 1H), 3.60-3.57 (m, 3H), 3.09-3.02 (m, 3H), 2.90 (s, 3H), 2.63-2.60 (m, 1H), 2.36-2.33 (m, 2H), 2.03 (s, 3H), 1.99-1.95 (m, 1H), 1.83-1.72 (m, 5H), 1.67-1.64 (m, 2H).

### Example 141: (S)-4-fluoro-3-((3-(4-hydroxy-4-methylpiperidin-1-yl)-1-oxa-8-azaspiro[4.5]decan-8-yl)sulfonyl)benzonitrile

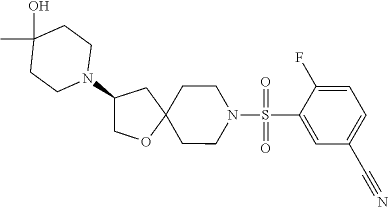 [SMILES: CC1(O)CCN([C@@H]2COC3(CCN(S(=O)(=O)c4cc(C#N)ccc4F)CC3)C2)CC1]

### 1. Synthesis of 4-fluoro-3-((3-oxo-1-oxa-8-azaspiro[4.5]decan-8-yl)sulfonyl)benzonitrile

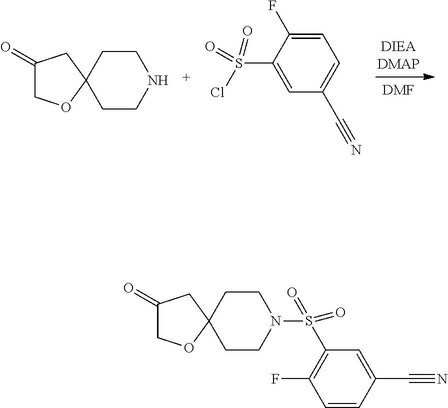

To a vial containing 1-oxa-8-azaspiro[4.5]decan-3-one (250 mg, 1.30 mmol, Hydrochloride) in anhydrous DMF (2 mL) was added Hunigs base (842.92 mg, 6.52 mmol, 1.14 mL). After 5 minutes, 5-cyano-2-fluoro-benzenesulfonyl chloride (286.48 mg, 1.30 mmol) was added. Upon complete addition of sulfonyl chloride, DMAP (15.94 mg, 130.44 umol) was added and the reaction was stirred at room temperature. After 60 minutes, the reaction was quenched with water and the mixture was extracted three times with ethyl acetate. The organics were pooled and washed twice with saturated aqueous sodium bicarbonate solution. The organic layer was separated then dried over anhydrous sodium sulfate. Crude material purified by silica gel column (0-25% [EtOAc to 3:1 EtOAc:EtOH]) to afford the desired compound (220 mg, 50%). ¹H NMR (CHLOROFORM-d, 400 MHz) δ 8.21 (dd, 1H, J=2.1, 6.1 Hz), 7.90 (ddd, 1H, J=2.1, 4.3, 8.6 Hz), 7.39 (t, 1H, J=8.9 Hz), 3.98 (s, 2H), 3.6-3.8 (m, 2H), 3.0-3.2 (m, 2H), 2.39 (s, 2H), 1.8-2.0 (m, 4H).

### 2. Synthesis of (S)-4-fluoro-3-((3-(4-hydroxy-4-methylpiperidin-1-yl)-1-oxa-8-azaspiro[4.5]decan-8-yl)sulfonyl)benzonitrile

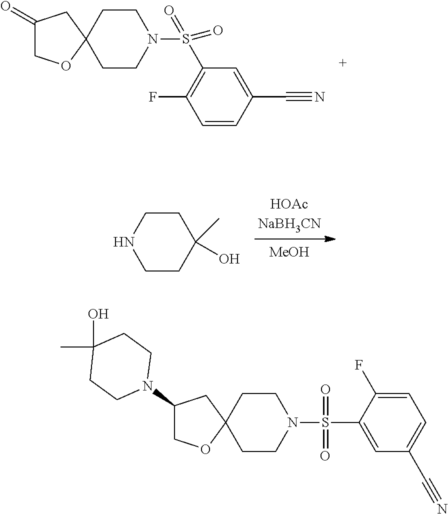

To a solution of 4-fluoro-3-[(3-oxo-1-oxa-8-azaspiro[4.5]decan-8-yl)sulfonyl]benzonitrile (500 mg, 1.48 mmol) and 4-methylpiperidin-4-ol (204.24 mg, 1.77 mmol) in MeOH (10 mL) was added acetic acid (88.74 mg, 1.48 mmol, 84.59 uL) and NaBH₃CN (464.32 mg, 7.39 mmol). The reaction mixture was stirred at 15° C. for 12 h. The mixture was diluted with water (10 mL), extracted with DCM (30 mL×3). The organic phase was washed with brine (20 mL), dried over anhydrous sodium sulfate, filtered and concentrated in vacuo. The crude material was purified by flash column (MeOH in DCM=6%˜15%) and SFC (DAICEL CHIRALPAK AD (250 mm*30 mm, 10 μm), Condition: 0.1% NH₃H₂O MeOH, 50%˜50%, Flow Rate (ml/min): 60) to give the title compound (186.36 mg, 29%) as the first compound off the chiral column. Stereochemical assignment was chosen arbitrarily. LCMS m/z=438.2[M+H]⁺. ¹H NMR (400 MHz, CDCl3) δ (ppm) 8.17-8.15 (m, 1H), 7.86-7.84 (m, 1H), 7.35-7.31 (m, 1H), 3.99-3.96 (m, 1H), 3.62 (d, J=11.6 Hz, 3H), 3.03-2.93 (m, 3H), 2.67-2.31 (m, 4H), 2.05-1.96 (m, 1H), 1.86-1.62 (m, 9H), 1.25 (s, 3H).

### Example 142: 6-(8-((1,3-dimethyl-1H-pyrazol-5-yl)sulfonyl)-8-azaspiro[4.5]decan-2-yl)-2-oxa-6-azaspiro[3.3]heptane

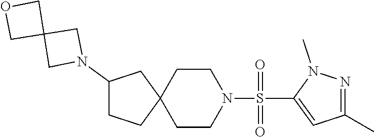 [SMILES: Cc1cc(S(=O)(=O)N2CCC3(CCC(N4CC5(COC5)C4)C3)CC2)n(C)n1]

### 1. Synthesis of 8-((1,3-dimethyl-1H-pyrazol-5-yl)sulfonyl)-8-azaspiro[4.5]decan-2-one

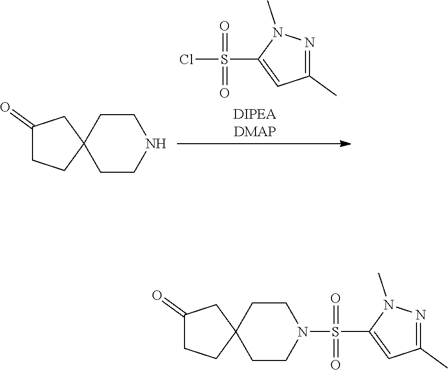 [SMILES: Cc1cc(S(=O)(=O)Cl)n(C)n1 ; O=C1CCC2(CCNCC2)C1 ; Cc1cc(S(=O)(=O)N2CCC3(CCC(=O)C3)CC2)n(C)n1]

A solution of 8-azaspiro[4.5]decan-3-one hydrochloride (2.5 g, 13.1 mmol) in DCM (50 mL) was cooled in an ice bath to <5° C., then DIPEA (9 mL 51.7 mmol) and DMAP (123 mg, 1.0 mmol) were added. After 5 min, 2,5-dimethylpyrazole-3-sulfonyl chloride (3.4 g, 17.5 mmol) in DCM (50 mL) was added. The reaction mixture was stirred at RT for 30 min. The reaction mixture was quenched with aq. sat. NaHCO₃ solution, stirred for 10 min, and extracted with DCM (3×). The combined organic layers were dried over MgSO₄, filtered, and concentrated under reduced pressure. The resulting residue was purified by silica gel column eluting with (15-80% EtOAc in heptane) to afford 8-((1,3-dimethyl-1H-pyrazol-5-yl)sulfonyl)-8-azaspiro[4.5]decan-2-one (3.49 g, 81%) as a white solid. LCMS m/z=312.1 (M+H)⁺. ¹H-NMR (500 MHz, DCM-d₂) δ (ppm): 6.46 (s, 1H), 4.01 (s, 3H), 3.37-3.32 (m, 2H), 2.95 (ddd, J=4.0, 8.4, 12.1 Hz, 2H), 2.27-2.23 (m, 5H), 2.06 (s, 2H), 1.83 (t, J=7.9 Hz, 2H), 1.71-1.63 (m, 4H).

### 2. Synthesis of 6-(8-((1,3-dimethyl-1H-pyrazol-5-yl)sulfonyl)-8-azaspiro[4.5]decan-2-yl)-2-oxa-6-azaspiro[3.3]heptane

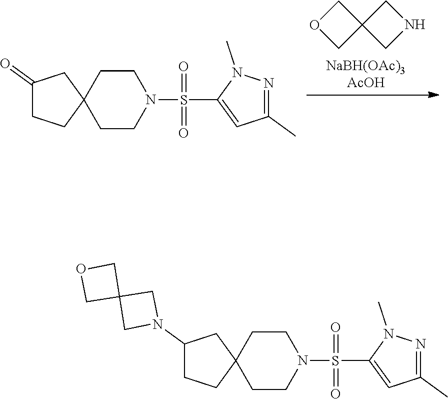 [SMILES: Cc1cc(S(=O)(=O)N2CCC3(CCC(=O)C3)CC2)n(C)n1 ; C1NCC12COC2 ; Cc1cc(S(=O)(=O)N2CCC3(CCC(N4CC5(COC5)C4)C3)CC2)n(C)n1]

A solution of 8-((1,3-dimethyl-1H-pyrazol-5-yl)sulfonyl)-8-azaspiro[4.5]decan-2-one (3.5 g, 11.1 mmol) in DCM (40 mL) was cooled to <5° C. After 10 min, AcOH (400 μL, 7.0 mmol) and a solution of 2-oxa-6-azaspiro[3.3]heptane (2.3 g, 23.2 mmol) in DCM (10 mL) were sequentially added dropwise at <5° C. After 20 min, NaBH(OAc)₃ (8.6 g, 40 mmol) was added batchwise, and the mixture was warmed to RT and stirred for 2.5 h. The reaction was quenched with aq. sat. NaHCO₃ solution. After 20 min, the biphasic mixture was extracted with DCM (3×). The combined organic extracts were dried over MgSO₄, filtered, and concentrated under reduced pressure. The resulting residue was purified by silica gel column chromatography (40-90% 3:1 EtOAc: EtOH in heptane) to afford 6-(8-((1,3-dimethyl-1H-pyrazol-5-yl)sulfonyl)-8-azaspiro[4.5]decan-2-yl)-2-oxa-6-azaspiro[3.3]heptane as a colorless oil (3.1 g, 67%). LCMS m/z=395.3 (M+H)⁺. ¹H-NMR (500 MHz, DMSO-d₆) δ (ppm): 6.58 (s, 1H), 4.55 (s, 4H), 3.92 (s, 3H), 3.20-3.10 (m, 4H), 3.06-2.99 (m, 4H), 2.62-2.54 (m, 1H), 2.18 (s, 3H), 1.58-1.51 (m, 3H), 1.46-1.39 (m, 4H), 1.35-1.26 (m, 2H), 1.07 (dd, J=5.2, 13.1 Hz, 1H).

### Example 143: 8-((4-(difluoromethoxy)phenyl)sulfonyl)-3-(6-oxa-2-azaspiro[3.5]nonan-2-yl)-1-oxa-8-azaspiro[4.5]decane

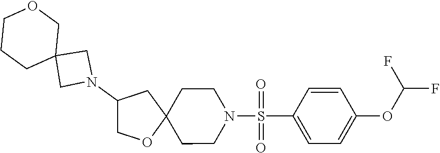 [SMILES: O=S(=O)(c1ccc(OC(F)F)cc1)N1CCC2(CC1)CC(N1CC3(CCCOC3)C1)CO2]

The synthesis was performed in a similar manner to that described in Example 71 step 1, using 8-((4-(difluoromethoxy)phenyl)sulfonyl)-1-oxa-8-azaspiro[4.5]decan-3-one and 6-oxa-2-azaspiro[3.5]nonane (9 mg, 15%). LCMS m/z=472.8 [M+H]⁺, R_{f}=1.61 min.

### Example 144: 6-(8-(quinolin-7-ylsulfonyl)-8-azaspiro[4.5]decan-2-yl)-2-oxa-6-azaspiro[3.3]heptane

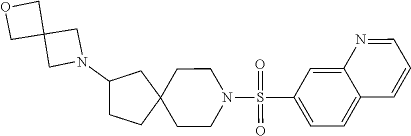 [SMILES: O=S(=O)(c1ccc2cccnc2c1)N1CCC2(CCC(N3CC4(COC4)C3)C2)CC1]

The synthesis was performed in a similar manner to that described in Example 112 using 6-(8-azaspiro[4.5]decan-2-yl)-2-oxa-6-azaspiro[3.3]heptane and quinoline-7-sulfonyl fluoride (2.1 mg, 0.5%). LCMS m/z=428.3 [M+H]⁺, R_{f}=1.47 min.

### Assays

### EBP Functional Assay

The EBP immunoaffinity (IA) LC-MS assay measures the potency of small molecule inhibitors of EBP by quantifying their concentration-dependent changes in the enzyme's substrate and product using liquid chromatography atmospheric pressure chemical ionization multiple reaction monitoring mass spectrometry (LC-APCI MRM MS). HEK293T cells were utilized as the source of EBP enzyme. The enzyme was incubated with the small molecule inhibitors at variable concentrations for 30 min. Deuterated form of EBP substrate, zymosterol-d5 (Avanti Polar Lipids, Cat #700068P-1 mg), was then added and the plate was incubated at 37° C. for 4 h. Finally, the sterol isomers were extracted and injected to LC-APCI MRM MS. MRM transition used for the quantification for both zymosterol and dihydrolathosterol (substrate and product of EBP enzymatic reaction, respectively) is 372.3-203.2, CE 30 and DP 80 in positive ion mode. Percent conversion of the zymosterol-d5 to dehydrolathosterol-d5 was used to derive IC₅₀ curves. Tasin-1 (1′-[(4-Methoxyphenyl)sulfonyl]-4-methyl-1,4′-bipiperidine, CAS 792927-06-1) was used as the reference small molecule inhibitor.

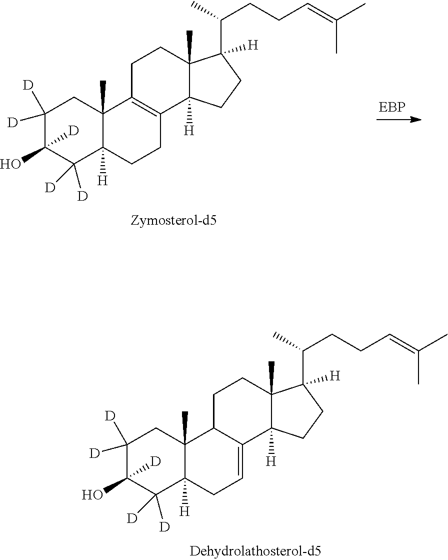

Percent conversion versus the compound concentration data were fit to the following 4-parameter logistic model to generate IC₅₀ curves:

$$y = LA + \frac{UA - LA}{1 + {10}^{( {logIC}_{50} - x ) \cdot Slope}}$$

### Data for Examples

| | EBP |
|---|---|
| Example | Functional |
| Number | Assay |
| 1 | NT |
| 2 | NT |
| 3 | NT |
| 4 | + |
| 5 | NT |
| 6 | NT |
| 7 | + |
| 8 | + |
| 9 | + |
| 10 | ++ |
| 11 | ++ |
| 12 | +++ |
| 13 | ++ |
| 14 | + |
| 15a | NT |
| 15b | + |
| 16 | + |
| 17 | + |
| 18 | ++ |
| 19 | + |
| 20 | + |
| 21 | ++ |
| 22a | + |
| 22b | ++ |
| 23 | ++ |
| 24 | + |
| 25 | ++ |
| 26 | +++ |
| 27 | +++ |
| 28 | + |
| 29 | + |
| 30a | NT |
| 30b | + |
| 30c | NT |
| 30d | NT |
| 31 | ++ |
| 32 | + |
| 33 | +++ |
| 34 | NT |
| 35 | NT |
| 36 | NT |
| 37 | NT |
| 38 | NT |
| 39 | NT |
| 40 | ++ |
| 41 | ++ |
| 42 | +++ |
| 43 | +++ |
| 44 | + |
| 45 | + |
| 46 | + |
| 47 | ++ |
| 48 | ++ |
| 49 | NT |
| 50 | ++ |
| 51 | +++ |
| 52 | ++ |
| 53a | +++ |
| 53b | ++ |
| 54 | + |
| 55 | + |
| 56 | ++ |
| 57a | + |
| 57b | + |
| 58 | + |
| 59 | + |
| 60 | + |
| 61 | + |
| 62 | + |
| 63a | +++ |
| 63b | NT |
| 64 | + |
| 65 | ++ |
| 66 | + |
| 67 | + |
| 68a | ++ |
| 68b | ++ |
| 69 | + |
| 70 | + |
| 71 | NT |
| 72a | ++ |
| 72b | NT |
| 72c | NT |
| 72d | NT |
| 73 | NT |
| 74 | NT |
| 75 | NT |
| 76 | NT |
| 77 | NT |
| 78 | NT |
| 79 | NT |
| 80 | NT |
| 81 | NT |
| 82 | NT |
| 83 | NT |
| 84 | ++ |
| 85a | ++ |
| 85b | +++ |
| 86a | +++ |
| 86b | +++ |
| 87 | + |
| 88 | ++ |
| 89 | ++ |
| 90a | ++ |
| 90b | +++ |
| 91 | + |
| 92 | ++ |
| 93 | ++ |
| 94 | +++ |
| 95 | +++ |
| 96 | + |
| 97 | ++ |
| 98 | + |
| 99 | +++ |
| 100 | +++ |
| 101 | +++ |
| 102 | ++ |
| 103 | +++ |
| 104 | +++ |
| 105 | + |
| 106 | +++ |
| 107 | +++ |
| 108 | + |
| 109 | ++ |
| 110 | ++ |
| 111 | + |
| 112 | +++ |
| 113 | +++ |
| 114 | + |
| 114 | + |
| 115 | + |
| 116 | + |
| 117 | + |
| 118 | ++ |
| 119a | +++ |
| 119b | ++ |
| 120 | +++ |
| 121 | +++ |
| 122 | +++ |
| 123 | +++ |
| 124 | +++ |
| 125 | +++ |
| 126 | ++ |
| 127 | + |
| 128 | +++ |
| 129 | +++ |
| 130 | ++ |
| 131 | ++ |
| 132 | +++ |
| 133 | ++ |
| 134 | + |
| 135 | ++ |
| 136 | ++ |
| 137 | + |
| 138 | + |
| 139 | ++ |
| 140 | +++ |
| 141 | + |
| 142 | ++ |
| 143 | ++ |
| 144 | ++ |

*+ means >250 nM and ≤1500 nM; ++ means 100-250 nM; +++ means <100 nM; NT = Not Tested

What is claimed is:

1. A compound represented by Formula (I): 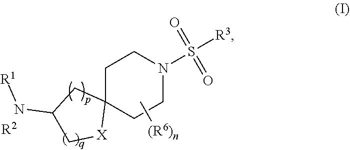
or a pharmaceutically acceptable salt thereof, wherein:
X is O, CH₂, or a bond, provided when X is a bond, p and q are 1;
p is 0 or 1, provided that when p is 0, q is 1;
q is 0 or 1, provided that when q is 0, p is 1;
R¹ and R² are each independently selected from H, C₁₋₆alkyl, C₃₋₈cycloalkyl, C₃₋₈cycloalkenyl, 6 to 10-membered bicyclic heterocycyl, and 4 to 6-membered monocyclic heterocyclyl, wherein the C₁₋₆alkyl, C₃₋₈cycloalkyl, C₃₋₈cycloalkenyl, 6 to 10-membered bicyclic heterocycyl, and 4 to 6-membered monocyclic heterocyclyl are each optionally substituted with one or more R⁴, provided at least one of R¹ and R² is not H;
or R¹ and R² together with the N atom from which they are attached form a 4 to 7-membered monocyclic heterocycle or 6 to 10-membered bicyclic heterocycle, each of which is optionally substituted with one or more R⁴;
each R⁴ is independently selected from OR^{4a}, halo, C₁₋₃alkyl, C₃₋₈cycloalkyl, and 4 to 6-membered monocyclic heterocyclyl;
R^{4a} is selected from H and C₁₋₃alkyl, wherein the C₁₋₃alkyl is optionally substituted with one or more halo;
R³ is phenyl, 5 or 6-membered monocyclic heteroaryl, 9 or 10-membered bicyclic heteroaryl, or 6 to 10 membered bicyclic heterocycle, wherein the phenyl, 5 or 6-membered monocyclic heteroaryl, 9 or 10-membered bicyclic heteroaryl, and 6 to 10 membered bicyclic heterocycle are each optionally substituted with one or more R⁵;
each R⁵ is independently selected from C₁₋₃alkyl, C₃₋₆cycloalkyl, OR^{5a}, cyano, halo, and 5 or 6-membered monocyclic heteroaryl, wherein the C₁₋₃alkyl, C₃₋₆ cycloalkyl, and heteroaryl are optionally substituted with one or more R^{5b};
R^{5a} is selected from H, C₁₋₃alkyl, and C₃₋₆cycloalkyl, wherein the C₁₋₃alkyl is optionally substituted with one or more halo;
R^{5b} is selected from OR^{5a}, C₁₋₃alkyl, cyano, and halo;
R⁶, for each occurrence, is independently halo or C₁₋₃alkyl, or two R⁶ together to form a —C₁₋₃alkylene
n is 0, 1, or 2;
m is 0, 1, or 2;
provided that the compound is not any one of the compounds shown in Table I.

2. The compound of claim 1, wherein the compound is represented by Formula (I): 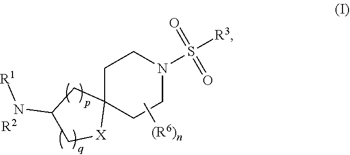
or a pharmaceutically acceptable salt thereof, wherein:
X is O, CH₂, or a bond, provided when X is a bond, p and q are 1;
p is 0 or 1, provided that when p is 0, q is 1;
q is 0 or 1, provided that when q is 0, p is 1;
R¹ and R² are each independently selected from H, C₁₋₆alkyl, C₃₋₈cycloalkyl, C₃₋₈cycloalkenyl, and 4 to 6-membered monocyclic heterocyclyl, wherein the C₁₋₆alkyl, C₃₋₈cycloalkyl, C₃₋₈cycloalkenyl, and 4 to 6-membered monocyclic heterocyclyl are each optionally substituted with one or more R⁴, provided at least one of R¹ and R² is not H;
or R¹ and R² together with the N atom from which they are attached form a 4 to 7-membered monocyclic heterocycle or 6 to 10-membered bicyclic heterocycle, each of which is optionally substituted with one or more R⁴;
R⁴ is selected from OR^{4a}, halo, C₁₋₃alkyl, or C₃₋₈cycloalkyl;
R^{4a} is selected from H and C₁₋₃alkyl, wherein the C₁₋₃alkyl is optionally substituted with one or more halo;
R³ is phenyl, 5 or 6-membered monocyclic heteroaryl, 9 or 10-membered bicyclic heteroaryl, or 6 to 10 membered bicyclic heterocycle, wherein the phenyl, 5 or 6-membered monocyclic heteroaryl, 9 or 10-membered bicyclic heteroaryl, and 6 to 10 membered bicyclic heterocycle are each optionally substituted with one or more R⁵;
each R⁵ is independently selected from C₁₋₃alkyl, C₃₋₆cycloalkyl, OR^{5a}, cyano, halo, and 5 or 6-membered monocyclic heteroaryl, wherein the C₁₋₃alkyl, C₃₋₆cycloalkyl, and heteroaryl are optionally substituted with one or more R^{5b};
R^{5a} is selected from H, C₁₋₃alkyl, and C₃₋₆cycloalkyl, wherein the C₁₋₃alkyl is optionally substituted with one or more halo;
R^{5b} is selected from OR^{5a}, C₁₋₃alkyl, cyano, and halo;
R⁶, for each occurrence, is independently halo or C₁₋₃alkyl, or two R⁶ together to form a —C₁₋₃alkylene
n is 0, 1, or 2;
m is 0, 1, or 2;
provided that the compound is not any one of the compounds shown in Table I.

3. The compound according to claim 1, or a pharmaceutically acceptable salt thereof, wherein (i) when X is O, and R¹ and R² together with the N atom from which they are attached form unsubstituted morpholine, unsubstituted pyrrolidine, or unsubstituted N-methylpiperazine, then R³ is selected from a 9 or 10-membered bicyclic heteroaryl or a 6 to 10 membered bicyclic heterocycle each optionally substituted with one or more substituent R⁵ or a phenyl or a 5 or 6-membered monocyclic heteroaryl each substituted with at least two R⁵ groups, or one R⁵ group that is OR^{5a}; (ii) when X is O, and R¹ and R² are both C₁₋₆alkyl, then at least one of the C₁₋₆alkyl represented by R¹ and R² is substituted by one or more R⁴ selected from OR^{4a}, halo, C₃₋₈cycloalkyl, and 4 to 6-membered monocyclic heterocyclyl; (iii) when X is a bond, and R¹ and R² are both C₁₋₆alkyl, then at least one of the C₁₋₆alkyl represented by R¹ and R² is substituted by one or more R⁴; or (iv) when p is 0 and q is 1, then X is O.

4. The compound according to claim 1 or 2, or a pharmaceutically acceptable salt thereof, wherein (i) when X is O, and R¹ and R² together with the N atom from which they are attached form unsubstituted morpholine, unsubstituted pyrrolidine, or unsubstituted N-methylpiperazine, then R³ is selected from a 9 or 10-membered bicyclic heteroaryl or a 6 to 10 membered bicyclic heterocycle each optionally substituted with one or more substituent R⁵ or a phenyl or a 5 or 6-membered monocyclic heteroaryl each substituted with at least two R⁵ groups, or one R⁵ group that is OR^{5a}; (ii) when X is O, and R¹ and R² are both C₁₋₆alkyl, then at least one of the C₁₋₆alkyl represented by R¹ and R² is substituted by one or more R⁴ selected from OR^{4a}, halo, and C₃₋₈cycloalkyl; (iii) when X is a bond, and R¹ and R² are both C₁₋₆ alkyl, then at least one of the C₁₋₆alkyl represented by R¹ and R² is substituted by one or more R⁴; or (iv) when p is 0 and q is 1, then X is O.

5. The compound of any one of claims 1 to 4, wherein the compound is represented by Formula (II): 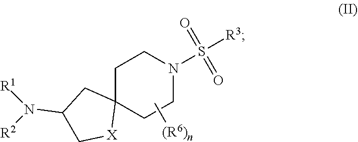
or a pharmaceutically acceptable salt thereof.

6. The compound of claim 5, wherein the compound is represented by Formula (IIA) or (IIB): 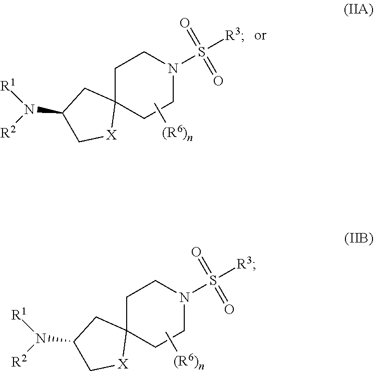
or a pharmaceutically acceptable salt thereof.

7. The compound of claim 5, wherein the compound is represented by Formula (III) or (IV): 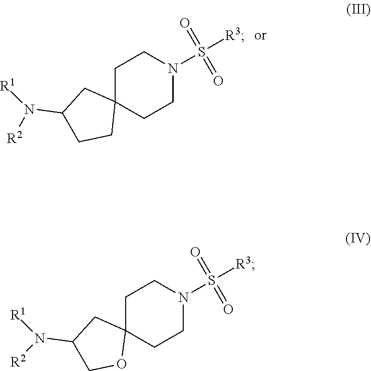
or a pharmaceutically acceptable salt thereof.

8. The compound of claim 7, wherein the compound is represented by Formula (IIIA), (IIIB), (IVA), or (IVB): 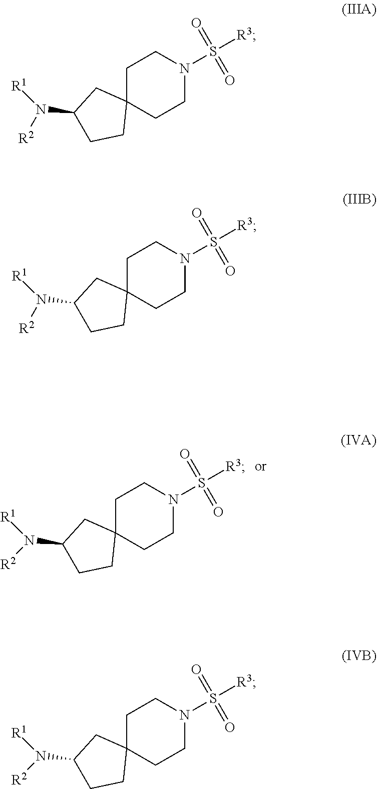
or a pharmaceutically acceptable salt thereof.

9. The compound of any one of claims 1 to 8, or a pharmaceutically acceptable salt thereof, wherein R³ is phenyl, 5 or 6-membered monocyclic heteroaryl, 9 to 10 membered bicyclic heteroaryl or 8 to 10 membered bicyclic heterocycle, wherein the phenyl, 5 or 6-membered monocyclic heteroaryl, 9 to 10 membered bicyclic heteroaryl and 8 to 10 membered bicyclic heterocycle are each optionally substituted with one to three R⁵.

10. The compound of claim 9, or a pharmaceutically acceptable salt thereof, wherein R³ is selected from the group consisting of pyridyl, pyrimidinyl, phenyl, thiazolyl, pyrazolyl, pyrazinyl, triazoyl, imidazopyridinyl, quinolinyl, tetrahydropyranopyrazolyl, thiophenyl, benzothiophenyl, furanyl, indazolyl, indolizinyl, pyrazolo[1,5-a]pyridinyl, 2,3-dihydrobenzo[b][1,4]dioxinyl, and benzofuranyl, each of which are each optionally substituted with one to three R⁵.

11. The compound of claim 9, or a pharmaceutically acceptable salt thereof, wherein R³ is selected from the group consisting of pyridyl, phenyl, thiazolyl, pyrazolyl, pyrazinyl, imidazopyridinyl, quinolinyl, tetrahydropyranopyrazolyl, thiophenyl, benzothiophenyl, furanyl, indazolyl, indolizinyl, and benzofuranyl, each of which are each optionally substituted with one to three R⁵.

12. The compound of claim 10, or a pharmaceutically acceptable salt thereof, wherein R³ is selected from the group consisting of pyridyl, phenyl, pyrazoyl, thiophenyl, thiazolyl, quinolinyl, tetrahydropyranopyrazolyl, and benzofuranyl, each of which are each optionally substituted with one to three R⁵.

13. The compound claim 9, or a pharmaceutically acceptable salt thereof, wherein R³ is represented by the following formula: 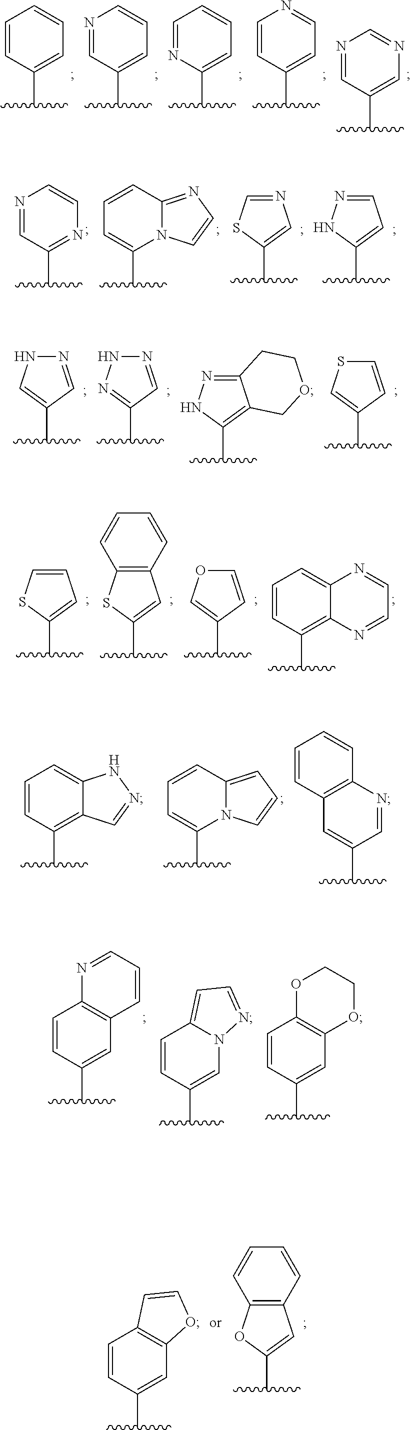
wherein each of the formula depicted above is optionally substituted with one to three R⁵.

14. The compound claim 9, or a pharmaceutically acceptable salt thereof, wherein R³ is represented by the following formula: 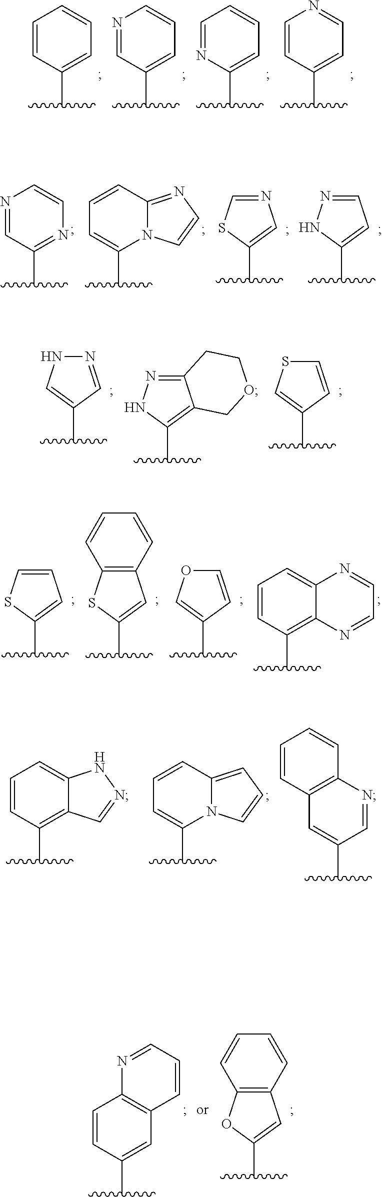
wherein each of the formula depicted above is optionally substituted with one to three R⁵.

15. The compound of claim 14, or a pharmaceutically acceptable salt thereof, wherein R³ is represented by the following formula: 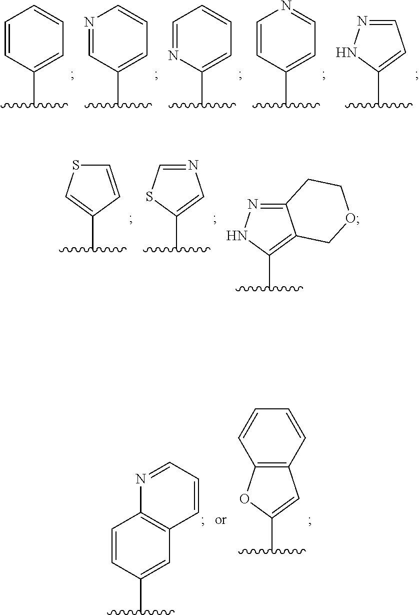
wherein each of the formula depicted above is optionally substituted with one to three R⁵.

16. The compound of claim 13, or a pharmaceutically acceptable salt thereof, wherein R³ is represented by the following formula:

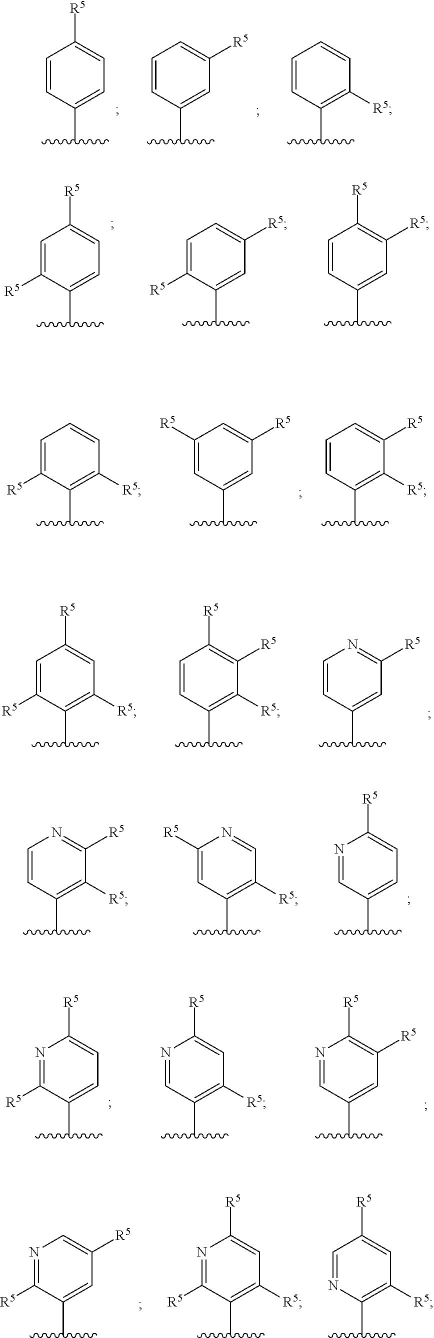

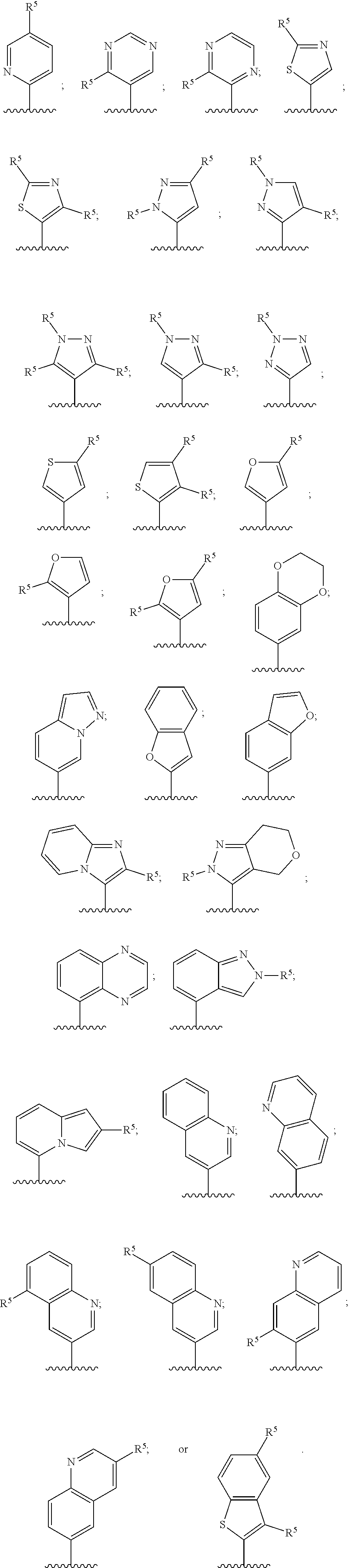

17. The compound of claim 14, or a pharmaceutically acceptable salt thereof, wherein R³ is represented by the following formula:

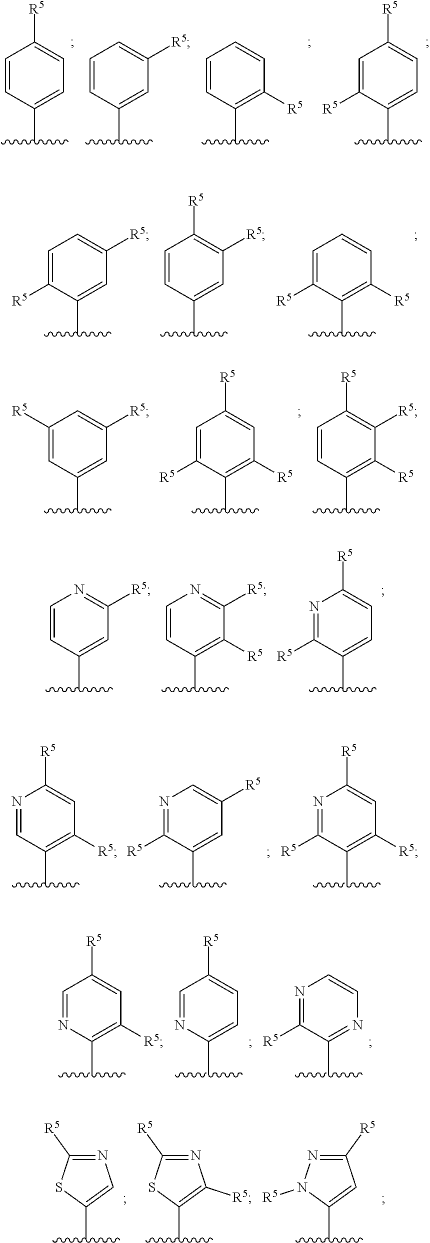

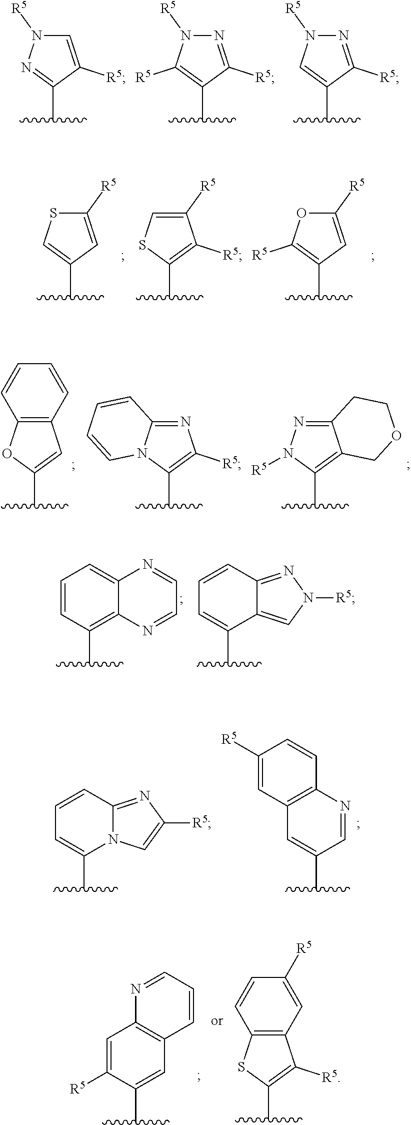

18. The compound of claim 17, or a pharmaceutically acceptable salt thereof, wherein R³ is represented by the following formula:

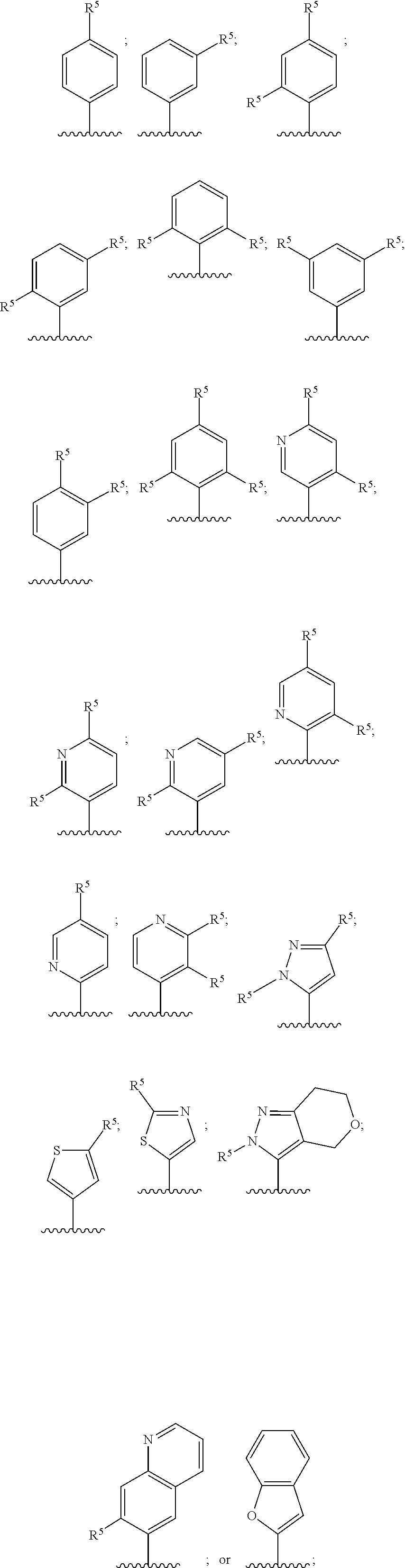

19. The compound of any one of claims 1 to 18, or a pharmaceutically acceptable salt thereof, wherein:
R⁵, for each occurrence, is independently selected from cyano, C₁₋₄alkyl, C₃₋₆cycloalkyl, 5 or 6-membered monocyclic heteroaryl, OR^{5a}, and halo, wherein the C₁₋₄alkyl is optionally substituted with one to three R^{5b}, and the 5 or 6-membered monocyclic heteroaryl is optionally substituted with C₁₋₃alkyl;
R^{5a} is H, C₁₋₃alkyl or C₃₋₆cycloalkyl, wherein C₁₋₃alkyl is optionally substituted with one to three halo;
R^{5b}, for each occurrence, is independently selected from halo and —OR^{5a}.

20. The compound of claim 19, or a pharmaceutically acceptable salt thereof, wherein R⁵, for each occurrence, is independently selected from —CH₃, —CHF₂, —C(CH₃)₃, —CH₂CH₃, —CH₂CN, —CF₃, —CH₂OCH₃, —OCH₃, —OCHF₂, —OCF₃, —OCH₂CH₃, —OCH(CH₃)₂, —OH, —F, —Cl, cyclopropyl, cyclopropyloxy, 4-methyloxazol-2-yl, and —CN.

21. The compound of claim 19, or a pharmaceutically acceptable salt thereof, wherein R⁵, for each occurrence, is independently selected from —CH₃, —C(CH₃)₃, —CH₂CH₃, —CH₂CN, —CF₃, —CH₂OCH₃, —OCH₃, —OCHF₂, —OCF₃, —OCH₂CH₃, —OH, —F, —Cl, cyclopropyl, cyclopropyloxy, 4-methyloxazol-2-yl, and —CN.

22. The compound of any one of claims 1 to 21, or a pharmaceutically acceptable salt thereof, wherein R¹ and R² together with the N atom from which they are attached form a 4 to 6-membered monocyclic heterocycle or 6 to 10-membered bicyclic heterocycle, each of which is optionally substituted with one or two R⁴.

23. The compound of claim 22, or a pharmaceutically acceptable salt thereof, wherein R¹ and R² together with the nitrogen atom from which they are attached form groups represented by the following formula: 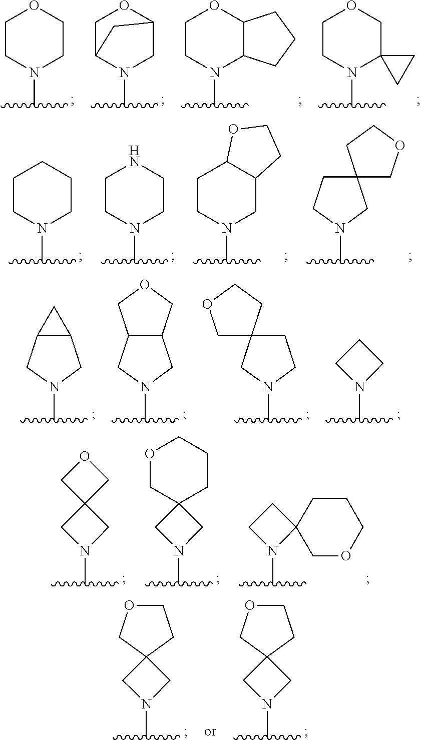
wherein each of the formula depicted above is optionally substituted with one or two R⁴.

24. The compound of claim 22, or a pharmaceutically acceptable salt thereof, wherein R¹ and R² together with the nitrogen atom from which they are attached form groups represented by the following formula: 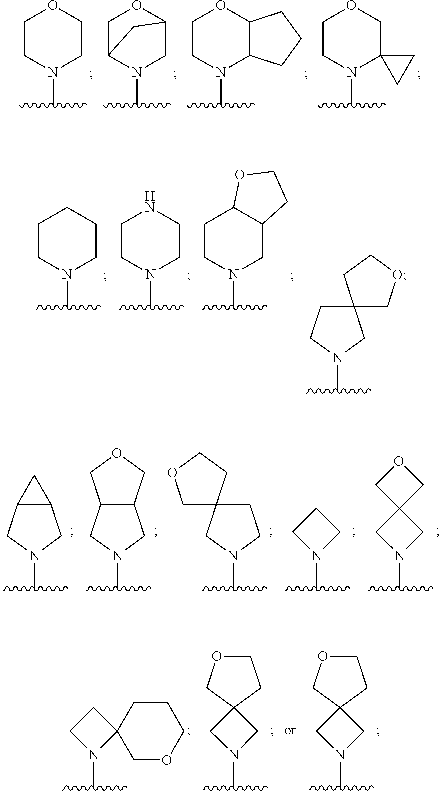
wherein each of the formula depicted above is optionally substituted with one or two R⁴.

25. The compound of claim 24, or a pharmaceutically acceptable salt thereof, wherein R¹ and R² together with the nitrogen atom from which they are attached form groups represented by the following formula: 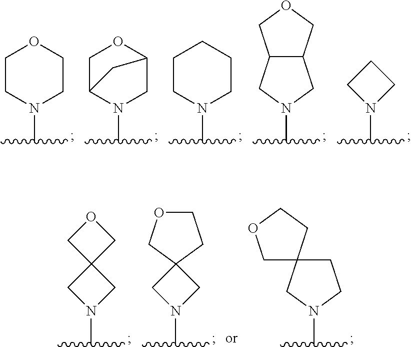
wherein each of the formula depicted above is optionally substituted with one or two R⁴.

26. The compound of claim 23, or a pharmaceutically acceptable salt thereof, wherein R¹ and R² together with the nitrogen atom from which they are attached form groups represented by the following formula:

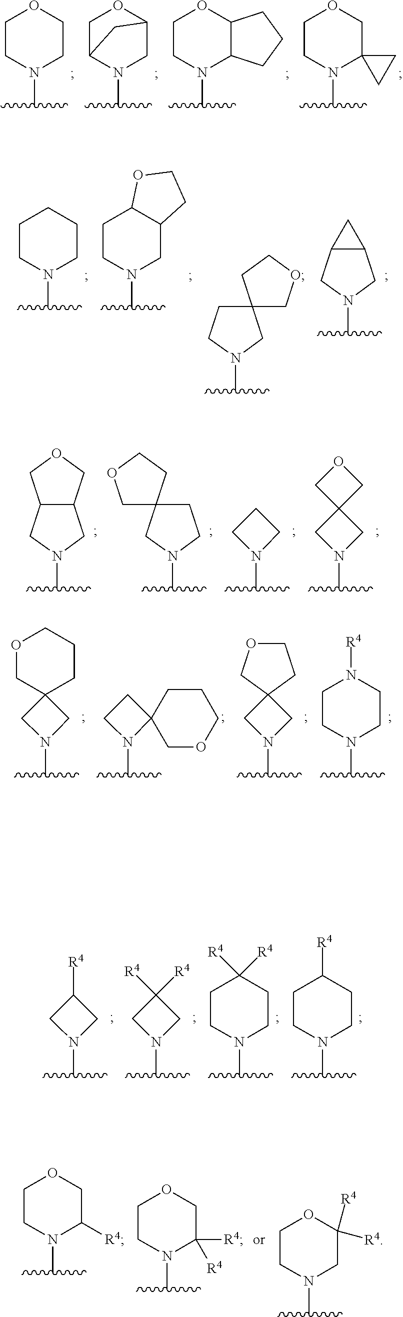

27. The compound of claim 24, or a pharmaceutically acceptable salt thereof, wherein R¹ and R² together with the nitrogen atom from which they are attached form groups represented by the following formula:

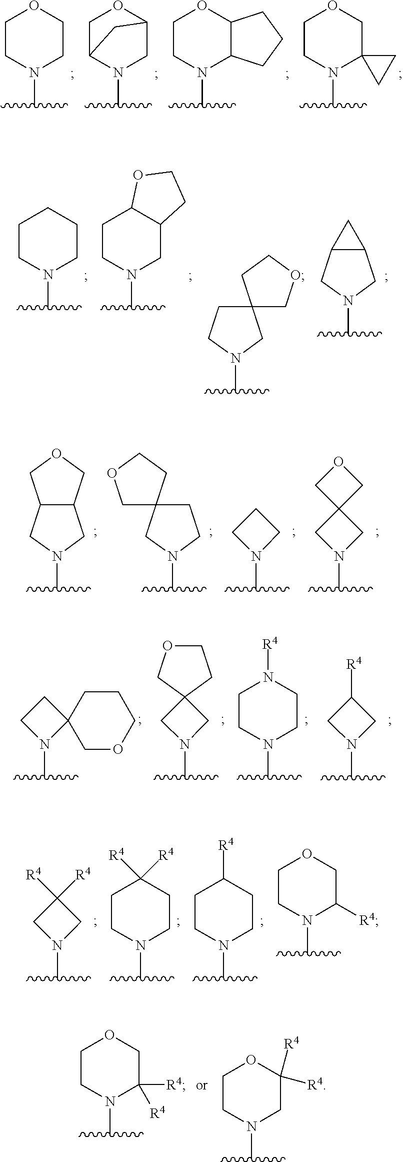

28. The compound of claim 27, or a pharmaceutically acceptable salt thereof, wherein R¹ and R² together with the nitrogen atom from which they are attached form groups represented by the following formula:

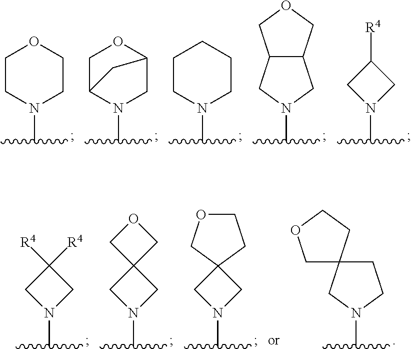

29. The compound of any one of claims 1 to 28, or a pharmaceutically acceptable salt thereof, wherein R⁴, for each occurrence, is independently selected from halo, C₁₋₃ alkyl and —OR⁴a; and R^{4a} is H or C₁₋₃alkyl.

30. The compound of claim 29, or a pharmaceutically acceptable salt thereof, R⁴, for each occurrence, is independently selected from —OCH₃, F, —OH, and —CH₃.

31. The compound of any one of claims 1 to 21, or a pharmaceutically acceptable salt thereof, wherein:
R¹ is H or C₁₋₃alkyl optionally substituted with one to three R⁴; and
R² is C₁₋₃alkyl, C₃₋₆cycloalkyl, C₃₋₆cycloalkenyl 6 to 10-membered bicyclic heterocyclyl, or 4 to 6-membered monocyclic heterocyclyl, wherein the C₁₋₃alkyl, C₃₋₆cycloalkyl, C₃₋₆cycloalkenyl, 6 to 10-membered bicyclic heterocyclyl, and 4 to 6-membered monocyclic heterocyclyl are each optionally substituted with one or two substituents independently selected from C₁₋₃alkyl, C₁₋₃alkoxy, halo, and 4 to 6-membered monocyclic heterocyclyl.

32. The compound of any one of claims 1 to 21, or a pharmaceutically acceptable salt thereof, wherein:
R¹ is H or C₁₋₃alkyl optionally substituted with one to three R⁴; and
R² is C₁₋₃alkyl, C₃₋₆cycloalkyl, C₃₋₆cycloalkenyl or 4 to 6-membered monocyclic heterocyclyl, wherein the C₁₋₃alkyl, C₃₋₆cycloalkyl, C₃₋₆cycloalkenyl and 4 to 6-membered monocyclic heterocyclyl are each optionally substituted with one or two substituents independently selected from C₁₋₃alkyl, C₁₋₃alkoxy, and halo.

33. The compound of claim 31, or a pharmaceutically acceptable salt thereof, wherein R¹ is H or C₁₋₃alkyl optionally substituted one to three R⁴; and R² is C₁₋₃alkyl substituted with C₁₋₃alkoxy or a 4 to 6-membered monocyclic heterocyclyl, C₃₋₆cycloalkyl optionally substituted with one to two halo, 6 to 10-membered bicyclic heterocyclyl, or a 4 to 6-membered monocyclic heterocyclyl optionally substituted with one or two C₁₋₃alkyl.

34. The compound of claim 32, or a pharmaceutically acceptable salt thereof, wherein R¹ is H or C₁₋₃alkyl optionally substituted one to three R⁴; and R² is C₁₋₃alkyl substituted with C₁₋₃alkoxy, C₃₋₆cycloalkyl optionally substituted with one to two halo, or a 4 to 6-membered monocyclic heterocyclyl optionally substituted with one or two C₁₋₃alkyl.

35. The compound of claim 31, or a pharmaceutically acceptable salt thereof, wherein R¹ is H or methyl; and R² is 2-methoxyethyl, —CH₂-tetrahydropyranyl, 4,4-difluorocyclohexyl, 4-fluorocyclohex-3-en-1-yl, 2,2-difluoroethyl, 4-methylpiperidinyl, tetrahydro-2H-pyran-4-yl, oxetan-3-yl, 3-methyloxetan-3-yl, tetrahydrofuran-3-yl, 3-methyltetrahydrofuran-3-yl, and 2-oxaspiro[3.3]heptan-6-yl.

36. The compound of claim 32, or a pharmaceutically acceptable salt thereof, wherein R¹ is H or methyl; and R² is 2-methoxyethyl, 4,4-difluorocyclohexyl, 4-fluorocyclohex-3-en-1-yl, 2,2-difluoroethyl, 4-methylpiperidinyl, tetrahydro-2H-pyran-4-yl, 3-methyloxetan-3-yl, tetrahydrofuran-3-yl and 2-oxaspiro[3.3]heptan-6-yl.

37. The compound of any one of claims 1 to 4, or a pharmaceutically acceptable salt thereof, wherein the compound is represented by the following formula: 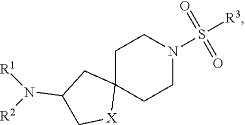
or a pharmaceutically acceptable salt thereof, wherein:
X is O or CH₂;
R¹ is H or C₁₋₃alkyl optionally substituted with one to three R⁴;
R² is C₁₋₃alkyl substituted with C₁₋₃alkoxy or a 4 to 6-membered monocyclic heterocyclyl, C₃₋₆cycloalkyl optionally substituted with one to two halo, 6 to 10-membered bicyclic heterocyclyl, or a 4 to 6-membered monocyclic heterocyclyl optionally substituted with one or two C₁₋₃alkyl;
or R¹ and R² together with the N atom from which they are attached form a 4 to 6-membered monocyclic heterocycle, 7 to 9-membered bicyclic heterocycle, each of which is optionally substituted with one or two R⁴;
R⁴, for each occurrence, is independently selected from C₁₋₃alkoxy, OH, halo, and C₁₋₃alkyl;
R³ is phenyl, 5 or 6-membered monocyclic heteroaryl, 9 or 10-membered bicyclic heteroaryl, or 9 or 10-membered bicyclic heterocycle, wherein the phenyl, 5 or 6-membered monocyclic heteroaryl, 9 or 10-membered bicyclic heteroaryl and 9 or 10-membered bicyclic heterocycle are each optionally substituted with one to three R⁵;
R⁵ is cyano, C₁₋₄alkyl, C₃₋₆cycloalkyl, OR^{5a}, and halo, wherein the C₁₋₄alkyl is optionally substituted with one to three R^{5b};
R^{5a} is C₁₋₃alkyl is optionally substituted with one to three halo.

38. The compound of claim 37, or a pharmaceutically acceptable salt thereof, wherein:
X is O or CH₂;
R¹ is H or C₁₋₃alkyl optionally substituted with one to three R⁴;
R² is C₁₋₃alkyl substituted with C₁₋₃alkoxy, C₃₋₆cycloalkyl optionally substituted with one to two halo, or a 4 to 6-membered monocyclic heterocyclyl optionally substituted with one or two C₁₋₃alkyl;
or R¹ and R² together with the N atom from which they are attached form a 4 to 6-membered monocyclic heterocycle, 7 to 9-membered bicyclic heterocycle, each of which is optionally substituted with one or two R⁴;
R⁴, for each occurrence, is independently selected from C₁₋₃alkoxy, OH, halo, and C₁₋₃alkyl;
R³ is phenyl, 5 or 6-membered monocyclic heteroaryl, 9 or 10-membered bicyclic heteroaryl, or 9 or 10-membered bicyclic heterocycle, wherein the phenyl, 5 or 6-membered monocyclic heteroaryl, 9 or 10-membered bicyclic heteroaryl and 9 or 10-membered bicyclic heterocycle are each optionally substituted with one to three R⁵;
R⁵ is cyano, C₁₋₄alkyl, C₃₋₆cycloalkyl, OR^{5a}, and halo, wherein the C₁₋₄alkyl is optionally substituted with one to three R^{5b};
R^{5a} is C₁₋₃alkyl is optionally substituted with one to three halo.

39. The compound of claim 37, or a pharmaceutically acceptable salt thereof, wherein:
R¹ is H or C₁₋₃alkyl optionally substituted one to three R⁴ and R² is C₁₋₃alkyl substituted with C₁₋₃alkoxy or a 4 to 6-membered monocyclic heterocyclyl, 6 to 10-membered bicyclic heterocyclyl, or a 4 to 6-membered monocyclic heterocyclyl optionally substituted with one or two substituents independently selected from halo and C₁₋₃alkyl;
or R¹ and R² together with the N atom from which they are attached form the groups represented by the following formula: 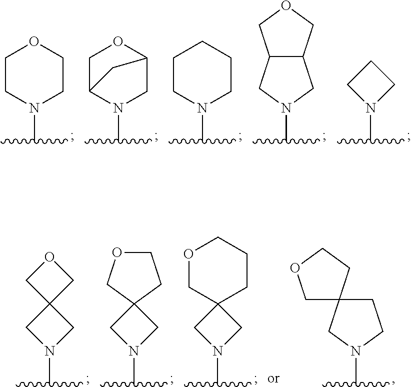
each of which is optionally substituted with one or two R⁴;
R³ is 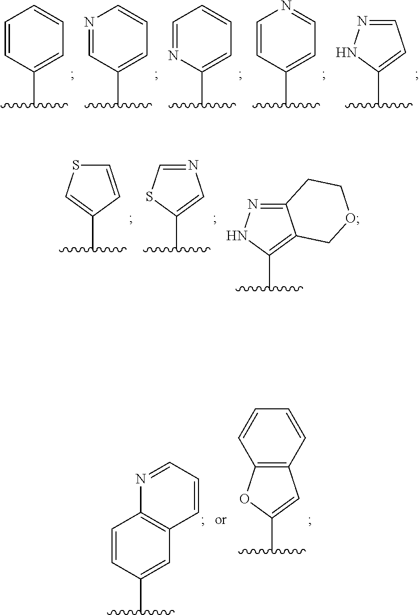
each of which is optionally substituted with one to three R⁵.

40. The compound of claim 38, or a pharmaceutically acceptable salt thereof, wherein:
R¹ is H or C₁₋₃alkyl optionally substituted one to three R⁴ and R² is C₁₋₃alkyl substituted with C₁₋₃alkoxy, or a 4 to 6-membered monocyclic heterocyclyl optionally substituted with one or two substituents independently selected from halo and C₁₋₃alkyl;
or R¹ and R² together with the N atom from which they are attached form the groups represented by the following formula: 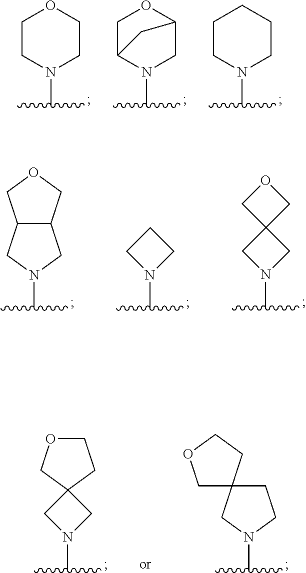
each of which is optionally substituted with one or two R⁴; 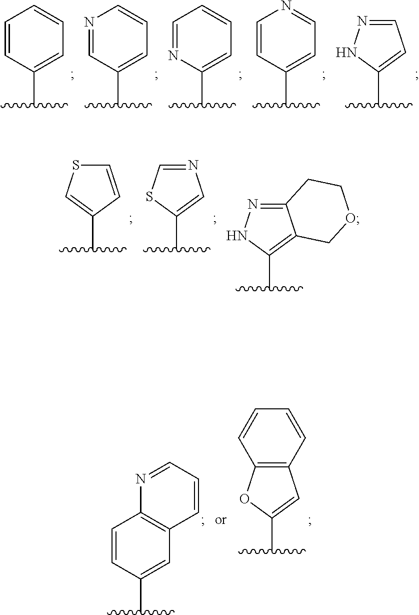
each of which is optionally substituted with one to three R⁵.

41. The compound of any one of claims 37 to 40, or a pharmaceutically acceptable salt thereof, wherein R³ is represented by the following formula: 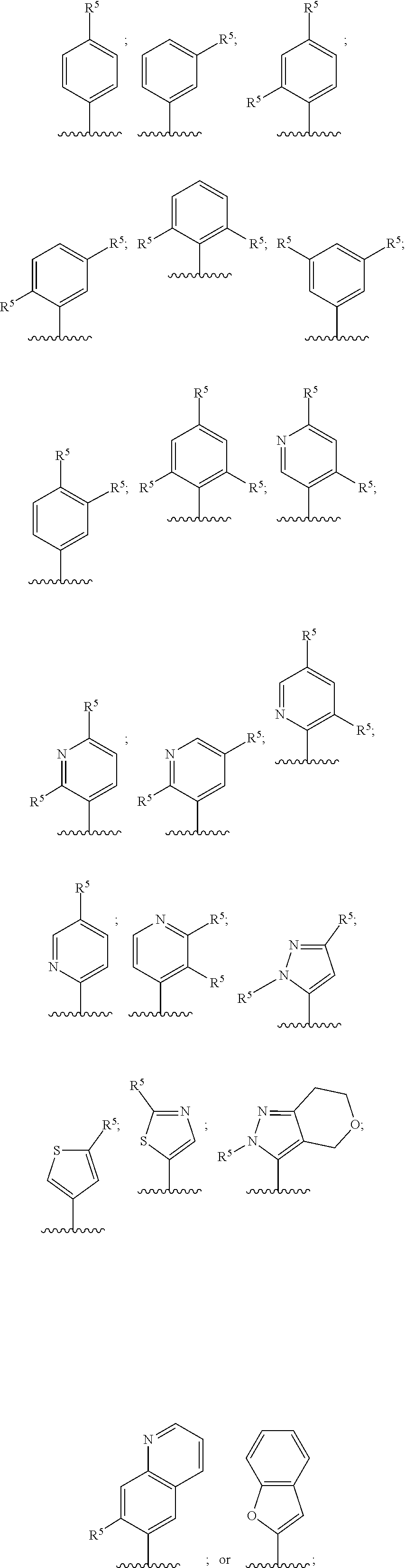
and wherein:
(1) R¹ is H or CH₃ and R² is 2-methoxyethyl, oxatan-3-ylmethyl, 3-methyltetrahydrofuran-3-yl, tetrahydro-2H-pyran-4-yl, —CH₂-tetrahydropyranyl, or 2-oxaspiro[3.3]heptan-6-yl; or
(2) R¹ and R² together with the nitrogen atom from which they are attached form groups represented by the following formula:

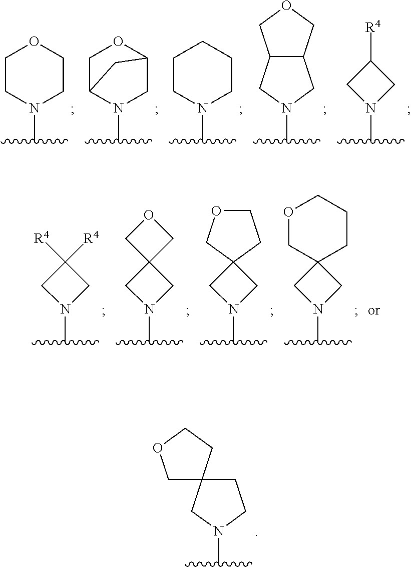

42. The compound of any one of claims 37 to 40, or a pharmaceutically acceptable salt thereof, wherein R³ is represented by the following formula: 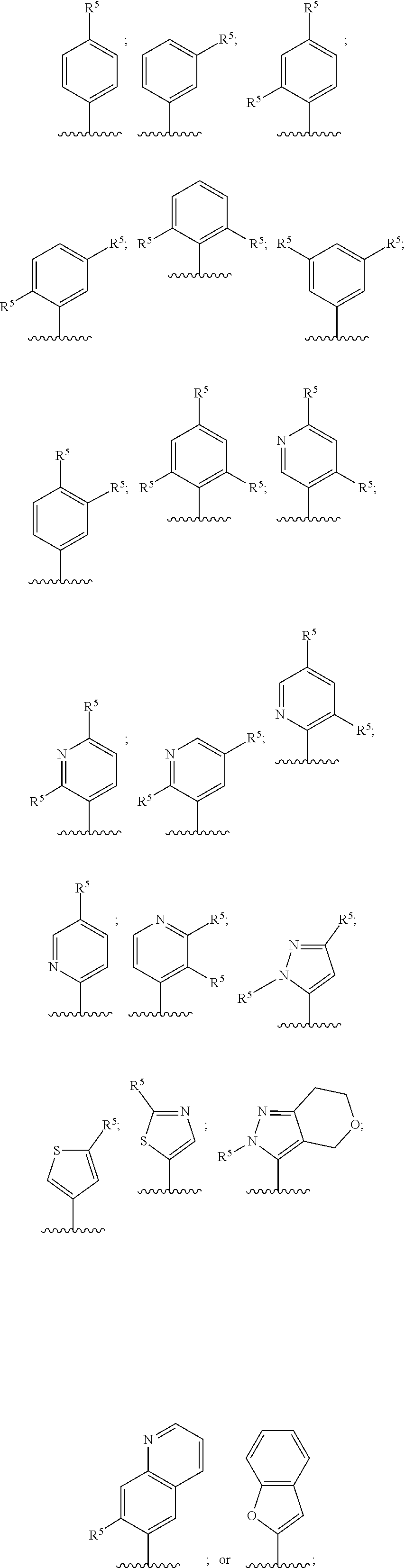
and wherein R¹ and R² together with the nitrogen atom from which they are attached form groups represented by the following formula:

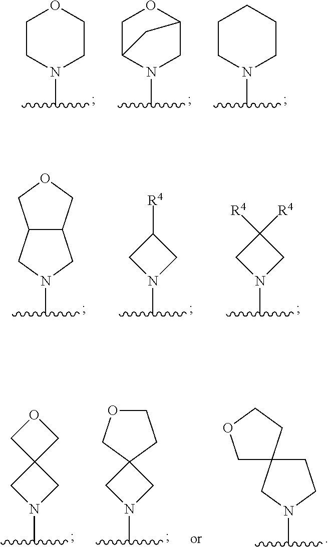

43. The compound of any one of claims 37 to 42, or a pharmaceutically acceptable salt thereof, wherein R⁴, for each occurrence, is independently selected from halo, C₁₋₃ alkyl and —OR⁴a; and R^{4a} is H or C₁₋₃alkyl.

44. The compound of claim 43, or a pharmaceutically acceptable salt thereof, wherein R⁴, for each occurrence, is independently selected from —OCH₃, F, —OH, or —CH₃.

45. The compound of any one of claims 37 to 44, or a pharmaceutically acceptable salt thereof, wherein
R⁵, for each occurrence, is independently selected from cyano, C₁₋₄alkyl, C₃₋₆cycloalkyl, OR^{5a}, and halo, wherein the C₁₋₄alkyl is optionally substituted with one to three R^{5b};
R^{5a} is H, C₃₋₆cycloalkyl or C₁₋₃alkyl optionally substituted with one to three halo;
R^{5b}, for each occurrence, is independently selected from halo and C₁₋₃alkoxy.

46. The compound of claim 45, or a pharmaceutically acceptable salt thereof, wherein R⁵, for each occurrence, is independently selected from —CH₃, —CF₃, —OCH₃, —OCHF₂, —OCF₃, —OH, —CN, F, Cl, —CH₂OCH₃, cyclopropyl, and cyclopropyloxy.

47. The compound of any one of claims 1 to 4, wherein the compound is represented by Formula (V): 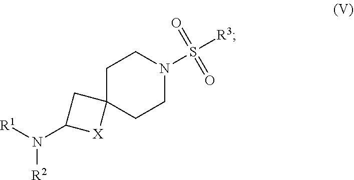
or a pharmaceutically acceptable salt thereof.

48. The compound of claim 47, wherein the compound is represented by Formula (VIA) or (VIB): 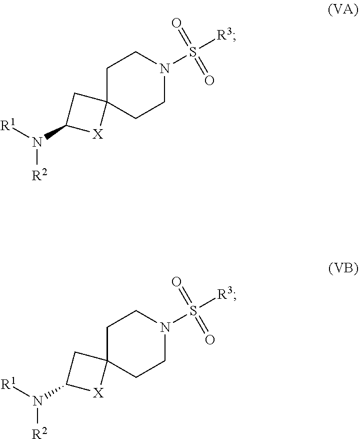
or a pharmaceutically acceptable salt thereof.

49. The compound of claims 47 or 48, wherein X is CH₂.

50. The compound of any one of claims 47 to 49, or a pharmaceutically acceptable salt thereof, wherein R³ is phenyl or 5 or 6-membered monocyclic heteroaryl, wherein the phenyl or 5 or 6-membered monocyclic heteroaryl are each optionally substituted with one to two substituent R⁵.

51. The compound of claim 50, or a pharmaceutically acceptable salt thereof, wherein R³ is selected from the group consisting of pyridyl, phenyl, and pyrazolyl.

52. The compound claim 50, or a pharmaceutically acceptable salt thereof, wherein R³ is represented by the following formula: 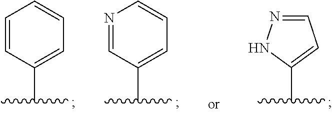
wherein each of the formula depicted above is optionally substituted with one to two R⁵.

53. The compound of claim 52, or a pharmaceutically acceptable salt thereof, wherein R³ is represented by the following formula:

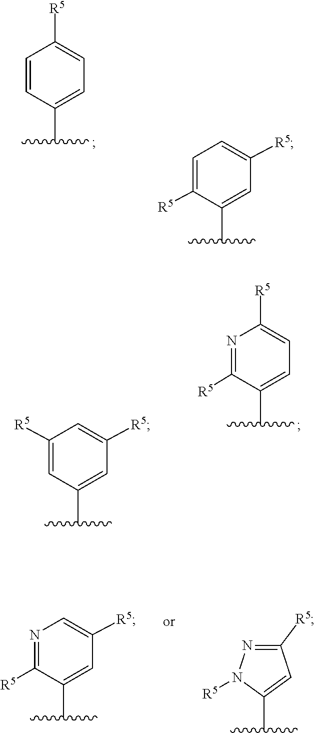

54. The compound of claim 52, or a pharmaceutically acceptable salt thereof, wherein R³ is represented by the following formula:

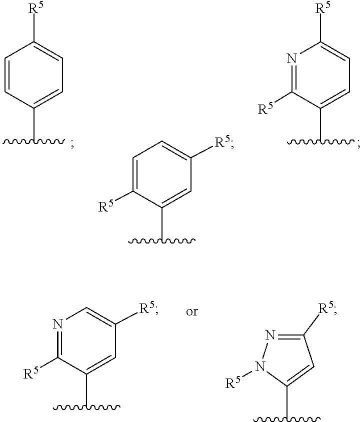

55. The compound of any one of claims 47 to 54, or a pharmaceutically acceptable salt thereof, wherein:
R⁵, for each occurrence, is independently selected from cyano, C₁₋₄alkyl, OR^{5a}, C₃₋₄cycloalkyl, and halo, wherein the C₁₋₄alkyl is optionally substituted with one to three R^{5b};
R^{5a} is C₁₋₃alkyl optionally substituted with one to three halo;
R^{5b}, for each occurrence, is halo.

56. The compound of any one of claims 47 to 54, or a pharmaceutically acceptable salt thereof, wherein:
R⁵, for each occurrence, is independently selected from cyano, C₁₋₄alkyl, OR^{5a}, and halo, wherein the C₁₋₄alkyl is optionally substituted with one to three R^{5b};
R^{5a} is C₁₋₃alkyl optionally substituted with one to three halo;
R^{5b}, for each occurrence, is halo.

57. The compound of claim 55, or a pharmaceutically acceptable salt thereof, wherein R⁵, for each occurrence, is independently selected from —CH₃, —CHF₂, —CH₂CH₃, —CF₃, —OCH₃, —OCHF₂, —OCF₃, —F, —Cl, cyclopropyl, and —CN.

58. The compound of claim 56, or a pharmaceutically acceptable salt thereof, wherein R⁵, for each occurrence, is independently selected from —CH₃, —CH₂CH₃, —CF₃, —OCH₃, —OCHF₂, —OCF₃, —F, and —CN.

59. The compound of any one of claims 47 to 58, or a pharmaceutically acceptable salt thereof, wherein if R¹ and R² are both C₁₋₆alkyl, at least one of said C₁₋₆alkyl is further substituted by at least one R⁴.

60. The compound of any one of claims 47 to 58, or a pharmaceutically acceptable salt thereof, wherein R¹ is H or C₁₋₃alkyl, and R² is C₁₋₃alkyl optionally substituted with C₁₋₃alkoxy or 4- to 6-membered heterocyclyl.

61. The compound of any one of claims 47 to 58, or a pharmaceutically acceptable salt thereof, wherein R¹ is H or C₁₋₃alkyl, and R² is C₁₋₃alkyl optionally substituted with C₁₋₃alkoxy.

62. The compound of claim 61, or a pharmaceutically acceptable salt thereof, wherein R¹ is H or —CH₃ and R² is —CH₂CH₂OCH₃ or tetrahydropyranyl.

63. The compound of claim 61, or a pharmaceutically acceptable salt thereof, wherein R¹ is —CH₃ and R² is —CH₂OCH₃.

64. The compound of any one of claims 47 to 58, or a pharmaceutically acceptable salt thereof, wherein R¹ and R² together with the N atom from which they are attached form a 4 to 6-membered monocyclic heterocycle or a 6 or 7-membered bicyclic heterocycle, each of which is optionally substituted with one or two R⁴.

65. The compound of claim 64, or a pharmaceutically acceptable salt thereof, wherein R¹ and R² together with the nitrogen atom from which they are attached form groups represented by the following formula:

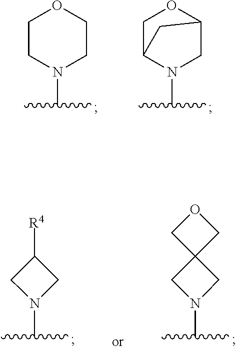

66. The compound of claim 64, or a pharmaceutically acceptable salt thereof, wherein R¹ and R² together with the nitrogen atom from which they are attached form groups represented by the following formula:

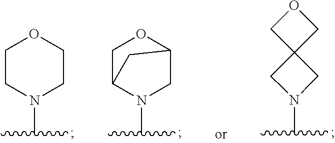 [SMILES: *N1CCOCC1 ; *N1CC2CC1CO2 ; *N1CC2(COC2)C1]

67. The compound of any one of claims 64 to 66, or a pharmaceutically acceptable salt thereof, wherein each R⁴ is independently —OH.

68. The compound of any one of claims 1 to 4, wherein the compound is represented by Formula (VI): 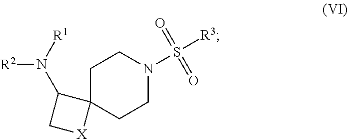
or a pharmaceutically acceptable salt thereof.

69. The compound of claim 68, wherein the compound is represented by Formula (VIA) or (VIB): 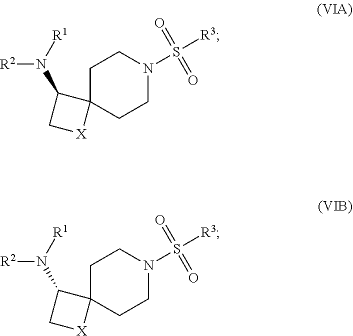
or a pharmaceutically acceptable salt thereof.

70. The compound of claim 68 or 69, wherein X is O.

71. The compound of any one of claims 68 to 70, wherein R³ is phenyl optionally substituted with one to two R⁴.

72. The compound of claim 71, wherein R³ is represented by the following formula:

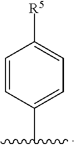

73. The compound of any one of claims 68 to 72, wherein and R¹ and R², together with the nitrogen atom from which they are attached form the group represented by the following formula:

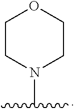 [SMILES: *N1CCOCC1]

74. The compound of any one of claims to 1 to 4, wherein the compound is represented by Formula (VII): 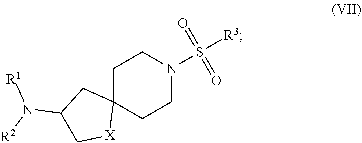
or a pharmaceutically acceptable salt thereof, wherein:
X is O, CH₂, or a bond;
R¹ is H and R² is 4 to 6-membered monocyclic heterocyclyl or 6 to 10-membered bicyclic heterocyclyl;
or R¹ and R² together with the N atom from which they are attached form a 4 to 7-membered monocyclic heterocycle or 6 to 10-membered bicyclic heterocycle;
R³ is phenyl or 5 or 6-membered monocyclic heteroaryl, each of which is optionally substituted with one or two R⁵;
each R⁵ is independently selected from C₁₋₃alkyl, C₁₋₃haloalkyl, C₃₋₄ cycloalkyl, OR^{5a}, cyano, and halo;
R^{5a} is C₁₋₃haloalkyl.

75. The compound of claim 74, wherein the compound is represented by Formula (III) or (IV): 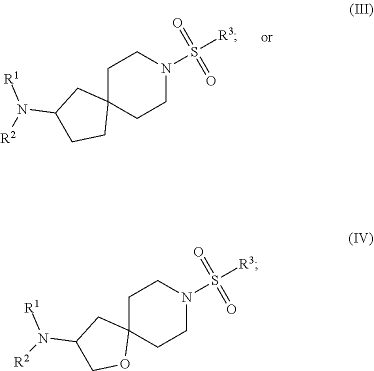
or a pharmaceutically acceptable salt thereof.

76. The compound of claim 75, wherein the compound is represented by Formula (IIIA), (IIIB), (IVA), or (IVB): 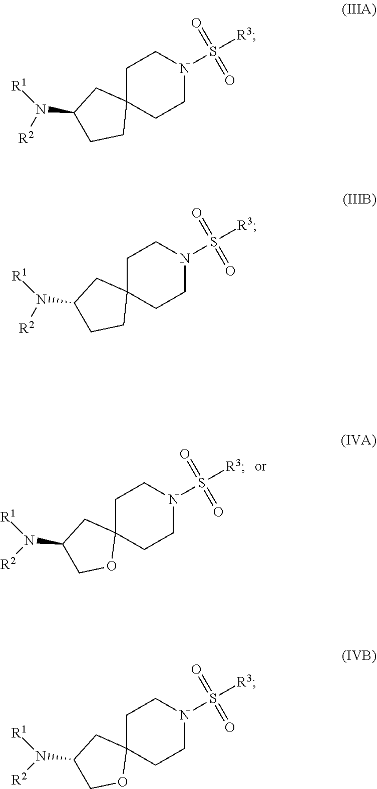
or a pharmaceutically acceptable salt thereof.

77. The compound of any one of claims 74 to 76, or a pharmaceutically acceptable salt thereof, wherein R³ is phenyl, pyrazoyl, or pyridinyl, each of which is optionally substituted by one or two R⁵.

78. The compound claim 77, or a pharmaceutically acceptable salt thereof, wherein R³ is represented by the following formula: 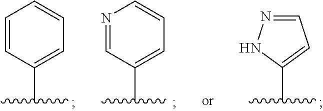
wherein each of the formula depicted above is optionally substituted with one to two R⁵.

79. The compound claim 77, or a pharmaceutically acceptable salt thereof, wherein R³ is represented by the following formula:

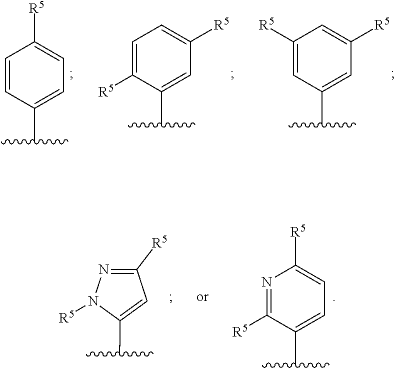

80. The compound of any one of claims 74 to 79, or a pharmaceutically acceptable salt thereof, wherein:
(1) R² is represented by the following formula: 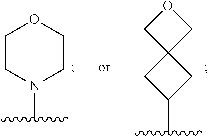 [SMILES: *N1CCOCC1 ; *C1CC2(COC2)C1]
or
(2) R¹ and R² together with the N atom from which they are attached form a group represented by the following formula:

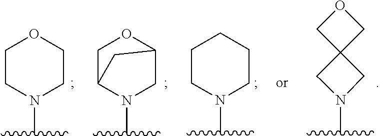

81. The compound of any one of claims 74 to 79, or a pharmaceutically acceptable salt thereof, wherein each R⁵ is independently selected from —CH₃, —CF₃, —F, —CN, and —OCHF₂.

82. The compound of claim 74, wherein the compound is represented by Formula (VIII): 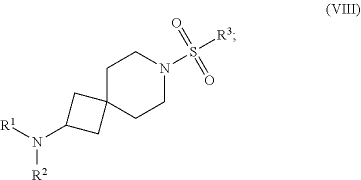
or a pharmaceutically acceptable salt thereof.

83. The compound of claim 82, wherein the compound is represented by Formula (VIIIA) or (VIIIB): 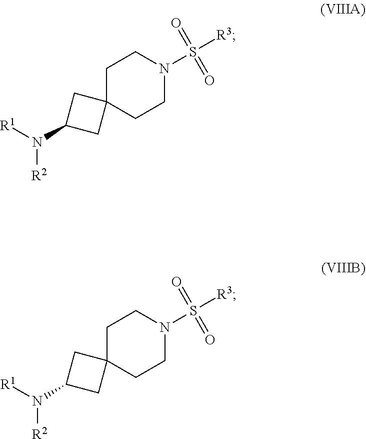
or a pharmaceutically acceptable salt thereof.

84. The compound of any one of claims 74, 82, or 83, or a pharmaceutically acceptable salt thereof, wherein R³ is pyrazoyl or pyridinyl, each of which is optionally substituted by one or two R⁵.

85. The compound claim 84, or a pharmaceutically acceptable salt thereof, wherein R³ is represented by the following formula: 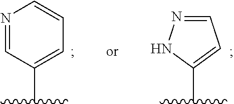 [SMILES: *c1cccnc1 ; *c1ccn[nH]1]
wherein each of the formula depicted above is optionally substituted with one to two R⁵.

86. The compound claim 85, or a pharmaceutically acceptable salt thereof, wherein R³ is represented by the following formula:

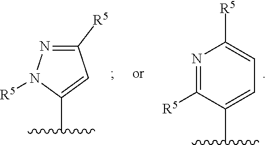

87. The compound of any one of claims 74, or 82 to 86, or a pharmaceutically acceptable salt thereof, wherein:
(1) R² is represented by the following formula: 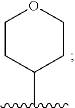 [SMILES: *C1CCOCC1]
or
(2) R¹ and R² together with the N atom from which they are attached form a group represented by the following formula:

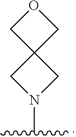 [SMILES: *N1CC2(COC2)C1]

88. The compound of any one of claims 74, or 82 to 87, or a pharmaceutically acceptable salt thereof, wherein each R⁵ is independently selected from —CH₃, —CF₃, and cyclopropyl.

89. The compound of claim 1, or a pharmaceutically acceptable salt thereof, wherein the compound is selected from:
8-(Benzofuran-2-ylsulfonyl)-3-morpholino-1-oxa-8-azaspiro[4.5]decane;
8-((3-Chloro-5-(methoxymethyl)phenyl)sulfonyl)-3-morpholino-1-oxa-8-azaspiro[4.5]decane;
2-((3-Morpholino-1-oxa-8-azaspiro[4.5]decan-8-yl)sulfonyl)-3-(trifluoromethoxy)phenol;
8-((4-Methyl-6-(trifluoromethyl)pyridin-3-yl)sulfonyl)-3-morpholino-1-oxa-8-azaspiro[4.5]decane;
8-((5-methylthiophen-3-yl)sulfonyl)-3-morpholino-1-oxa-8-azaspiro[4.5]decane;
(R)- or (S)-8-((1-methyl-3-(trifluoromethyl)-1H-pyrazol-5-yl)sulfonyl)-3-morpholino-1-oxa-8-azaspiro[4.5]decane;
8-((4-Fluorophenyl)sulfonyl)-3-(2-oxa-6-azaspiro[3.3]heptan-6-yl)-1-oxa-8-azaspiro[4.5]decane;
4-((3-(2-Oxa-6-azaspiro[3.3]heptan-6-yl)-1-oxa-8-azaspiro[4.5]decan-8-yl)sulfonyl)-3-chlorobenzonitrile;
6-(8-((3-Fluoro-4-methoxyphenyl)sulfonyl)-8-azaspiro[4.5]decan-2-yl)-2-oxa-6-azaspiro[3.3]heptane;
3-((2-(2-Oxa-6-azaspiro[3.3]heptan-6-yl)-8-azaspiro[4.5]decan-8-yl)sulfonyl)benzonitrile;
6-(8-((4-Fluorophenyl)sulfonyl)-8-azaspiro[4.5]decan-2-yl)-2-oxa-6-azaspiro[3.3]heptane;
3-((2-(2-Oxa-6-azaspiro[3.3]heptan-6-yl)-8-azaspiro[4.5]decan-8-yl)sulfonyl)-5-fluorobenzonitrile;
8-((4-(Difluoromethoxy)phenyl)sulfonyl)-3-(piperidin-1-yl)-1-oxa-8-azaspiro[4.5]decane;
4-Methyl-3-((3-morpholino-1-oxa-8-azaspiro[4.5]decan-8-yl)sulfonyl)benzonitrile;
(R)-4-Methyl-3-((3-morpholino-1-oxa-8-azaspiro[4.5]decan-8-yl)sulfonyl)benzonitrile;
(S)-4-methyl-3-((3-morpholino-1-oxa-8-azaspiro[4.5]decan-8-yl)sulfonyl)benzonitrile;
4-Fluoro-3-((3-(piperidin-1-yl)-1-oxa-8-azaspiro[4.5]decan-8-yl)sulfonyl)benzonitrile;
8-((2-Methyl-4-(trifluoromethyl)phenyl)sulfonyl)-3-morpholino-1-oxa-8-azaspiro[4.5]decane;
8-((2-Fluoro-5-methylphenyl)sulfonyl)-3-morpholino-1-oxa-8-azaspiro[4.5]decane;
8-((2,5-Difluorophenyl)sulfonyl)-3-morpholino-1-oxa-8-azaspiro[4.5]decane;
8-((6-Methoxy-2-methylpyridin-3-yl)sulfonyl)-3-morpholino-1-oxa-8-azaspiro[4.5]decane;
4-(8-((1,3-Dimethyl-1H-pyrazol-5-yl)sulfonyl)-8-azaspiro[4.5]decan-2-yl)morpholine;
(R)-4-(8-((1,3-Dimethyl-1H-pyrazol-5-yl)sulfonyl)-8-azaspiro[4.5]decan-2-yl)morpholine;
(S)-4-(8-((1,3-dimethyl-1H-pyrazol-5-yl)sulfonyl)-8-azaspiro[4.5]decan-2-yl)morpholine;
4-(8-((4-(Difluoromethoxy)phenyl)sulfonyl)-8-azaspiro[4.5]decan-2-yl)morpholine;
2-Methyl-3-((2-morpholino-8-azaspiro[4.5]decan-8-yl)sulfonyl)-2,4,6,7-tetrahydropyrano[4,3-c]pyrazole;
4-(8-((3,5-Dimethylpyridin-2-yl)sulfonyl)-8-azaspiro[4.5]decan-2-yl)morpholine;
4-(8-((3-Methoxy-1-methyl-1H-pyrazol-5-yl)sulfonyl)-8-azaspiro[4.5]decan-2-yl)morpholine;
5-(8-((4-(Difluoromethoxy)phenyl)sulfonyl)-8-azaspiro[4.5]decan-2-yl)-2-oxa-5-azabicyclo[2.2.1]heptane;
(1R,4R)-5-(8-((4-(Difluoromethoxy)phenyl)sulfonyl)-8-azaspiro[4.5]decan-2-yl)-2-oxa-5-azabicyclo[2.2.1]heptane;
8-((4,6-Dimethylpyridin-3-yl)sulfonyl)-3-(2-oxa-7-azaspiro[4.4]nonan-7-yl)-1-oxa-8-azaspiro[4.5]decane;
8-((1,3-Dimethyl-1H-pyrazol-5-yl)sulfonyl)-3-(2-oxa-7-azaspiro[4.4]nonan-7-yl)-1-oxa-8-azaspiro[4.5]decane;
8-((4-(Difluoromethoxy)phenyl)sulfonyl)-3-(2-oxa-7-azaspiro[4.4]nonan-7-yl)-1-oxa-8-azaspiro[4.5]decane;
(S)-8-((4-(Difluoromethoxy)phenyl)sulfonyl)-3-((R)-2-oxa-7-azaspiro[4.4]nonan-7-yl)-1-oxa-8-azaspiro[4.5]decane;
(S)-8-((4-(difluoromethoxy)phenyl)sulfonyl)-3-((S)-2-oxa-7-azaspiro[4.4]nonan-7-yl)-1-oxa-8-azaspiro[4.5]decane;
(R)-8-((4-(difluoromethoxy)phenyl)sulfonyl)-3-((R)-2-oxa-7-azaspiro[4.4]nonan-7-yl)-1-oxa-8-azaspiro[4.5]decane;
(R)-8-((4-(difluoromethoxy)phenyl)sulfonyl)-3-((S)-2-oxa-7-azaspiro[4.4]nonan-7-yl)-1-oxa-8-azaspiro[4.5]decane;
4-(8-((7-fluoroquinolin-6-yl)sulfonyl)-8-azaspiro[4.5]decan-2-yl)morpholine;
4-(8-((5-Cyclopropoxypyridin-2-yl)sulfonyl)-8-azaspiro[4.5]decan-2-yl)morpholine;
4-(8-((3-Fluoro-2-methoxypyridin-4-yl)sulfonyl)-8-azaspiro[4.5]decan-2-yl)morpholine;
4-(7-((1-Ethyl-3-methyl-1H-pyrazol-5-yl)sulfonyl)-7-azaspiro[3.5]nonan-2-yl)morpholine;
4-(7-((2-Methyl-6-(trifluoromethyl)pyridin-3-yl)sulfonyl)-7-azaspiro[3.5]nonan-2-yl)morpholine;
4-(7-((2-Methoxy-5-methylpyridin-3-yl)sulfonyl)-7-azaspiro[3.5]nonan-2-yl)morpholine;
4-(7-((3-Cyclopropyl-1-methyl-1H-pyrazol-5-yl)sulfonyl)-7-azaspiro[3.5]nonan-2-yl)morpholine;
4-(8-((2-Methyl-6-(trifluoromethyl)pyridin-3-yl)sulfonyl)-8-azaspiro[bicyclo[3.2.1]octane-3,1′-cyclobutan]-3′-yl)morpholine;
7-((4-(Difluoromethoxy)phenyl)sulfonyl)-3-morpholino-1-oxa-7-azaspiro[3.5]nonane;
8-((2-Methoxy-5-methylpyridin-3-yl)sulfonyl)-N-(2-methoxyethyl)-N-methyl-8-azaspiro[4.5]decan-2-amine;
8-((2-Methoxy-5-methylpyridin-3-yl)sulfonyl)-N-(3-methyloxetan-3-yl)-8-azaspiro[4.5]decan-2-amine;
8-((2-Methoxy-5-methylpyridin-3-yl)sulfonyl)-N-(3-methyltetrahydrofuran-3-yl)-8-azaspiro[4.5]decan-2-amine;
2-(8-((2-Methoxy-5-methylpyridin-3-yl)sulfonyl)-8-azaspiro[4.5]decan-2-yl)-6-oxa-2-azaspiro[3.4]octane;
4-Fluoro-3-((3-((2-methoxyethyl)amino)-1-oxa-8-azaspiro[4.5]decan-8-yl)sulfonyl)benzonitrile;
3-((3-((1R,4R)-2-oxa-5-azabicyclo[2.2.1]heptan-5-yl)-1-oxa-8-azaspiro[4.5]decan-8-yl)sulfonyl)-4-fluorobenzonitrile;
3-((3-(2-Oxa-6-azaspiro[3.3]heptan-6-yl)-1-oxa-8-azaspiro[4.5]decan-8-yl)sulfonyl)-4-fluorobenzonitrile;
4-Fluoro-3-((3-(piperidin-1-yl)-1-oxa-8-azaspiro[4.5]decan-8-yl)sulfonyl)benzonitrile;
3-((3-((4,4-Difluorocyclohexyl)amino)-1-oxa-8-azaspiro[4.5]decan-8-yl)sulfonyl)-4-fluorobenzonitrile;
8-((2-Chloro-4-methylphenyl)sulfonyl)-3-(2-oxa-6-azaspiro[3.3]heptan-6-yl)-1-oxa-8-azaspiro[4.5]decane;
1-(8-((2-Chloro-4-methylphenyl)sulfonyl)-1-oxa-8-azaspiro[4.5]decan-3-yl)-3-methylazetidin-3-ol;
8-((2-Chloro-4-methylphenyl)sulfonyl)-N-(tetrahydro-2H-pyran-4-yl)-1-oxa-8-azaspiro[4.5]decan-3-amine;
1-(8-((2-Chloro-4-methylphenyl)sulfonyl)-1-oxa-8-azaspiro[4.5]decan-3-yl)azetidin-3-ol;
(R)-1-(8-((2-Chloro-4-methylphenyl)sulfonyl)-1-oxa-8-azaspiro[4.5]decan-3-yl)azetidin-3-ol;
(S)-1-(8-((2-chloro-4-methylphenyl)sulfonyl)-1-oxa-8-azaspiro[4.5]decan-3-yl)azetidin-3-ol;
8-((2-Methyl-4-(trifluoromethoxy)phenyl)sulfonyl)-3-morpholino-1-oxa-8-azaspiro[4.5]decane;
8-((2-Cyclopropylthiazol-5-yl)sulfonyl)-3-(3-methoxyazetidin-1-yl)-1-oxa-8-azaspiro[4.5]decane;
8-((3-Cyclopropyl-1-methyl-1H-pyrazol-5-yl)sulfonyl)-3-(2-oxa-6-azaspiro[3.3]heptan-6-yl)-1-oxa-8-azaspiro[4.5]decane;
(R)-6-(8-((1,3-dimethyl-1H-pyrazol-5-yl)sulfonyl)-8-azaspiro[4.5]decan-2-yl)-2-oxa-6-azaspiro[3.3]heptane;
(S)-6-(8-((1,3-dimethyl-1H-pyrazol-5-yl)sulfonyl)-8-azaspiro[4.5]decan-2-yl)-2-oxa-6-azaspiro[3.3]heptane;
8-((3-Cyclopropyl-1-methyl-1H-pyrazol-5-yl)sulfonyl)-3-(3-methoxyazetidin-1-yl)-1-oxa-8-azaspiro[4.5]decane;
8-((3-Cyclopropyl-1-methyl-1H-pyrazol-5-yl)sulfonyl)-N-(2-methoxyethyl)-1-oxa-8-azaspiro[4.5]decan-3-amine;
8-((3-Cyclopropyl-1-methyl-1H-pyrazol-5-yl)sulfonyl)-N-(2-methoxyethyl)-N-methyl-1-oxa-8-azaspiro[4.5]decan-3-amine;
8-((5-Chloro-2-methoxypyridin-3-yl)sulfonyl)-3-(3-methoxyazetidin-1-yl)-1-oxa-8-azaspiro[4.5]decane;
8-((5-Chloro-2-methoxypyridin-3-yl)sulfonyl)-3-(2-oxa-6-azaspiro[3.3]heptan-6-yl)-1-oxa-8-azaspiro[4.5]decane;
(R)-8-((5-Chloro-2-methoxypyridin-3-yl)sulfonyl)-3-(2-oxa-6-azaspiro[3.3]heptan-6-yl)-1-oxa-8-azaspiro[4.5]decane;
(S)-8-((5-chloro-2-methoxypyridin-3-yl)sulfonyl)-3-(2-oxa-6-azaspiro[3.3]heptan-6-yl)-1-oxa-8-azaspiro[4.5]decane;
3-((3-(2-Oxa-6-azaspiro[3.3]heptan-6-yl)-1-oxa-8-azaspiro[4.5]decan-8-yl)sulfonyl)-4-chlorobenzonitrile;
8-((2-Methoxy-5-methylpyridin-3-yl)sulfonyl)-N-(2-methoxyethyl)-1-oxa-8-azaspiro[4.5]decan-3-amine;
8-((2-Methoxy-5-methylpyridin-3-yl)sulfonyl)-N-(2-methoxyethyl)-N-(methyl-d3)-1-oxa-8-azaspiro[4.5]decan-3-amine;
8-((2-Methoxy-5-methylpyridin-3-yl)sulfonyl)-N-(2-methoxyethyl)-N-methyl-1-oxa-8-azaspiro[4.5]decan-3-amine;
6-(8-((1,3-dimethyl-1H-pyrazol-5-yl)sulfonyl)-8-azaspiro[4.5]decan-2-yl)-2-oxa-6-azaspiro[3.3]heptane;
(R)-6-(8-((1,3-dimethyl-1H-pyrazol-5-yl)sulfonyl)-8-azaspiro[4.5]decan-2-yl)-2-oxa-6-azaspiro[3.3]heptane;
(S)-6-(8-((1,3-dimethyl-1H-pyrazol-5-yl)sulfonyl)-8-azaspiro[4.5]decan-2-yl)-2-oxa-6-azaspiro[3.3]heptane;
8-((4-(Difluoromethoxy)phenyl)sulfonyl)-3-(6-oxa-2-azaspiro[3.4]octan-2-yl)-1-oxa-8-azaspiro[4.5]decane;
8-((4-(Difluoromethoxy)phenyl)sulfonyl)-3-(2-oxa-7-azaspiro[4.4]nonan-7-yl)-1-oxa-8-azaspiro[4.5]decane;
8-((4-(Difluoromethoxy)phenyl)sulfonyl)-3-((3aR,6aS)-tetrahydro-1H-furo[3,4-c]pyrrol-5(3H)-yl)-1-oxa-8-azaspiro[4.5]decane;
8-((1,3-Dimethyl-1H-pyrazol-5-yl)sulfonyl)-3-(2-oxa-7-azaspiro[4.4]nonan-7-yl)-1-oxa-8-azaspiro[4.5]decane;
(S)-8-((1,3-Dimethyl-1H-pyrazol-5-yl)sulfonyl)-3-((R)-2-oxa-7-azaspiro[4.4]nonan-7-yl)-1-oxa-8-azaspiro[4.5]decane;
(S)-8-((1,3-dimethyl-1H-pyrazol-5-yl)sulfonyl)-3-((S)-2-oxa-7-azaspiro[4.4]nonan-7-yl)-1-oxa-8-azaspiro[4.5]decane;
(R)-8-((1,3-dimethyl-1H-pyrazol-5-yl)sulfonyl)-3-((R)-2-oxa-7-azaspiro[4.4]nonan-7-yl)-1-oxa-8-azaspiro[4.5]decane;
(S)-8-((1,3-dimethyl-1H-pyrazol-5-yl)sulfonyl)-3-((S)-2-oxa-7-azaspiro[4.4]nonan-7-yl)-1-oxa-8-azaspiro[4.5]decane;
4-(7-((4-(Difluoromethoxy)phenyl)sulfonyl)-7-azaspiro[3.5]nonan-2-yl)morpholine;
4-(7-((1,3-Dimethyl-1H-pyrazol-5-yl)sulfonyl)-7-azaspiro[3.5]nonan-2-yl)morpholine;
6-(7-((6-Methoxy-2-methylpyridin-3-yl)sulfonyl)-7-azaspiro[3.5]nonan-2-yl)-2-oxa-6-azaspiro[3.3]heptane;
6-(7-((2-Methyl-6-(trifluoromethyl)pyridin-3-yl)sulfonyl)-7-azaspiro[3.5]nonan-2-yl)-2-oxa-6-azaspiro[3.3]heptane;
6-(7-((6-Chloro-2-methylpyridin-3-yl)sulfonyl)-7-azaspiro[3.5]nonan-2-yl)-2-oxa-6-azaspiro[3.3]heptane;
6-(7-((4-Methyl-6-(trifluoromethyl)pyridin-3-yl)sulfonyl)-7-azaspiro[3.5]nonan-2-yl)-2-oxa-6-azaspiro[3.3]heptane;
6-(7-((1-Methyl-3-(trifluoromethyl)-1H-pyrazol-5-yl)sulfonyl)-7-azaspiro[3.5]nonan-2-yl)-2-oxa-6-azaspiro[3.3]heptane;
6-(7-((3-Cyclopropyl-1-methyl-1H-pyrazol-5-yl)sulfonyl)-7-azaspiro[3.5]nonan-2-yl)-2-oxa-6-azaspiro[3.3]heptane;
5-(7-((6-Methoxy-2-methylpyridin-3-yl)sulfonyl)-7-azaspiro[3.5]nonan-2-yl)-2-oxa-5-azabicyclo[2.2.1]heptane;
(1R,4R)-5-(7-((6-Methoxy-2-methylpyridin-3-yl)sulfonyl)-7-azaspiro[3.5]nonan-2-yl)-2-oxa-5-azabicyclo[2.2.1]heptane;
5-(7-((1-methyl-3-(trifluoromethyl)-1H-pyrazol-5-yl)sulfonyl)-7-azaspiro[3.5]nonan-2-yl)-2-oxa-5-azabicyclo[2.2.1]heptane;
(1R,4R)-5-(7-((1-methyl-3-(trifluoromethyl)-1H-pyrazol-5-yl)sulfonyl)-7-azaspiro[3.5]nonan-2-yl)-2-oxa-5-azabicyclo[2.2.1]heptane;
7-((1-Methyl-3-(trifluoromethyl)-1H-pyrazol-5-yl)sulfonyl)-N-(tetrahydro-2H-pyran-4-yl)-7-azaspiro[3.5]nonan-2-amine;
7-((4-Methyl-6-(trifluoromethyl)pyridin-3-yl)sulfonyl)-N-(tetrahydro-2H-pyran-4-yl)-7-azaspiro[3.5]nonan-2-amine;
1-(8-((1,3-dimethyl-1H-pyrazol-5-yl)sulfonyl)-8-azaspiro[4.5]decan-2-yl)-4-methylpiperidin-4-ol;
(R)-3-((2-(2-oxa-6-azaspiro[3.3]heptan-6-yl)-8-azaspiro[4.5]decan-8-yl)sulfonyl)-5-fluorobenzonitrile;
(S)-3-((2-(2-oxa-6-azaspiro[3.3]heptan-6-yl)-8-azaspiro[4.5]decan-8-yl)sulfonyl)-5-fluorobenzonitrile;
(1R,4R)-5-((R)-8-((4-(difluoromethoxy)phenyl)sulfonyl)-8-azaspiro[4.5]decan-2-yl)-2-oxa-5-azabicyclo[2.2.1]heptane;
(1S,4S)-5-((R)-8-((1,3-dimethyl-1H-pyrazol-5-yl)sulfonyl)-8-azaspiro[4.5]decan-2-yl)-2-oxa-5-azabicyclo[2.2.1]heptane;
(1R,4R)-5-((R)-8-((1,3-dimethyl-1H-pyrazol-5-yl)sulfonyl)-8-azaspiro[4.5]decan-2-yl)-2-oxa-5-azabicyclo[2.2.1]heptane;
(1S,4S)-5-((R)-8-((4-(difluoromethoxy)phenyl)sulfonyl)-8-azaspiro[4.5]decan-2-yl)-2-oxa-5-azabicyclo[2.2.1]heptane;
(1S,4S)-5-((S)-8-((4-(difluoromethoxy)phenyl)sulfonyl)-8-azaspiro[4.5]decan-2-yl)-2-oxa-5-azabicyclo[2.2.1]heptane;
(1S,4S)-5-((R)-8-((4-(difluoromethoxy)phenyl)sulfonyl)-8-azaspiro[4.5]decan-2-yl)-2-oxa-5-azabicyclo[2.2.1]heptane;
7-((1,3-dimethyl-1H-pyrazol-5-yl)sulfonyl)-N-(2-methoxyethyl)-N-methyl-7-azaspiro[3.5]nonan-2-amine;
6-(7-((1,3-dimethyl-1H-pyrazol-5-yl)sulfonyl)-7-azaspiro[3.5]nonan-2-yl)-2-oxa-6-azaspiro[3.3]heptane;
6-(7-((2-methoxy-5-methylpyridin-3-yl)sulfonyl)-7-azaspiro[3.5]nonan-2-yl)-2-oxa-6-azaspiro[3.3]heptane;
(1R,4R)-5-(7-((6-chloro-2-methylpyridin-3-yl)sulfonyl)-7-azaspiro[3.5]nonan-2-yl)-2-oxa-5-azabicyclo[2.2.1]heptane;
1-(7-((2-methyl-6-(trifluoromethyl)pyridin-3-yl)sulfonyl)-7-azaspiro[3.5]nonan-2-yl)azetidin-3-ol;
3-((2-(2-oxa-6-azaspiro[3.3]heptan-6-yl)-7-azaspiro[3.5]nonan-7-yl)sulfonyl)-5-fluorobenzonitrile;
3-(((S)-2-((1R,4R)-2-oxa-5-azabicyclo[2.2.1]heptan-5-yl)-8-azaspiro[4.5]decan-8-yl)sulfonyl)-5-fluorobenzonitrile;
3-fluoro-5-((3-(((tetrahydro-2H-pyran-4-yl)methyl)amino)-1-oxa-8-azaspiro[4.5]decan-8-yl)sulfonyl)benzonitrile;
1-(8-((2-methoxy-6-methyl-2H-112-pyridin-5-yl)sulfonyl)-8-azaspiro[4.5]decan-2-yl)azetidin-3-ol;
8-((2-methoxy-6-methyl-2H-112-pyridin-5-yl)sulfonyl)-N-(2-methoxyethyl)-N-methyl-8-azaspiro[4.5]decan-2-amine;
6-(8-((5-(trifluoromethyl)furan-3-yl)sulfonyl)-8-azaspiro[4.5]decan-2-yl)-2-oxa-6-azaspiro[3.3]heptane;
6-(8-((7-fluoroquinolin-6-yl)sulfonyl)-8-azaspiro[4.5]decan-2-yl)-2-oxa-6-azaspiro[3.3]heptane;
6-(8-(quinolin-3-ylsulfonyl)-8-azaspiro[4.5]decan-2-yl)-2-oxa-6-azaspiro[3.3]heptane;
3-((2-(2-oxa-6-azaspiro[3.3]heptan-6-yl)-8-azaspiro[4.5]decan-8-yl)sulfonyl)-2-fluorobenzonitrile;
6-(8-((2-methylfuran-3-yl)sulfonyl)-8-azaspiro[4.5]decan-2-yl)-2-oxa-6-azaspiro[3.3]heptane;
6-(8-((5-chloro-6-methoxypyridin-3-yl)sulfonyl)-8-azaspiro[4.5]decan-2-yl)-2-oxa-6-azaspiro[3.3]heptane;
6-(8-((2,3-dihydrobenzo[b][1,4]dioxin-6-yl)sulfonyl)-8-azaspiro[4.5]decan-2-yl)-2-oxa-6-azaspiro[3.3]heptane;
6-(8-((4-ethoxyphenyl)sulfonyl)-8-azaspiro[4.5]decan-2-yl)-2-oxa-6-azaspiro[3.3]heptane;
6-(8-((5,6-dimethylpyridin-3-yl)sulfonyl)-8-azaspiro[4.5]decan-2-yl)-2-oxa-6-azaspiro[3.3]heptane;
6-(8-((6-methoxypyridin-3-yl)sulfonyl)-8-azaspiro[4.5]decan-2-yl)-2-oxa-6-azaspiro[3.3]heptane;
6-(8-((2-ethyl-2H-1,2,3-triazol-4-yl)sulfonyl)-8-azaspiro[4.5]decan-2-yl)-2-oxa-6-azaspiro[3.3]heptane;
6-(8-((2-methoxypyridin-4-yl)sulfonyl)-8-azaspiro[4.5]decan-2-yl)-2-oxa-6-azaspiro[3.3]heptane;
6-(8-((5-fluoro-2-methoxypyridin-4-yl)sulfonyl)-8-azaspiro[4.5]decan-2-yl)-2-oxa-6-azaspiro[3.3]heptane;
8-((5-chloro-6-methoxypyridin-3-yl)sulfonyl)-3-(2-oxa-6-azaspiro[3.3]heptan-6-yl)-1-oxa-8-azaspiro[4.5]decane;
6-(8-((5-(trifluoromethoxy)pyridin-2-yl)sulfonyl)-8-azaspiro[4.5]decan-2-yl)-2-oxa-6-azaspiro[3.3]heptane;
8-((5-fluoro-2-methoxypyridin-4-yl)sulfonyl)-3-(2-oxa-6-azaspiro[3.3]heptan-6-yl)-1-oxa-8-azaspiro[4.5]decane;
8-((2,4-dimethylphenyl)sulfonyl)-N-(2-methoxyethyl)-1-oxa-8-azaspiro[4.5]decan-3-amine;
8-((2-methoxy-5-methylpyridin-3-yl)sulfonyl)-3-(2-oxa-6-azaspiro[3.3]heptan-6-yl)-1-oxa-8-azaspiro[4.5]decane;
Rac-8-((3,5-difluorophenyl)sulfonyl)-3-(2-oxa-6-azaspiro[3.3]heptan-6-yl)-1-oxa-8-azaspiro[4.5]decane;
(R)-8-((3,5-difluorophenyl)sulfonyl)-3-(2-oxa-6-azaspiro[3.3]heptan-6-yl)-1-oxa-8-azaspiro[4.5]decane;
(S)-8-((3,5-difluorophenyl)sulfonyl)-3-(2-oxa-6-azaspiro[3.3]heptan-6-yl)-1-oxa-8-azaspiro[4.5]decane;
7-((3-cyclopropyl-1-methyl-1H-pyrazol-5-yl)sulfonyl)-N-(tetrahydro-2H-pyran-4-yl)-7-azaspiro[3.5]nonan-2-amine;
6-(7-((6-(difluoromethoxy)-2-methyl-2,3,4,5-tetrahydropyridin-3-yl)sulfonyl)-7-azaspiro[3.5]nonan-2-yl)-2-oxa-6-azaspiro[3.3]heptane;
6-(7-((3-(difluoromethyl)-1-methyl-1H-pyrazol-5-yl)sulfonyl)-7-azaspiro[3.5]nonan-2-yl)-2-oxa-6-azaspiro[3.3]heptane;
6-(7-((1-cyclopropyl-3-(difluoromethyl)-1H-pyrazol-5-yl)sulfonyl)-7-azaspiro[3.5]nonan-2-yl)-2-oxa-6-azaspiro[3.3]heptane;
6-(8-((3-methoxy-1-methyl-1H-pyrazol-5-yl)sulfonyl)-8-azaspiro[4.5]decan-2-yl)-2-oxa-6-azaspiro[3.3]heptane;
6-(8-((3-(difluoromethyl)-1-methyl-1H-pyrazol-5-yl)sulfonyl)-8-azaspiro[4.5]decan-2-yl)-2-oxa-6-azaspiro[3.3]heptane;
4-((2-(2-oxa-6-azaspiro[3.3]heptan-6-yl)-8-azaspiro[4.5]decan-8-yl)sulfonyl)-3-chlorobenzonitrile;
5-((2-(2-oxa-6-azaspiro[3.3]heptan-6-yl)-8-azaspiro[4.5]decan-8-yl)sulfonyl)-2-fluorobenzonitrile;
5-((2-(2-oxa-6-azaspiro[3.3]heptan-6-yl)-8-azaspiro[4.5]decan-8-yl)sulfonyl)-2-methylbenzonitrile;
6-(8-((2-fluoro-4-methoxyphenyl)sulfonyl)-8-azaspiro[4.5]decan-2-yl)-2-oxa-6-azaspiro[3.3]heptane;
6-(8-((6-isopropoxypyridin-3-yl)sulfonyl)-8-azaspiro[4.5]decan-2-yl)-2-oxa-6-azaspiro[3.3]heptane;
6-(8-(pyrazolo[1,5-a]pyridin-6-ylsulfonyl)-8-azaspiro[4.5]decan-2-yl)-2-oxa-6-azaspiro[3.3]heptane;
6-(8-((5-fluoroquinolin-3-yl)sulfonyl)-8-azaspiro[4.5]decan-2-yl)-2-oxa-6-azaspiro[3.3]heptane;
6-(8-((3-fluoroquinolin-6-yl)sulfonyl)-8-azaspiro[4.5]decan-2-yl)-2-oxa-6-azaspiro[3.3]heptane;
6-(8-((4-cyclopropoxyphenyl)sulfonyl)-8-azaspiro[4.5]decan-2-yl)-2-oxa-6-azaspiro[3.3]heptane;
6-(8-(benzofuran-6-ylsulfonyl)-8-azaspiro[4.5]decan-2-yl)-2-oxa-6-azaspiro[3.3]heptane;
8-((2-fluoro-4-methoxyphenyl)sulfonyl)-N-methyl-N-(2-oxaspiro[3.3]heptan-6-yl)-1-oxa-8-azaspiro[4.5]decan-3-amine;
8-((2-methyl-6-(trifluoromethyl)pyridin-3-yl)sulfonyl)-N-(2-oxaspiro[3.3]heptan-6-yl)-1-oxa-8-azaspiro[4.5]decan-3-amine;
8-((2-methyl-6-(trifluoromethyl)pyridin-3-yl)sulfonyl)-N-(tetrahydro-2H-pyran-4-yl)-1-oxa-8-azaspiro[4.5]decan-3-amine;
8-((1-methyl-3-(trifluoromethyl)-1H-pyrazol-5-yl)sulfonyl)-N-(2-oxaspiro[3.3]heptan-6-yl)-1-oxa-8-azaspiro[4.5]decan-3-amine;
N-methyl-8-((4-methyl-2-(trifluoromethyl)pyrimidin-5-yl)sulfonyl)-N-(2-oxaspiro[3.3]heptan-6-yl)-1-oxa-8-azaspiro[4.5]decan-3-amine;
(S)-4-fluoro-3-((3-(4-hydroxy-4-methylpiperidin-1-yl)-1-oxa-8-azaspiro[4.5]decan-8-yl)sulfonyl)benzonitrile;
6-(8-((1,3-dimethyl-1H-pyrazol-5-yl)sulfonyl)-8-azaspiro[4.5]decan-2-yl)-2-oxa-6-azaspiro[3.3]heptane;
8-((4-(difluoromethoxy)phenyl)sulfonyl)-3-(6-oxa-2-azaspiro[3.5]nonan-2-yl)-1-oxa-8-azaspiro[4.5]decane; and
6-(8-(quinolin-7-ylsulfonyl)-8-azaspiro[4.5]decan-2-yl)-2-oxa-6-azaspiro[3.3]heptane;
or a or a pharmaceutically acceptable salt thereof.

90. A pharmaceutical composition comprising a compound of any one of claims 1 to 89, or a pharmaceutically acceptable salt thereof, and a pharmaceutically acceptable excipient.

91. A method of treating a disease mediated by Emopamil-Binding Protein comprising administering to a subject an effective amount of a compound of any one of claims 1 to 89, or a pharmaceutically acceptable salt thereof, or the pharmaceutical composition of claim 90.

92. A method of treating a disease mediated by Emopamil-Binding Protein, comprising administering to a subject an effective amount of a compound represented by Formula (I): 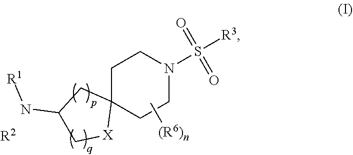
or a pharmaceutically acceptable salt thereof, wherein:
X is O, CH₂, or a bond, provided when X is a bond, p and q are 1;
p is 0 or 1, provided that when p is 0, q is 1;
q is 0 or 1, provided that when q is 0, p is 1;
R¹ and R² are each independently selected from H, C₁₋₆alkyl, C₃₋₈cycloalkyl, C₃₋₈cycloalkenyl, 6 to 10-membered bicyclic heterocycle, and 4 to 6-membered monocyclic heterocyclyl, wherein the C₁₋₆alkyl, C₃₋₈cycloalkyl, C₃₋₈cycloalkenyl, and 4 to 6-membered monocyclic heterocyclyl are each optionally substituted with one or more R⁴, provided at least one of R¹ and R² is not H;
or R¹ and R² together with the N atom from which they are attached form a 4 to 7-membered monocyclic heterocycle or 6 to 10-membered bicyclic heterocycle, each of which is optionally substituted with one or more R⁴;
each R⁴ is independently selected from OR^{4a}, halo, C₁₋₃alkyl, C₃₋₈cycloalkyl, and 4 to 6-membered monocyclic heterocyclyl;
R^{4a} is selected from H and C₁₋₃alkyl, wherein the C₁₋₃alkyl is optionally substituted with one or more halo;
R³ is phenyl, 5 or 6-membered monocyclic heteroaryl, 9 or 10-membered bicyclic heteroaryl, or 6 to 10 membered bicyclic heterocycle, wherein the phenyl, 5 or 6-membered monocyclic heteroaryl, 9 or 10-membered bicyclic heteroaryl, and 6 to 10 membered bicyclic heterocycle are each optionally substituted with one or more substituent R⁵;
each R⁵ is independently selected from C₁₋₃alkyl, C₃₋₆cycloalkyl, OR^{5a}, cyano, halo, and 5 or 6-membered monocyclic heteroaryl, wherein the C₁₋₃alkyl, C₃₋₆ cycloalkyl, and heteroaryl are optionally substituted with one or more R^{5b};
R^{5a} is selected from H, C₁₋₃alkyl, and C₃₋₆cycloalkyl, wherein the C₁₋₃alkyl is optionally substituted with one or more halo;
R^{5b} is selected from OR^{5a}, C₁₋₃alkyl, cyano, and halo;
R⁶, for each occurrence, is independently halo or C₁₋₃alkyl, or two R⁶ together to form a —C₁₋₃alkylene;
n is 0, 1, or 2;
m is 0, 1, or 2.

93. A method of treating a disease mediated by Emopamil-Binding Protein, comprising administering to a subject an effective amount of a compound represented by Formula (I): 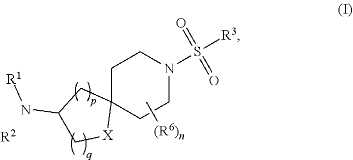
or a pharmaceutically acceptable salt thereof, wherein:
X is O, CH₂, or a bond, provided when X is a bond, p and q are 1;
p is 0 or 1, provided that when p is 0, q is 1;
q is 0 or 1, provided that when q is 0, p is 1;
R¹ and R² are each independently selected from H, C₁₋₆alkyl, C₃₋₈cycloalkyl, C₃₋₈cycloalkenyl, and 4 to 6-membered monocyclic heterocyclyl, wherein the C₁₋₆alkyl, C₃₋₈cycloalkyl, C₃₋₈cycloalkenyl, and 4 to 6-membered monocyclic heterocyclyl are each optionally substituted with one or more R⁴, provided at least one of R¹ and R² is not H;
or R¹ and R² together with the N atom from which they are attached form a 4 to 7-membered monocyclic heterocycle or 6 to 10-membered bicyclic heterocycle, each of which is optionally substituted with one or more R⁴;
R⁴ is selected from OR^{4a}, halo, C₁₋₃alkyl, or C₃₋₈cycloalkyl;
R^{4a} is selected from H and C₁₋₃alkyl, wherein the C₁₋₃alkyl is optionally substituted with one or more halo;
R³ is phenyl, 5 or 6-membered monocyclic heteroaryl, 9 or 10-membered bicyclic heteroaryl, or 6 to 10 membered bicyclic heterocycle, wherein the phenyl, 5 or 6-membered monocyclic heteroaryl, 9 or 10-membered bicyclic heteroaryl, and 6 to 10 membered bicyclic heterocycle are each optionally substituted with one or more substituent R⁵;
R⁵ is selected from C₁₋₃alkyl, C₃₋₆cycloalkyl, OR^{5a}, cyano, halo, and 5 or 6-membered monocyclic heteroaryl, wherein the C₁₋₃alkyl, C₃₋₆cycloalkyl, and heteroaryl are optionally substituted with one or more R^{5b};
R^{5a} is selected from H, C₁₋₃alkyl, and C₃₋₆cycloalkyl, wherein the C₁₋₃alkyl is optionally substituted with one or more halo;
R^{5b} is selected from OR^{5a}, C₁₋₃alkyl, cyano, and halo;
R⁶, for each occurrence, is independently halo or C₁₋₃alkyl, or two R⁶ together to form a —C₁₋₃alkylene;
n is 0, 1, or 2;
m is 0, 1, or 2.

94. A method of treating an autoimmune disease in a subject comprising administering to the subject an effective amount of a compound represented by Formula (I): 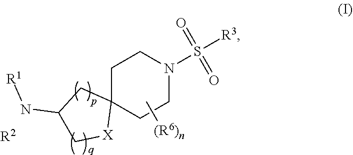
or a pharmaceutically acceptable salt thereof, wherein:
X is O, CH₂, or a bond, provided when X is a bond, p and q are 1;
p is 0 or 1, provided that when p is 0, q is 1;
q is 0 or 1, provided that when q is 0, p is 1;
R¹ and R² are each independently selected from H, C₁₋₆alkyl, C₃₋₈cycloalkyl, C₃₋₈cycloalkenyl, 6 to 10-membered bicyclic heterocycle, and 4 to 6-membered monocyclic heterocyclyl, wherein the C₁₋₆alkyl, C₃₋₈cycloalkyl, C₃₋₈cycloalkenyl, and 4 to 6-membered monocyclic heterocyclyl are each optionally substituted with one or more R⁴, provided at least one of R¹ and R² is not H;
or R¹ and R² together with the N atom from which they are attached form a 4 to 7-membered monocyclic heterocycle or 6 to 10-membered bicyclic heterocycle, each of which is optionally substituted with one or more R⁴;
each R⁴ is independently selected from OR^{4a}, halo, C₁₋₃alkyl, C₃₋₈cycloalkyl, and 4- to 6-membered heterocyclyl;
R^{4a} is selected from H and C₁₋₃alkyl, wherein the C₁₋₃alkyl is optionally substituted with one or more halo;
R³ is phenyl, 5 or 6-membered monocyclic heteroaryl, 9 or 10-membered bicyclic heteroaryl, or 6 to 10 membered bicyclic heterocycle, wherein the phenyl, 5 or 6-membered monocyclic heteroaryl, 9 or 10-membered bicyclic heteroaryl, and 6 to 10 membered bicyclic heterocycle are each optionally substituted with one or more substituent R⁵;
each R⁵ is independently selected from C₁₋₃alkyl, C₃₋₆cycloalkyl, OR^{5a}, cyano, halo, and 5 or 6-membered monocyclic heteroaryl, wherein the C₁₋₃alkyl, C₃₋₆ cycloalkyl, and heteroaryl are optionally substituted with one or more R^{5b};
R^{5a} is selected from H, C₁₋₃alkyl, and C₃₋₆cycloalkyl, wherein the C₁₋₃alkyl is optionally substituted with one or more halo;
R^{5b} is selected from OR^{5a}, C₁₋₃alkyl, cyano, and halo;
R⁶, for each occurrence, is independently halo or C₁₋₃alkyl, or two R⁶ together to form a —C₁₋₃alkylene;
n is 0, 1, or 2;
m is 0, 1, or 2.

95. A method of treating an autoimmune disease in a subject comprising administering to the subject an effective amount of a compound represented by Formula (I): 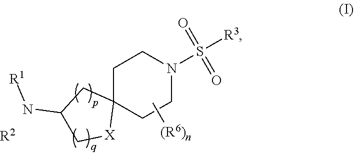
or a pharmaceutically acceptable salt thereof, wherein:
X is O, CH₂, or a bond, provided when X is a bond, p and q are 1;
p is 0 or 1, provided that when p is 0, q is 1;
q is 0 or 1, provided that when q is 0, p is 1;
R¹ and R² are each independently selected from H, C₁₋₆alkyl, C₃₋₈cycloalkyl, C₃₋₈cycloalkenyl, and 4 to 6-membered monocyclic heterocyclyl, wherein the C₁₋₆alkyl, C₃₋₈cycloalkyl, C₃₋₈cycloalkenyl, and 4 to 6-membered monocyclic heterocyclyl are each optionally substituted with one or more R⁴, provided at least one of R¹ and R² is not H;
or R¹ and R² together with the N atom from which they are attached form a 4 to 7-membered monocyclic heterocycle or 6 to 10-membered bicyclic heterocycle, each of which is optionally substituted with one or more R⁴;
R⁴ is selected from OR^{4a}, halo, C₁₋₃alkyl, or C₃₋₈cycloalkyl;
R^{4a} is selected from H and C₁₋₃alkyl, wherein the C₁₋₃alkyl is optionally substituted with one or more halo;
R³ is phenyl, 5 or 6-membered monocyclic heteroaryl, 9 or 10-membered bicyclic heteroaryl, or 6 to 10 membered bicyclic heterocycle, wherein the phenyl, 5 or 6-membered monocyclic heteroaryl, 9 or 10-membered bicyclic heteroaryl, and 6 to 10 membered bicyclic heterocycle are each optionally substituted with one or more substituent R⁵;
R⁵ is selected from C₁₋₃alkyl, C₃₋₆cycloalkyl, OR^{5a}, cyano, halo, and 5 or 6-membered monocyclic heteroaryl, wherein the C₁₋₃alkyl, C₃₋₆cycloalkyl, and heteroaryl are optionally substituted with one or more R^{5b};
R^{5a} is selected from H, C₁₋₃alkyl, and C₃₋₆cycloalkyl, wherein the C₁₋₃alkyl is optionally substituted with one or more halo;
R^{5b} is selected from OR^{5a}, C₁₋₃alkyl, cyano, and halo;
R⁶, for each occurrence, is independently halo or C₁₋₃alkyl, or two R⁶ together to form a —C₁₋₃alkylene;
n is 0, 1, or 2;
m is 0, 1, or 2.

96. The method of any one of claims 91 to 95, wherein said autoimmune disease is multiple sclerosis.

97. The method of claim 96, wherein said compound or pharmaceutical composition repairs or forms new myelin sheaths in said subject.
